(19) United States
(12) Patent Application Publication
(10) Pub. No.: US 2024/0141387 A1
(43) Pub. Date: May 2, 2024

(54) COMPOSITIONS AND METHODS FOR HUMAN GENOMIC SAFE HARBOR SITE INTEGRATION

(71) Applicants: President and Fellows of Harvard College, Cambridge, MA (US); ETH Zurich, Zuurich (CH)

(72) Inventors: Denitsa M. Milanova, Cambridge, MA (US); Erik Aznauryan, Basel (CH); George M. Church, Cambridge, MA (US); Sai Reddy, Basel (CH)

(73) Assignees: President and Fellows of Harvard College, Cambridge, MA (US); ETH Zurich, Zuurich (CH)

(21) Appl. No.: 18/279,582

(22) PCT Filed: Mar. 1, 2022
(86) PCT No.: PCT/US2022/018246

§ 371 Date: Aug. 30, 2023

Related U.S. Application Data
(60) Provisional application No. 63/155,504, filed on Mar. 2, 2021.

(51) Int. Cl. C12N 15/90; C12N 9/22; C12N 15/11; C12N 15/86

(52) U.S. Cl. CPC C12N 15/907; C12N 9/22; C12N 15/11; C12N 15/86; C12N 2310/20; C12N 2750/14143; C12N 2800/80; C12N 2800/90

(57) ABSTRACT

Provided herein, in some embodiments, are engineered nucleic acid targeting vectors that include a sequence of interest flanked by homology arms, each homology arm comprising a sequence homologous to a sequence in a safe harbor site in the human genome in any one of the following loci: 1q31, 3p24, 7q35, and Xq21. Also provided herein are methods of using and compositions the comprising engineered nucleic acid targeting vectors.

### RELATED APPLICATION

This application claims the benefit under 35 U.S.C. § 119(e) of U.S. provisional application No. 63/155,504, filed Mar. 2, 2021, which is incorporated by reference herein in its entirety.

### REFERENCE TO A SEQUENCE LISTING SUBMITTED AS A TEXT FILE

The instant application contains a Sequence Listing that has been submitted in ASCII format and is hereby incorporated by reference in its entirety. Said ASCII copy, created on Mar. 1, 2022, is named H049870729W000-SEQ-FL.TXT and is 88,611 bytes in size.

### BACKGROUND

Existing approaches for the integration and expression of genes of interest in a desired human cellular context are marred by the safety concerns related to either the random nature of viral-mediated integration or unpredictable pattern of gene expression in currently employed targeted genomic integration sites. Disadvantages of these methods lead to their limited use in clinical practice, thus encouraging future research in identifying novel human genomic sites that allow for predictable and safe expression of genes of interest.

### SUMMARY

Provided herein, in some aspects, are methods and compositions for targeting novel genomic safe harbor sites in the human genome. A bioinformatic search was conducted followed by experimental validation of these genomic safe harbor sites, including at least two that demonstrated stable expression of integrated reporter and therapeutic genes without detrimental changes to cellular transcriptome. The cell-type agnostic criteria used in the bioinformatic search described herein suggest wide-scale applicability of the newly-identified sites for engineering of, for example, a diverse range of tissues for therapeutic as well as enhancement purposes, including modified T-cells for cancer therapy and engineered skin cells to ameliorate inherited diseases and aging. Additionally, the stable and robust levels of gene expression from identified sites enable their use, for example, in industry-scale biomanufacturing of desired proteins in human cells.

Some aspects of the present disclosure provide an engineered nucleic acid targeting vector comprising a sequence of interest flanked by homology arms, each homology arm comprising a sequence homologous to a sequence in a safe harbor site in the human genome in any one of the following loci: 1q31, 3p24, 7q35, and Xq21.

In some embodiments, the safe harbor site is at position 31 on the long arm of chromosome 1 (1q31). For example, the safe harbor site may be at position 31.3 on the long arm of chromosome 1 (1q31.3). In some embodiments, the safe harbor site is within coordinates 195,338,589-195,818,588[GRCh38/hg38] of 1q31.3.

In some embodiments, the safe harbor site is at position 24 on the short arm of chromosome 3 (3p24). For example, the safe harbor site may be at position 24.3 on the short arm of chromosome 3 (3p24.3). In some embodiments, the safe harbor site is within coordinates 22,720,711-22,761,389[GRCh38/hg38] of 3p24.3.

In some embodiments, the safe harbor site is at position 35 of the long arm of chromosome 7 (7q35). For example, the safe harbor site may be within coordinates 145,090,941-145,219,513[GRCh38/hg38] of 7q35. As another example, the safe harbor site may be within coordinates 145,320,384-145,525,881[GRCh38/hg38] of 7q35.

In some embodiments, the safe harbor site is at position 21 in the long arm of chromosome X (Xq21). For example, the safe harbor site may be at position 21.31 in the long arm of chromosome X (Xq21.31). In some embodiments, the safe harbor site is within coordinates 89,174,426-89,179,074[GRCh38/hg38] of Xq21.31.

In some embodiments, the sequence of interest comprises an open reading frame.

In some embodiments, the vector comprises a promoter operably linked to the sequence of interest.

In some embodiments, the sequence of interest comprises or is within a gene of interest. In some embodiments, the gene of interest is selected from Table 2.

In some embodiments, the vector is a double-stranded DNA vector. In some embodiments, the sequence of interest is flanked by regions that enable circularization, for example, via trans-splicing or other means upon expression. See, e.g., Santer L et al. Mol Ther. 2019 Aug. 7; 27(8):1350-1363 and Meganck R M et al. Mol Ther Nucleic Acids. 2021 Jan. 16; 23:821-834, each of which is incorporated by reference herein.

In some embodiments, each homology arm has a length of about 200 to about 500 base pairs (bp), optionally 300 bp.

In some embodiments, each homology arm is a microhomology arm having a length of about 5 to 50 bp, optionally 40 bp.

In some embodiments, the vector further comprises a sequence encoding at least one guide RNA that specifically targets the sequence in the safe harbor site and/or specifically targets a sequence in or near the homology arms.

In some embodiments, the vector further comprises a sequence encoding a programmable nuclease.

Other aspects of the present disclosure provide a delivery system, for example, a viral vector (e.g., adeno-associated virus (AAV)) or a non-viral vector, such as a synthetic lipid nanoparticle or liposome, comprising the vector of any one of the preceding embodiments.

In some embodiments, the delivery system further comprising a programmable nuclease or a nucleic acid encoding the programmable nuclease.

In some embodiments, the programmable nuclease is selected from ZFNs, TALENs, DNA-guided nucleases, and RNA-guided nucleases.

In some embodiments, the programmable nuclease is an RNA-guided nuclease. In some embodiments, the RNA-guided nuclease is a CRISPR Cas nuclease and the delivery system further comprises a guide RNA or a nucleic acid encoding the gRNA. In some embodiments, the CRISPR Cas nuclease is a Cas9 nuclease or a Cas12 nuclease. In some embodiments, the gRNA specifically targets the sequence in the safe harbor site and/or specifically targets a sequence in or near the homology arms. In some embodiments, the delivery system includes a cationic polymer conjugated to a ribonuclear protein (RNP) (e.g., Cas enzyme, such as Cas9, bound to a gRNA).

Yet other aspects of the present disclosure provide a method comprising delivering to a human cell the delivery system of any one of the preceding embodiments.

Still other aspects of the present disclosure provide a method comprising delivering to a human cell the engineered targeting vector any one of the preceding embodiments.

In some embodiments, a method further comprises delivering to the human cell a programmable nuclease or a nucleic acid encoding the programmable nuclease.

In some embodiments, a method further comprises incubating the human cell to modify the safe harbor site to include the sequence of interest.

In some embodiments, the human cell is a stem cell (e.g., an induced pluripotent stem cell (iPSC)), an immune cell (e.g., T cell), or a mesenchymal cell (e.g., fibroblast). In some embodiments, the human cell is a stem cell. In some embodiments, the human cell is an iPSC. In some embodiments, the human cell is a hematopoietic stem cell. In some embodiments, the human cell is a fibroblast (e.g., primary human dermal fibroblast). In some embodiments, the human cell is an embryonic kidney cell (e.g., HEK293T cell). In some embodiments, the human cell is a Jurkat cell. In some embodiments, the human cell is an immune cell. In some embodiments, the human cell is a T cell (e.g., a primary human T cell). In some embodiments, the human cell is a B cell. In some embodiments, the human cell is an NK cell. In some embodiments, the human cell is a mesenchymal cell. In some embodiments, the human cell is a mesenchymal stem cell. In some embodiments, the human cell is a fibroblast.

Still other aspects of the present disclosure provide a method comprising delivering to a subject the delivery system of any one of the preceding embodiments.

Other aspects of the present disclosure provide a method comprising delivering to a subject the engineered targeting vector any one of the preceding embodiments.

In some embodiments, a method further comprises delivering to the subject a programmable nuclease or a nucleic acid encoding the programmable nuclease.

In some embodiments, the programmable nuclease delivered to the subject is selected from ZFNs, TALENs, DNA-guided nucleases, and RNA-guided nucleases. In some embodiments, the programmable nuclease is an RNA-guided nuclease. In some embodiments, the RNA-guided nuclease is a CRISPR Cas nuclease and the delivery system further comprises a guide RNA or a nucleic acid encoding the gRNA. In some embodiments, the CRISPR Cas nuclease is a Cas9 nuclease or a Cas12 nuclease. In some embodiments, the gRNA specifically targets the sequence in the safe harbor site and/or specifically targets a sequence in or near the homology arms.

In some embodiments, the subject has a medical condition selected from Table 1. In some embodiments, the gene of interest is selected from Table 1. In some embodiments, the gene of interest is a variant of a gene selected from Table 1.

Some aspects of the present disclosure provide a guide RNA comprising a sequence homologous to a sequence in a safe harbor site in the human genome in any one of the following loci: 1q31, 3p24, 7q35, and Xq21.

Other aspects of the present disclosure provide a delivery system comprising the guide RNA of the preceding paragraph.

Some aspects of the present disclosure provide a method comprising genetically modifying a safe harbor site in the human genome in any one of the following loci: 1q31, 3p24, 7q35, and Xq21.

Other aspects of the present disclosure provide a engineered nucleic acid targeting vector comprising a sequence of interest flanked by homology arms, wherein each homology arm comprises a sequence homologous to a safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

Yet other aspects of the present disclosure provide a method comprising identifying a safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

Still other aspects of the present disclosure provide a method comprising amplifying sequence from safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

Further aspects of the present disclosure provide a method comprising modifying sequence in safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

Other aspects provide a method comprising introducing a polynucleotide (e.g., gene of interest) into a safe harbor site in a human cell ex vivo and producing a polypeptide (e.g., protein encoded by the gene of interest), wherein the safe harbor site is selected from any one of Table 1, optionally 1q31, 3p24, 7q35, or Xq21.

In some embodiments, the polynucleotide (e.g., gene of interest) encodes a therapeutic protein. In some embodiments, the therapeutic protein is an antibody, for example, selected from a human antibody, a humanized antibody, and a chimeric antibody. An antibody may be a whole antibody or a fragment. In some embodiments, the antibody is a monoclonal antibody. In some embodiments, the antibody is a NANOBODY® or a camelid antibody. Other antibodies are contemplated herein.

In some embodiments, the polynucleotide comprises a viral polynucleotide (e.g., encoding a viral protein). The viral polynucleotide may be, for example, an adenovirus protein, an adeno-associated virus (AAV) protein, a retrovirus protein, or a Herpes virus protein. In some embodiments, the polynucleotide is a gene therapy vector (e.g., a recombinant AAV vector). For example, the polynucleotide may include one or more of a promoter, enhancer, intron, exon, stop signals, polyadenylation signals, inverted terminal repeat (ITR) sequences, replication (rep) genes, capsid (cap) coding sequences, helper genes, or other sequences used in producing a gene therapy vector, such as a recombinant AAV vector.

### BRIEF DESCRIPTION OF THE DRAWINGS

FIGS. 1A-1D show bioinformatic identification of novel genomic safe harbor sites. FIG. 1A shows GSH criteria, rationale and databases used to computationally predict GSH sites in the human genome. FIG. 1B is a schematic representation of candidate GSH sites, showing linear distances from different encoding and regulatory elements in the genome according to the established criteria. FIG. 1C shows chromosomal locations and lengths of five candidate GSH sites, which were subsequently experimentally tested. FIG. 1D shows chromosomal coordinates of five candidate GSH sites and the gRNA sequences used for subsequent CRISPR/Cas9 genome editing.

FIGS. 2A-2H show experimental validation of candidate GSH sites by targeted genome editing in HEK293T and Jurkat cells. FIG. 2A shows that PITCh plasmid is generated by cloning an mRuby-bearing insert with micro-homologies against specific GSH into a backbone possessing PITCh gRNA target sites, needed for the liberation of the insert inside the engineered cell by Cas9. FIG. 2B that shows once inside the cell, the mRuby insert is integrated into a desired site by the MMEJpathway following a Cas9-induced double-stranded break of the targeted site. FIGS. 2C-2D show flow cytometry demonstrating the isolation of clonal populations expressing the mRuby transgene from GSH1 locus in HEK293T cells and GSH2 locus in Jurkat cells using pooled and single-cell flow cytometry mediated sortings. The highest expressing GSH1-HEK293T clone and GSH2-Jurkat clone were expanded in cell culture and flow cytometry measurements at day 45, 60 and 90 demonstrated stable levels of transgene expression. FIGS. 2E-2F show genotyping of the GSH1 site in HEK293T cells and GSH2 site in Jurkat cells using primers spanning the junction between integration site and the transgene show mRuby integration into the predicted locus. FIG. 2G shows that mRuby transgene integration into each of the tested GSH sites in HEK293T shows stable expression from GSH1, GSH2, GSH7, and GSH31. Data are represented as mean±SEM, N=2. FIG. 2H shows mRuby transgene integration into each of the tested GSH sites in Jurkat show stable expression from GSH1 and GSH2. Data were represented as mean±SEM, N=2.

FIGS. 3A-3E show RNA sequencing and transcriptome analysis of HEK293T and Jurkat cells following mRuby integration into GSH2. FIG. 3A shows a pipeline of bulk RNA-seq experiment on GSH2 integrated and non-integrated HEK293T and Jurkat cells. FIG. 3B shows Principal component analysis (PCA) of two biological replicates of HEK293T and Jurkat cells with and without mRuby integration into GSH2. FIG. 3C shows differential expression of genes following GSH2 integration in HEK293T and Jurkat and comparison of HEK293T and Jurkat non-integrated cells. FIG. 3D shows chromosomal distribution of differentially expressed genes in HEK293T and Jurkat cells. Genes with an adjusted p-value of less than 0.05 were considered differentially expressed. FIG. 3E shows correlation of gene expression either between biological replicates without GSH2 integration or within a biological replicate with or without integration in GSH2.

FIGS. 4A-4F show targeted transgene integration into GSH1 and GSH2 in primary human cells. FIG. 4A shows targeted integration of mRuby into GSH1 and GSH2 in primary human T cells by Cas9 HDR. FIG. 4B shows flow cytometry plots demonstrating mRuby expression in both GSH1 and GSH2 in primary human T cells following two rounds of pooled sorting. FIG. 4C shows PCR-based genotyping of GSH1 and GSH2 sites by using primers spanning the junction of targeted site and the inserted transgene indicate correct integration of mRuby in primary human T cells. FIG. 4D shows targeted integration of LAMB3-T2A-GFP into GSH1 and GSH2 in primary human dermal fibroblasts by Cas9 HDR. FIG. 4E shows flow cytometry plots demonstrating GFP expression in both GSH1 and GSH2 in primary human dermal fibroblasts following two rounds of pooled sorting. FIG. 4F shows PCR-based genotyping of GSH1 and GSH2 sites by using primers spanning the junction of targeted site and the inserted transgene indicate correct integration of LAMB3-T2A-GFP in primary human dermal fibroblasts.

FIGS. 5A-5F show single-cell RNA-seq of primary human T-cells following targeted transgene integration into GSH1 site. FIG. 5A shows a pipeline of the RNA-seq experiment following Cas9 HDR targeted integration of mRuby into GSH1 (GSH1-mRuby cells) and T-cell activation. FIG. 5B shows a number of differentially expressed genes GSH1-mRuby T-cells and WT T-cells (non-integrated) from donor 1 and GSH1-mRuby T-cells from donor 1 and WT T-cells from donor 2. FIG. 5C shows Uniform Manifold Approximation and Projection (UMAP) analysis comparing transcriptional clusters of GSH1-mRuby and WT T-cells from donor 1 and WT T-cells from donor 2. Each point represents a unique cell barcode and each color corresponds to cluster identity. FIG. 5D shows expression of genes determining the seven largest clusters. Intensity corresponds to normalized gene expression. FIG. 5E shows distribution of GSH1-mRuby-and WT T-cells from donor 1 and WT T-cells from donor 2 across different clusters. FIG. 5F shows formalized expression for selected differentially expressed genes between GSH1mRuby and WT T-cells from donor 1.

FIG. 6 shows targeted integration of therapeutic or enhancing genes into genomic safe harbors in skin stem cells, allowing for safe, long-term expression of a desired gene in epidermis.

FIG. 7 shows experimental validation of bioinformatically identified genomic safe harbors in HEK293T cells and primary human T-cells. The graph shows a comparison of reporter gene mRuby expression from three discovered safe harbor sites and the AAVS1 site that shows an order of magnitude increase in expression from the newly identified safe harbor sites.

FIG. 8 shows verification of integration of desired therapeutic LAMB3 gene into identified genomic safe harbor sites using PCR on genomic DNA extracted from sorted GFP+ cells.

FIGS. 9A-9D show reporter integration into GSH1 and GSH2 in iPSCs. FIG. 9A shows a schematic of an eGFP coding sequence operably linked to an EF1α promoter region flanked by 300 bp homology arms. FIGS. 9B and 9C show flow cytometry plots demonstrating eGFP expression in both GSH1 and GSH2 in human iPSCs 1 day post lipofection (FIG. 9B) and 7 days post lipofection (FIG. 9C). FIG. 9D shows a genotyping with primers spanning 5′ and 3′ integration junction (in/out) and primers upstream and downstream of integration (out/out): two sets of primers for each.

### DETAILED DESCRIPTION

Development of technologies for predictable, durable and safe expression of desired genetic constructs (e.g., transgenes) in human cells will contribute significantly to the improvement of gene and cell therapies (Bestor, 2000; Ellis, 2005), as well as for protein manufacturing (Lee et al., 2019). One prominent beneficiary of such technologies are genetically engineered T-cell therapies, which require genomic integration of transgenes encoding novel immune receptors (Chen et al., 2020; Richardson et al., 2019); another example is gene therapy for highly proliferating tissues, such as inherited skin disorders, in which entire wild-type gene copies have to be integrated into epidermal stem cells (Droz-Georget Lathion et al., 2015; Hirsch et al., 2017). Advances in genome editing using targeted integration tools (Maeder and Gersbach, 2016) already allow precise genomic delivery and sustained expression of transgenes in certain cellular contexts, such as chimeric antigen receptors (CARs) integrated into the T cell receptor alpha chain locus in T-cells (Eyquem et al., 2017), and coagulation factors delivered to hepatocytes using recombinant adeno-associated viral (rAAV) vectors (Barzel et al., 2015). These applications, however, are limited to specific cell types and cause disruption to the endogenous genes, limiting the diversity of cellular engineering applications. Specific loci in the human genome that support stable and efficient transgene expression, without detrimentally altering cellular functions are known as Genomic Safe Harbor (GSH) sites. Thus, precise integration of functional genetic constructs into GSH sites greatly enhances genome engineering safety and efficacy for clinical and biotechnology applications.

Empirical studies have identified three sites that support long-term expression of transgenes: AAVS1, CCR5 and hRosa26—all of which were established without any a priori safety assessment of the genomic loci in which they reside (Papapetrou and Schambach, 2016). The AAVS1 site, located in an intron of PPP1R12C gene region, has been observed to be a region for rare genomic integration events of the Adeno-associated virus's payload (Oceguera-Yanez et al., 2016). Despite being successfully implemented for durable transgene expression in numerous cell types (Hong et al., 2017), the AAVS1 site location is in a gene-dense region, suggesting potential disruption of expression profiles of genes located in the vicinity of this loci (Sadelain et al., 2012). Additionally, studies indicated frequent transgene silencing and decrease in growth rate following transgene integration into AAVS1 (Ordovas et al., 2015; Shin et al., 2020), which represents a liability for clinical gene therapy. The second site lies within the CCR5 gene, which encodes a protein involved in chemotaxis and also serves as co-receptor for HIV cellular entry in T cells (Jiao et al., 2019). Serendipitously, researchers identified that the naturally-occurring CCR5-delta-32 mutation present in people of Scandanavian-origin results in an HIV-resistant phenotype (Silva and Stumpf, 2004). This finding suggested disposability of this gene and applicability of CCR5 locus for targeted genome engineering, especially for T cell therapies (Lombardo et al., 2011; Sather et al.). However, similar to AAVS1, the CCR5 locus is located in a gene-rich region, surrounded by tumor associated genes (Sadelain et al., 2012), thus severely limiting its safe use for therapeutic purposes. Additionally, CCR5 expression has been associated with promoting functional recovery following stroke (Joy et al., 2019), thus disrupting CCR5 may be undesirable in clinical practice. The third site, human Rosa26 (hRosa26) locus, was computationally predicted by searching the human genome for orthologous sequences of mouse Rosa26 (mRosa26) locus (Trion et al., 2007). The mRosa26 was originally identified in mouse embryonic stem cells by using random integration by lentiviral-mediated delivery of gene trapping constructs consisting of promotorless transgenes ((3-galactosidase and neomycin phosphotransferase), resulting in sustainable expression of these transgenes throughout embryonic development (Friedrich and Soriano, 1991; Zambrowicz et al., 1997). Similar to the other two currently employed GSH sites, hRosa26 is located in an intron of a coding gene THUMPD3 (Trion et al., 2007), the function of which is still not fully characterized. This site is also surrounded by proto-oncogenes in its immediate vicinity (Sadelain et al., 2012), which may be upregulated following transgene insertion, thus potentially limiting the use of hRosa26 in clinical settings.

Attempts have been made to identify new human GSH sites that would satisfy various safety criteria, thus avoiding the disadvantages of existing sites. One approach developed by Sadelein and colleagues used lentiviral transfection of beta-globin and green fluorescence protein (GFP) genes into induced pluripotent stem cells (iPSCs), followed by the assessment of the integration sites in terms of their linear distance from various coding and regulatory elements in the genome, such as cancer genes, miRNAs and ultraconserved regions (Papapetrou et al., 2011). They discovered one lentiviral integration site that satisfied all of the proposed criteria, demonstrating sustainable expression upon erythroid differentiation of iPSCs. However, global transcriptome profile alterations of cells with transgenes integrated into this site were not assessed. A similar approach by Weiss and colleagues used lentiviral integration in Chinese hamster ovary (CHO) cells to identify sites supporting long-term protein expression for biotechnological applications (e.g., recombinant monoclonal antibody production) (Gaidukov et al., 2018). Although this study led to the evaluation of multiple sites for durable, high-level transgene expression in CHO cells, no extrapolation to human genomic sites was determined. Another study aimed at identifying novel GSHs through bioinformatic search of mCreI sites residing in loci that satisfy GSH criteria (Pellenz et al., 2019). Similarly, to previous work, several stably expressing sites were identified and proposed for synthetic biology applications in humans. However, local and global gene expression profiling following integration events in these sites has not yet been assessed.

All of the potential new GSH sites possess a shared limitation of being narrowed by lentiviral- or Cre-based integration. Additionally, safety assessments of these newly identified sites, as well as previously established AAVS1, CCR5 and Rosa26, were carried out by evaluating the differential gene expression of genes located solely in the vicinity of these integration sites, without observing global transcriptomic changes following integration. A more comprehensive bioinformatic-guided and genome-wide search of GSH sites based on established criteria, followed by experimental assessment of transgene expression durability in various cell types and safety assessment using global transcriptome profiling would, thus, lead to the identification of a more reliable and clinically useful genomic region.

In the studies described herein, bioinformatic screening was used to rationally identify multiple sites that satisfy established as well as newly introduced GSH criteria. CRISPR/Cas9 targeted genome editing was used to individually integrate a reporter gene into these sites to monitor long-term expression of the transgene in HEK293T and Jurkat cells. This experimental evaluation in cell lines was followed by testing of two promising candidate sites in primary human T-cells and human dermal fibroblasts using reporter and therapeutic transgenes, respectively. Finally, bulk and single-cell RNA-sequencing experiments were performed to analyze the transcriptomic effects of such integrations into these two newly established GSH sites.

### Genomic Safe Harbor Sites

A genome is an organism's complete set of deoxyribonucleic acid (DNA), which contains the genetic instructions needed to develop and direct the activities of every organism. The genes encoded by DNA reside in chromosomes, which are organized packages of DNA found in the nucleus of the cell. Different organisms have different numbers of chromosomes. The human genome contains 23 pairs of chromosomes within the nucleus of all cells: 22 pairs of numbered chromosomes (autosomes); and one pair of sex chromosomes, X and Y.

A gene's cytogenetic location is described in a standardized way, based on the position of a particular band on a stained chromosome, or as a range of bands, if less is known about the exact location. The combination of numbers and letters provide a gene's “address” on a chromosome. This address is made up of several parts, including:
(1) The chromosome on which the gene can be found. The first number or letter used to describe a gene's location represents the chromosome. Chromosomes 1 through 22 (the autosomes) are designated by their chromosome number. The sex chromosomes are designated by X or Y;
(2) The arm of the chromosome. Each chromosome is divided into two sections (arms) based on the location of a narrowing (constriction) called the centromere. By convention, the shorter arm is called p, and the longer arm is called q. The chromosome arm is the second part of the gene's address. For example, 5q is the long arm of chromosome 5, and Xp is the short arm of the X chromosome; and
(3) The position of the gene on the p or q arm. The position of a gene is based on a distinctive pattern of light and dark bands that appear when the chromosome is stained in a certain way. The position is usually designated by two digits (representing a region and a band), which are sometimes followed by a decimal point and one or more additional digits (representing sub-bands within a light or dark area). The number indicating the gene position increases with distance from the centromere. For example, 1q31 represents position 31 on the long arm of chromosome 1, 3p24 represents position 24 on the short arm of chromosome 3, 7q35 represents position 35 on the long arm of chromosome 7, and Xq21 represents position 21 on the long arm of chromosome X.

A genomic safe harbor site (SHS or GSH site) is a genomic location where new genes or genetic elements (e.g., promoter, enhancer, etc.) can be introduced into a genome without disrupting the expression or regulation of adjacent genes. These GSH sites are important, inter alia, for effective human disease gene therapies; for investigating gene structure, function and regulation; and for cell marking and tracking. The most widely used human GSH sites were identified by serendipity (e.g., the AAVS1 adeno-associated virus insertion site on chromosome 19); by homology with useful SHS in other species (e.g., the human homolog of the murine Rosa26 locus); and most recently by recognition of the dispensability of a subset of human genes in most or all individuals (e.g., the CCR5 chemokine receptor gene, that when deleted confers resistance to HIV infection)

Provided herein are newly-identified genomic safe harbor sites that may be targeted for stable gene expression without detrimental changes to the cellular transcriptome, for example. Thus, the present disclosure provides, in some embodiments, compositions and methods for targeting any one or more for the genomic safe harbor site(s) identified in Table 1.

In some embodiments, the genomic safe harbor site is on chromosome 1. In some embodiments, the genomic safe harbor site is on the long arm of chromosome 1. In some embodiments, the genomic safe harbor site is at position 31 on the long arm of chromosome 1. For example, the genomic safe harbor site may be at position 31.3 on the long arm of chromosome 1. In some embodiments, the genomic safe harbor site is at position 31.3, coordinates 195,338,589-195,818,588[GRCh38/hg38], on the long arm of chromosome 1.

In some embodiments, the genomic safe harbor site is on chromosome 3. In some embodiments, the genomic safe harbor site is on the short arm of chromosome 3. In some embodiments, the genomic safe harbor site is at position 24 on the short arm of chromosome 3. For example, the genomic safe harbor site may be at position 24.3 on the short arm of chromosome 3. In some embodiments, the genomic safe harbor site is at position 24.3, coordinates 22,720,711-22,761,389[GRCh38/hg38], on the short arm of chromosome 3.

In some embodiments, the genomic safe harbor site is on chromosome 7. In some embodiments, the genomic safe harbor site is on the long arm of chromosome 7. In some embodiments, the genomic safe harbor site is at position 35 on the long arm of chromosome 7. For example, the genomic safe harbor site may be at position 35, coordinates 145,090,941-145,219,513[GRCh38/hg38], on the long arm of chromosome 7. In some embodiments, the genomic safe harbor site may be at position 35, coordinates 145,320,384-145,525,881[GRCh38/hg38], on the long arm of chromosome 7.

In some embodiments, the genomic safe harbor site is on chromosome X. In some embodiments, the genomic safe harbor site is on the long arm of chromosome X. In some embodiments, the genomic safe harbor site is at position 21 on the long arm of chromosome X. For example, the genomic safe harbor site may be at position 21.31 on the long arm of chromosome X. In some embodiments, the genomic safe harbor site is at position 21.31, coordinates 89,174,426-89,179,074[GRCh38/hg38], on the long arm of chromosome X.

TABLE 1
Human Genomic Safe Harbor Sites (based on GRCh38/hg38 genome assembly)

| Chr. | Start | End | Size | Chr. | Start | End | Size |
|---|---|---|---|---|---|---|---|
| chr1 | 195338589 | 195818588 | 479999 | chr3 | 166231820 | 166243755 | 11935 |
| GSH1 | | | | | | | |
| chr3 | 22720711 | 22761389 | 40678 | chr3 | 166344353 | 166403764 | 59411 |
| GSH2 | | | | | | | |
| chrX | 89174426 | 89179074 | 4648 | chr3 | 167127968 | 167190286 | 62318 |
| GSH31 | | | | | | | |
| chr7 | 145090941 | 145219513 | 128572 | chr3 | 176164537 | 176430381 | 265844 |
| GSH7 | | | | | | | |
| chr7 | 145320384 | 145525881 | 205497 | chr3 | 180087053 | 180111885 | 24832 |
| GSH8 | | | | | | | |
| chr1 | 4105262 | 4125527 | 20265 | chr3 | 180263108 | 180264173 | 1065 |
| chr1 | 4225899 | 4262026 | 36127 | chr3 | 190942429 | 191022620 | 80191 |
| chr1 | 5240899 | 5342977 | 102078 | chr3 | 191740356 | 191844429 | 104073 |
| chr1 | 14541575 | 14548703 | 7128 | chr4 | 10937739 | 11233990 | 296251 |
| chr1 | 34327292 | 34369582 | 42290 | chr4 | 11274736 | 11318826 | 44090 |
| chr1 | 38646034 | 38658930 | 12896 | chr4 | 12069688 | 12073450 | 3762 |
| chr1 | 60299679 | 60353058 | 53379 | chr4 | 12401286 | 12487904 | 86618 |
| chr1 | 61512793 | 61535520 | 22727 | chr4 | 12528612 | 12589085 | 60473 |
| chr1 | 61576366 | 61579030 | 2664 | chr4 | 12691009 | 12709100 | 18091 |
| chr1 | 64321297 | 64334397 | 13100 | chr4 | 13098670 | 13283413 | 184743 |
| chr1 | 65691559 | 65705302 | 13743 | chr4 | 17370882 | 17377695 | 6813 |
| chr1 | 66424579 | 66431399 | 6820 | chr4 | 18071876 | 18267885 | 196009 |
| chr1 | 66472315 | 66483382 | 11067 | chr4 | 18308625 | 18338061 | 29436 |
| chr1 | 68688627 | 68713014 | 24387 | chr4 | 18639508 | 18943516 | 304008 |
| chr1 | 68753786 | 68905897 | 152111 | chr4 | 20087972 | 20177548 | 89576 |
| chr1 | 72362970 | 72598920 | 235950 | chr4 | 23954089 | 24114582 | 160493 |
| chr1 | 73933822 | 73976014 | 42192 | chr4 | 24155388 | 24371175 | 215787 |
| chr1 | 78800659 | 78839763 | 39104 | chr4 | 26019550 | 26061864 | 42314 |
| chr1 | 79574181 | 79843196 | 269015 | chr4 | 27432225 | 27535144 | 102919 |
| chr1 | 79883946 | 79964942 | 80996 | chr4 | 27639900 | 27817722 | 177822 |
| chr1 | 80796788 | 81029014 | 232226 | chr4 | 28135063 | 28212278 | 77215 |
| chr1 | 81149005 | 81158567 | 9562 | chr4 | 28761476 | 28761499 | 23 |
| chr1 | 82042436 | 82065219 | 22783 | chr4 | 29517850 | 29625988 | 108138 |
| chr1 | 82411133 | 82547687 | 136554 | chr4 | 29666718 | 29698756 | 32038 |
| chr1 | 82588475 | 82610029 | 21554 | chr4 | 29800310 | 29857658 | 57348 |
| chr1 | 82710119 | 82753182 | 43063 | chr4 | 30058316 | 30445181 | 386865 |
| chr1 | 86206943 | 86233686 | 26743 | chr4 | 30485913 | 30626256 | 140343 |
| chr1 | 86274496 | 86296823 | 22327 | chr4 | 32503220 | 32533016 | 29796 |
| chr1 | 87521655 | 87655285 | 133630 | chr4 | 33139016 | 33188238 | 49222 |
| chr1 | 88055714 | 88109103 | 53389 | chr4 | 33587877 | 33626346 | 38469 |
| chr1 | 88149895 | 88263152 | 113257 | chr4 | 34452621 | 34507605 | 54984 |
| chr1 | 88363302 | 88367432 | 4130 | chr4 | 34819432 | 34916240 | 96808 |
| chr1 | 90120300 | 90203455 | 83155 | chr4 | 35020772 | 35225900 | 205128 |
| chr1 | 90303559 | 90360909 | 57350 | chr4 | 35266638 | 35279259 | 12621 |
| chr1 | 91074804 | 91123611 | 48807 | chr4 | 35319967 | 35437811 | 117844 |
| chr1 | 91164419 | 91210765 | 46346 | chr4 | 35546003 | 35559041 | 13038 |
| chr1 | 96524125 | 96533208 | 9083 | chr4 | 35599773 | 35898220 | 298447 |
| chr1 | 98423499 | 98475475 | 51976 | chr4 | 36791900 | 36851815 | 59915 |
| chr1 | 102047030 | 102049738 | 2708 | chr4 | 43648844 | 43791974 | 143130 |
| chr1 | 102539630 | 102572439 | 32809 | chr4 | 45201652 | 45273252 | 71600 |
| chr1 | 102613189 | 102613321 | 132 | chr4 | 45530136 | 45734030 | 203894 |
| chr1 | 103158496 | 103197336 | 38840 | chr4 | 45774754 | 45945118 | 170364 |
| chr1 | 103238162 | 103264878 | 26716 | chr4 | 46585213 | 46673829 | 88616 |
| chr1 | 103841108 | 103876566 | 35458 | chr4 | 52221075 | 52390493 | 169418 |
| chr1 | 103979438 | 104022982 | 43544 | chr4 | 57874655 | 58005165 | 130510 |
| chr1 | 104301393 | 104593886 | 292493 | chr4 | 58045913 | 58051571 | 5658 |
| chr1 | 104634632 | 104744867 | 110235 | chr4 | 58168070 | 58222809 | 54739 |
| chr1 | 104793981 | 104826208 | 32227 | chr4 | 58263557 | 58351258 | 87701 |
| chr1 | 104866968 | 104965109 | 98141 | chr4 | 59326146 | 59401141 | 74995 |
| chr1 | 105005885 | 105088762 | 82877 | chr4 | 59942114 | 59988268 | 46154 |
| chr1 | 105129498 | 105197411 | 67913 | chr4 | 60088586 | 60423724 | 335138 |
| chr1 | 105238169 | 105383993 | 145824 | chr4 | 60464476 | 60600585 | 136109 |
| chr1 | 105768958 | 105777623 | 8665 | chr4 | 60972684 | 60993655 | 20971 |
| chr1 | 106274564 | 106317447 | 42883 | chr4 | 62342337 | 62348324 | 5987 |
| chr1 | 106358207 | 106494341 | 136134 | chr4 | 62389068 | 62439494 | 50426 |
| chr1 | 106594744 | 106668238 | 73494 | chr4 | 62645462 | 62766825 | 121363 |
| chr1 | 106988167 | 107006678 | 18511 | chr4 | 62868794 | 62978310 | 109516 |
| chr1 | 113249258 | 113278135 | 28877 | chr4 | 63293881 | 63300113 | 6232 |
| chr1 | 113318971 | 113340748 | 21777 | chr4 | 63644278 | 63801790 | 157512 |
| chr1 | 118314489 | 118524655 | 210166 | chr4 | 63842526 | 64115500 | 272974 |
| chr1 | 118763678 | 118801408 | 37730 | chr4 | 64156254 | 64225256 | 69002 |
| chr1 | 163559595 | 163719338 | 159743 | chr4 | 64483215 | 64556417 | 73202 |
| chr1 | 163825577 | 163873669 | 48092 | chr4 | 64695525 | 64717129 | 21604 |
| chr1 | 163973873 | 164256545 | 282672 | chr4 | 65267433 | 65269562 | 2129 |
| chr1 | 164438361 | 164488657 | 50296 | chr4 | 66576941 | 66847261 | 270320 |
| chr1 | 165033992 | 165060626 | 26634 | chr4 | 66947371 | 67053523 | 106152 |
| chr1 | 187793860 | 187894353 | 100493 | chr4 | 67094295 | 67267304 | 173009 |
| chr1 | 187935143 | 188017297 | 82154 | chr4 | 71109321 | 71137285 | 27964 |
| chr1 | 189187262 | 189616148 | 428886 | chr4 | 71622087 | 71636498 | 14411 |
| chr1 | 190043097 | 190047661 | 4564 | chr4 | 71973980 | 71975889 | 1909 |
| chr1 | 190951658 | 191001509 | 49851 | chr4 | 72618799 | 72719551 | 100752 |
| chr1 | 191378467 | 191382046 | 3579 | chr4 | 72858192 | 72915177 | 56985 |
| chr1 | 191422804 | 191708706 | 285902 | chr4 | 85161277 | 85196156 | 34879 |
| chr1 | 193877035 | 193937569 | 60534 | chr4 | 85296912 | 85425113 | 128201 |
| chr1 | 193978345 | 193999472 | 21127 | chr4 | 89358010 | 89401355 | 43345 |
| chr1 | 194538205 | 194625045 | 86840 | chr4 | 90004629 | 90018098 | 13469 |
| chr1 | 194665799 | 194668794 | 2995 | chr4 | 90058878 | 90077534 | 18656 |
| chr1 | 194769236 | 195076680 | 307444 | chr4 | 92054335 | 92110366 | 56031 |
| chr1 | 195176776 | 195297813 | 121037 | chr4 | 93880964 | 93934000 | 53036 |
| chr1 | 195859392 | 195865261 | 5869 | chr4 | 95715032 | 95790018 | 74986 |
| chr1 | 197985478 | 198020764 | 35286 | chr4 | 95918764 | 96069139 | 150375 |
| chr1 | 198061596 | 198106962 | 45366 | chr4 | 96968864 | 96970700 | 1836 |
| chr1 | 199543307 | 199565187 | 21880 | chr4 | 103774534 | 103811615 | 37081 |
| chr1 | 199665280 | 199702490 | 37210 | chr4 | 106603448 | 106760090 | 156642 |
| chr1 | 199802890 | 199826977 | 24087 | chr4 | 107485220 | 107508869 | 23649 |
| chr1 | 208305376 | 208457099 | 151723 | chr4 | 110995799 | 111013582 | 17783 |
| chr1 | 213324773 | 213342300 | 17527 | chr4 | 111114227 | 111252705 | 138478 |
| chr1 | 214180901 | 214194171 | 13270 | chr4 | 111381518 | 111490494 | 108976 |
| chr1 | 214714588 | 214812154 | 97566 | chr4 | 114154225 | 114356742 | 202517 |
| chr1 | 217192388 | 217293778 | 101390 | chr4 | 115193303 | 115578811 | 385508 |
| chr1 | 217334568 | 217376991 | 42423 | chr4 | 115709755 | 115714098 | 4343 |
| chr1 | 218675978 | 218742429 | 66451 | chr4 | 116665146 | 116671495 | 6349 |
| chr1 | 221057985 | 221083864 | 25879 | chr4 | 116712251 | 116789278 | 77027 |
| chr1 | 232335643 | 232347964 | 12321 | chr4 | 116908973 | 117033553 | 124580 |
| chr1 | 233517478 | 233564003 | 46525 | chr4 | 117135576 | 117164596 | 29020 |
| chr1 | 233722512 | 233765758 | 43246 | chr4 | 121501967 | 121506685 | 4718 |
| chr1 | 238688305 | 238727048 | 38743 | chr4 | 121547465 | 121614584 | 67119 |
| chr1 | 238829200 | 238933993 | 104793 | chr4 | 122022848 | 122027839 | 4991 |
| chr1 | 238974783 | 239002747 | 27964 | chr4 | 124762732 | 124896927 | 134195 |
| chr1 | 242574696 | 242621139 | 46443 | chr4 | 124937733 | 125016398 | 78665 |
| chr1 | 242721576 | 242722619 | 1043 | chr4 | 125902793 | 125904779 | 1986 |
| chr1 | 242823103 | 242832065 | 8962 | chr4 | 126222004 | 126513508 | 291504 |
| chr10 | 1787476 | 1853593 | 66117 | chr4 | 126614776 | 126682714 | 67938 |
| chr10 | 2717239 | 2739038 | 21799 | chr4 | 126723508 | 126893429 | 169921 |
| chr10 | 2839125 | 2860530 | 21405 | chr4 | 129455022 | 129574170 | 119148 |
| chr10 | 9123468 | 9125832 | 2364 | chr4 | 130105368 | 130226228 | 120860 |
| chr10 | 9437057 | 9495470 | 58413 | chr4 | 130554344 | 130658506 | 104162 |
| chr10 | 9536210 | 9608782 | 72572 | chr4 | 131035641 | 131055836 | 20195 |
| chr10 | 15421062 | 15424596 | 3534 | chr4 | 131171976 | 131223241 | 51265 |
| chr10 | 15910520 | 15955807 | 45287 | chr4 | 133299116 | 133510855 | 211739 |
| chr10 | 16121809 | 16128700 | 6891 | chr4 | 133625208 | 133658225 | 33017 |
| chr10 | 18990953 | 18998770 | 7817 | chr4 | 134607852 | 134609398 | 1546 |
| chr10 | 20438691 | 20497170 | 58479 | chr4 | 135273779 | 135321176 | 47397 |
| chr10 | 20602046 | 20615848 | 13802 | chr4 | 135421924 | 135515116 | 93192 |
| chr10 | 20656646 | 20721318 | 64672 | chr4 | 135617157 | 135642383 | 25226 |
| chr10 | 22053769 | 22068073 | 14304 | chr4 | 137362799 | 137375425 | 12626 |
| chr10 | 22844639 | 22878023 | 33384 | chr4 | 141787842 | 141894067 | 106225 |
| chr10 | 23513123 | 23595999 | 82876 | chr4 | 141934845 | 141973159 | 38314 |
| chr10 | 23636767 | 23644745 | 7978 | chr4 | 144277425 | 144355899 | 78474 |
| chr10 | 26371378 | 26388202 | 16824 | chr4 | 147180072 | 147255486 | 75414 |
| chr10 | 29031898 | 29062443 | 30545 | chr4 | 153923451 | 153992121 | 68670 |
| chr10 | 29103311 | 29239060 | 135749 | chr4 | 154931873 | 154995845 | 63972 |
| chr10 | 29786781 | 29841031 | 54250 | chr4 | 156056988 | 156120966 | 63978 |
| chr10 | 29941467 | 29962799 | 21332 | chr4 | 156161746 | 156246560 | 84814 |
| chr10 | 36239389 | 36288615 | 49226 | chr4 | 156346646 | 156435978 | 89332 |
| chr10 | 36676138 | 36790108 | 113970 | chr4 | 157416075 | 157422489 | 6414 |
| chr10 | 44094466 | 44108413 | 13947 | chr4 | 159955124 | 160082155 | 127031 |
| chr10 | 47094274 | 47162503 | 68229 | chr4 | 160122911 | 160188736 | 65825 |
| chr10 | 47203419 | 47250385 | 46966 | chr4 | 160229510 | 160389253 | 159743 |
| chr10 | 53125815 | 53141071 | 15256 | chr4 | 160736817 | 160768421 | 31604 |
| chr10 | 53508288 | 53552028 | 43740 | chr4 | 160868869 | 160953876 | 85007 |
| chr10 | 53592810 | 53716399 | 123589 | chr4 | 161053958 | 161193979 | 140021 |
| chr10 | 55796908 | 56001781 | 204873 | chr4 | 162223684 | 162272187 | 48503 |
| chr10 | 56042531 | 56307227 | 264696 | chr4 | 162373907 | 162471681 | 97774 |
| chr10 | 56425945 | 56545962 | 120017 | chr4 | 162572041 | 162590667 | 18626 |
| chr10 | 56646031 | 56682644 | 36613 | chr4 | 162908393 | 162934794 | 26401 |
| chr10 | 56723410 | 57004458 | 281048 | chr4 | 164464722 | 164488905 | 24183 |
| chr10 | 57614587 | 57845133 | 230546 | chr4 | 164529711 | 164547718 | 18007 |
| chr10 | 57885939 | 57905294 | 19355 | chr4 | 166153895 | 166177764 | 23869 |
| chr10 | 58032993 | 58056422 | 23429 | chr4 | 166676119 | 166683383 | 7264 |
| chr10 | 61176420 | 61196196 | 19776 | chr4 | 167458789 | 167598429 | 139640 |
| chr10 | 61237038 | 61302638 | 65600 | chr4 | 167792287 | 167796673 | 4386 |
| chr10 | 64220850 | 64453296 | 232446 | chr4 | 175129381 | 175196357 | 66976 |
| chr10 | 64494060 | 64570373 | 76313 | chr4 | 176470458 | 176487450 | 16992 |
| chr10 | 64670922 | 64712882 | 41960 | chr4 | 177030605 | 177030882 | 277 |
| chr10 | 76710994 | 76738043 | 27049 | chr4 | 177071686 | 177092538 | 20852 |
| chr10 | 80828088 | 80836317 | 8229 | chr4 | 178196844 | 178236694 | 39850 |
| chr10 | 80877055 | 80890664 | 13609 | chr4 | 178337285 | 178351454 | 14169 |
| chr10 | 80991201 | 81086539 | 95338 | chr4 | 178456830 | 178556086 | 99256 |
| chr10 | 81194731 | 81212450 | 17719 | chr4 | 178596846 | 178635885 | 39039 |
| chr10 | 81253198 | 81544430 | 291232 | chr4 | 178770774 | 179019436 | 248662 |
| chr10 | 81585166 | 81676009 | 90843 | chr4 | 179119594 | 179239133 | 119539 |
| chr10 | 81716779 | 81728607 | 11828 | chr4 | 179615305 | 179806852 | 191547 |
| chr10 | 81804364 | 81818896 | 14532 | chr4 | 179907581 | 180488011 | 580430 |
| chr10 | 83118071 | 83261627 | 143556 | chr4 | 180909038 | 180914088 | 5050 |
| chr10 | 83364475 | 83387033 | 22558 | chr4 | 181415029 | 181472665 | 57636 |
| chr10 | 83487129 | 83522405 | 35276 | chr4 | 181573001 | 181670018 | 97017 |
| chr10 | 84611263 | 84617278 | 6015 | chr4 | 183802847 | 183803430 | 583 |
| chr10 | 84658092 | 84813264 | 155172 | chr4 | 187854609 | 187920272 | 65663 |
| chr10 | 84913362 | 84954690 | 41328 | chr5 | 2331966 | 2334507 | 2541 |
| chr10 | 84995456 | 85043420 | 47964 | chr5 | 2375323 | 2393165 | 17842 |
| chr10 | 90213960 | 90252520 | 38560 | chr5 | 2433931 | 2507656 | 73725 |
| chr10 | 92783115 | 92784712 | 1597 | chr5 | 2548446 | 2562590 | 14144 |
| chr10 | 100609998 | 100616694 | 6696 | chr5 | 3769789 | 3862707 | 92918 |
| chr10 | 105315235 | 105320229 | 4994 | chr5 | 5566044 | 5869028 | 302984 |
| chr10 | 105401249 | 105457043 | 55794 | chr5 | 12204043 | 12225343 | 21300 |
| chr10 | 105584765 | 105602777 | 18012 | chr5 | 12347504 | 12392204 | 44700 |
| chr10 | 105969197 | 105990263 | 21066 | chr5 | 13356366 | 13454346 | 97980 |
| chr10 | 106338722 | 106500105 | 161383 | chr5 | 14064557 | 14093701 | 29144 |
| chr10 | 107214534 | 107251341 | 36807 | chr5 | 18105857 | 18128629 | 22772 |
| chr10 | 107292077 | 107411559 | 119482 | chr5 | 18169379 | 18186122 | 16743 |
| chr10 | 107511669 | 107544972 | 33303 | chr5 | 18286196 | 18497311 | 211115 |
| chr10 | 108347849 | 108397974 | 50125 | chr5 | 19292346 | 19348800 | 56454 |
| chr10 | 108991310 | 109134933 | 143623 | chr5 | 19389544 | 19422950 | 33406 |
| chr10 | 109289746 | 109403875 | 114129 | chr5 | 21087691 | 21142474 | 54783 |
| chr10 | 109444653 | 109478118 | 33465 | chr5 | 23164527 | 23212158 | 47631 |
| chr10 | 109584990 | 109611813 | 26823 | chr5 | 23386237 | 23393585 | 7348 |
| chr10 | 109731603 | 109757650 | 26047 | chr5 | 23578597 | 23610626 | 32029 |
| chr10 | 111155248 | 111199938 | 44690 | chr5 | 23739386 | 23801347 | 61961 |
| chr10 | 111548941 | 111594994 | 46053 | chr5 | 27171150 | 27235023 | 63873 |
| chr10 | 111635790 | 111908711 | 272921 | chr5 | 27646401 | 27680442 | 34041 |
| chr10 | 118515103 | 118522870 | 7767 | chr5 | 27721204 | 27936174 | 214970 |
| chr10 | 120174226 | 120304700 | 130474 | chr5 | 28036285 | 28136388 | 100103 |
| chr10 | 125312971 | 125423685 | 110714 | chr5 | 28437665 | 28574656 | 136991 |
| chr10 | 126829774 | 126838094 | 8320 | chr5 | 29650868 | 29745937 | 95069 |
| chr10 | 128508017 | 128704118 | 196101 | chr5 | 30298893 | 30315404 | 16511 |
| chr10 | 128744864 | 128762878 | 18014 | chr5 | 30415684 | 30450614 | 34930 |
| chr10 | 129110062 | 129128250 | 18188 | chr5 | 30491390 | 30943976 | 452586 |
| chr10 | 130633154 | 130840784 | 207630 | chr5 | 34308767 | 34315742 | 6975 |
| chr10 | 130881570 | 130912587 | 31017 | chr5 | 34423850 | 34476153 | 52303 |
| chr11 | 15314207 | 15328530 | 14323 | chr5 | 35280589 | 35281708 | 1119 |
| chr11 | 20285465 | 20313684 | 28219 | chr5 | 35519379 | 35528483 | 9104 |
| chr11 | 21629550 | 21695849 | 66299 | chr5 | 39771513 | 39781461 | 9948 |
| chr11 | 21736599 | 21780742 | 44143 | chr5 | 39822291 | 39838575 | 16284 |
| chr11 | 21821466 | 21963447 | 141981 | chr5 | 40417657 | 40604962 | 187305 |
| chr11 | 23571202 | 23580587 | 9385 | chr5 | 41643224 | 41680064 | 36840 |
| chr11 | 23954598 | 24011392 | 56794 | chr5 | 42022241 | 42028383 | 6142 |
| chr11 | 24052158 | 24085476 | 33318 | chr5 | 42341523 | 42373776 | 32253 |
| chr11 | 25218770 | 25301913 | 83143 | chr5 | 44116731 | 44121372 | 4641 |
| chr11 | 25342649 | 25487277 | 144628 | chr5 | 44162148 | 44238731 | 76583 |
| chr11 | 26773427 | 26837648 | 64221 | chr5 | 51700000 | 51712176 | 12176 |
| chr11 | 26878458 | 26897185 | 18727 | chr5 | 51752936 | 51813991 | 61055 |
| chr11 | 36773176 | 37228321 | 455145 | chr5 | 52230987 | 52232566 | 1579 |
| chr11 | 37328417 | 37652124 | 323707 | chr5 | 52332859 | 52406378 | 73519 |
| chr11 | 37776456 | 37788600 | 12144 | chr5 | 52447134 | 52525192 | 78058 |
| chr11 | 38262799 | 38273729 | 10930 | chr5 | 53265126 | 53352487 | 87361 |
| chr11 | 38314471 | 38348994 | 34523 | chr5 | 58277953 | 58368299 | 90346 |
| chr11 | 38856655 | 38930053 | 73398 | chr5 | 62036129 | 62075014 | 38885 |
| chr11 | 38970797 | 39111452 | 140655 | chr5 | 62196946 | 62198958 | 2012 |
| chr11 | 39311213 | 39523985 | 212772 | chr5 | 62239748 | 62256161 | 16413 |
| chr11 | 39564727 | 39680811 | 116084 | chr5 | 62678582 | 62726876 | 48294 |
| chr11 | 39780908 | 39811505 | 30597 | chr5 | 62827263 | 62965255 | 137992 |
| chr11 | 39924388 | 39957243 | 32855 | chr5 | 63073489 | 63245463 | 171974 |
| chr11 | 42412427 | 42541371 | 128944 | chr5 | 63352056 | 63678541 | 326485 |
| chr11 | 42582099 | 42726050 | 143951 | chr5 | 63719267 | 63807892 | 88625 |
| chr11 | 42766770 | 42837854 | 71084 | chr5 | 71881410 | 71998538 | 117128 |
| chr11 | 42878598 | 42889774 | 11176 | chr5 | 72039390 | 72057233 | 17843 |
| chr11 | 42995111 | 43013636 | 18525 | chr5 | 84679015 | 84949811 | 270796 |
| chr11 | 48678493 | 48702710 | 24217 | chr5 | 85999199 | 86037525 | 38326 |
| chr11 | 48767083 | 48777549 | 10466 | chr5 | 86137847 | 86155232 | 17385 |
| chr11 | 48818309 | 48829318 | 11009 | chr5 | 86531426 | 86567903 | 36477 |
| chr11 | 49501793 | 49503211 | 1418 | chr5 | 87902908 | 88118894 | 215986 |
| chr11 | 50483245 | 50521348 | 38103 | chr5 | 91589085 | 91793686 | 204601 |
| chr11 | 79762300 | 79816804 | 54504 | chr5 | 91894529 | 91932596 | 38067 |
| chr11 | 80118708 | 80171619 | 52911 | chr5 | 98461792 | 98526340 | 64548 |
| chr11 | 80477911 | 80477959 | 48 | chr5 | 99145013 | 99209933 | 64920 |
| chr11 | 80578066 | 80601199 | 23133 | chr5 | 99250735 | 99399431 | 148696 |
| chr11 | 81007634 | 81072518 | 64884 | chr5 | 99727957 | 99784671 | 56714 |
| chr11 | 81172586 | 81326649 | 154063 | chr5 | 99825429 | 99898294 | 72865 |
| chr11 | 81367365 | 81502225 | 134860 | chr5 | 100203779 | 100251440 | 47661 |
| chr11 | 81606425 | 81719315 | 112890 | chr5 | 100953266 | 100988158 | 34892 |
| chr11 | 91533289 | 91644361 | 111072 | chr5 | 101028876 | 101238711 | 209835 |
| chr11 | 93900531 | 93915469 | 14938 | chr5 | 101338821 | 101513433 | 174612 |
| chr11 | 96866364 | 96950273 | 83909 | chr5 | 101632128 | 101766474 | 134346 |
| chr11 | 97409987 | 97604938 | 194951 | chr5 | 101866780 | 101926295 | 59515 |
| chr11 | 97707582 | 97728780 | 21198 | chr5 | 103691985 | 103929910 | 237925 |
| chr11 | 98181198 | 98490106 | 308908 | chr5 | 105542970 | 105661169 | 118199 |
| chr11 | 100408885 | 100512911 | 104026 | chr5 | 105701919 | 105872993 | 171074 |
| chr11 | 101359591 | 101401563 | 41972 | chr5 | 105973108 | 106057621 | 84513 |
| chr11 | 104214379 | 104264148 | 49769 | chr5 | 106098375 | 106312893 | 214518 |
| chr11 | 104759321 | 104819533 | 60212 | chr5 | 106353625 | 106365575 | 11950 |
| chr11 | 106361791 | 106449170 | 87379 | chr5 | 106467241 | 106493065 | 25824 |
| chr11 | 106489976 | 106624011 | 134035 | chr5 | 106596803 | 106665196 | 68393 |
| chr11 | 107068524 | 107126285 | 57761 | chr5 | 107245332 | 107326888 | 81556 |
| chr11 | 110927177 | 110939869 | 12692 | chr5 | 108432098 | 108537055 | 104957 |
| chr11 | 113562117 | 113563491 | 1374 | chr5 | 110034751 | 110132157 | 97406 |
| chr11 | 113604341 | 113620392 | 16051 | chr5 | 110172953 | 110239232 | 66279 |
| chr11 | 114756933 | 114876250 | 119317 | chr5 | 110815161 | 110817992 | 2831 |
| chr11 | 114917014 | 115078217 | 161203 | chr5 | 113645285 | 113906005 | 260720 |
| chr11 | 116092995 | 116247623 | 154628 | chr5 | 114303754 | 114310944 | 7190 |
| chr11 | 116288463 | 116346898 | 58435 | chr5 | 114818410 | 114881272 | 62862 |
| chr11 | 121683693 | 121799738 | 116045 | chr5 | 117188569 | 117251103 | 62534 |
| chr11 | 127487033 | 127520739 | 33706 | chr5 | 119754048 | 120095447 | 341399 |
| chr11 | 127561471 | 127607567 | 46096 | chr5 | 120970837 | 120990089 | 19252 |
| chr11 | 127722973 | 127817842 | 94869 | chr5 | 121515314 | 121525847 | 10533 |
| chr11 | 127858606 | 127890912 | 32306 | chr5 | 121721872 | 121801954 | 80082 |
| chr11 | 127991654 | 128030426 | 38772 | chr5 | 123286867 | 123294884 | 8017 |
| chr11 | 133582519 | 133633670 | 51151 | chr5 | 123716806 | 123897334 | 180528 |
| chr12 | 14031957 | 14066589 | 34632 | chr5 | 125752227 | 125812745 | 60518 |
| chr12 | 16094359 | 16129188 | 34829 | chr5 | 127229900 | 127240830 | 10930 |
| chr12 | 16937845 | 16939125 | 1280 | chr5 | 129164028 | 129234960 | 70932 |
| chr12 | 17172460 | 17308983 | 136523 | chr5 | 129275718 | 129309558 | 33840 |
| chr12 | 17765463 | 17794760 | 29297 | chr5 | 130327693 | 130336620 | 8927 |
| chr12 | 17835492 | 17950268 | 114776 | chr5 | 130436680 | 130440049 | 3369 |
| chr12 | 17991018 | 18030868 | 39850 | chr5 | 130648779 | 130714443 | 65664 |
| chr12 | 18887878 | 18946395 | 58517 | chr5 | 130764499 | 130935678 | 171179 |
| chr12 | 18987229 | 18997073 | 9844 | chr5 | 131058742 | 131109026 | 50284 |
| chr12 | 23401499 | 23479499 | 78000 | chr5 | 144009884 | 144090878 | 80994 |
| chr12 | 24037334 | 24063255 | 25921 | chr5 | 144585670 | 144797592 | 211922 |
| chr12 | 28631511 | 28671974 | 40463 | chr5 | 144838336 | 144934707 | 96371 |
| chr12 | 29854842 | 30050982 | 196140 | chr5 | 145055789 | 145072609 | 16820 |
| chr12 | 30542302 | 30578987 | 36685 | chr5 | 153373543 | 153410582 | 37039 |
| chr12 | 38379728 | 38394176 | 14448 | chr5 | 155162324 | 155347290 | 184966 |
| chr12 | 39242874 | 39243227 | 353 | chr5 | 161109279 | 161180049 | 70770 |
| chr12 | 58294865 | 58344595 | 49730 | chr5 | 162205536 | 162274377 | 68841 |
| chr12 | 59862576 | 59942863 | 80287 | chr5 | 162812313 | 162862264 | 49951 |
| chr12 | 60266384 | 60291823 | 25439 | chr5 | 163644058 | 163659209 | 15151 |
| chr12 | 60502439 | 60797346 | 294907 | chr5 | 163937827 | 163963507 | 25680 |
| chr12 | 60838088 | 61016401 | 178313 | chr5 | 165955608 | 165978497 | 22889 |
| chr12 | 61057153 | 61181158 | 124005 | chr5 | 166305439 | 166329123 | 23684 |
| chr12 | 61282937 | 61450148 | 167211 | chr5 | 166432599 | 166737416 | 304817 |
| chr12 | 61490900 | 61550066 | 59166 | chr5 | 175145513 | 175200265 | 54752 |
| chr12 | 61650843 | 61658258 | 7415 | chr5 | 175341252 | 175390038 | 48786 |
| chr12 | 72878844 | 72965956 | 87112 | chr6 | 8152578 | 8179654 | 27076 |
| chr12 | 73358317 | 73524928 | 166611 | chr6 | 8935445 | 8974220 | 38775 |
| chr12 | 73565680 | 73598665 | 32985 | chr6 | 9311777 | 9546109 | 234332 |
| chr12 | 74778851 | 74870968 | 92117 | chr6 | 15713058 | 15844943 | 131885 |
| chr12 | 80153333 | 80159452 | 6119 | chr6 | 16952302 | 17004584 | 52282 |
| chr12 | 82143133 | 82152287 | 9154 | chr6 | 17181372 | 17183461 | 2089 |
| chr12 | 83322740 | 83470569 | 147829 | chr6 | 17224271 | 17231345 | 7074 |
| chr12 | 83837208 | 84119904 | 282696 | chr6 | 18621880 | 18686170 | 64290 |
| chr12 | 84499878 | 84569465 | 69587 | chr6 | 18986242 | 19057280 | 71038 |
| chr12 | 84610257 | 84621597 | 11340 | chr6 | 19991329 | 19992454 | 1125 |
| chr12 | 84722026 | 84809487 | 87461 | chr6 | 23181780 | 23187710 | 5930 |
| chr12 | 85492912 | 85517877 | 24965 | chr6 | 23496560 | 23599495 | 102935 |
| chr12 | 85697320 | 85731891 | 34571 | chr6 | 23699774 | 23739576 | 39802 |
| chr12 | 87220429 | 87239902 | 19473 | chr6 | 23780340 | 23804443 | 24103 |
| chr12 | 87280706 | 87538342 | 257636 | chr6 | 23906818 | 23921878 | 15060 |
| chr12 | 87579094 | 87645732 | 66638 | chr6 | 24052433 | 24076121 | 23688 |
| chr12 | 88249887 | 88325888 | 76001 | chr6 | 40066342 | 40121565 | 55223 |
| chr12 | 88366768 | 88380441 | 13673 | chr6 | 40923167 | 40942754 | 19587 |
| chr12 | 88752195 | 88836752 | 84557 | chr6 | 44703281 | 44759316 | 56035 |
| chr12 | 90450340 | 90467758 | 17418 | chr6 | 45745212 | 45848450 | 103238 |
| chr12 | 92642091 | 92652842 | 10751 | chr6 | 47092363 | 47118964 | 26601 |
| chr12 | 94700562 | 94722001 | 21439 | chr6 | 47159904 | 47181531 | 21627 |
| chr12 | 94762887 | 94784146 | 21259 | chr6 | 48264794 | 48368502 | 103708 |
| chr12 | 102613491 | 102631581 | 18090 | chr6 | 48409266 | 48651490 | 242224 |
| chr12 | 105477017 | 105514885 | 37868 | chr6 | 48751872 | 48804320 | 52448 |
| chr12 | 108014562 | 108015750 | 1188 | chr6 | 48845116 | 48902069 | 56953 |
| chr12 | 108056548 | 108079470 | 22922 | chr6 | 49002781 | 49027711 | 24930 |
| chr13 | 20346005 | 20353666 | 7661 | chr6 | 49195664 | 49223599 | 27935 |
| chr13 | 22426524 | 22566319 | 139795 | chr6 | 50249282 | 50364034 | 114752 |
| chr13 | 22607091 | 22613952 | 6861 | chr6 | 50963256 | 51262582 | 299326 |
| chr13 | 26455238 | 26456543 | 1305 | chr6 | 51303354 | 51335281 | 31927 |
| chr13 | 26810785 | 26855361 | 44576 | chr6 | 54535850 | 54552486 | 16636 |
| chr13 | 29645688 | 29683397 | 37709 | chr6 | 54675488 | 54690117 | 14629 |
| chr13 | 31587999 | 31648309 | 60310 | chr6 | 63033170 | 63143071 | 109901 |
| chr13 | 31689151 | 31689541 | 390 | chr6 | 65888039 | 66043430 | 155391 |
| chr13 | 36540441 | 36558115 | 17674 | chr6 | 66144909 | 66327791 | 182882 |
| chr13 | 36598889 | 36623911 | 25022 | chr6 | 66368553 | 66660241 | 291688 |
| chr13 | 37296718 | 37296818 | 100 | chr6 | 66778904 | 66785752 | 6848 |
| chr13 | 39907227 | 39929105 | 21878 | chr6 | 66826542 | 67099508 | 272966 |
| chr13 | 42419657 | 42419778 | 121 | chr6 | 67260480 | 67292395 | 31915 |
| chr13 | 42460658 | 42512735 | 52077 | chr6 | 67333171 | 67406345 | 73174 |
| chr13 | 42658013 | 42660365 | 2352 | chr6 | 67675464 | 67737645 | 62181 |
| chr13 | 47006299 | 47257081 | 250782 | chr6 | 69439511 | 69478654 | 39143 |
| chr13 | 47357301 | 47408940 | 51639 | chr6 | 69519436 | 69571380 | 51944 |
| chr13 | 47509234 | 47675329 | 166095 | chr6 | 71007038 | 71024679 | 17641 |
| chr13 | 52898641 | 52918249 | 19608 | chr6 | 71708398 | 71794120 | 85722 |
| chr13 | 52959099 | 52978758 | 19659 | chr6 | 71834886 | 71836702 | 1816 |
| chr13 | 53562629 | 53665418 | 102789 | chr6 | 72453143 | 72472640 | 19497 |
| chr13 | 54282866 | 54390703 | 107837 | chr6 | 74907925 | 75034325 | 126400 |
| chr13 | 54491315 | 54755866 | 264551 | chr6 | 76142940 | 76213667 | 70727 |
| chr13 | 55311490 | 55385696 | 74206 | chr6 | 76254447 | 76372450 | 118003 |
| chr13 | 55733524 | 55949199 | 215675 | chr6 | 76925860 | 77191704 | 265844 |
| chr13 | 56050448 | 56164372 | 113924 | chr6 | 77232498 | 77293556 | 61058 |
| chr13 | 56266942 | 56269506 | 2564 | chr6 | 77393654 | 77412204 | 18550 |
| chr13 | 56310276 | 56697084 | 386808 | chr6 | 77546671 | 77583732 | 37061 |
| chr13 | 56737866 | 56835283 | 97417 | chr6 | 77624514 | 77640657 | 16143 |
| chr13 | 56935602 | 57044266 | 108664 | chr6 | 77976974 | 78285060 | 308086 |
| chr13 | 57085040 | 57090917 | 5877 | chr6 | 78325830 | 78454466 | 128636 |
| chr13 | 57441518 | 57482758 | 41240 | chr6 | 80649370 | 80908496 | 259126 |
| chr13 | 58427774 | 58477654 | 49880 | chr6 | 80949286 | 80972631 | 23345 |
| chr13 | 58581026 | 58701046 | 120020 | chr6 | 81013419 | 81317680 | 304261 |
| chr13 | 58802233 | 58887272 | 85039 | chr6 | 81358472 | 81377101 | 18629 |
| chr13 | 59121803 | 59212679 | 90876 | chr6 | 82420828 | 82480745 | 59917 |
| chr13 | 59313391 | 59430069 | 116678 | chr6 | 82521567 | 82720910 | 199343 |
| chr13 | 59546803 | 59615582 | 68779 | chr6 | 82761692 | 82782600 | 20908 |
| chr13 | 61091768 | 61149797 | 58029 | chr6 | 84859578 | 84916396 | 56818 |
| chr13 | 61249880 | 61274688 | 24808 | chr6 | 85016625 | 85086501 | 69876 |
| chr13 | 61577946 | 61672979 | 95033 | chr6 | 85919464 | 85943020 | 23556 |
| chr13 | 62481515 | 62522284 | 40769 | chr6 | 86264576 | 86382243 | 117667 |
| chr13 | 63478094 | 63517680 | 39586 | chr6 | 86486138 | 86502227 | 16089 |
| chr13 | 64226011 | 64380109 | 154098 | chr6 | 86542993 | 86679707 | 136714 |
| chr13 | 64420871 | 64662007 | 241136 | chr6 | 86818932 | 86844392 | 25460 |
| chr13 | 64762072 | 64908096 | 146024 | chr6 | 87963067 | 87987725 | 24658 |
| chr13 | 65009396 | 65080215 | 70819 | chr6 | 90637045 | 90928534 | 291489 |
| chr13 | 65120981 | 65259854 | 138873 | chr6 | 90969312 | 91240312 | 271000 |
| chr13 | 65360953 | 65716173 | 355220 | chr6 | 91866428 | 91948745 | 82317 |
| chr13 | 66028219 | 66153870 | 125651 | chr6 | 92064734 | 92069422 | 4688 |
| chr13 | 67529976 | 67694296 | 164320 | chr6 | 92169633 | 92237840 | 68207 |
| chr13 | 67735064 | 67740406 | 5342 | chr6 | 92538827 | 92573002 | 34175 |
| chr13 | 67961951 | 68029024 | 67073 | chr6 | 94587706 | 94720790 | 133084 |
| chr13 | 68069790 | 68239678 | 169888 | chr6 | 95370790 | 95425182 | 54392 |
| chr13 | 68381613 | 68584189 | 202576 | chr6 | 95727450 | 95865096 | 137646 |
| chr13 | 69043573 | 69051831 | 8258 | chr6 | 95905848 | 95941106 | 35258 |
| chr13 | 69472101 | 69490952 | 18851 | chr6 | 98549872 | 98679966 | 130094 |
| chr13 | 69531728 | 69650593 | 118865 | chr6 | 99002922 | 99032448 | 29526 |
| chr13 | 70289429 | 70409463 | 120034 | chr6 | 99739898 | 99747293 | 7395 |
| chr13 | 70614437 | 70851150 | 236713 | chr6 | 99788061 | 99843943 | 55882 |
| chr13 | 71347235 | 71387965 | 40730 | chr6 | 101032987 | 101114808 | 81821 |
| chr13 | 72028206 | 72050896 | 22690 | chr6 | 101155590 | 101348787 | 193197 |
| chr13 | 72091692 | 72403901 | 312209 | chr6 | 102129555 | 102378087 | 248532 |
| chr13 | 72510601 | 72517212 | 6611 | chr6 | 102503792 | 102778885 | 275093 |
| chr13 | 72636810 | 72653692 | 16882 | chr6 | 102878976 | 102891013 | 12037 |
| chr13 | 74715445 | 74778552 | 63107 | chr6 | 102931773 | 102952513 | 20740 |
| chr13 | 74880080 | 75040075 | 159995 | chr6 | 103053897 | 103180016 | 126119 |
| chr13 | 76185225 | 76192521 | 7296 | chr6 | 103220766 | 103287807 | 67041 |
| chr13 | 76233313 | 76488512 | 255199 | chr6 | 103333588 | 103435917 | 102329 |
| chr13 | 76529316 | 76570483 | 41167 | chr6 | 103476661 | 103533134 | 56473 |
| chr13 | 76636499 | 76728497 | 91998 | chr6 | 103633268 | 103967632 | 334364 |
| chr13 | 77377050 | 77410884 | 33834 | chr6 | 104076763 | 104572363 | 495600 |
| chr13 | 78960542 | 78960600 | 58 | chr6 | 104613203 | 104637240 | 24037 |
| chr13 | 79001360 | 79040782 | 39422 | chr6 | 105453084 | 105462666 | 9582 |
| chr13 | 79238190 | 79256308 | 18118 | chr6 | 105782196 | 105786319 | 4123 |
| chr13 | 80178283 | 80266345 | 88062 | chr6 | 112688473 | 112775938 | 87465 |
| chr13 | 80419534 | 80569097 | 149563 | chr6 | 112876470 | 112921492 | 45022 |
| chr13 | 80707027 | 80838347 | 131320 | chr6 | 113062876 | 113172165 | 109289 |
| chr13 | 81452407 | 81457182 | 4775 | chr6 | 114685875 | 114728508 | 42633 |
| chr13 | 81497874 | 81639910 | 142036 | chr6 | 114916976 | 115308497 | 391521 |
| chr13 | 81741072 | 81746649 | 5577 | chr6 | 115408752 | 115483541 | 74789 |
| chr13 | 81787399 | 82108887 | 321488 | chr6 | 115784191 | 115830509 | 46318 |
| chr13 | 82149609 | 82310917 | 161308 | chr6 | 115871255 | 115881148 | 9893 |
| chr13 | 82351667 | 82704995 | 353328 | chr6 | 116982163 | 116988299 | 6136 |
| chr13 | 82745745 | 82773130 | 27385 | chr6 | 118367676 | 118368695 | 1019 |
| chr13 | 82874136 | 83095539 | 221403 | chr6 | 119424062 | 119435325 | 11263 |
| chr13 | 83206604 | 83248070 | 41466 | chr6 | 119502017 | 119595033 | 93016 |
| chr13 | 83348167 | 83503704 | 155537 | chr6 | 119635837 | 119715159 | 79322 |
| chr13 | 83544448 | 83718958 | 174510 | chr6 | 120315277 | 120334808 | 19531 |
| chr13 | 83759700 | 83821010 | 61310 | chr6 | 120461606 | 120476293 | 14687 |
| chr13 | 83957781 | 83970326 | 12545 | chr6 | 120576400 | 120636620 | 60220 |
| chr13 | 84070762 | 84126859 | 56097 | chr6 | 120831512 | 120930059 | 98547 |
| chr13 | 84167605 | 84175563 | 7958 | chr6 | 120970831 | 121029493 | 58662 |
| chr13 | 84216307 | 84220027 | 3720 | chr6 | 121734272 | 121808178 | 73906 |
| chr13 | 84318209 | 84412363 | 94154 | chr6 | 121908768 | 122161647 | 252879 |
| chr13 | 84713236 | 84721753 | 8517 | chr6 | 122261811 | 122286788 | 24977 |
| chr13 | 84762517 | 84915086 | 152569 | chr6 | 122920704 | 122945970 | 25266 |
| chr13 | 85702170 | 85742789 | 40619 | chr6 | 123122927 | 123124309 | 1382 |
| chr13 | 86552979 | 86563494 | 10515 | chr6 | 123165051 | 123166338 | 1287 |
| chr13 | 86604252 | 86675499 | 71247 | chr6 | 126411618 | 126434630 | 23012 |
| chr13 | 87087108 | 87169003 | 81895 | chr6 | 126733847 | 126816611 | 82764 |
| chr13 | 87269269 | 87277199 | 7930 | chr6 | 128820674 | 128833140 | 12466 |
| chr13 | 87981545 | 87992866 | 11321 | chr6 | 129911547 | 129919526 | 7979 |
| chr13 | 88386082 | 88390863 | 4781 | chr6 | 129960338 | 129963698 | 3360 |
| chr13 | 88780397 | 88843424 | 63027 | chr6 | 130493063 | 130523995 | 30932 |
| chr13 | 88884182 | 88928446 | 44264 | chr6 | 131333535 | 131405418 | 71883 |
| chr13 | 89028785 | 89064846 | 36061 | chr6 | 131519536 | 131523143 | 3607 |
| chr13 | 89719230 | 89733882 | 14652 | chr6 | 137269449 | 137337878 | 68429 |
| chr13 | 89887237 | 89910246 | 23009 | chr6 | 137378748 | 137442201 | 63453 |
| chr13 | 90281682 | 90343286 | 61604 | chr6 | 140255326 | 140280210 | 24884 |
| chr13 | 90685080 | 90731765 | 46685 | chr6 | 140321006 | 140477012 | 156006 |
| chr13 | 103197632 | 103275199 | 77567 | chr6 | 140517896 | 140610622 | 92726 |
| chr13 | 103577685 | 103793592 | 215907 | chr6 | 141070026 | 141132812 | 62786 |
| chr13 | 103834366 | 104546463 | 712097 | chr6 | 141173644 | 141297010 | 123366 |
| chr13 | 104587247 | 104764326 | 177079 | chr6 | 141705976 | 141819882 | 113906 |
| chr13 | 104895642 | 105060434 | 164792 | chr6 | 141860694 | 141938232 | 77538 |
| chr13 | 105101234 | 105301794 | 200560 | chr6 | 144903034 | 145032722 | 129688 |
| chr13 | 105947643 | 106078370 | 130727 | chr6 | 153497488 | 153615706 | 118218 |
| chr13 | 106205490 | 106226562 | 21072 | chr6 | 153731553 | 153775767 | 44214 |
| chr13 | 108023372 | 108081768 | 58396 | chr6 | 153816521 | 153888432 | 71911 |
| chr13 | 108417734 | 108429428 | 11694 | chr6 | 154655954 | 154656642 | 688 |
| chr13 | 108531817 | 108546151 | 14334 | chr6 | 155531183 | 155572809 | 41626 |
| chr13 | 109551641 | 109578281 | 26640 | chr6 | 155705026 | 155760736 | 55710 |
| chr13 | 111394249 | 111403855 | 9606 | chr6 | 155801496 | 155896796 | 95300 |
| chr14 | 25482234 | 25526429 | 44195 | chr6 | 156000910 | 156162866 | 161956 |
| chr14 | 25567183 | 25670365 | 103182 | chr6 | 156203616 | 156328749 | 125133 |
| chr14 | 26293305 | 26352198 | 58893 | chr6 | 163909841 | 163934022 | 24181 |
| chr14 | 26392978 | 26393092 | 114 | chr6 | 164589220 | 164677749 | 88529 |
| chr14 | 27895286 | 27940004 | 44718 | chr6 | 164981630 | 165056634 | 75004 |
| chr14 | 28040115 | 28055168 | 15053 | chr6 | 165167524 | 165215613 | 48089 |
| chr14 | 28095910 | 28168140 | 72230 | chr6 | 169960102 | 170012516 | 52414 |
| chr14 | 38461930 | 38504387 | 42457 | chr7 | 4319000 | 4412941 | 93941 |
| chr14 | 38545177 | 38555254 | 10077 | chr7 | 4453707 | 4461958 | 8251 |
| chr14 | 39708732 | 39901479 | 192747 | chr7 | 4562033 | 4617022 | 54989 |
| chr14 | 39942237 | 40236251 | 294014 | chr7 | 6989603 | 7016610 | 27007 |
| chr14 | 40552786 | 40624044 | 71258 | chr7 | 8802963 | 8804896 | 1933 |
| chr14 | 40664806 | 40761335 | 96529 | chr7 | 8845616 | 8883074 | 37458 |
| chr14 | 40802083 | 40804897 | 2814 | chr7 | 9339857 | 9400122 | 60265 |
| chr14 | 41125877 | 41246285 | 120408 | chr7 | 9440840 | 9544616 | 103776 |
| chr14 | 42002560 | 42096519 | 93959 | chr7 | 10167780 | 10172796 | 5016 |
| chr14 | 42858112 | 42939200 | 81088 | chr7 | 10273011 | 10299819 | 26808 |
| chr14 | 43039483 | 43224823 | 185340 | chr7 | 11882198 | 12001492 | 119294 |
| chr14 | 43383632 | 43490882 | 107250 | chr7 | 12042260 | 12161240 | 118980 |
| chr14 | 43646683 | 43845780 | 199097 | chr7 | 12804985 | 12826683 | 21698 |
| chr14 | 46701821 | 46727786 | 25965 | chr7 | 15992360 | 16037526 | 45166 |
| chr14 | 46768530 | 46789628 | 21098 | chr7 | 17110743 | 17129833 | 19090 |
| chr14 | 47945092 | 47974327 | 29235 | chr7 | 20481747 | 20492669 | 10922 |
| chr14 | 48096216 | 48246507 | 150291 | chr7 | 21271428 | 21325391 | 53963 |
| chr14 | 48641767 | 48769359 | 127592 | chr7 | 21996084 | 22014374 | 18290 |
| chr14 | 48869593 | 48907712 | 38119 | chr7 | 22055190 | 22068237 | 13047 |
| chr14 | 49227505 | 49323965 | 96460 | chr7 | 24454328 | 24487331 | 33003 |
| chr14 | 49424383 | 49436578 | 12195 | chr7 | 25034020 | 25059908 | 25888 |
| chr14 | 54041995 | 54156477 | 114482 | chr7 | 31169572 | 31204131 | 34559 |
| chr14 | 54197355 | 54320708 | 123353 | chr7 | 31244885 | 31264644 | 19759 |
| chr14 | 61133733 | 61137558 | 3825 | chr7 | 32349329 | 32360362 | 11033 |
| chr14 | 62289973 | 62405414 | 115441 | chr7 | 32401114 | 32406962 | 5848 |
| chr14 | 62446172 | 62464871 | 18699 | chr7 | 37499249 | 37535499 | 36250 |
| chr14 | 65794121 | 65807296 | 13175 | chr7 | 41283507 | 41327447 | 43940 |
| chr14 | 77985012 | 78009941 | 24929 | chr7 | 41368251 | 41517167 | 148916 |
| chr14 | 82180349 | 82353254 | 172905 | chr7 | 42287870 | 42314453 | 26583 |
| chr14 | 82480156 | 82492619 | 12463 | chr7 | 42355249 | 42511725 | 156476 |
| chr14 | 83189602 | 83360867 | 171265 | chr7 | 45236302 | 45253619 | 17317 |
| chr14 | 83401625 | 83700383 | 298758 | chr7 | 49080068 | 49080136 | 68 |
| chr14 | 84065050 | 84122840 | 57790 | chr7 | 49404816 | 49610896 | 206080 |
| chr14 | 84227088 | 84321709 | 94621 | chr7 | 51439114 | 51464250 | 25136 |
| chr14 | 84362479 | 84689941 | 327462 | chr7 | 51780870 | 51820334 | 39464 |
| chr14 | 84790052 | 84853130 | 63078 | chr7 | 51920402 | 52015234 | 94832 |
| chr14 | 84893892 | 84901273 | 7381 | chr7 | 52342913 | 52473400 | 130487 |
| chr14 | 85049296 | 85052848 | 3552 | chr7 | 52514124 | 52788252 | 274128 |
| chr14 | 85704426 | 85717126 | 12700 | chr7 | 52828972 | 52841836 | 12864 |
| chr14 | 85757898 | 85784709 | 26811 | chr7 | 52947807 | 52950610 | 2803 |
| chr14 | 86551285 | 86755777 | 204492 | chr7 | 53086924 | 53098029 | 11105 |
| chr14 | 97308736 | 97308815 | 79 | chr7 | 53238938 | 53266148 | 27210 |
| chr15 | 37640808 | 37800591 | 159783 | chr7 | 57705392 | 57717855 | 12463 |
| chr15 | 46150392 | 46291353 | 140961 | chr7 | 62806779 | 62894826 | 88047 |
| chr15 | 46401746 | 46543287 | 141541 | chr7 | 67490024 | 67541057 | 51033 |
| chr15 | 47007617 | 47078073 | 70456 | chr7 | 67847029 | 67870252 | 23223 |
| chr15 | 49706232 | 49729603 | 23371 | chr7 | 68455723 | 68530628 | 74905 |
| chr15 | 53279698 | 53296254 | 16556 | chr7 | 68571368 | 68590797 | 19429 |
| chr15 | 53337014 | 53381959 | 44945 | chr7 | 68774048 | 68838357 | 64309 |
| chr15 | 54683414 | 54731040 | 47626 | chr7 | 68879113 | 68890151 | 11038 |
| chr15 | 54771768 | 54880721 | 108953 | chr7 | 70932540 | 70939928 | 7388 |
| chr15 | 60268320 | 60297133 | 28813 | chr7 | 70980660 | 71082168 | 101508 |
| chr15 | 68482162 | 68485076 | 2914 | chr7 | 79740199 | 79751344 | 11145 |
| chr15 | 68525972 | 68528968 | 2996 | chr7 | 79795117 | 79862103 | 66986 |
| chr15 | 72950260 | 73001709 | 51449 | chr7 | 79962208 | 80046599 | 84391 |
| chr15 | 87197161 | 87234809 | 37648 | chr7 | 81825833 | 81859176 | 33343 |
| chr15 | 87275603 | 87282057 | 6454 | chr7 | 82493798 | 82539847 | 46049 |
| chr15 | 96601517 | 96622004 | 20487 | chr7 | 83714625 | 83752307 | 37682 |
| chr15 | 96933312 | 97041261 | 107949 | chr7 | 83793063 | 83869601 | 76538 |
| chr16 | 8044395 | 8126262 | 81867 | chr7 | 84734322 | 84769513 | 35191 |
| chr16 | 15307020 | 15308977 | 1957 | chr7 | 85236855 | 85271121 | 34266 |
| chr16 | 19981202 | 19981484 | 282 | chr7 | 85686344 | 85754923 | 68579 |
| chr16 | 25574169 | 25642025 | 67856 | chr7 | 85795677 | 85848647 | 52970 |
| chr16 | 26879126 | 26916706 | 37580 | chr7 | 85889379 | 85955083 | 65704 |
| chr16 | 48900015 | 48951036 | 51021 | chr7 | 85995813 | 86166784 | 170971 |
| chr16 | 48991794 | 49009507 | 17713 | chr7 | 86267733 | 86330304 | 62571 |
| chr16 | 51521232 | 51595755 | 74523 | chr7 | 89644262 | 89692942 | 48680 |
| chr16 | 52837005 | 52884812 | 47807 | chr7 | 94227157 | 94244340 | 17183 |
| chr16 | 54520699 | 54584757 | 64058 | chr7 | 94285226 | 94344560 | 59334 |
| chr16 | 54625569 | 54697246 | 71677 | chr7 | 96759891 | 96805140 | 45249 |
| chr16 | 55104665 | 55106644 | 1979 | chr7 | 97341596 | 97378543 | 36947 |
| chr16 | 59258974 | 59355627 | 96653 | chr7 | 97487896 | 97548932 | 61036 |
| chr16 | 59396383 | 59490125 | 93742 | chr7 | 97649260 | 97651481 | 2221 |
| chr16 | 59590266 | 59605601 | 15335 | chr7 | 98529622 | 98567296 | 37674 |
| chr16 | 60203973 | 60209841 | 5868 | chr7 | 104039516 | 104040064 | 548 |
| chr16 | 60706649 | 60719978 | 13329 | chr7 | 109102666 | 109172319 | 69653 |
| chr16 | 60822775 | 60875732 | 52957 | chr7 | 109814357 | 109909217 | 94860 |
| chr16 | 60978541 | 60994187 | 15646 | chr7 | 110049624 | 110165890 | 116266 |
| chr16 | 61118418 | 61281959 | 163541 | chr7 | 110266380 | 110282238 | 15858 |
| chr16 | 61322713 | 61483039 | 160326 | chr7 | 111612517 | 111626459 | 13942 |
| chr16 | 61523777 | 61541755 | 17978 | chr7 | 113601402 | 113698317 | 96915 |
| chr16 | 62090697 | 62137539 | 46842 | chr7 | 113739083 | 113826776 | 87693 |
| chr16 | 62876048 | 62906785 | 30737 | chr7 | 114782603 | 114853577 | 70974 |
| chr16 | 66013784 | 66251808 | 238024 | chr7 | 115381355 | 115453366 | 72011 |
| chr17 | 14571026 | 14575728 | 4702 | chr7 | 118546171 | 118811399 | 265228 |
| chr17 | 52210017 | 52240514 | 30497 | chr7 | 119001259 | 119104011 | 102752 |
| chr17 | 52685701 | 52712120 | 26419 | chr7 | 119229149 | 119336389 | 107240 |
| chr17 | 53192892 | 53303426 | 110534 | chr7 | 119377091 | 119389543 | 12452 |
| chr17 | 53587604 | 53631629 | 44025 | chr7 | 119430265 | 119434324 | 4059 |
| chr17 | 53875213 | 53902935 | 27722 | chr7 | 119534409 | 119554555 | 20146 |
| chr17 | 54005324 | 54188536 | 183212 | chr7 | 121491261 | 121493333 | 2072 |
| chr17 | 54229310 | 54427795 | 198485 | chr7 | 123290461 | 123306628 | 16167 |
| chr17 | 54528168 | 54624908 | 96740 | chr7 | 125616401 | 125680869 | 64468 |
| chr17 | 55660938 | 55669626 | 8688 | chr7 | 125721613 | 125767870 | 46257 |
| chr17 | 56645590 | 56673190 | 27600 | chr7 | 126090300 | 126166625 | 76325 |
| chr17 | 56714138 | 56738879 | 24741 | chr7 | 126207357 | 126228969 | 21612 |
| chr17 | 70518916 | 70578916 | 60000 | chr7 | 144900084 | 144901840 | 1756 |
| chr17 | 70929230 | 70947774 | 18544 | chr7 | 144942556 | 144955057 | 12501 |
| chr17 | 71352177 | 71387531 | 35354 | chr7 | 152911921 | 152983400 | 71479 |
| chr17 | 71428351 | 71446337 | 17986 | chr7 | 153194588 | 153223089 | 28501 |
| chr17 | 71747279 | 71794893 | 47614 | chr7 | 153578218 | 153698593 | 120375 |
| chr17 | 71835735 | 71871850 | 36115 | chr7 | 153791306 | 153815801 | 24495 |
| chr18 | 2390847 | 2411171 | 20324 | chr7 | 156116343 | 156136121 | 19778 |
| chr18 | 4509458 | 4625053 | 115595 | chr8 | 2264124 | 2343875 | 79751 |
| chr18 | 7351153 | 7452767 | 101614 | chr8 | 5192707 | 5201508 | 8801 |
| chr18 | 7493531 | 7516781 | 23250 | chr8 | 5242246 | 5313370 | 71124 |
| chr18 | 22499352 | 22536464 | 37112 | chr8 | 21600084 | 21640402 | 40318 |
| chr18 | 25652190 | 25666386 | 14196 | chr8 | 23939487 | 23940050 | 563 |
| chr18 | 25707176 | 25716252 | 9076 | chr8 | 23980894 | 24019956 | 39062 |
| chr18 | 27745164 | 27804518 | 59354 | chr8 | 26188738 | 26204882 | 16144 |
| chr18 | 28321109 | 28601731 | 280622 | chr8 | 26917273 | 26936075 | 18802 |
| chr18 | 28953393 | 28998619 | 45226 | chr8 | 34496731 | 34572496 | 75765 |
| chr18 | 29698241 | 29850144 | 151903 | chr8 | 34613222 | 34634051 | 20829 |
| chr18 | 29978708 | 30288909 | 310201 | chr8 | 35039784 | 35104343 | 64559 |
| chr18 | 30329649 | 30563274 | 233625 | chr8 | 36371404 | 36387390 | 15986 |
| chr18 | 34378791 | 34443289 | 64498 | chr8 | 41111472 | 41125114 | 13642 |
| chr18 | 37723176 | 37752988 | 29812 | chr8 | 48114708 | 48143849 | 29141 |
| chr18 | 37793742 | 37838050 | 44308 | chr8 | 48849490 | 48863915 | 14425 |
| chr18 | 37878792 | 38087046 | 208254 | chr8 | 48988934 | 49004310 | 15376 |
| chr18 | 38127786 | 38361247 | 233461 | chr8 | 50910062 | 50965020 | 54958 |
| chr18 | 38401995 | 38505208 | 103213 | chr8 | 51077250 | 51107687 | 30437 |
| chr18 | 38837998 | 38966046 | 128048 | chr8 | 54919903 | 54985202 | 65299 |
| chr18 | 40249233 | 40262826 | 13593 | chr8 | 56826874 | 56867083 | 40209 |
| chr18 | 40303596 | 40484833 | 181237 | chr8 | 59271346 | 59296961 | 25615 |
| chr18 | 40525563 | 40791328 | 265765 | chr8 | 59337783 | 59405884 | 68101 |
| chr18 | 40832070 | 41066819 | 234749 | chr8 | 62194769 | 62198590 | 3821 |
| chr18 | 41107571 | 41215390 | 107819 | chr8 | 70840371 | 70954781 | 114410 |
| chr18 | 41256132 | 41315782 | 59650 | chr8 | 70995603 | 71005456 | 9853 |
| chr18 | 41869421 | 41902704 | 33283 | chr8 | 75616843 | 75665409 | 48566 |
| chr18 | 43165691 | 43184864 | 19173 | chr8 | 75706199 | 75736364 | 30165 |
| chr18 | 43327650 | 43348851 | 21201 | chr8 | 75836460 | 76082780 | 246320 |
| chr18 | 43649836 | 43782158 | 132322 | chr8 | 76123548 | 76215542 | 91994 |
| chr18 | 43882265 | 43989470 | 107205 | chr8 | 77064028 | 77206049 | 142021 |
| chr18 | 44121701 | 44173435 | 51734 | chr8 | 77246793 | 77249071 | 2278 |
| chr18 | 50508795 | 50510077 | 1282 | chr8 | 77687290 | 77998100 | 310810 |
| chr18 | 51793939 | 51795006 | 1067 | chr8 | 78990522 | 79047362 | 56840 |
| chr18 | 51895628 | 51929966 | 34338 | chr8 | 79147453 | 79147716 | 263 |
| chr18 | 51970718 | 52040171 | 69453 | chr8 | 82362904 | 82364567 | 1663 |
| chr18 | 53835903 | 53854401 | 18498 | chr8 | 83150846 | 83150951 | 105 |
| chr18 | 53895159 | 54036585 | 141426 | chr8 | 83558900 | 83645348 | 86448 |
| chr18 | 56341262 | 56477100 | 135838 | chr8 | 83686086 | 83760726 | 74640 |
| chr18 | 56517888 | 56547207 | 29319 | chr8 | 88958908 | 89100764 | 141856 |
| chr18 | 61052726 | 61283581 | 230855 | chr8 | 89141544 | 89193400 | 51856 |
| chr18 | 64589723 | 64627795 | 38072 | chr8 | 89293793 | 89362620 | 68827 |
| chr18 | 64732203 | 64747816 | 15613 | chr8 | 97408478 | 97412034 | 3556 |
| chr18 | 64788524 | 64955872 | 167348 | chr8 | 97452960 | 97525044 | 72084 |
| chr18 | 66119647 | 66293770 | 174123 | chr8 | 97565882 | 97574202 | 8320 |
| chr18 | 66334502 | 66451082 | 116580 | chr8 | 106820245 | 106930966 | 110721 |
| chr18 | 66703501 | 66822483 | 118982 | chr8 | 107032796 | 107123199 | 90403 |
| chr18 | 66863227 | 66896115 | 32888 | chr8 | 107548055 | 107597278 | 49223 |
| chr18 | 66996222 | 67031583 | 35361 | chr8 | 110155964 | 110184742 | 28778 |
| chr18 | 67187751 | 67246281 | 58530 | chr8 | 111906826 | 111922927 | 16101 |
| chr18 | 70800744 | 70850476 | 49732 | chr8 | 113766958 | 113900885 | 133927 |
| chr18 | 72094577 | 72315145 | 220568 | chr8 | 114000998 | 114064535 | 63537 |
| chr18 | 72355877 | 72486679 | 130802 | chr8 | 114105327 | 114132066 | 26739 |
| chr18 | 73499878 | 73537294 | 37416 | chr8 | 114445839 | 114449618 | 3779 |
| chr18 | 77774515 | 77813463 | 38948 | chr8 | 114490410 | 114741652 | 251242 |
| chr18 | 78328332 | 78330548 | 2216 | chr8 | 114841929 | 114938688 | 96759 |
| chr19 | 56998556 | 57015333 | 16777 | chr8 | 114979496 | 115358495 | 378999 |
| chr2 | 2477110 | 2491988 | 14878 | chr8 | 117366630 | 117470712 | 104082 |
| chr2 | 4318190 | 4464203 | 146013 | chr8 | 121269754 | 121330136 | 60382 |
| chr2 | 4877084 | 4895276 | 18192 | chr8 | 121430465 | 121489292 | 58827 |
| chr2 | 4995383 | 5263739 | 268356 | chr8 | 128714679 | 128754772 | 40093 |
| chr2 | 5364134 | 5399779 | 35645 | chr8 | 131482869 | 131555927 | 73058 |
| chr2 | 6151275 | 6156750 | 5475 | chr8 | 131596765 | 131662511 | 65746 |
| chr2 | 13157013 | 13251173 | 94160 | chr8 | 131762980 | 131854087 | 91107 |
| chr2 | 13291919 | 13387672 | 95753 | chr8 | 134052407 | 134061864 | 9457 |
| chr2 | 15057664 | 15116908 | 59244 | chr8 | 135792495 | 135897020 | 104525 |
| chr2 | 17173140 | 17234291 | 61151 | chr8 | 136316122 | 136339447 | 23325 |
| chr2 | 17436417 | 17460583 | 24166 | chr8 | 137155458 | 137300849 | 145391 |
| chr2 | 22688288 | 22811551 | 123263 | chr8 | 137341653 | 137374903 | 33250 |
| chr2 | 35335689 | 35421404 | 85715 | chr8 | 137475021 | 137646723 | 171702 |
| chr2 | 35521841 | 35674758 | 152917 | chr8 | 139280344 | 139310061 | 29717 |
| chr2 | 35775142 | 35859017 | 83875 | chr8 | 141668737 | 141719409 | 50672 |
| chr2 | 35899783 | 36032833 | 133050 | chr8 | 141760153 | 141792644 | 32491 |
| chr2 | 36073581 | 36127654 | 54073 | chr8 | 141833534 | 141857872 | 24338 |
| chr2 | 40917452 | 41093779 | 176327 | chr9 | 1214867 | 1278663 | 63796 |
| chr2 | 41207555 | 41409362 | 201807 | chr9 | 1383953 | 1524026 | 140073 |
| chr2 | 41450144 | 41546385 | 96241 | chr9 | 1564758 | 1891899 | 327141 |
| chr2 | 41646492 | 41727073 | 80581 | chr9 | 7246243 | 7326529 | 80286 |
| chr2 | 49745122 | 49787429 | 42307 | chr9 | 7367325 | 7427044 | 59719 |
| chr2 | 49828195 | 49828622 | 427 | chr9 | 7528320 | 7547822 | 19502 |
| chr2 | 53060020 | 53372418 | 312398 | chr9 | 11115685 | 11125313 | 9628 |
| chr2 | 56536171 | 56620202 | 84031 | chr9 | 11426314 | 11568495 | 142181 |
| chr2 | 56660940 | 56700299 | 39359 | chr9 | 11668621 | 11948659 | 280038 |
| chr2 | 56800563 | 56825336 | 24773 | chr9 | 12350451 | 12550099 | 199648 |
| chr2 | 56866094 | 56998349 | 132255 | chr9 | 13637511 | 13659215 | 21704 |
| chr2 | 57129606 | 57142735 | 13129 | chr9 | 13700045 | 13777970 | 77925 |
| chr2 | 57277535 | 57379547 | 102012 | chr9 | 16117879 | 16153934 | 36055 |
| chr2 | 57480693 | 57494534 | 13841 | chr9 | 17873829 | 17907452 | 33623 |
| chr2 | 57597597 | 57658431 | 60834 | chr9 | 17948188 | 18057806 | 109618 |
| chr2 | 57699219 | 57705427 | 6208 | chr9 | 18098548 | 18210596 | 112048 |
| chr2 | 57856010 | 57857650 | 1640 | chr9 | 22364672 | 22396840 | 32168 |
| chr2 | 71736768 | 71831032 | 94264 | chr9 | 22989185 | 23116051 | 126866 |
| chr2 | 71871940 | 71934181 | 62241 | chr9 | 23156761 | 23332812 | 176051 |
| chr2 | 72062103 | 72077774 | 15671 | chr9 | 23373558 | 23485191 | 111633 |
| chr2 | 75870018 | 75945202 | 75184 | chr9 | 24048054 | 24235985 | 187931 |
| chr2 | 75985958 | 76056015 | 70057 | chr9 | 24276735 | 24395951 | 119216 |
| chr2 | 76157324 | 76208033 | 50709 | chr9 | 24741993 | 24822711 | 80718 |
| chr2 | 76311661 | 76395078 | 83417 | chr9 | 24955735 | 25038810 | 83075 |
| chr2 | 76495410 | 76545412 | 50002 | chr9 | 25155409 | 25605610 | 450201 |
| chr2 | 76645678 | 76697718 | 52040 | chr9 | 26268408 | 26459107 | 190699 |
| chr2 | 78277806 | 78362792 | 84986 | chr9 | 27660745 | 27679275 | 18530 |
| chr2 | 78749406 | 78832446 | 83040 | chr9 | 29188976 | 29204074 | 15098 |
| chr2 | 78932734 | 78975685 | 42951 | chr9 | 29368952 | 29586485 | 217533 |
| chr2 | 81020190 | 81110775 | 90585 | chr9 | 29706885 | 29774741 | 67856 |
| chr2 | 81251184 | 81278137 | 26953 | chr9 | 29876711 | 30234997 | 358286 |
| chr2 | 81716728 | 81824150 | 107422 | chr9 | 30882225 | 30885484 | 3259 |
| chr2 | 82155700 | 82218426 | 62726 | chr9 | 31040572 | 31115617 | 75045 |
| chr2 | 82688711 | 82764983 | 76272 | chr9 | 31556615 | 31594575 | 37960 |
| chr2 | 82907384 | 82943946 | 36562 | chr9 | 31697139 | 32028225 | 331086 |
| chr2 | 82984704 | 83168889 | 184185 | chr9 | 32068965 | 32172507 | 103542 |
| chr2 | 83269105 | 83367962 | 98857 | chr9 | 32213243 | 32243557 | 30314 |
| chr2 | 83722912 | 83741214 | 18302 | chr9 | 38197561 | 38210426 | 12865 |
| chr2 | 83791812 | 83860186 | 68374 | chr9 | 71496904 | 71530659 | 33755 |
| chr2 | 83900940 | 83981139 | 80199 | chr9 | 73220393 | 73241119 | 20726 |
| chr2 | 84090720 | 84117065 | 26345 | chr9 | 73341227 | 73411490 | 70263 |
| chr2 | 84157829 | 84165107 | 7278 | chr9 | 73801077 | 73820895 | 19818 |
| chr2 | 87876489 | 87878271 | 1782 | chr9 | 73923297 | 74014495 | 91198 |
| chr2 | 88236636 | 88244591 | 7955 | chr9 | 74055255 | 74213492 | 158237 |
| chr2 | 101636822 | 101646849 | 10027 | chr9 | 75248177 | 75304983 | 56806 |
| chr2 | 103326260 | 103444240 | 117980 | chr9 | 75345775 | 75351888 | 6113 |
| chr2 | 103673830 | 103706658 | 32828 | chr9 | 75804738 | 75840643 | 35905 |
| chr2 | 105538880 | 105574913 | 36033 | chr9 | 78380093 | 78539157 | 159064 |
| chr2 | 105626192 | 105657411 | 31219 | chr9 | 78639539 | 78691719 | 52180 |
| chr2 | 107013674 | 107104690 | 91016 | chr9 | 78792744 | 78985059 | 192315 |
| chr2 | 114311226 | 114370124 | 58898 | chr9 | 79295674 | 79316958 | 21284 |
| chr2 | 115895752 | 115943905 | 48153 | chr9 | 80184555 | 80307028 | 122473 |
| chr2 | 115984649 | 116017095 | 32446 | chr9 | 80446964 | 80513570 | 66606 |
| chr2 | 116057819 | 116326486 | 268667 | chr9 | 80618618 | 80684241 | 65623 |
| chr2 | 116367222 | 116637217 | 269995 | chr9 | 81059553 | 81326120 | 266567 |
| chr2 | 116868761 | 116873840 | 5079 | chr9 | 82794792 | 82929584 | 134792 |
| chr2 | 117231562 | 117263174 | 31612 | chr9 | 84440131 | 84455045 | 14914 |
| chr2 | 117303924 | 117512754 | 208830 | chr9 | 84495803 | 84554110 | 58307 |
| chr2 | 117553498 | 117607562 | 54064 | chr9 | 85077070 | 85109681 | 32611 |
| chr2 | 118336386 | 118387391 | 51005 | chr9 | 85209950 | 85266406 | 56456 |
| chr2 | 118428207 | 118581111 | 152904 | chr9 | 85307188 | 85442578 | 135390 |
| chr2 | 118634952 | 118683699 | 48747 | chr9 | 87346392 | 87373633 | 27241 |
| chr2 | 121946702 | 121990535 | 43833 | chr9 | 88802160 | 88821637 | 19477 |
| chr2 | 122031355 | 122316710 | 285355 | chr9 | 100637906 | 100681239 | 43333 |
| chr2 | 122357448 | 122524046 | 166598 | chr9 | 100782355 | 100785716 | 3361 |
| chr2 | 122564794 | 122789586 | 224792 | chr9 | 100826476 | 100862319 | 35843 |
| chr2 | 122830346 | 122915320 | 84974 | chr9 | 101843756 | 102005251 | 161495 |
| chr2 | 123223481 | 123293265 | 69784 | chr9 | 102105741 | 102108735 | 2994 |
| chr2 | 123594445 | 123618804 | 24359 | chr9 | 102149501 | 102206603 | 57102 |
| chr2 | 125039325 | 125063312 | 23987 | chr9 | 102345464 | 102369635 | 24171 |
| chr2 | 125177154 | 125285389 | 108235 | chr9 | 102807893 | 102872573 | 64680 |
| chr2 | 125326117 | 125340095 | 13978 | chr9 | 103579892 | 103639472 | 59580 |
| chr2 | 125409658 | 125511662 | 102004 | chr9 | 103774248 | 103927566 | 153318 |
| chr2 | 125552384 | 125560968 | 8584 | chr9 | 105147061 | 105194621 | 47560 |
| chr2 | 125915842 | 125960098 | 44256 | chr9 | 105901425 | 105911353 | 9928 |
| chr2 | 128731576 | 128733431 | 1855 | chr9 | 108408929 | 108551369 | 142440 |
| chr2 | 128774259 | 128844756 | 70497 | chr9 | 109589950 | 109590787 | 837 |
| chr2 | 128887115 | 128913845 | 26730 | chr9 | 115452644 | 115550741 | 98097 |
| chr2 | 129423848 | 129446296 | 22448 | chr9 | 118133382 | 118321903 | 188521 |
| chr2 | 129572394 | 129605793 | 33399 | chr9 | 118362681 | 118537751 | 175070 |
| chr2 | 129646593 | 129719192 | 72599 | chr9 | 118895312 | 119024380 | 129068 |
| chr2 | 133708227 | 133904696 | 196469 | chr9 | 119065154 | 119103457 | 38303 |
| chr2 | 133945522 | 134069982 | 124460 | chr9 | 119674125 | 119754190 | 80065 |
| chr2 | 136440669 | 136494370 | 53701 | chr9 | 120143969 | 120194978 | 51009 |
| chr2 | 136695192 | 136715544 | 20352 | chrX | 3434653 | 3439804 | 5151 |
| chr2 | 137798620 | 137882679 | 84059 | chrX | 3480584 | 3482116 | 1532 |
| chr2 | 138952747 | 139216164 | 263417 | chrX | 4087855 | 4156772 | 68917 |
| chr2 | 142431701 | 142493417 | 61716 | chrX | 4197510 | 4268236 | 70726 |
| chr2 | 142593975 | 142651522 | 57547 | chrX | 4308946 | 4335637 | 26691 |
| chr2 | 142692282 | 142782218 | 89936 | chrX | 4376357 | 4419412 | 43055 |
| chr2 | 146244821 | 146427426 | 182605 | chrX | 4460160 | 4477199 | 17039 |
| chr2 | 146639310 | 146687748 | 48438 | chrX | 4783572 | 4852125 | 68553 |
| chr2 | 147048218 | 147141228 | 93010 | chrX | 4892895 | 4939845 | 46950 |
| chr2 | 147182024 | 147358341 | 176317 | chrX | 5000311 | 5004274 | 3963 |
| chr2 | 150009191 | 150021422 | 12231 | chrX | 5045054 | 5062351 | 17297 |
| chr2 | 150785348 | 150832505 | 47157 | chrX | 5234661 | 5249106 | 14445 |
| chr2 | 152927767 | 153021986 | 94219 | chrX | 5359974 | 5360121 | 147 |
| chr2 | 153812646 | 153821912 | 9266 | chrX | 6311412 | 6315122 | 3710 |
| chr2 | 155139538 | 155213457 | 73919 | chrX | 6906167 | 6939328 | 33161 |
| chr2 | 155380527 | 155419025 | 38498 | chrX | 7641199 | 7661548 | 20349 |
| chr2 | 155459783 | 155861616 | 401833 | chrX | 7716699 | 7750997 | 34298 |
| chr2 | 156663735 | 156727705 | 63970 | chrX | 7791721 | 7792261 | 540 |
| chr2 | 156828372 | 156882167 | 53795 | chrX | 11907718 | 11973846 | 66128 |
| chr2 | 157065257 | 157160846 | 95589 | chrX | 14157623 | 14194381 | 36758 |
| chr2 | 160543794 | 160699312 | 155518 | chrX | 14294928 | 14312114 | 17186 |
| chr2 | 162947972 | 163102250 | 154278 | chrX | 14352888 | 14396441 | 43553 |
| chr2 | 163203307 | 163205272 | 1965 | chrX | 14437215 | 14465263 | 28048 |
| chr2 | 163339096 | 163543395 | 204299 | chrX | 15033374 | 15062268 | 28894 |
| chr2 | 166574772 | 166632704 | 57932 | chrX | 15167768 | 15172192 | 4424 |
| chr2 | 166673504 | 166766100 | 92596 | chrX | 16351560 | 16385627 | 34067 |
| chr2 | 166806854 | 166838486 | 31632 | chrX | 17212514 | 17232636 | 20122 |
| chr2 | 168298141 | 168331519 | 33378 | chrX | 19172637 | 19212872 | 40235 |
| chr2 | 168372321 | 168401323 | 29002 | chrX | 19253642 | 19293892 | 40250 |
| chr2 | 175604640 | 175685068 | 80428 | chrX | 20502431 | 20568496 | 66065 |
| chr2 | 175725878 | 175747335 | 21457 | chrX | 20609244 | 20767129 | 157885 |
| chr2 | 180057113 | 180329187 | 272074 | chrX | 20807865 | 20951123 | 143258 |
| chr2 | 180369993 | 180421711 | 51718 | chrX | 20991875 | 21158043 | 166168 |
| chr2 | 182573192 | 182599021 | 25829 | chrX | 21264743 | 21324417 | 59674 |
| chr2 | 182639825 | 182666040 | 26215 | chrX | 23454372 | 23503771 | 49399 |
| chr2 | 183395652 | 183557441 | 161789 | chrX | 23603896 | 23614261 | 10365 |
| chr2 | 183658119 | 183754528 | 96409 | chrX | 25118625 | 25144380 | 25755 |
| chr2 | 184126147 | 184328974 | 202827 | chrX | 25279932 | 25327705 | 47773 |
| chr2 | 184429044 | 184548365 | 119321 | chrX | 25368447 | 25541352 | 172905 |
| chr2 | 184989492 | 185243498 | 254006 | chrX | 25696327 | 25710003 | 13676 |
| chr2 | 185284264 | 185497134 | 212870 | chrX | 26043998 | 26065188 | 21190 |
| chr2 | 189361967 | 189391432 | 29465 | chrX | 26396242 | 26408336 | 12094 |
| chr2 | 191501092 | 191528807 | 27715 | chrX | 26840286 | 26866150 | 25864 |
| chr2 | 191569695 | 191628067 | 58372 | chrX | 28031355 | 28055741 | 24386 |
| chr2 | 192245709 | 192387721 | 142012 | chrX | 28096511 | 28161437 | 64926 |
| chr2 | 192428475 | 192479918 | 51443 | chrX | 28202199 | 28326962 | 124763 |
| chr2 | 192926899 | 193038100 | 111201 | chrX | 30067881 | 30093955 | 26074 |
| chr2 | 193078850 | 193206482 | 127632 | chrX | 30134699 | 30159422 | 24723 |
| chr2 | 193327614 | 193454889 | 127275 | chrX | 30365178 | 30407074 | 41896 |
| chr2 | 193495631 | 193680554 | 184923 | chrX | 30447830 | 30508823 | 60993 |
| chr2 | 194078164 | 194079353 | 1189 | chrX | 33482051 | 33530301 | 48250 |
| chr2 | 195176370 | 195298531 | 122161 | chrX | 34567358 | 34569667 | 2309 |
| chr2 | 200061159 | 200118358 | 57199 | chrX | 34855042 | 34869301 | 14259 |
| chr2 | 200159196 | 200255880 | 96684 | chrX | 35177750 | 35215260 | 37510 |
| chr2 | 208970664 | 209022596 | 51932 | chrX | 35256014 | 35319974 | 63960 |
| chr2 | 209232018 | 209249794 | 17776 | chrX | 35360718 | 35392230 | 31512 |
| chr2 | 209349862 | 209374057 | 24195 | chrX | 35433016 | 35502183 | 69167 |
| chr2 | 210800493 | 211054933 | 254440 | chrX | 35542937 | 35544172 | 1235 |
| chr2 | 220947893 | 221031427 | 83534 | chrX | 36627491 | 36669917 | 42426 |
| chr2 | 221213053 | 221326149 | 113096 | chrX | 38981436 | 39031179 | 49743 |
| chr2 | 221366897 | 221368026 | 1129 | chrX | 39584824 | 39611215 | 26391 |
| chr2 | 221791813 | 221868162 | 76349 | chrX | 40985864 | 41026998 | 41134 |
| chr2 | 224117706 | 224119663 | 1957 | chrX | 42882117 | 42899595 | 17478 |
| chr2 | 225131858 | 225249709 | 117851 | chrX | 43473492 | 43544224 | 70732 |
| chr2 | 225912112 | 225930297 | 18185 | chrX | 43585042 | 43591682 | 6640 |
| chr2 | 226335371 | 226357843 | 22472 | chrX | 46123935 | 46131267 | 7332 |
| chr2 | 226398699 | 226595983 | 197284 | chrX | 46172047 | 46175923 | 3876 |
| chr2 | 228266700 | 228302119 | 35419 | chrX | 54618560 | 54621984 | 3424 |
| chr2 | 235328905 | 235355750 | 26845 | chrX | 55368682 | 55401474 | 32792 |
| chr20 | 6278948 | 6296722 | 17774 | chrX | 56431127 | 56432514 | 1387 |
| chr20 | 6886136 | 6919613 | 33477 | chrX | 64784665 | 64837781 | 53116 |
| chr20 | 7517632 | 7583561 | 65929 | chrX | 66728578 | 66763120 | 34542 |
| chr20 | 7746977 | 7781797 | 34820 | chrX | 66835072 | 66874970 | 39898 |
| chr20 | 12068677 | 12093894 | 25217 | chrX | 66915726 | 67039890 | 124164 |
| chr20 | 12466485 | 12521563 | 55078 | chrX | 67080636 | 67166512 | 85876 |
| chr20 | 12562313 | 12699417 | 137104 | chrX | 67207204 | 67236530 | 29326 |
| chr20 | 16121643 | 16173749 | 52106 | chrX | 68892147 | 68980952 | 88805 |
| chr20 | 17033033 | 17137509 | 104476 | chrX | 69021872 | 69029556 | 7684 |
| chr20 | 20788731 | 20804120 | 15389 | chrX | 69359924 | 69389685 | 29761 |
| chr20 | 39403023 | 39594216 | 191193 | chrX | 73325515 | 73326380 | 865 |
| chr20 | 39635016 | 39635720 | 704 | chrX | 74981824 | 74989402 | 7578 |
| chr20 | 40276357 | 40322813 | 46456 | chrX | 75635506 | 75657016 | 21510 |
| chr20 | 54300327 | 54324810 | 24483 | chrX | 76118667 | 76122935 | 4268 |
| chr20 | 54701171 | 54809687 | 108516 | chrX | 77313145 | 77324211 | 11066 |
| chr20 | 54909812 | 55014224 | 104412 | chrX | 78377691 | 78445173 | 67482 |
| chr20 | 55125140 | 55258897 | 133757 | chrX | 78604751 | 78606068 | 1317 |
| chr20 | 55655215 | 55671852 | 16637 | chrX | 78849296 | 78895331 | 46035 |
| chr20 | 55712620 | 55933702 | 221082 | chrX | 79012877 | 79034935 | 22058 |
| chr20 | 56055472 | 56106737 | 51265 | chrX | 79227668 | 79239922 | 12254 |
| chr20 | 56881460 | 56883613 | 2153 | chrX | 79420165 | 79439960 | 19795 |
| chr20 | 56924497 | 56950086 | 25589 | chrX | 79480702 | 79599475 | 118773 |
| chr21 | 17023691 | 17160468 | 136777 | chrX | 79640261 | 79713916 | 73655 |
| chr21 | 19453739 | 19543840 | 90101 | chrX | 79754718 | 79823682 | 68964 |
| chr21 | 19677962 | 19689708 | 11746 | chrX | 79864430 | 79904567 | 40137 |
| chr21 | 20049755 | 20106751 | 56996 | chrX | 80151308 | 80178488 | 27180 |
| chr21 | 20480762 | 20547952 | 67190 | chrX | 80986540 | 81011215 | 24675 |
| chr21 | 22670058 | 22832581 | 162523 | chrX | 81051949 | 81063700 | 11751 |
| chr21 | 23640724 | 23702283 | 61559 | chrX | 81477276 | 81550071 | 72795 |
| chr21 | 26727697 | 26739375 | 11678 | chrX | 81675056 | 81746388 | 71332 |
| chr21 | 27089742 | 27093343 | 3601 | chrX | 81787154 | 81872539 | 85385 |
| chr21 | 27193949 | 27208884 | 14935 | chrX | 81913283 | 81951219 | 37936 |
| chr21 | 30931555 | 30941663 | 10108 | chrX | 82051327 | 82306196 | 254869 |
| chr21 | 30982419 | 30988158 | 5739 | chrX | 82346938 | 82447668 | 100730 |
| chr21 | 40914473 | 40976395 | 61922 | chrX | 82618001 | 82625637 | 7636 |
| chr22 | 34359262 | 34432605 | 73343 | chrX | 82745216 | 82791682 | 46466 |
| chr22 | 34473363 | 34491178 | 17815 | chrX | 82931349 | 82948698 | 17349 |
| chr22 | 49048386 | 49114200 | 65814 | chrX | 83051035 | 83122532 | 71497 |
| chr3 | 1454217 | 1500781 | 46564 | chrX | 83235442 | 83311449 | 76007 |
| chr3 | 1541537 | 1545776 | 4239 | chrX | 83352227 | 83356022 | 3795 |
| chr3 | 1655855 | 1680069 | 24214 | chrX | 83659214 | 83698824 | 39610 |
| chr3 | 1780474 | 1812380 | 31906 | chrX | 83799939 | 83811125 | 11186 |
| chr3 | 5406761 | 5741809 | 335048 | chrX | 83936699 | 83953549 | 16850 |
| chr3 | 5782557 | 5812841 | 30284 | chrX | 83994305 | 84008345 | 14040 |
| chr3 | 7791533 | 7802804 | 11271 | chrX | 84582423 | 84635891 | 53468 |
| chr3 | 13123117 | 13176688 | 53571 | chrX | 84676643 | 84705135 | 28492 |
| chr3 | 13217582 | 13249130 | 31548 | chrX | 84827377 | 84884150 | 56773 |
| chr3 | 15929571 | 15975750 | 46179 | chrX | 85429743 | 85433273 | 3530 |
| chr3 | 16016640 | 16119549 | 102909 | chrX | 85474021 | 85551606 | 77585 |
| chr3 | 19070401 | 19098453 | 28052 | chrX | 85592358 | 85603608 | 11250 |
| chr3 | 19585646 | 19700692 | 115046 | chrX | 86938816 | 86986177 | 47361 |
| chr3 | 19741444 | 19829471 | 88027 | chrX | 87026953 | 87037216 | 10263 |
| chr3 | 22432929 | 22606803 | 173874 | chrX | 87196804 | 87236546 | 39742 |
| chr3 | 23041582 | 23045069 | 3487 | chrX | 87277298 | 87375582 | 98284 |
| chr3 | 26066369 | 26147819 | 81450 | chrX | 87907341 | 87988148 | 80807 |
| chr3 | 26266877 | 26296680 | 29803 | chrX | 88238653 | 88319342 | 80689 |
| chr3 | 26420318 | 26426470 | 6152 | chrX | 88439520 | 88480487 | 40967 |
| chr3 | 26467190 | 26469329 | 2139 | chrX | 88521267 | 88617358 | 96091 |
| chr3 | 26863364 | 26889059 | 25695 | chrX | 88658074 | 88697224 | 39150 |
| chr3 | 26929753 | 27040591 | 110838 | chrX | 89057485 | 89133724 | 76239 |
| chr3 | 28178604 | 28191583 | 12979 | chrX | 93162476 | 93163627 | 1151 |
| chr3 | 28908340 | 28961241 | 52901 | chrX | 93479686 | 93485123 | 5437 |
| chr3 | 30084156 | 30088247 | 4091 | chrX | 93827618 | 93953814 | 126196 |
| chr3 | 30189033 | 30254319 | 65286 | chrX | 94053922 | 94128004 | 74082 |
| chr3 | 30994142 | 31111703 | 117561 | chrX | 94228106 | 94260440 | 32334 |
| chr3 | 31319406 | 31402831 | 83425 | chrX | 94370025 | 94586662 | 216637 |
| chr3 | 33947681 | 34009333 | 61652 | chrX | 94691794 | 94701178 | 9384 |
| chr3 | 34712877 | 34822640 | 109763 | chrX | 94969916 | 95013140 | 43224 |
| chr3 | 34937040 | 34948862 | 11822 | chrX | 95206004 | 95324289 | 118285 |
| chr3 | 34989574 | 35125135 | 135561 | chrX | 95365041 | 95403368 | 38327 |
| chr3 | 35326882 | 35329835 | 2953 | chrX | 95771144 | 95904266 | 133122 |
| chr3 | 35370571 | 35444459 | 73888 | chrX | 96260261 | 96263560 | 3299 |
| chr3 | 36044577 | 36102073 | 57496 | chrX | 96460782 | 96564210 | 103428 |
| chr3 | 36220448 | 36330343 | 109895 | chrX | 97792589 | 97797262 | 4673 |
| chr3 | 39579479 | 39593637 | 14158 | chrX | 98028641 | 98039208 | 10567 |
| chr3 | 39694705 | 39758913 | 64208 | chrX | 98079946 | 98169691 | 89745 |
| chr3 | 45305620 | 45321503 | 15883 | chrX | 99044930 | 99059389 | 14459 |
| chr3 | 66687412 | 66782220 | 94808 | chrX | 99100133 | 99104084 | 3951 |
| chr3 | 66822992 | 66900619 | 77627 | chrX | 99144824 | 99207232 | 62408 |
| chr3 | 66941383 | 66948306 | 6923 | chrX | 99247960 | 99335194 | 87234 |
| chr3 | 67100510 | 67146299 | 45789 | chrX | 99435276 | 99510511 | 75235 |
| chr3 | 70462726 | 70610236 | 147510 | chrX | 99551261 | 99581991 | 30730 |
| chr3 | 70650982 | 70654692 | 3710 | chrX | 99937691 | 100036638 | 98947 |
| chr3 | 71883989 | 71885299 | 1310 | chrX | 100460273 | 100460386 | 113 |
| chr3 | 73291896 | 73332432 | 40536 | chrX | 100501134 | 100523329 | 22195 |
| chr3 | 74186892 | 74190829 | 3937 | chrX | 104304933 | 104324061 | 19128 |
| chr3 | 74584871 | 74806797 | 221926 | chrX | 104364867 | 104422693 | 57826 |
| chr3 | 74847533 | 75034154 | 186621 | chrX | 104478931 | 104490217 | 11286 |
| chr3 | 75265636 | 75278548 | 12912 | chrX | 109104590 | 109141428 | 36838 |
| chr3 | 78444731 | 78475100 | 30369 | chrX | 109182192 | 109256172 | 73980 |
| chr3 | 79817815 | 79904288 | 86473 | chrX | 110573021 | 110578181 | 5160 |
| chr3 | 80008142 | 80038727 | 30585 | chrX | 112148233 | 112166608 | 18375 |
| chr3 | 80079455 | 80113326 | 33871 | chrX | 112207348 | 112209492 | 2144 |
| chr3 | 80267234 | 80392080 | 124846 | chrX | 112564290 | 112580647 | 16357 |
| chr3 | 80492305 | 80574000 | 81695 | chrX | 113133867 | 113252899 | 119032 |
| chr3 | 82613675 | 82622867 | 9192 | chrX | 113293661 | 113297412 | 3751 |
| chr3 | 82663605 | 82756514 | 92909 | chrX | 113397619 | 113453959 | 56340 |
| chr3 | 82858021 | 83313380 | 455359 | chrX | 115357556 | 115368181 | 10625 |
| chr3 | 83354112 | 83887044 | 532932 | chrX | 116232207 | 116257779 | 25572 |
| chr3 | 83988236 | 84024941 | 36705 | chrX | 116900945 | 116913048 | 12103 |
| chr3 | 84065691 | 84240111 | 174420 | chrX | 117013676 | 117051406 | 37730 |
| chr3 | 84340253 | 84421413 | 81160 | chrX | 117092152 | 117290569 | 198417 |
| chr3 | 86646996 | 86690034 | 43038 | chrX | 117331317 | 117347937 | 16620 |
| chr3 | 86730806 | 86766739 | 35933 | chrX | 117468882 | 117543007 | 74125 |
| chr3 | 86867036 | 86887968 | 20932 | chrX | 117583711 | 117589751 | 6040 |
| chr3 | 87394015 | 87449540 | 55525 | chrX | 117681258 | 117727215 | 45957 |
| chr3 | 94300000 | 94417255 | 117255 | chrX | 121255014 | 121282553 | 27539 |
| chr3 | 94557620 | 94788171 | 230551 | chrX | 121421053 | 121438696 | 17643 |
| chr3 | 95343238 | 95604422 | 261184 | chrX | 121525902 | 121538667 | 12765 |
| chr3 | 95733193 | 95997862 | 264669 | chrX | 121579437 | 121599376 | 19939 |
| chr3 | 96038594 | 96116812 | 78218 | chrX | 121640216 | 121692289 | 52073 |
| chr3 | 96295335 | 96299553 | 4218 | chrX | 122061612 | 122077130 | 15518 |
| chr3 | 96410039 | 96469868 | 59829 | chrX | 122392616 | 122421724 | 29108 |
| chr3 | 96510618 | 96516356 | 5738 | chrX | 122589460 | 122790425 | 200965 |
| chr3 | 99266266 | 99276376 | 10110 | chrX | 122894170 | 122967818 | 73648 |
| chr3 | 101048966 | 101090109 | 41143 | chrX | 125029921 | 125076835 | 46914 |
| chr3 | 102823913 | 102836123 | 12210 | chrX | 125117577 | 125149150 | 31573 |
| chr3 | 102876879 | 103109959 | 233080 | chrX | 125408655 | 125513133 | 104478 |
| chr3 | 103291230 | 103474032 | 182802 | chrX | 125553867 | 125612903 | 59036 |
| chr3 | 103682337 | 104004707 | 322370 | chrX | 125712994 | 125821962 | 108968 |
| chr3 | 104310751 | 104452699 | 141948 | chrX | 126602851 | 126647208 | 44357 |
| chr3 | 104553266 | 104692179 | 138913 | chrX | 126924001 | 126943052 | 19051 |
| chr3 | 104817388 | 105057658 | 240270 | chrX | 126983804 | 127041465 | 57661 |
| chr3 | 105157756 | 105316908 | 159152 | chrX | 127082211 | 127101032 | 18821 |
| chr3 | 106169552 | 106275736 | 106184 | chrX | 127174729 | 127225372 | 50643 |
| chr3 | 106376293 | 106465896 | 89603 | chrX | 127846733 | 127901293 | 54560 |
| chr3 | 106565998 | 106622905 | 56907 | chrX | 127942051 | 128000961 | 58910 |
| chr3 | 110027767 | 110261912 | 234145 | chrX | 128765790 | 128779194 | 13404 |
| chr3 | 110302684 | 110377481 | 74797 | chrX | 128888834 | 128910786 | 21952 |
| chr3 | 115435347 | 115508532 | 73185 | chrX | 128951532 | 128991535 | 40003 |
| chr3 | 117189389 | 117195940 | 6551 | chrX | 129093183 | 129108134 | 14951 |
| chr3 | 117236680 | 117255611 | 18931 | chrX | 129148912 | 129187703 | 38791 |
| chr3 | 117296405 | 117494204 | 197799 | chrX | 129247490 | 129306540 | 59050 |
| chr3 | 118707668 | 118793072 | 85404 | chrX | 129347278 | 129358381 | 11103 |
| chr3 | 135589888 | 135715523 | 125635 | chrX | 137081674 | 137274971 | 193297 |
| chr3 | 135756305 | 135875890 | 119585 | chrX | 137375049 | 137391257 | 16208 |
| chr3 | 137132196 | 137455880 | 323684 | chrX | 137737994 | 137758849 | 20855 |
| chr3 | 137586142 | 137621948 | 35806 | chrX | 137799621 | 138047924 | 248303 |
| chr3 | 144098719 | 144136904 | 38185 | chrX | 138088688 | 138299890 | 211202 |
| chr3 | 144237020 | 144292210 | 55190 | chrX | 138340672 | 138347350 | 6678 |
| chr3 | 144595844 | 144848538 | 252694 | chrX | 139287990 | 139312039 | 24049 |
| chr3 | 144889286 | 145041354 | 152068 | chrX | 141330360 | 141330822 | 462 |
| chr3 | 145082088 | 145473537 | 391449 | chrX | 141974140 | 141981244 | 7104 |
| chr3 | 145574415 | 145634571 | 60156 | chrX | 142255290 | 142314186 | 58896 |
| chr3 | 145735092 | 145774256 | 39164 | chrX | 142490110 | 142513047 | 22937 |
| chr3 | 146740325 | 146759684 | 19359 | chrX | 142553777 | 142556859 | 3082 |
| chr3 | 147641835 | 147694122 | 52287 | chrX | 142656945 | 142659373 | 2428 |
| chr3 | 152969796 | 152977250 | 7454 | chrX | 142700129 | 142876752 | 176623 |
| chr3 | 157727749 | 157826560 | 98811 | chrX | 142917488 | 142975917 | 58429 |
| chr3 | 161607442 | 161666908 | 59466 | chrX | 143978133 | 143997554 | 19421 |
| chr3 | 161971908 | 162059899 | 87991 | chrX | 144102222 | 144104085 | 1863 |
| chr3 | 162100645 | 162436540 | 335895 | chrX | 144775606 | 144779469 | 3863 |
| chr3 | 162536949 | 162551626 | 14677 | chrX | 144891660 | 145007107 | 115447 |
| chr3 | 162651792 | 162684758 | 32966 | chrX | 145383619 | 145396623 | 13004 |
| chr3 | 162725552 | 162757340 | 31788 | chrX | 145437377 | 145475008 | 37631 |
| chr3 | 162798092 | 162803261 | 5169 | chrX | 145534396 | 145538627 | 4231 |
| chr3 | 163629365 | 163675565 | 46200 | chrX | 146376553 | 146377455 | 902 |
| chr3 | 163716313 | 163951605 | 235292 | chrX | 147641909 | 147698519 | 56610 |
| chr3 | 164052465 | 164121470 | 69005 | chrX | 147739265 | 147750177 | 10912 |
| chr3 | 164221556 | 164510902 | 289346 | chrX | 148102746 | 148137129 | 34383 |
| chr3 | 165887472 | 165923051 | 35579 | chrX | 148270860 | 148292128 | 21268 |
| chr3 | 165963821 | 166110020 | 146199 | chrX | 149107166 | 149262232 | 155066 |
| | | | | chrX | 151332855 | 151346514 | 13659 |

The coordinates for the GSH sites in Table 1 were extracted from the GRCh38/hg38 genome assembly (UCSC Genome Browser on Human December 2013 (GRCh38/hg38) Assembly).

### Engineered Targeting Vectors

Provided herein, in some aspects, are engineered targeting vectors. A targeting vector is a nucleic acid used to deliver foreign genetic material into a cell. A targeting vector may include DNA, RNA or a combination of DNA and RNA. It may be single-stranded or double stranded, depending on the particular use of the vector. In some embodiments, the targeting vector is a double stranded DNA vector.

An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′-extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

The targeting vectors provided herein include a sequence of interest. A sequence of interest may be any nucleotide sequence, engineered (e.g., recombinant or synthetic), modified or unmodified (e.g., cloned from the genome of an organism without or with modification). In some embodiments, the sequence of interest comprises an open reading frame. An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame. In some embodiments, the vector comprises a promoter operably linked to the sequence of interest. A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal. Promoters may be constitutive or inducible (e.g., temporally or spatially). A targeting vector may also include, for example, other genetic elements, such as enhancers, termination sequences and the like to enable and/or facilitate gene expression.

A sequence of interest of a targeting vector provided herein, in some embodiments, is flanked by homology arms. Homology arms, herein, refer to regions of a targeting vector that are homologous to regions of genomic DNA located in a safe harbor site (e.g., of Table 1). One homology arm is located to the left (5′) of a sequence of interest (the left homology arm) and another homology arm is located to the right (3′) of the sequence of interest (the right homology arm). These homology arms enable homologous recombination between regions of the targeting vector and the genomic safe harbor locus, resulting in insertion of the sequence of interest into the genomic safe harbor site (e.g., via programmable nuclease-mediated) (e.g., CRISPR/Cas9-mediated) homology directed repair (HDR)).

The homology arms may vary in length. For example, each homology arm (the left arm and the right homology arm) may have a length of 5 nucleotide base pairs to 1000 nucleotide base pairs, depending in part on the intended use of the targeting vector. In some embodiments, each homology arm has a length of 50 to 1000, 50 to 900, 50 to 800, 50 to 700, 50 to 600, 50 to 500, 50 to 400, 50 to 300, 50 to 200, 50 to 100, 100 to 1000, 100 to 900, 100 to 800, 100 to 700, 100 to 600, 100 to 500, 100 to 400, 100 to 300, 100 to 200, 150 to 1000, 150 to 900, 150 to 800, 150 to 700, 150 to 600, 150 to 500, 150 to 400, 150 to 300, 150 to 200, 200 to 1000, 200 to 900, 200 to 800, 200 to 700, 200 to 600, 200 to 500, 200 to 400, or 200 to 300 nucleotide base pairs. In other embodiments, for example, in the context of gene modification using the CRIS-PITCh or TAL-PITCh systems (see, e.g., Sakuma T et al. Nat Protoc. 2016 January; 11(1):118-33), each homology arm has a length of 5 to 100, 5 to 90, 5 to 80, 5 to 70, 5 to 60, 5 to 50, 5 to 40, 5 to 30, 5 to 20, 10 to 100, 10 to 90, 10 to 80, 10 to 70, 10 to 60, 10 to 50, 10 to 40, 10 to 30, 10 to 20, 15 to 100, 15 to 90, 15 to 80, 15 to 70, 15 to 60, 15 to 50, 15 to 40, 15 to 30, or 15 to 20 nucleotide base pairs. In some embodiments, each homology arm has a length of about 5, 10, 15, 20, 25, 30, 35, 40, 45, 50, 60, 70, 80, 90, 100, 200, 300, 400, 500, 600, 700, 800, 900, or 1000 nucleotide bases. Longer homology arms are contemplated herein. In some embodiments, the length of one homology arm differs from the length of the other homology arm. For example, one homology arm may have a length of 200 nucleotide bases, and the other homology arm may have a length of 300 nucleotide bases.

Each homology arm comprises a sequence homologous to a sequence in a safe harbor site in the human genome selected from Table 1, for example. As is understood in the art, each homology arm flanking a gene of interest, for example, includes a sequence that is homologous to a target site in the genome such that the homology arms can function to facilitate insertion of that gene into the target site via a homologous recombination mechanism. Non-limiting examples of homology arm sequences are provided elsewhere herein.

The left homology arm, in some embodiments, may comprise a sequence that has at least 70%, at least 75%, at least 80%, at least 85%, at least 90%, at least 95%, or 100% identity to the sequence of any one of SEQ ID NOs: 25-44. In some embodiments, the left homology arm comprises a sequence that has at least 70%, at least 75%, at least 80%, at least 85%, at least 90%, at least 95%, or 100% identity to the sequence of SEQ ID NO: 25. In some embodiments, the left homology arm comprises a sequence that has at least 70%, at least 75%, at least 80%, at least 85%, at least 90%, at least 95%, or 100% identity to the sequence of SEQ ID NO: 26. In some embodiments, the left homology arm comprises a sequence that has at least 70%, at least 75%, at least 80%, at least 85%, at least 90%, at least 95%, or 100% identity to the sequence of SEQ ID NO: 27. In some embodiments, the left homology arm comprises a sequence that has at least 70%, at least 75%, at least 80%, at least 85%, at least 90%, at least 95%, or 100% identity to the sequence of SEQ ID NO: 28. In some embodiments, the left homology arm comprises a sequence that has at least 70%, at least 75%, at least 80%, at least 85%, at least 90%, at least 95%, or 100% identity to the sequence of SEQ ID NO: 29. In some embodiments, the left homology arm comprises a sequence that has at least 70%, at least 75%, at least 80%, at least 85%, at least 90%, at least 95%, or 100% identity to the sequence of SEQ ID NO: 30. In some embodiments, the left homology arm comprises a sequence that has at least 70%, at least 75%, at least 80%, at least 85%, at least 90%, at least 95%, or 100% identity to the sequence of SEQ ID NO: 31. In some embodiments, the left homology arm comprises a sequence that has at least 70%, at least 75%, at least 80%, at least 85%, at least 90%, at least 95%, or 100% identity to the sequence of SEQ ID NO: 32. In some embodiments, the left homology arm comprises a sequence that has at least 70%, at least 75%, at least 80%, at least 85%, at least 90%, at least 95%, or 100% identity to the sequence of SEQ ID NO: 33. In some embodiments, the left homology arm comprises a sequence that has at least 70%, at least 75%, at least 80%, at least 85%, at least 90%, at least 95%, or 100% identity to the sequence of SEQ ID NO: 34. In some embodiments, the left homology arm comprises a sequence that has at least 70%, at least 75%, at least 80%, at least 85%, at least 90%, at least 95%, or 100% identity to the sequence of SEQ ID NO: 35. In some embodiments, the left homology arm comprises a sequence that has at least 70%, at least 75%, at least 80%, at least 85%, at least 90%, at least 95%, or 100% identity to the sequence of SEQ ID NO: 36. In some embodiments, the left homology arm comprises a sequence that has at least 70%, at least 75%, at least 80%, at least 85%, at least 90%, at least 95%, or 100% identity to the sequence of SEQ ID NO: 37. In some embodiments, the left homology arm comprises a sequence that has at least 70%, at least 75%, at least 80%, at least 85%, at least 90%, at least 95%, or 100% identity to the sequence of SEQ ID NO: 38 In some embodiments, the left homology arm comprises a sequence that has at least 70%, at least 75%, at least 80%, at least 85%, at least 90%, at least 95%, or 100% identity to the sequence of SEQ ID NO: 39. In some embodiments, the left homology arm comprises a sequence that has at least 70%, at least 75%, at least 80%, at least 85%, at least 90%, at least 95%, or 100% identity to the sequence of SEQ ID NO: 40. In some embodiments, the left homology arm comprises a sequence that has at least 70%, at least 75%, at least 80%, at least 85%, at least 90%, at least 95%, or 100% identity to the sequence of SEQ ID NO: 41. In some embodiments, the left homology arm comprises a sequence that has at least 70%, at least 75%, at least 80%, at least 85%, at least 90%, at least 95%, or 100% identity to the sequence of SEQ ID NO: 42. In some embodiments, the left homology arm comprises a sequence that has at least 70%, at least 75%, at least 80%, at least 85%, at least 90%, at least 95%, or 100% identity to the sequence of SEQ ID NO: 43. In some embodiments, the left homology arm comprises a sequence that has at least 70%, at least 75%, at least 80%, at least 85%, at least 90%, at least 95%, or 100% identity to the sequence of SEQ ID NO: 44.

The right homology arm, in some embodiments, may comprise a sequence that has at least 70%, at least 75%, at least 80%, at least 85%, at least 90%, at least 95%, or 100% identity to the sequence of any one of SEQ ID NOs: 45-64. In some embodiments, the right homology arm comprises a sequence that has at least 70%, at least 75%, at least 80%, at least 85%, at least 90%, at least 95%, or 100% identity to the sequence of SEQ ID NO: 45. In some embodiments, the right homology arm comprises a sequence that has at least 70%, at least 75%, at least 80%, at least 85%, at least 90%, at least 95%, or 100% identity to the sequence of SEQ ID NO: 46. In some embodiments, the right homology arm comprises a sequence that has at least 70%, at least 75%, at least 80%, at least 85%, at least 90%, at least 95%, or 100% identity to the sequence of SEQ ID NO: 47. In some embodiments, the right homology arm comprises a sequence that has at least 70%, at least 75%, at least 80%, at least 85%, at least 90%, at least 95%, or 100% identity to the sequence of SEQ ID NO: 48. In some embodiments, the right homology arm comprises a sequence that has at least 70%, at least 75%, at least 80%, at least 85%, at least 90%, at least 95%, or 100% identity to the sequence of SEQ ID NO: 49. In some embodiments, the right homology arm comprises a sequence that has at least 70%, at least 75%, at least 80%, at least 85%, at least 90%, at least 95%, or 100% identity to the sequence of SEQ ID NO: 50. In some embodiments, the right homology arm comprises a sequence that has at least 70%, at least 75%, at least 80%, at least 85%, at least 90%, at least 95%, or 100% identity to the sequence of SEQ ID NO: 51. In some embodiments, the right homology arm comprises a sequence that has at least 70%, at least 75%, at least 80%, at least 85%, at least 90%, at least 95%, or 100% identity to the sequence of SEQ ID NO: 52. In some embodiments, the right homology arm comprises a sequence that has at least 70%, at least 75%, at least 80%, at least 85%, at least 90%, at least 95%, or 100% identity to the sequence of SEQ ID NO: 53. In some embodiments, the right homology arm comprises a sequence that has at least 70%, at least 75%, at least 80%, at least 85%, at least 90%, at least 95%, or 100% identity to the sequence of SEQ ID NO: 54. In some embodiments, the right homology arm comprises a sequence that has at least 70%, at least 75%, at least 80%, at least 85%, at least 90%, at least 95%, or 100% identity to the sequence of SEQ ID NO: 55. In some embodiments, the right homology arm comprises a sequence that has at least 70%, at least 75%, at least 80%, at least 85%, at least 90%, at least 95%, or 100% identity to the sequence of SEQ ID NO: 56. In some embodiments, the right homology arm comprises a sequence that has at least 70%, at least 75%, at least 80%, at least 85%, at least 90%, at least 95%, or 100% identity to the sequence of SEQ ID NO: 57. In some embodiments, the right homology arm comprises a sequence that has at least 70%, at least 75%, at least 80%, at least 85%, at least 90%, at least 95%, or 100% identity to the sequence of SEQ ID NO: 58. In some embodiments, the right homology arm comprises a sequence that has at least 70%, at least 75%, at least 80%, at least 85%, at least 90%, at least 95%, or 100% identity to the sequence of SEQ ID NO: 59. In some embodiments, the right homology arm comprises a sequence that has at least 70%, at least 75%, at least 80%, at least 85%, at least 90%, at least 95%, or 100% identity to the sequence of SEQ ID NO: 60. In some embodiments, the right homology arm comprises a sequence that has at least 70%, at least 75%, at least 80%, at least 85%, at least 90%, at least 95%, or 100% identity to the sequence of SEQ ID NO: 61. In some embodiments, the right homology arm comprises a sequence that has at least 70%, at least 75%, at least 80%, at least 85%, at least 90%, at least 95%, or 100% identity to the sequence of SEQ ID NO: 62. In some embodiments, the right homology arm comprises a sequence that has at least 70%, at least 75%, at least 80%, at least 85%, at least 90%, at least 95%, or 100% identity to the sequence of SEQ ID NO: 63. In some embodiments, the right homology arm comprises a sequence that has at least 70%, at least 75%, at least 80%, at least 85%, at least 90%, at least 95%, or 100% identity to the sequence of SEQ ID NO: 64.

In some embodiments, each homology arm comprises a sequence homologous to a genomic safe harbor site on chromosome 1. In some embodiments, each homology arm comprises a sequence homologous to a genomic safe harbor site on the long arm of chromosome 1. In some embodiments, each homology arm comprises a sequence homologous to a genomic safe harbor site at position 31 on the long arm of chromosome 1. For example, homology arms may comprise sequences homologous to a genomic safe harbor site at position 31.3 on the long arm of chromosome 1. In some embodiments, each homology arm comprises a sequence homologous to a genomic safe harbor site at position 31.3, coordinates 195,338,589-195,818,588[GRCh38/hg38], on the long arm of chromosome 1.

In some embodiments, each homology arm comprises a sequence homologous to a genomic safe harbor site on chromosome 3. In some embodiments, each homology arm comprises a sequence homologous to a genomic safe harbor site on the short arm of chromosome 3. In some embodiments, each homology arm comprises a sequence homologous to a genomic safe harbor site at position 24 on the short arm of chromosome 3. For example, homology arms may comprise sequences homologous to a genomic safe harbor site at position 24.3 on the short arm of chromosome 3. In some embodiments, each homology arm comprises a sequence homologous to a genomic safe harbor site at position 24.3, coordinates 22,720,711-22,761,389[GRCh38/hg38], on the short arm of chromosome 3.

In some embodiments, each homology arm comprises a sequence homologous to a genomic safe harbor site on chromosome 7. In some embodiments, each homology arm comprises a sequence homologous to a genomic safe harbor site on the long arm of chromosome 7. In some embodiments, each homology arm comprises a sequence homologous to a genomic safe harbor site at position 35 on the long arm of chromosome 7. For example, homology arms may comprise sequences homologous to a genomic safe harbor site at position 35, coordinates 145,090,941-145,219,513[GRCh38/hg38], on the long arm of chromosome 7. In some embodiments, homology arms may comprise sequences homologous to a genomic safe harbor site at position 35, coordinates 145,320,384-145,525,881[GRCh38/hg38], on the long arm of chromosome 7.

In some embodiments, each homology arm comprises a sequence homologous to a genomic safe harbor site on chromosome X. In some embodiments, each homology arm comprises a sequence homologous to a genomic safe harbor site on the long arm of chromosome X. In some embodiments, each homology arm comprises a sequence homologous to a genomic safe harbor site at position 21 on the long arm of chromosome X. For example, homology arms may comprise sequences homologous to a genomic safe harbor site at position 21.31 on the long arm of chromosome X. In some embodiments, each homology arm comprises a sequence homologous to a genomic safe harbor site at position 21.31, coordinates 89,174,426-89,179,074[GRCh38/hg38], on the long arm of chromosome X.

Targeting vectors of the present disclosure, in some embodiments, further comprise a sequence encoding at least one guide RNA that specifically targets (e.g., specifically binds to) the sequence in the safe harbor site and/or specifically targets a sequence in or near the homology arms. Specific binding refers to the gRNA binding with high specificity with a particular nucleic acid, as compared with other nucleic acid for which the gRNA has a lower affinity to bind (through Watson-Crick base pairing). Non-limiting examples of guide RNA sequences are described elsewhere herein. In some embodiments, a target vector further comprises a sequence encoding a programmable nuclease, such as a Cas nuclease, a zinc finger nuclease, or a TAL-effector nuclease. These programmable nuclease systems are discussed below.

### Genes of Interest

In some embodiments, a sequence of interest comprises a gene of interest. A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences). Non-limiting examples of genes of interest are provided in Table 2 below.

Any one or more of the gene(s) of interest in Table 2, for example, may be knocked into any one or more of the genomic safe harbor sites provided herein, ex vivo or in vivo, to treat a particular disease or condition, such as those listed in Table 2. The gene of interest may be modified (e.g., mutated) or unmodified, depending on the particular therapeutic application.

TABLE 2
Examples of Genes of Interest

| Indication | Gene | Gene:Locus[GRCh38/hg38] |
|---|---|---|
| Netherton Syndrome | SPINK5 | SPINK5:5q31-q32 |
| Xeroderma pigmentosum | XPA/XPB/XPC/XPD/XPE/XPG/ | XPA/XPB/XPC/XPD/XPE/XPG/ |
| | XPV/POLH | XPV/POLH |
| Xeroderma pigmentosum variant | POLH | POLH:6p21.1-p12 |
| Xeroderma pigmentosum- | ERCC3/ERCC2/ERCC5 | ERCC3:2q21/ERCC2:19q13.3/ |
| Cockayne syndrome complex | | ERCC5:13q22-q34 |
| Sjogren-Larsson syndrome | ALDH3A2 | ALDH3A2 |
| Harlequin Ichthyosis | ABCA12 | ABCA12 |
| Lamellar Ichthyosis | TGM1/ABCA12/ALOX12B/ | TGM1/ABCA12/ALOX12B/ |
| | NIPAL4/TGM1/ABCA12/ABC/ | NIPAL4/TGM1/ABCA12/ABC/ |
| | ALOX12B/NIPAL4 | ALOX12B/NIPAL4 |
| Hailey Hailey | ATP2C1 | ATP2C1:3q21-q24 |
| Darier's disease | ATP2A2 | ATP2A2:12q23-q24.1 |
| Erythrokeratoderma variabilis | GJB4/GJB3/GJA1 | GJB4:1p35- |
| progressiva | | p34/GJB3:1p34/GJA1:6922.31 |
| Acquired epidermolysis bullosa | anti-COL7A1 Ab | anti-COL7A1 Ab |
| Epidermolysis bullosa simplex | COL7A1 | COL7A1 |
| Epidermolysis bullosa simplex | PKP1 | PKP1:1q32 |
| due to plakophilin deficiency | | |
| Epidermolysis bullosa simplex | COL7A1 | COL7A1:3p21.3 |
| superficialis | | |
| Epidermolysis bullosa simplex | KRT5 | KRT5:12q13.13 |
| with circinate migratory erythema | | |
| Epidermolysis bullosa simplex | KRT5 | KRT5:12q13.13 |
| with mottled pigmentation | | |
| Epidermolysis bullosa simplex | PLEC | PLEC:8q24 |
| with muscular dystrophy | | |
| Epidermolysis bullosa simplex | PLEC | PLEC:8q24 |
| with pyloric atresia | | |
| Epidermolysis bullosa simplex, | PLEC | PLEC:8q24 |
| Ogna type | | |
| Epidermolysis bullosa simplex, | KRT14 | KRT14:17q12-q21 |
| autosomal recessive K14 | | |
| Epidermolysis bullosa simplex, | KRT5/KRT14 | KRT5:12q13.13/KRT14:17q12- |
| generalized intermediate | | q21 |
| Epidermolysis bullosa simplex, | KRT5/KRT14 | KRT5:12q13.13/KRT14:17q12- |
| generalized severe | | q21 |
| Localized epidermolysis bullosa | KRT5/KRT14 | KRT5:12q13.13/KRT14:17q12- |
| simplex | | q21 |
| Junctional epidermolysis bullosa | COL17A1/ITGA6/ITGB4/ | COL17A1/ITGA6/ITGB4/ |
| | LAMA3/LAMB3/LAMC2 | LAMA3/LAMB3/LAMC2 |
| Junctional epidermolysis bullosa | LAMC2 | LAMC2:1q25-q31 |
| inversa | | |
| Junctional epidermolysis bullosa, | COL17A1/LAMA3/LAMB3/ | COL17A1:10q24.3/LAMA3:18q11.2/ |
| generalized intermediate | LAMC2 | LAMB3:1q32/LAMC2:1q25-q31 |
| Junctional epidermolysis bullosa, | LAMA3/LAMB3/LAMC2 | LAMA3:18q11.2/LAMB3:1q32/ |
| generalized severe | | LAMC2:1q25-q31 |
| Junctional epidermolysis bullosa, | LAMA3/LAMB3/LAMC2 | LAMA3:18q11.2/LAMB3:1q32/ |
| non-Herlitz type | | LAMC2:1q25-q31 |
| Junctional epidermolysis bullosa- | ITGA6/ITGB4 | ITGA6:2q31.1/ITGB4:17q11-qter |
| pyloric atresia syndrome | | |
| Late-onset junctional | COL17A1 | COL17A1 |
| epidermolysis bullosa | | |
| Localized junctional | COL17A1 | COL17A1:10q24.3 |
| epidermolysis bullosa, non-Herlitz | | |
| type | | |
| Dystrophic epidermolysis bullosa | COL7A1 | COL7A1:3p21.31 |
| Acral dystrophic epidermolysis | COL7A1 | COL7A1 |
| bullosa | | |
| Generalized dominant dystrophic | COL7A1 | COL7A1 |
| epidermolysis bullosa | | |
| Centripetalis recessive dystrophic | COL7A1 | COL7A1 |
| epidermolysis bullosa | | |
| Dystrophic epidermolysis bullosa | COL7A1 | COL7A1 |
| pruriginosa | | |
| Pretibial dystrophic epidermolysis | COL7A1 | COL7A1 |
| bullosa | | |
| Recessive dystrophic | COL7A1 | COL7A1 |
| epidermolysis bullosa inversa | | |
| Recessive dystrophic | COL7A1 | COL7A1 |
| epidermolysis bullosa, generalized | | |
| intermediate | | |
| Severe generalized recessive | COL7A1 | COL7A1 |
| dystrophic epidermolysis bullosa | | |
| Kindler syndrome | FERMT1 | FERMT1:20p12.3 |
| Acral peeling skin syndrome | TGM5 | TGM5:15q15 |
| Acrodermatitis enteropathica | SLC39A4 | SLC39A4:8q24.3 |
| Arthrochalasia Ehlers-Danlos | COL1A1/COL1A2 | COL1A1/COL1A2 |
| syndrome | | |
| Autosomal dominant hyper-IgE | STAT3/IgE | STAT3:17q21.31/IgE |
| syndrome | | |
| Bazex-Dupré-Christol syndrome | UBE2A | UBE2A:Xq24 |

The compositions and methods provided herein, in some embodiments, may be used for manufacturing/producing (e.g., on a large scale) therapeutic proteins from human cells ex vivo. Thus, in some embodiments, a gene of interest encodes a therapeutic protein (see, e.g., Dimitrov D S Methods Mol Biol. 2012; 899: 1-26, incorporated herein by reference). Non-limiting examples of therapeutic proteins include antibodies, Fc fusion proteins, anticoagulants, blood factors, bone morphogenetic proteins, engineered protein scaffolds, enzymes, growth factors, hormones, interferons, interleukins, and thrombolytics. In some embodiments, the therapeutic protein is an antibody. Therapeutic proteins may also be classified based on mechanism of activity, for example, (a) binding non-covalently to target, e.g., mAbs; (b) affecting covalent bonds, e.g., enzymes; and (c) exerting activity without specific interactions, e.g., serum albumin.

Non-limiting examples of antibodies that may be produced using the compositions (e.g., targeting vectors) and/or methods of the present disclosure include: abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, amivantamab, anatumomab mafenatox, andecaliximab, anetumab ravtansine, anifrolumab, ansuvimab, anrukinzumab, apolizumab, aprutumab ixadotin, arcitumomab, ascrinvacumab, aselizumab, atezolizumab, atidortoxumab, atinumab, atoltivimab, atoltivimab/maftivimab/odesivimab, atorolimumab, avelumab, azintuxizumab vedotin, bamlanivimab, bapineuzumab, basiliximab, bavituximab, bcd-, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, casirivimab, capromab, carlumab, carotuximab, catumaxomab, cbr-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, cr, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, dinutuximab beta, diridavumab, domagrozumab, dorlimomab aritox, dostarlimab, drozitumab, ds-, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epcoritamab, epitumomab cituxetan, epratuzumab, eptinezumab, erenumab, erlizumab, ertumaxomab, etaracizumab, etesevimab, etigilimab, etrolizumab, evinacumab, evolocumab, exbivirumab, fanolesomab, faralimomab, faricimab, farletuzumab, fasinumab, fbta, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, ibi, ibritumomab tiuxetan, icrucumab, idarucizumab, ifabotuzumab, igovomab, iladatuzumab vedotin, imab, imalumab, imaprelimab, imciromab, imdevimab, imgatuzumab, inclacumab, indatuximab ravtansine, indusatumab vedotin, inebilizumab, infliximab, intetumumab, inolimomab, inotuzumab ozogamicin, ipilimumab, iomab-b, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, loncastuximab tesirine, losatuxizumab vedotin, lilotomab satetraxetan, lintuzumab, lirilumab, lodelcizumab, lokivetmab, lorvotuzumab mertansine, lucatumumab, lulizumab pegol, lumiliximab, lumretuzumab, lupartumab, lupartumab amadotin, lutikizumab, maftivimab, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-cd, nacolomab tafenatox, namilumab, naptumomab estafenatox, naratuximab emtansine, narnatumab, natalizumab, navicixizumab, navivumab, naxitamab, nebacumab, necitumumab, nemolizumab, neod, nerelimomab, nesvacumab, netakimab, nimotuzumab, nirsevimab, nivolumab, nofetumomab merpentan, obiltoxaximab, obinutuzumab, ocaratuzumab, ocrelizumab, odesivimab, odulimomab, ofatumumab, olaratumab, oleclumab, olendalizumab, olokizumab, omalizumab, omburtamab, oms, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pdr, pembrolizumab, pemtumomab, perakizumab, pertuzumab, pexelizumab, pidilizumab, pinatuzumab vedotin, pintumomab, placulumab, prezalumab, plozalizumab, pogalizumab, polatuzumab vedotin, ponezumab, porgaviximab, prasinezumab, prezalizumab, priliximab, pritoxaximab, pritumumab, pro, quilizumab, racotumomab, radretumab, rafivirumab, ralpancizumab, ramucirumab, ranevetmab, ranibizumab, raxibacumab, ravagalimab, ravulizumab, refanezumab, regavirumab, regn-eb, relatlimab, remtolumab, reslizumab, rilotumumab, rinucumab, risankizumab, rituximab, rivabazumab pegol, robatumumab, rmab, roledumab, romilkimab, romosozumab, rontalizumab, rosmantuzumab, rovalpituzumab tesirine, rovelizumab, rozanolixizumab, ruplizumab, sa, sacituzumab govitecan, samalizumab, samrotamab vedotin, sarilumab, satralizumab, satumomab pendetide, secukinumab, selicrelumab, seribantumab, setoxaximab, setrusumab, sevirumab, sibrotuzumab, sgn-cda, shp, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, tafasitamab, talacotuzumab, talizumab, talquetamab, tamtuvetmab, tanezumab, taplitumomab paptox, tarextumab, tavolimab, teclistamab, tefibazumab, telimomab aritox, telisotuzumab, telisotuzumab vedotin, tenatumomab, teneliximab, teplizumab, tepoditamab, teprotumumab, tesidolumab, tetulomab, tezepelumab, tgn, tibulizumab, tildrakizumab, tigatuzumab, timigutuzumab, timolumab, tiragolumab, tiragotumab, tislelizumab, tisotumab vedotin, tocilizumab, tomuzotuximab, toralizumab, tosatoxumab, tositumomab, tovetumab, tralokinumab, trastuzumab, trastuzumab duocarmazine, trastuzumab emtansine, trbs, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, xmab-, zalutumumab, zanolimumab, zatuximab, zenocutuzumab, ziralimumab, zolbetuximab (claudiximab), and zolimomab aritox.

The compositions and methods provided herein, in other embodiments, may be used for manufacturing/producing (e.g., on a large scale) gene therapy vectors from human cells ex vivo. Thus, provided herein are methods comprising introducing one or more polynucleotide into a safe harbor site in a human cell ex vivo and producing a recombinant gene therapy vector or one or more components of a gene therapy vector encoded by the one or more polynucleotide. In some embodiments, the polynucleotide comprises a viral polynucleotide (e.g., encoding a viral protein). The viral polynucleotide may be, for example, an adenovirus protein, an adeno-associated virus protein (AAV), a retrovirus protein, or a Herpes virus protein. In some embodiments, the polynucleotide may include one or more of a promoter, enhancer, intron, exon, stop signals, polyadenylation signals, inverted terminal repeat (ITR) sequences, replication (rep) genes, capsid (cap) coding sequences, helper genes, or other sequences used in producing a gene therapy vector, such as a recombinant AAV vector.

### Genomic Editing Methods

Engineered nucleic acids (e.g., sequences of interest) may be introduced to a genomic safe harbor site using any suitable method. The present application contemplates the use of a variety of gene editing and other knock-in technologies, for example, to introduce nucleic acids into a genomic safe harbor site. Non-limiting examples include programmable nuclease-based systems, such as clustered regularly interspaced short palindromic repeat (CRISPR) systems (e.g., including Cas-based systems, prime editing (see, e.g., Anzalone A V et al. Nat Biotechnol. 2021 Dec. 9) and CRISPR-directed integrases (see, e.g., Ioannidi E I et al. bioRxiv, 2021 Nov. 1), zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit a genomic safe harbor site. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein, one or multiple guide RNAs (gRNAs), and/or a targeting vector may be used to introduce a sequence of interest into a genomic safe harbor site.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1 (Cas12a), C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2011; 471: 602-607, each of which is incorporated by reference herein.

In some embodiments, a guide RNA comprising a sequence homologous to a sequence in a safe harbor site in the human genome in any one of the loci listed in Table 1, e.g., 1q31, 3p24, 7q35, and Xq21. One skilled in the art can readily determine a gRNA sequence for specifically targeting the genomic safe harbor sites provided herein. Nonetheless, non-limited examples of gRNA sequences are provided as SEQ ID NOs: 5-24. The gRNA, in some embodiments, may comprise a sequence that has at least 70%, at least 75%, at least 80%, at least 85%, at least 90%, at least 95%, or 100% identity to any one of the gRNA sequences of SEQ ID NOs: 5-24. The gRNA, in some embodiments, may comprise a sequence that has at least 70%, at least 75%, at least 80%, at least 85%, at least 90%, at least 95%, or 100% identity to the gRNA sequence of SEQ ID NO: 5. The gRNA, in some embodiments, may comprise a sequence that has at least 70%, at least 75%, at least 80%, at least 85%, at least 90%, at least 95%, or 100% identity to the gRNA sequence of SEQ ID NO: 6. The gRNA, in some embodiments, may comprise a sequence that has at least 70%, at least 75%, at least 80%, at least 85%, at least 90%, at least 95%, or 100% identity to the gRNA sequence of SEQ ID NO: 7. The gRNA, in some embodiments, may comprise a sequence that has at least 70%, at least 75%, at least 80%, at least 85%, at least 90%, at least 95%, or 100% identity to the gRNA sequence of SEQ ID NO: 8. The gRNA, in some embodiments, may comprise a sequence that has at least 70%, at least 75%, at least 80%, at least 85%, at least 90%, at least 95%, or 100% identity to the gRNA sequence of SEQ ID NO: 9. The gRNA, in some embodiments, may comprise a sequence that has at least 70%, at least 75%, at least 80%, at least 85%, at least 90%, at least 95%, or 100% identity to the gRNA sequence of SEQ ID NO: 10. The gRNA, in some embodiments, may comprise a sequence that has at least 70%, at least 75%, at least 80%, at least 85%, at least 90%, at least 95%, or 100% identity to the gRNA sequence of SEQ ID NO: 11. The gRNA, in some embodiments, may comprise a sequence that has at least 70%, at least 75%, at least 80%, at least 85%, at least 90%, at least 95%, or 100% identity to the gRNA sequence of SEQ ID NO: 12. The gRNA, in some embodiments, may comprise a sequence that has at least 70%, at least 75%, at least 80%, at least 85%, at least 90%, at least 95%, or 100% identity to the gRNA sequence of SEQ ID NO: 13. The gRNA, in some embodiments, may comprise a sequence that has at least 70%, at least 75%, at least 80%, at least 85%, at least 90%, at least 95%, or 100% identity to the gRNA sequence of SEQ ID NO: 14. The gRNA, in some embodiments, may comprise a sequence that has at least 70%, at least 75%, at least 80%, at least 85%, at least 90%, at least 95%, or 100% identity to the gRNA sequence of SEQ ID NO: 15. The gRNA, in some embodiments, may comprise a sequence that has at least 70%, at least 75%, at least 80%, at least 85%, at least 90%, at least 95%, or 100% identity to the gRNA sequence of SEQ ID NO: 16. The gRNA, in some embodiments, may comprise a sequence that has at least 70%, at least 75%, at least 80%, at least 85%, at least 90%, at least 95%, or 100% identity to the gRNA sequence of SEQ ID NO: 17. The gRNA, in some embodiments, may comprise a sequence that has at least 70%, at least 75%, at least 80%, at least 85%, at least 90%, at least 95%, or 100% identity to the gRNA sequence of SEQ ID NO: 18. The gRNA, in some embodiments, may comprise a sequence that has at least 70%, at least 75%, at least 80%, at least 85%, at least 90%, at least 95%, or 100% identity to the gRNA sequence of SEQ ID NO: 19. The gRNA, in some embodiments, may comprise a sequence that has at least 70%, at least 75%, at least 80%, at least 85%, at least 90%, at least 95%, or 100% identity to the gRNA sequence of SEQ ID NO: 20. The gRNA, in some embodiments, may comprise a sequence that has at least 70%, at least 75%, at least 80%, at least 85%, at least 90%, at least 95%, or 100% identity to the gRNA sequence of SEQ ID NO: 21. The gRNA, in some embodiments, may comprise a sequence that has at least 70%, at least 75%, at least 80%, at least 85%, at least 90%, at least 95%, or 100% identity to the gRNA sequence of SEQ ID NO: 22. The gRNA, in some embodiments, may comprise a sequence that has at least 70%, at least 75%, at least 80%, at least 85%, at least 90%, at least 95%, or 100% identity to the gRNA sequence of SEQ ID NO: 23. The gRNA, in some embodiments, may comprise a sequence that has at least 70%, at least 75%, at least 80%, at least 85%, at least 90%, at least 95%, or 100% identity to the gRNA sequence of SEQ ID NO: 24.

In some embodiments, the RNA-guided nuclease and the gRNA are complexed to form a ribonucleoprotein (RNP), prior to delivery to a cell, for example.

The concentration of programmable nuclease or nucleic acid encoding the programmable nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

### Delivery Systems

The targeting vector, in some embodiments, is delivered to a subject and/or cell using a delivery system. A delivery system, herein, is any substance or combination of substances that can be used to bring (deliver) a targeting vector to a cell. Delivery systems are often used to effectively deliver nucleic acids to cells ex vivo and/or in vivo. Such delivery systems can protect the targeting vector from inactivation and/or degradation. Non-limiting examples of delivery systems include viral delivery systems and non-viral delivery systems.

In some embodiments, the delivery system is a viral delivery system. Viral delivery system typically includes viruses engineered to be replication deficient. Such viral delivery systems can be used to deliver a targeting vector to a cell by infecting the cell. Non-limiting examples of viral delivery systems include engineered adeno-associated viruses, adenoviruses and lentiviruses. Such viral delivery systems are well-known.

In other embodiments, the delivery system is a non-viral delivery system. Non-limiting examples of non-viral delivery systems include synthetic nanoparticles, such as lipid nanoparticles and liposomes. A lipid nanoparticle is typically spherical with an average diameter between 10 and 1000 nanometers. Lipid nanoparticles possess a solid lipid core matrix that can solubilize lipophilic molecules. The lipid core is stabilized by surfactants (emulsifiers). The surfactant used depends, in part, on the route of administration. The term lipid includes triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). All classes of emulsifiers (with respect to charge and molecular weight) have been used to stabilize lipid dispersions. Liposomes, by contrast, are small, spherical vesicles that have a phospholipid bilayer as coat, because the bulk of the interior of the particle is composed of aqueous substance. Such non-viral delivery systems are well-known.

Other non-viral biological agent delivery systems are also contemplated herein, including bacteria, bacteriophage, virus-like particles (VLPs), erythrocyte ghosts, and exosomes. See, e.g., Seow Y. et al. Mol Ther. 2009 May; 17(5):767-7.

### Methods of Use

The compositions provided herein may be used, in some embodiments, to deliver a targeting vector (with a modified or unmodified gene of interest, for example) to a genomic safe harbor site in a human cell, ex vivo or in vivo. Thus, provided herein are methods that comprise delivering to a human cell an engineered targeting vector or a delivery system comprising a targeting vector. The methods, in some embodiments, further comprise delivering to the human cell a programmable nuclease (e.g., RNA-guided nuclease and a (one, two, three, or more) gRNA, ZFN, and/or TALEN) or a nucleic acid encoding the programmable nuclease.

The method may also include incubating the human cell to modify the safe harbor site to include the sequence of interest. One of skill in the art can readily determine the incubation conditions to enable homologous recombination or non-homologous end joining to occur, depending on the configuration of the engineered targeting vector (e.g., homology arms v. microhomology arms) and the gene editing system of choice (e.g., RNA-guided nuclease and a (one, two, three, or more) gRNA, ZFN, and/or TALEN). In some embodiments, the human cell (e.g., containing an engineered targeting vector) is incubated for a time period of about 5 minutes to about 3 hours, e.g., 5, 10, 15, 20, 25, 30, 35, 40, 45, 50, 55, or 60 minutes, or 1.5, 2, 2.5, or 3 hours. In some embodiments, the human cell is incubated at a temperature of about 25° C. to about 95° C., e.g., 25° C., 37° C., 42° C. or 95° C.

Various therapies are also contemplated herein. Thus, the present disclosure provides methods of delivering to a subject an engineered targeting vector, a delivery system comprising the engineered targeting vector, or a cell modified using the engineered targeting vector. The subject may suffer from any one or more of the diseases or conditions listed in Table 2. The gene of interest will likely depend on the particular disease or condition, and guidance for selecting particular genes of interest, based on a particular diseases or conditions are provided in Table 2.

Also provided herein are methods comprising identifying a safe harbor site in the human genome that is at least 50 kb (e.g., at least 60, 70, 80, 90, or 100 kb) from any known gene, at least 20 kb (e.g., at least 30, 40, or 50 kb) from an enhanced region, at least 150 kb (e.g., at least 200, 300, 400, or 50 kb) from a long non-coding RNA (lncRNA) and a tRNA, at least 300 kb (e.g., at least 400 or 500 kb) from any known oncogene, at least 300 kb (e.g., at least 400 or 500 kb) from a miRNA, and at least 300 kb (e.g., at least 400 or 500 kb) from a telomere and a centromere.

Some aspects provide methods comprising amplifying sequence from safe harbor site in the human genome that is at least 50 kb (e.g., at least 60, 70, 80, 90, or 100 kb) from any known gene, at least 20 kb (e.g., at least 30, 40, or 50 kb) from an enhanced region, at least 150 kb (e.g., at least 200, 300, 400, or 50 kb) from a lncRNA and a tRNA, at least 300 kb (e.g., at least 400 or 500 kb) from any known oncogene, at least 300 kb (e.g., at least 400 or 500 kb) from a miRNA, and at least 300 kb (e.g., at least 400 or 500 kb) from a telomere and a centromere.

Other aspects provide methods comprising modifying sequence in safe harbor site in the human genome that is at least 50 kb (e.g., at least 60, 70, 80, 90, or 100 kb) from any known gene, at least 20 kb (e.g., at least 30, 40, or 50 kb) from an enhanced region, at least 150 kb (e.g., at least 200, 300, 400, or 50 kb) from a lncRNA and a tRNA, at least 300 kb (e.g., at least 400 or 500 kb) from any known oncogene, at least 300 kb (e.g., at least 400 or 500 kb) from a miRNA, and at least 300 kb (e.g., at least 400 or 500 kb) from a telomere and a centromere.

### Cell Delivery Methods

Multiple delivery methods are available for delivering nucleic acids into a cell in vivo or ex vivo. The method used depends, at least in part, on the delivery system chosen. For example, viral systems use the natural ability of viruses to infect cells that present cell surface receptors to the viral surface proteins. Once a virus attaches through its surface proteins to a cell surface receptor of a target cell, conformational changes occur in the viral proteins that lead either to penetration of the virus through the cell membrane (for non-enveloped viruses), or to fusion of the viral envelope with the cell membrane. Either process results in insertion of the viral genome, or viral payload, into the target cell. For non-viral systems, such as a liposome or an LNP, the payload carried by a particle, can be delivered into target cells through a variety of methods. Non-limiting examples include the fusion of the particle membrane (or coating) with the cell membrane leading to payload insertion into the cytoplasm, the endocytosis of the particle by engulfment into the cell, chemical transfection methods (e.g., calcium phosphate exposure), physical transfection methods (e.g., electroporation).

### Routes of Administration

Multiple routes of administration are available for delivering targeting vectors to a human subject. Exemplary routes of administration include, without limitation, oral, intravenous, intramuscular, intrathecal, sublingual, buccal, rectal, vaginal, ocular, otic, nasal, inhalation, nebulization, cutaneous/subcutaneous (for topical or systemic effect), and transdermal. Modified cells may also be delivered through select routes, including but not limited to intravenous.

### Cell Types

Cell therapy (e.g., allogeneic or autologous) is a therapy in which viable cells are injected, grafted or implanted into a patient in order to effectuate a medicinal effect, for example, by transplanting T-cells capable of fighting cancer cells via cell-mediated immunity in the course of immunotherapy, or grafting stem cells to regenerate diseased tissues. The present disclosure contemplates the modification of a myriad of cell types for cell therapy. Non-limiting examples include stem cells (e.g., an induced pluripotent stem cell (iPSC)), red blood cells (e.g., erythrocytes), white blood cells, platelets, nerve cells, muscle cells, cartilage cells (e.g., chondrocytes), bone cells, skin cells, endothelial cells, epithelial cells, fat cells, and sex cells. In embodiments in which red blood cells are contemplate, hematopoietic stem cells may be modified and then differentiated into red blood cells.

Examples of stem cells include, but are not limited to, human embryonic stem cells, human adult stem cells, neural stem cells, mesenchymal stem cells, and hematopoietic stem cells. The stem cells may be, in some embodiments, be induced pluripotent stem cells (iPSCs).

Examples of white blood cells include, but are not limited to, neutrophils, eosinophils, basophils, mast cells, monocytes, macrophages, dendritic cells, natural killer cells, and lymphocytes (B cells and T cells).

Examples of nerve cells include, but are not limited to, neurons and neuroglial cells.

Examples of muscle cells include, but are not limited to, skeletal, cardiac, and smooth muscle cells.

Examples of bone cells include, but are not limited to, osteoblasts, osteoclasts, osteocytes, and lining cells.

Examples of skin cells include, but are not limited to, keratinocytes, melanocytes, Merkel cells, and Langerhans cells.

Examples of fat cells include, but are not limited to, white adipocytes and brown adipocytes.

Particular cell therapies, such as adoptive cell transfer therapies are also provided herein, including, for example, chimeric antigen receptor (CAR) T cell therapy (e.g., for cancer therapy) and fibroblast cell therapy (e.g., to ameliorate inherited diseases and aging).

### Additional Embodiments

Additional embodiments of the present disclosure are encompassed by the following numbered paragraphs.
1. An engineered nucleic acid targeting vector comprising a sequence of interest flanked by homology arms, each homology arm comprising a sequence homologous to a sequence in a safe harbor site in the human genome in any one of the following loci of Table 1.
2. The vector of any one of the preceding paragraphs, wherein the sequence of interest comprises an open reading frame.
3. The vector of any one of the preceding paragraphs, wherein the vector comprises a promoter operably linked to the sequence of interest.
4. The vector of any one of the preceding paragraphs, wherein the sequence of interest comprises or is within a gene of interest, optionally selected from Table 2.
5. The vector of any one of the preceding paragraphs, wherein the vector is a double-stranded DNA vector, optionally wherein the sequence of interest is flanked by regions that enable circularization, preferably via trans-splicing, upon expression.
6. The vector of any one of the preceding paragraphs, wherein each homology arm has a length of about 200 to about 500 base pairs (bp), optionally 300 bp.
7. The vector of any one of the preceding paragraphs, wherein each homology arm is a microhomology arm having a length of about 5 to 50 bp, optionally 40 bp.
8. The vector of any one of the preceding paragraphs, further comprising a sequence encoding at least one guide RNA that specifically targets the sequence in the safe harbor site and/or specifically targets a sequence in or near the homology arms.
10. The vector of any one of the preceding paragraphs, further comprising a sequence encoding a programmable nuclease.
11. A delivery system, e.g., a lipid nanoparticle, comprising the vector of any one of the preceding paragraphs.
12. The delivery system of paragraph 11 further comprising a programmable nuclease or a nucleic acid encoding the programmable nuclease.
13. The delivery system of paragraph 12, wherein the programmable nuclease is selected from ZFNs, TALENs, DNA-guided nucleases, and RNA-guided nucleases.
14. The lipid nanoparticle of paragraph 13, wherein the programmable nuclease is an RNA-guided nuclease.
15. The delivery system of paragraph 14, wherein the RNA-guided nuclease is a CRISPR Cas nuclease and the delivery system further comprises a guide RNA or a nucleic acid encoding the gRNA.
16. The delivery system of paragraph 15, wherein the CRISPR Cas nuclease is a Cas9 nuclease or a Cas12 nuclease.
17. The delivery system of paragraph 15 or 16, wherein the gRNA specifically targets the sequence in the safe harbor site and/or specifically targets a sequence in or near the homology arms.
18. A method comprising delivering to a human cell the delivery system of any one of the preceding paragraphs.
19. A method comprising delivering to a human cell the engineered targeting vector any one of the preceding paragraphs.
20. The method of paragraph 19 further comprising delivering to the human cell a programmable nuclease or a nucleic acid encoding the programmable nuclease.
21. The method of any one of the preceding paragraphs further comprising incubating the human cell to modify the safe harbor site to include the sequence of interest.
22. The method of any one of the preceding paragraphs wherein the human cell is a stem cell, an immune cell (e.g., T cell), or a mesenchymal cell (e.g., fibroblast).
23. A method comprising delivering to a subject the delivery system of any one of the preceding paragraphs.
24. A method comprising delivering to a subject the engineered targeting vector any one of the preceding paragraphs.
25. The method of paragraph 24 further comprising delivering to the subject a programmable nuclease or a nucleic acid encoding the programmable nuclease.
26. The method of any one of the preceding paragraphs, wherein the programmable nuclease is selected from ZFNs, TALENs, DNA-guided nucleases, and RNA-guided nucleases.
27. The method of paragraph 26, wherein the programmable nuclease is an RNA-guided nuclease.
28. The method of paragraph 27, wherein the RNA-guided nuclease is a CRISPR Cas nuclease and the delivery system further comprises a guide RNA or a nucleic acid encoding the gRNA.
29. The method of paragraph 28, wherein the CRISPR Cas nuclease is a Cas9 nuclease or a Cas12 nuclease.
30. The method of paragraph 28 or 29, wherein the gRNA specifically targets the sequence in the safe harbor site and/or specifically targets a sequence in or near the homology arms.
31. The method of any one of paragraphs 23-30, wherein the subject has a medical condition selected from Table 2.
32. The method of paragraph 31, wherein the gene of interest is selected from Table 2.
33. The method of paragraph 32, wherein the gene of interest is a variant of a gene selected from Table 2.
34. A guide RNA comprising a sequence homologous to a sequence in a safe harbor site in the human genome in any one of the loci of Table 1.
35. A delivery system, e.g., lipid nanoparticle. comprising the guide RNA of paragraph 34.
36. A method comprising genetically modifying a safe harbor site in the human genome in any one of the loci of Table 1.

### EXAMPLES

### Example 1. Bioinformatic Search of Novel GSH Site

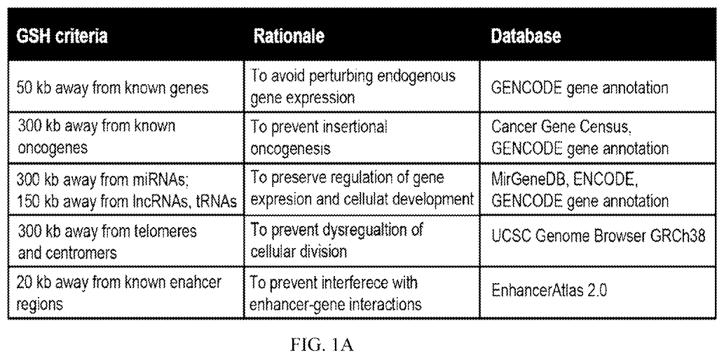

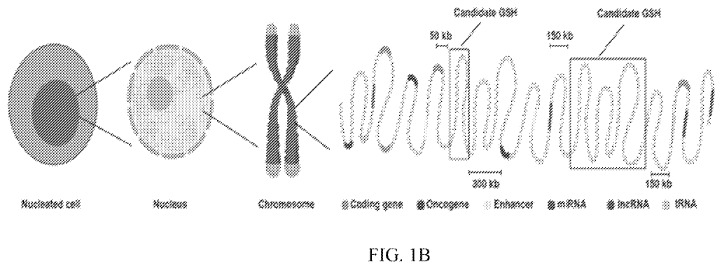

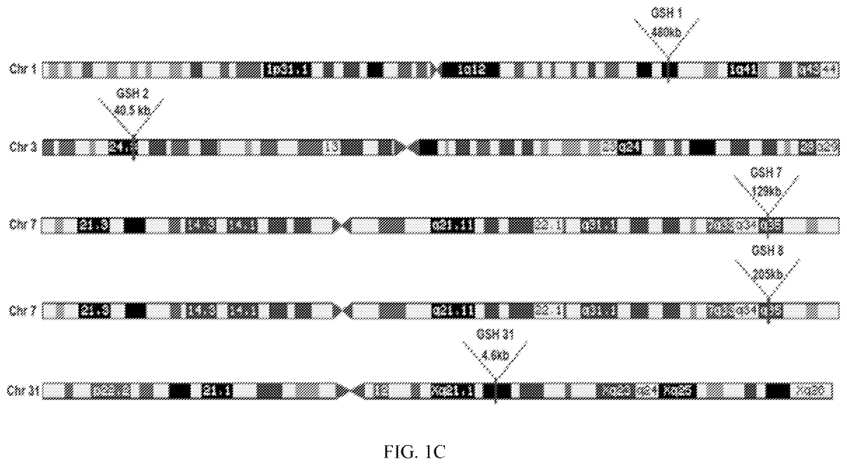

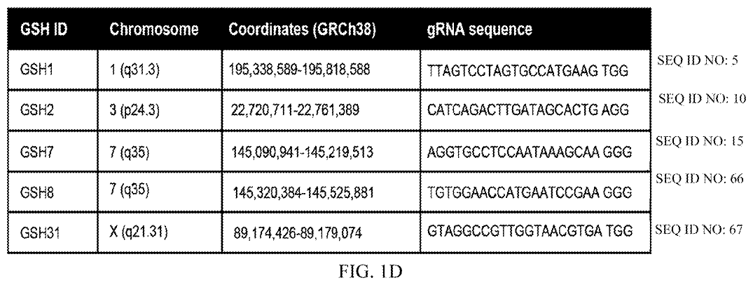

To identify novel sites that could serve as potential GSHs, a genome-wide bioinformatic search was first conducted based on previously established and widely accepted (Sadelain et al., 2012) as well as newly introduced criteria that would satisfy safe and stable gene expression (FIGS. 1A-1B). Gene-encoding sequences were eliminated and their flanking regions of 50 kb to thus avoid disruption of functional regions of gene expression. Oncogenes were identified and eliminated regions of 300 kb upstream and downstream to prevent insertional oncogenesis, a common complication of lentiviral integrations that may arise through unintended upregulation of an oncogene in the vicinity of the integration site (Hacein-Bey-Abina et al., 2008). Oncogenes from both tier 1 (extensive evidence of association with cancer available) and tier 2 (strong indications of the association exist) were used to decrease the likelihood of oncogene activation upon integration. Additionally, genes can be substantially regulated by mircoRNAs, which cleave and decay mature transcripts as well as inhibit translation machinery, thus modulating protein abundance (Filipowicz et al., 2008). Therefore, miRNA-encoding regions and 300 kb long regions around them were excluded. Apart from promoters and microRNAs, gene expression may depend on the presence of enhancers that could be located kilobases away (Schoenfelder and Fraser, 2019; Vangala et al., 2020). Enhancers as well 20 kb regions around them were excluded, which provides an overall distance of 70 kb from gene-enhancer units, decreasing the chance of altering physiological gene expression. Additionally, regions surrounding long non-coding RNAs and tRNAs were excluded as they are involved in differentiation and development programs determining cell fate and are essential for normal protein translation, respectively (Guttman et al., 2009; Chen et al., 2016; Schimmel, 2018). Finally, centromeric and telomeric regions were excluded to prevent alterations in DNA replication, cellular division and normal aging (Villasante et al., 2007).

Based on bioinformatic screening, close to two thousand sites were identified that satisfied all of the criteria (Table 1). Five sites that varied significantly in size (GSH1, 2, 7, 8, GSH31) were chosen and guide RNAs (gRNA) that showed the best scores in terms of on and off-target activities were designed and then characterized experimentally (FIGS. 1C-1D).

### Example 2. Experimental Validation of Bioinformatically Identified GSH Sites by Targeted Transgene Integration in Human Cell Lines

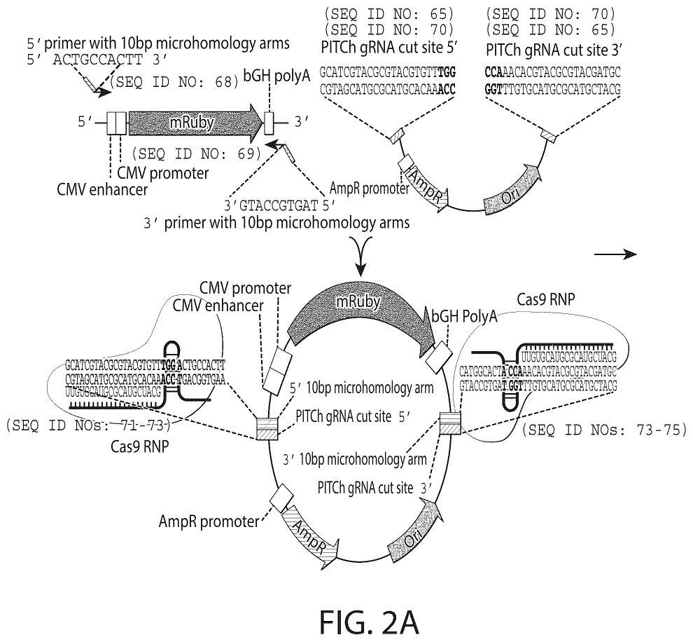

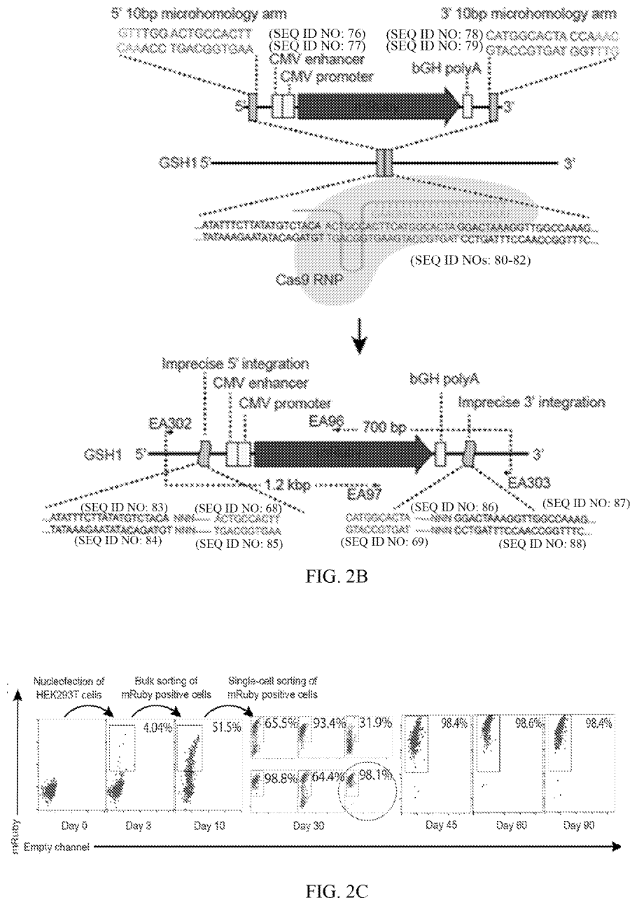

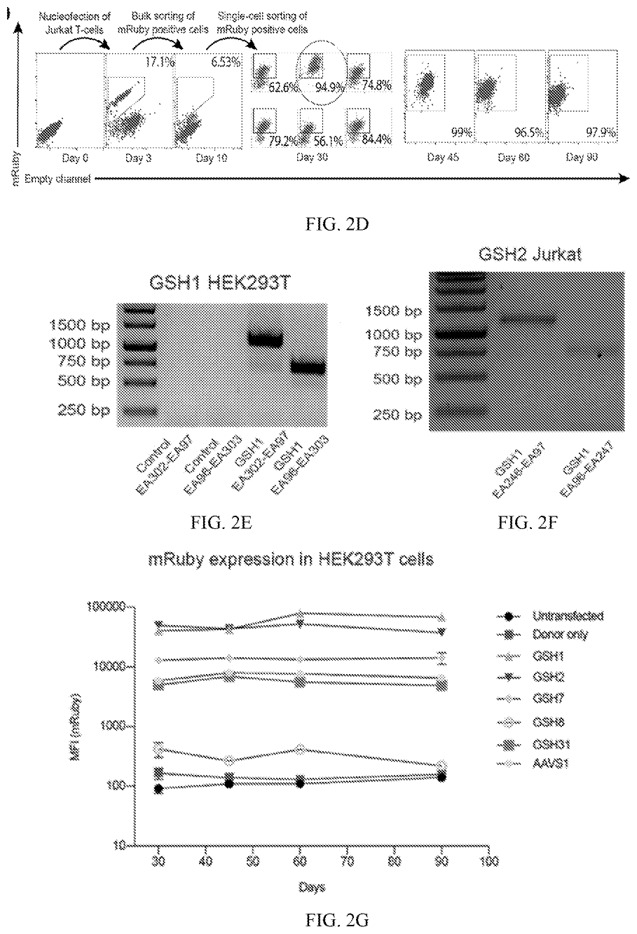

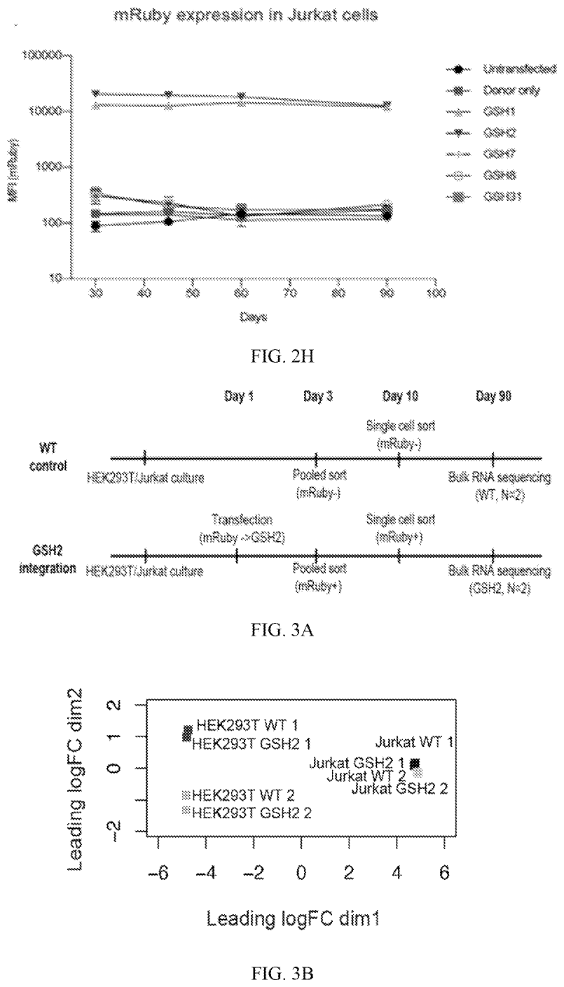

In order to experimentally assess transgene expression from the five predicted novel GSH sites, targeted integration of a gene construct encoding a red fluorescence reporter protein (mRuby) g into two common human cell lines—HEK293T and Jurkat cells was performed. HEK293 are commonly used for medium- to large-scale production of recombinant proteins (Chin et al., 2019), thus identifying GSH in HEK293 may be relevant for protein manufacturing. The Jurkat cell line was derived from T-cells of a pediatric patient with acute lymphoblastic leukemia (Abraham and Weiss, 2004) and has been used extensively for assessing the functionality of engineered immune receptors, thus discovery of GSH in this cell line supports applications in T cell therapies (Roybal et al., 2016; Vazquez-Lombardi et al., 2020). For integration of mRuby, a CRISPR/Cas9-based genome editing strategy was employed that used the Precise Integration into Target Chromosome (PITCh) method, assisted by microhomology-mediated end-joining (MMEJ) (Nakade et al., 2014; Sakuma et al., 2016; Sfeir and Symington, 2015). This approach utilizes a reporter-bearing plasmid possessing short microhomology sequences flanked by gRNA binding sites. Once inside the cells the reporter gene together with microhomologies directed against the candidate GSH site are liberated from the plasmid by Cas9-generated double-stranded breaks (DSB) at gRNA binding sites on the PITCh donor plasmid. A different gRNA-Cas9 pair generates DSBs at the candidate GSH locus, and the freed reporter gene with flanking micro-homologies is integrated by exploiting the MMEJ repair pathway (FIGS. 2A-2B). This PITCh MMEJ approach allowed us to rapidly generate donor plasmids targeted against different predicted safe harbor sites, in contrast to the more elaborate process of cloning long homology arms (i.e., >500 bp) required for homology-directed repair (HDR). The error-prone mechanism of MMEJ-mediated integration did not represent a substantial concern since the targeted sites are distanced from any identified coding or regulatory element and thus mutations arising following integration are unlikely to cause any detrimental changes.

Using the PITCh approach, mRuby transgene was transfected into the five candidate GSH sites using the best predicted gRNA sequence for each site (see Methods). A pooled selection of mRuby-expressing HEK293T and Jurkat cells was conducted by fluorescence-activated cell sorting (FACS), followed by expansion for one week and single-cell sorting to produce monoclonal populations of mRuby-expressing cells. In order to determine sites that support long-term stable transgene expression, clones with homogenous and high mRuby expression levels were monitored by performing flow cytometry at day 30, 45, 60 and 90 after integration.

Out of four candidate GSH sites, three sites in HEK293T cells—GSH1, 2 and 7 (FIGS. 2C and 2G)—and two sites in Jurkat cells—GSH1 and 2 (FIGS. 2D and 2H)—demonstrated stable mRuby expression levels 90 days after integration. Interestingly, expression from two sites in HEK293T cells—GSH1, GSH2—showed over an order of magnitude higher transgene levels than from the commonly used AAVS1 site throughout the 90-day duration of cell culture (FIG. 2G). Transgene integration into these sites was confirmed by genotyping using primer pairs amplifying the junction between tested GSH and the transgene (FIGS. 2E2F).

### Example 3. Transcriptome Profiling of Cell Lines Following Targeted Integration in GSH Sites

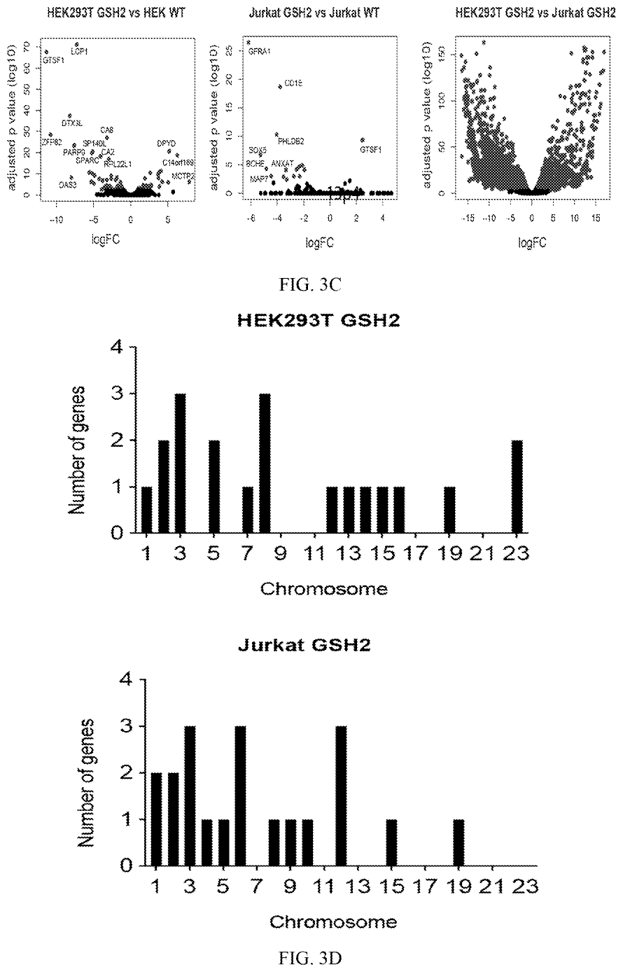

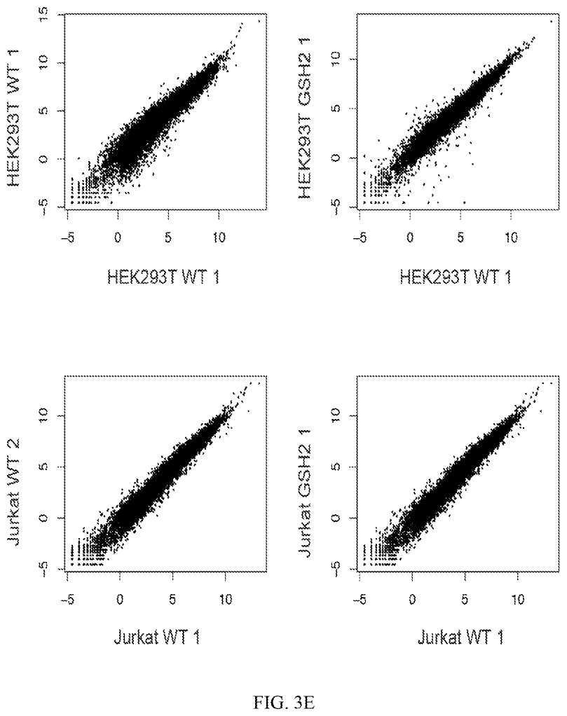

In order to assess whether targeted integration into the candidate GSH sites resulted in aberration of the global transcriptome profiles, bulk RNA-sequencing and analysis was performed. Following ninety days in culture the clone showing the highest GSH2-integrated mRuby levels was compared with untreated cells from the same culture for both HEK293T and Jurkat cells (FIG. 3A). Paired-end sequencing on Ilumina NextSeq500 with an average read length of 100 base-pairs and 30 million reads per sample was employed on two biological replicates of untreated and GSH2-mRuby cultures of HEK293T and Jurkat cells. A principal component analysis was first performed and visualized for each sample in two-dimensions using the first two principal components. This immediately revealed transcriptional similarity within the integrated and wild-type samples of the same biological replicate for both cell lines (FIG. 3B). While biological variation was observed between the HEK293T samples, the Jurkat samples, both treated and untreated, maintained conserved transcriptional profiles. Performing differential gene expression analysis revealed minor differences between integrated and unintegrated samples for both cell lines relative to the differences between the two cell types (FIG. 3C). It was additionally promising that the most differentially expressed genes were not shared between Jurkat and HEK293T cell lines, further suggesting integration in GSH2 does not systematically alter gene expression. Interestingly, differentially expressed genes were scattered across different chromosomes, again supporting that GSH2 integration does not induce systemic changes in the global transcriptomic signature (FIG. 3D). Furthermore, performing gene ontology analysis revealed no significant enrichment of cancer associated genes or pathways in both HEK and Jurkat cells (FIG. S1, S2), again supporting the potential safety of the GSH2 site. The differences in gene expression was quantified for both cell lines either across biological replicates without GSH2 integration versus within a biological replicate with or without GSH2 integration (FIG. 3E). Mirroring the principal component analysis (FIG. 3B), this analysis again supported that the differences in gene expression observed arose from biological variation between clones, not integration at GSH2.

### Example 4. Targeted Integration in Novel GSH Sites in Primary Human T-Cells and Primary Human Dermal Fibroblasts

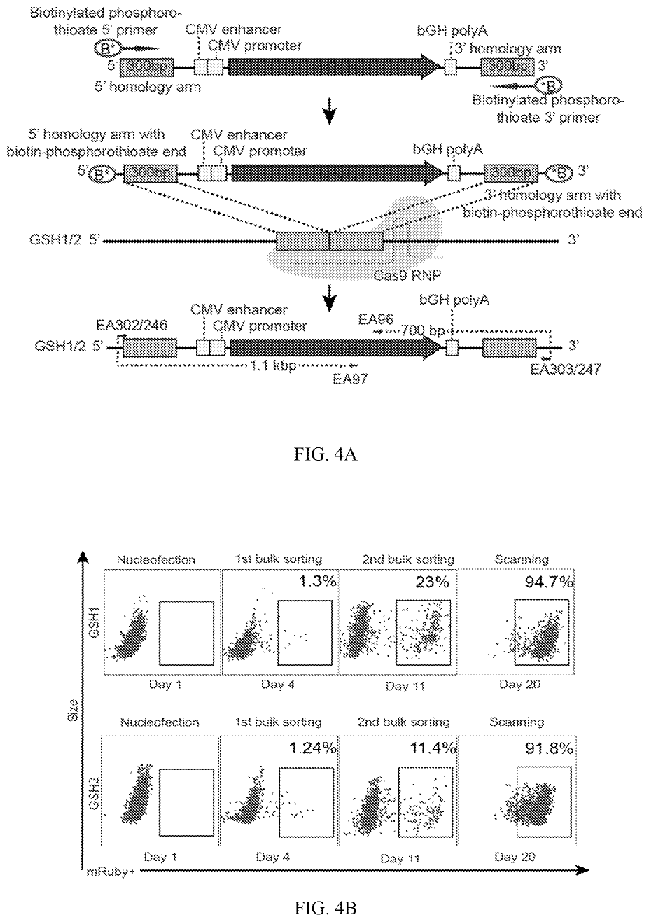

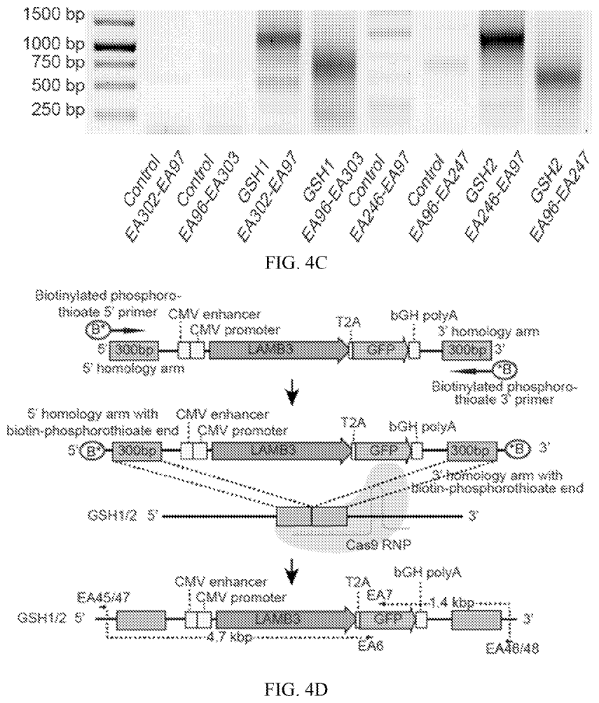

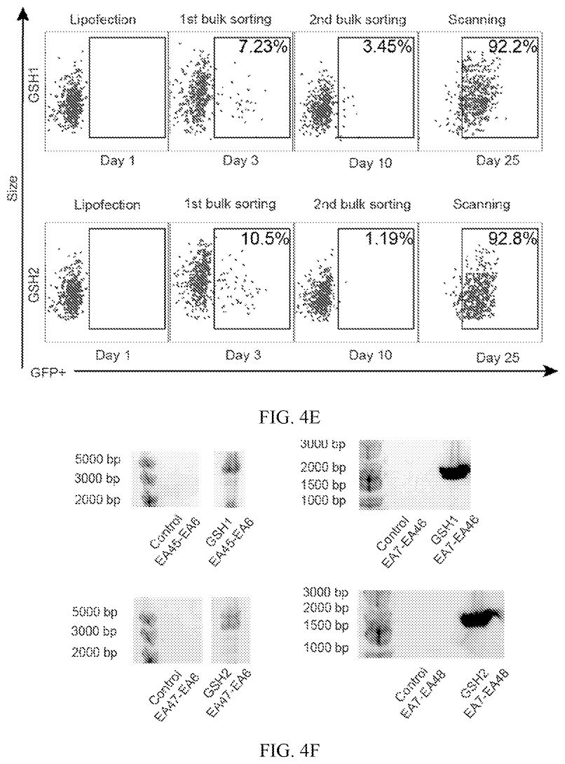

Next, targeted integration into GSH1 and GSH2 sites in primary human cells was characterized. One of the potential applications of targeted integration into novel GSH sites is for the ex-vivo engineering of human T-cells, which are being extensively explored for adoptive cell therapies in cancer and autoimmune disease. Thus, GSH1 and GSH2 were first tested in primary human T-cells isolated from peripheral blood of a healthy donor. These sites were targeted by employing an HDR-based integration approach using a linear double-stranded DNA donor template, which contained the mRuby transgene driven by a CMV promoter and with 300 bp homology arms (FIG. 4A). Phosphorothioate bonds and biotin groups were also added to 5′ and 3′ ends of the HDR template to increase its stability and prevent concatemerization, respectively (Gutierrez-Triana et al., 2018). Nucleofection of Cas9-gRNA ribonucleoprotein (RNP) complexes and HDR templates into primary T-cells resulted in mRuby-positive expression in 1.3% of cells for GSH1 and 1.24% of cells for GSH2. These mRuby-expressing cells were isolated by FACS on day three, cultured for another seven days; a second round of sorting was performed on the mRuby-positive populations. Following these two rounds of pooled sorting, a highly enriched population of T cells stably expressing the mRuby transgene was isolated and cultured for the duration of the experiment (up to day 20), with mRuby expression from GSH1 and GSH2 in 94.7% and 91.8% of cells, respectively (FIG. 4B). Correct integration into GSH1 and GSH2 was confirmed by genotyping and Sanger-sequencing using primers amplifying the junction between GSH1/GSH2 loci and the mRuby donor (FIG. 4C).

Another possible ex-vivo application of identified GSH sites includes engineering dermal fibroblasts and keratinocytes for autologous skin grafting in people with burns or inherited skin disorders. A group of genetic skin disorders named junctional epidermolysis bullosa (JEB) is associated primarily with mutations in a family of multi-subunit laminin proteins, which are involved in anchoring the epidermis layer of the skin to derma (Bardhan et al., 2020). Certain variants of JEB are specifically related to mutations in a beta subunit of laminin-5 protein, encoded by the LAMB3 gene (Robbins et al., 2001). Using a similar dsDNA HDR donor with 300 bp homology arms possessing phosphorothioate bond and biotin, Cas9 HDR was used to integrate the LAMB3 gene tagged with GFP (total insert size 5409 bp) into GSH1 and GSH2 sites in primary human dermal fibroblasts isolated from neonatal skin (FIG. 4D). After lipofection of fibroblasts with Cas9 and HDR templates, expression of GFP, which is indicative of LAMB3 expression, was observed in 7.23% (GSH1) and 10.5% (GSH2) of cells. These cells were sorted at day three, cultured for seven days and the GFP-positive population—3.45% for GSH1 and 1.19% for GSH2—was sorted again. Similar to T-cells, two rounds of pooled sorting led to over 92% enrichment of GFP-positive cells, with the expression of LAMB3-GFP transgene maintained for over 25 days (FIG. 4E). Genotyping and Sanger-sequencing confirmed successful integration into both loci by using primers amplifying the junction between GSH1/GSH2 and the LAMB3-GFP donor (FIG. 4F).

### Example 5. Single-Cell RNA Sequencing and Analysis of Primary Human T Cells Following Transgene Integration into a Novel GSH Site

Lastly, transcriptome-wide effects on a single-cell level following transgene integration into GSH1 in primary T-cells was assessed. Single-cell RNA sequencing was performed using the 10× Genomics protocol, which consists of encapsulating cells in gel beads bearing reverse transcription (RT) reaction mix with unique cell primers. Following the RT reaction, the cDNA is pooled, and the library is amplified for subsequent next-generation sequencing.

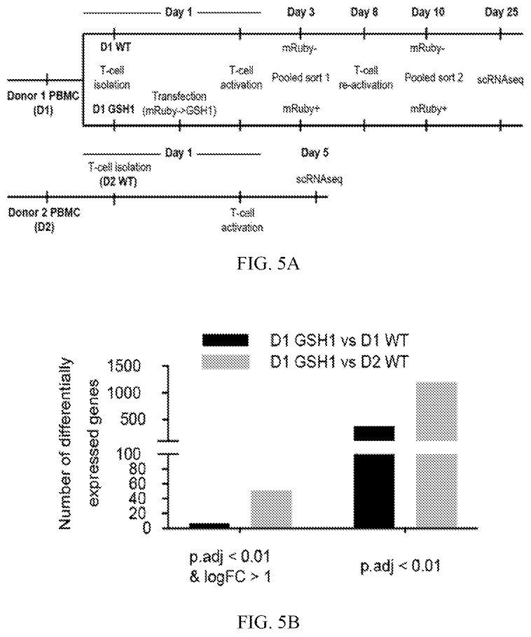

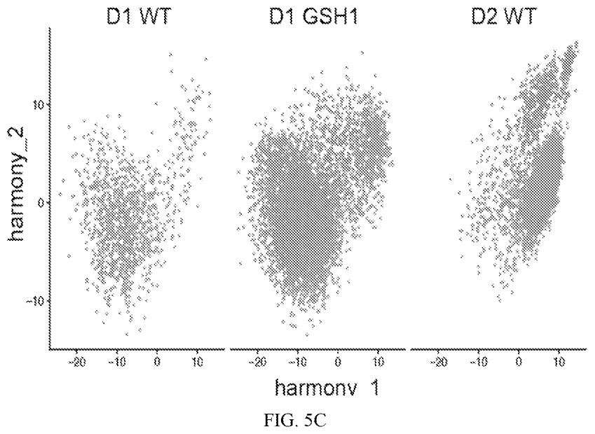

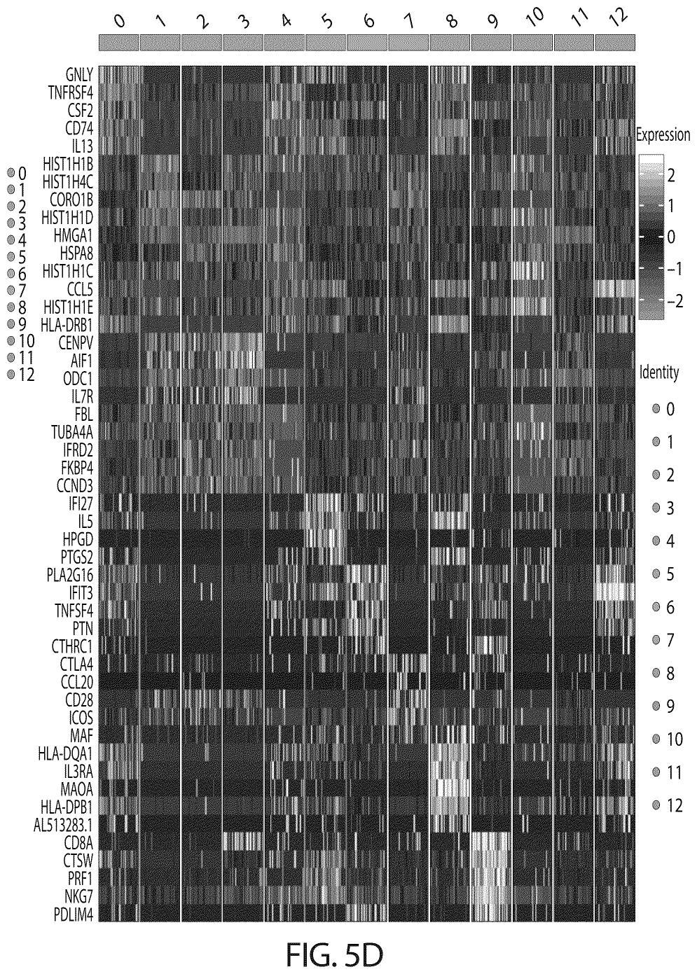

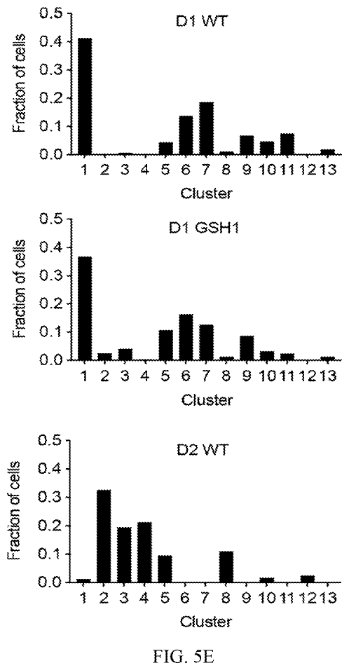

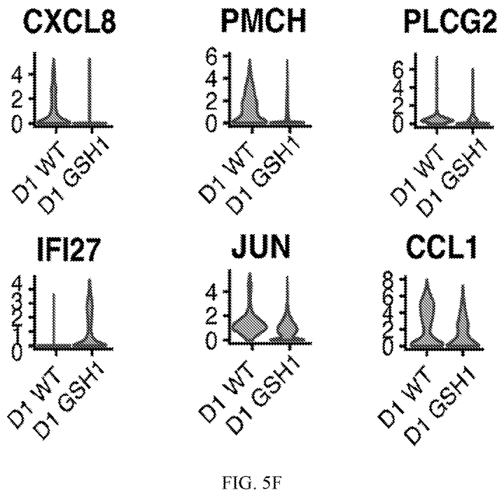

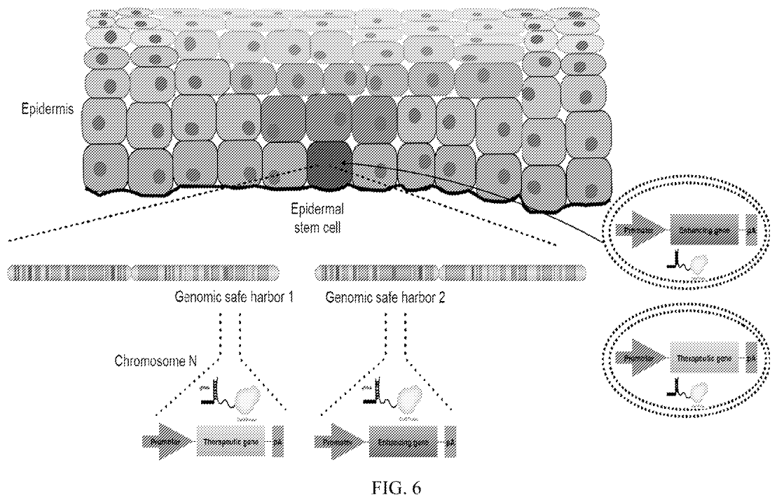

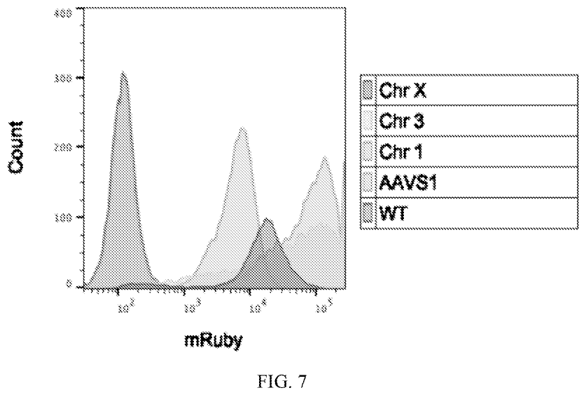

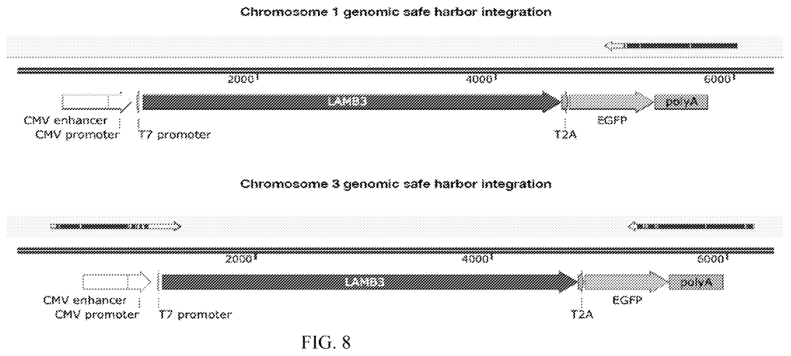

This single-cell sequencing workflow was applied to human T cells expressing mRuby in GSH1 after 25 days in culture, wildtype (non-transfected) cells were used as a control. These cells were also compared with wild-type controls from a different donor to again compare whether GSH integration resulted in more variability in gene expression relative to a biological replicate (FIG. 5A). Performing differential gene expression analysis across the three samples revealed fewer up- or downregulated genes following GSH1 integration relative to the untreated, second patient sample (FIG. 5B). Uniform manifold approximation projection (UMAP) paired with an unbiased clustering based on global gene expression were performed, which resulted in 13 distinct clusters (FIG. 5C). Many genes defining these clusters corresponded to typical T cell markers such as IL7R, ICOS, CD28, CCLS, CD74, and NKG7 (FIG. 5D). The proportion of cells per cluster for each sample was subsequently quantified, again demonstrating congruent gene expression signatures from cells arising from a single patient, regardless of whether integration in GSH1 occurred or not (FIG. 5E). Furthermore, similar to bulk RNA-sequencing results on cell lines, none of the most differentially expressed genes that were upregulated in cells with GSH1 transgene integration were associated with any cancer-related pathways (FIG. 5F). Interestingly, the expression of the Jun gene encoding the oncogenic c-Jun transcription factor is decreased in cells bearing transgene integration into GSH1. Taken together, both our single-cell and bulk RNA-sequencing data suggest that the computationally determined and experimentally validated GSHs have minimal influences on global gene expression.

### Example 6. Targeted Integration in GSH1 and GSH2 Sites in Human iPSCs

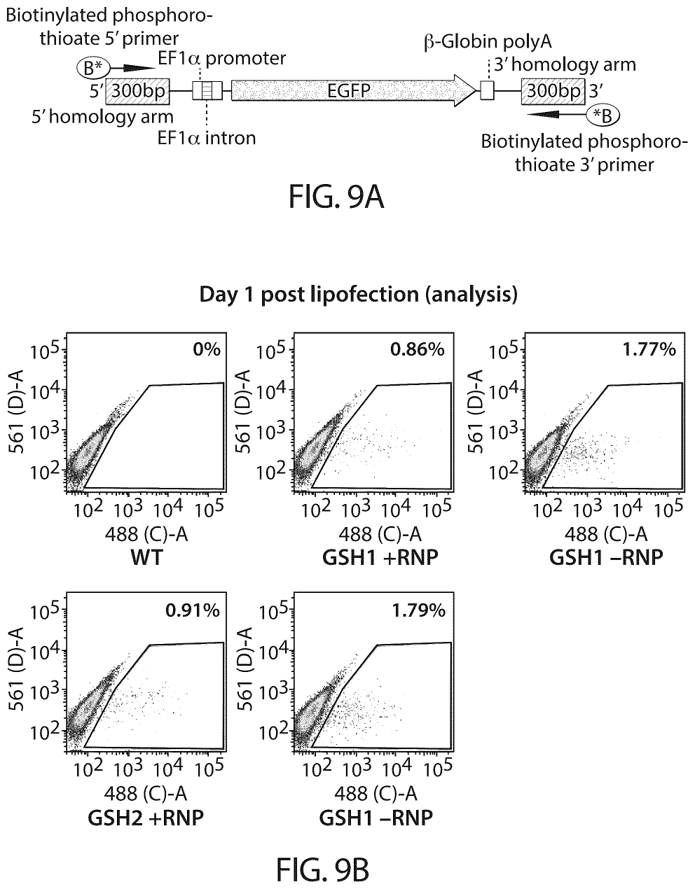

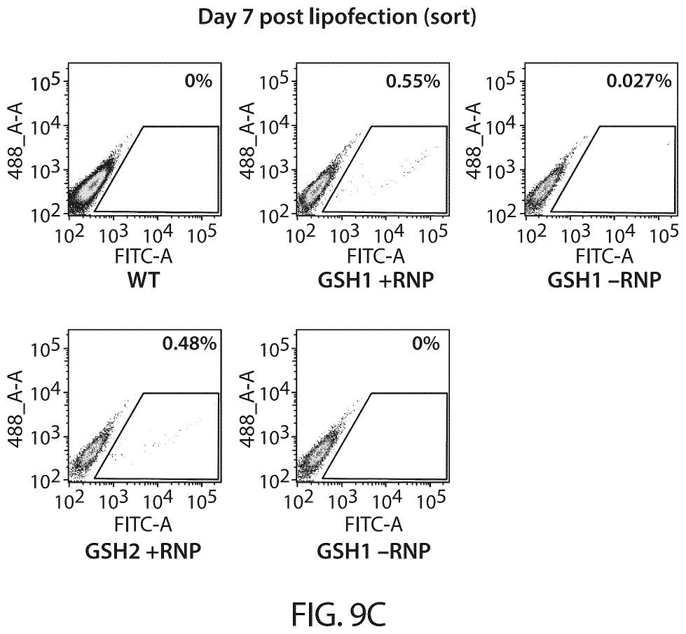

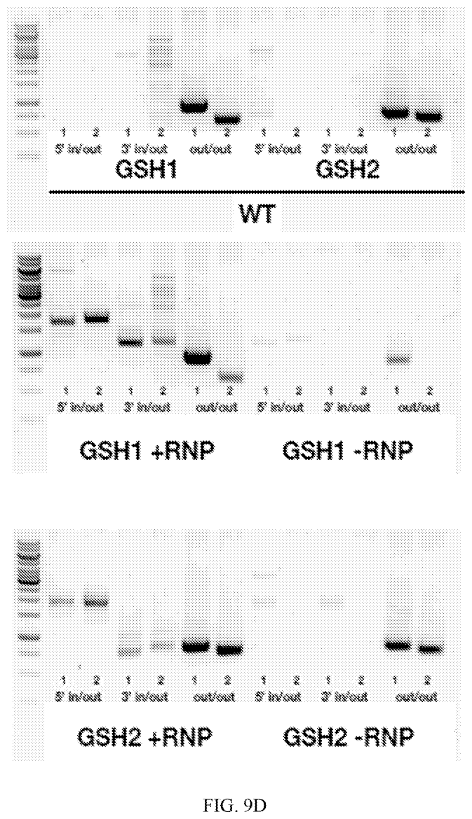

Next, targeted integration into GSH1 and GSH2 sites in human induced pluripotent stem cells (iPSCs) was characterized. These sites were targeted by employing an HDR-based integration approach using a linear double-stranded DNA donor template, which contained the eGFP transgene driven by an EF1α promoter and with 300 bp homology arms (FIG. 9A). Phosphorothioate bonds and biotin groups were also added to 5′ and 3′ ends of the HDR template to increase its stability and prevent concatemerization, respectively (Gutierrez-Triana et al., 2018). Nucleofection of Cas9-gRNA ribonucleoprotein (RNP) complexes and HDR templates into human iPSCs resulted in eGFP-positive expression in 0.86% of cells for GSH1 on Day 1 and 0.55% of cells for GSH1 on Day 7, and 0.91% of cells for GSH2 on Day 1 and 0.48% of cells for GSH2 on Day 7 (FIGS. 9B-9C). Importantly, GFP expression is still detectable on Day 7. Correct integration into GSH1 and GSH2 was confirmed by genotyping and Sanger-sequencing using primers amplifying the junction between GSH1/GSH2 loci and the eGFP donor (FIG. 9D).

### Methods

### Computational Search for GSH Sites

Previously established criteria (Sadelain et al., 2012) as well as newly introduced ones were used to predict genomic locations of novel GSHs. Specifically, coordinates of all known genes were extracted from GENCODE gene annotation (Release 24). A set of tier 1 and tier 2 oncogenes was obtained from Cancer Gene Census. The miRNA coordinates were obtained from MirGeneDB (Fromm et al., 2020). Enhancer regions were obtained from the EnhancerAtlas 2.0 database (Gao and Qian, 2019), coordinates were transposed into GRCh38/hg38 genome and union of enhancer sites was used. Genomic locations of sequences of tRNA and lncRNA were extracted from GENCODE gene annotation (Release 24). UCSC genome browser GRCh38/hg38 was used to get coordinates of telomeres and centromeres as well as unannotated regions. BEDTools (Quinlan and Hall, 2010) were used to determine flanking regions of each element of the criteria as well as to obtain union or difference between sets of coordinates. The source code for computational identification of novel safe harbors is available at https://github.com/elvirakinzina/GSH.

### Plasmids and HDR Donor Generation

PITCh plasmids were generated through standard cloning methods. CMV-mRuby-bGH insert was amplified from pcDNA3-mRuby2 plasmid (Addgene, Plasmid #40260) with primers containing mircohomology sequences against specific GSH and AAVS1 site with 10 bp of overlapping ends for the pcDNA3 backbone. The pcDNA3 backbone was amplified with primers containing sequences of PITCh gRNA cut site (GCATCGTACGCGTACGTGTTTGG SEQ ID NO: 65) on both 5′ and 3′ ends of the backbone. The insert and the backbone were assembled using Gibson Assembly Master Mix (New England Biolabs, #E2611L).

Plasmids encoding CMV-mRuby-bGH flanked by GSH1/GSH2 300 bp homology arms were ordered from Twist Biosciences in pENTR vector. HDR donors were amplified from these plasmids using biotinylated primers with phosphorothioate bonds between the first 5 nucleotides on both 5′ and 3′ ends. Plasmid encoding CMV-LAMB3-T2A-GFP-bGH was generated by overlap extension PCR of LAMB3 cDNA, purchased from Genscript (NM_000228.3), and GFP-bGH sequence from Addgene (Plasmid #11154). T2A sequence was added to 5′primer of GFP-bGH. Produced insert was cloned into pENTR vector from Twist Biosciences bearing GSH1 and GSH2 300 bp homology arms using Gibson Assembly Master Mix (NEB, #E2611L). HDR donors were amplified from these plasmids using biotinylated primers with phosphorothioate bonds between the first 5 nucleotides on both 5′ and 3′ ends. HDR donors were then purified from PCR mix using SPRI beads (Beckman Coulter, #B23318) at 0.4× beads to PCR mix ratio.

TABLE 4
HDR Donor Constructs

| Donor name | Donor sequence |
|---|---|
| GSH1 CMV-mRuby | CTGCATTTAAGTAGGATTCAATAATTTTAAAGTGCAGGGACAAAATTTCCTCATATGGCTC |
| | ACTAGCTACATTGCAAATTTCTTGAAATCAGAACACAGAAGTGCAGTCCTGTGCTCGCAAT |
| | GCAGACTTGCAGGGTGTAGAGGCATAAATGGCTCCAGAGCCAGGGACATGGGTCCAGAGGG |
| | GGGTAGTCTCCAGAAGACTCCTTTCGGGCCTATTACCATGCCTCAGAGGTCCAAGTGGGGC |
| | ATGGTGAATATATTATCCTTTATATTATATTTCTTATATGTCTACAACTGCCACTTGACAT |
| | TGATTATTGACTAGTTATTAATAGTAATCAATTACGGGGTCATTAGTTCATAGCCCATATA |
| | TGGAGTTCCGCGTTACATAACTTACGGTAAATGGCCCGCCTGGCTGACCGCCCAACGACCC |
| | CCGCCCATTGACGTCAATAATGACGTATGTTCCCATAGTAACGCCAATAGGGACTTTCCAT |
| | TGACGTCAATGGGTGGACTATTTACGGTAAACTGCCCACTTGGCAGTACATCAAGTGTATC |
| | ATATGCCAAGTACGCCCCCTATTGACGTCAATGACGGTAAATGGCCCGCCTGGCATTATGC |
| | CCAGTACATGACCTTATGGGACTTTCCTACTTGGCAGTACATCTACGTATTAGTCATCGCT |
| | ATTACCATGGTGATGCGGTTTTGGCAGTACATCAATGGGCGTGGATAGCGGTTTGACTCAC |
| | GGGGATTTCCAAGTCTCCACCCCATTGACGTCAATGGGAGTTTGTTTTGGCACCAAAATCA |
| | ACGGGACTTTCCAAAATGTCGTAACAACTCCGCCCCATTGACGCAAATGGGCGGTAGGCGT |
| | GTACGGTGGGAGGTCTATATAAGCAGAGCTCTCTGGCTAACTAGAGAACCCACTGCTTACT |
| | GGCTTATCGAAATTAATACGACTCACTATAGGGAGACCCAAGCTTGCGGCCGCCACCATGG |
| | TGCGGGGTTCTCATCATCATCATCATCATGGTATGGCTAGCATGACTGGTGGACAGCAAAT |
| | GGGTCGGGATCTGTACGACGATGACGATAAGGATCCGATGGTGTCTAAGGGCGAAGAGCTG |
| | ATCAAGGAAAATATGCGTATGAAGGTGGTCATGGAAGGTTCGGTCAACGGCCACCAATTCA |
| | AATGCACAGGTGAAGGAGAAGGCAATCCGTACATGGGAACTCAAACCATGAGGATCAAAGT |
| | CATCGAGGGAGGACCCCTGCCATTTGCCTTTGACATTCTTGCCACGTCGTTCATGTATGGC |
| | AGCCGTACTTTTATCAAGTACCCGAAAGGCATTCCTGATTTCTTTAAACAGTCCTTTCCTG |
| | AGGGTTTTACTTGGGAAAGAGTTACGAGATACGAAGATGGTGGAGTCGTCACCGTCATGCA |
| | GGACACCAGCCTTGAGGATGGCTGTCTCGTTTACCACGTCCAAGTCAGAGGGGTAAACTTT |
| | CCCTCCAATGGTCCCGTGATGCAGAAGAAGACCAAGGGTTGGGAGCCTAATACAGAGATGA |
| | TGTATCCAGCAGATGGTGGTCTGAGGGGATACACTCATATGGCACTGAAAGTTGATGGTGG |
| | TGGCCATCTGTCTTGCTCTTTCGTAACAACTTACAGGTCAAAAAAGACCGTCGGGAACATC |
| | AAGATGCCCGGTATCCATGCCGTTGATCACCGCCTGGAAAGGTTAGAGGAAAGTGACAATG |
| | AAATGTTCGTAGTACAACGCGAACACGCAGTTGCCAAGTTCGCCGGGCTTGGTGGTGGGAT |
| | GGACGAGCTGTACAAGTAAGAATTCTGCAGATATCCATCACACTGGCGGCCGCTCGAGCAT |
| | GCATCTAGAGGGCCCTATTCTATAGTGTCACCTAAATGCTAGAGCTCGCTGATCAGCCTCG |
| | ACTGTGCCTTCTAGTTGCCAGCCATCTGTTGTTTGCCCCTCCCCCGTGCCTTCCTTGACCC |
| | TGGAAGGTGCCACTCCCACTGTCCTTTCCTAATAAAATGAGGAAATTGCATCGCATTGTCT |
| | GAGTAGGTGTCATTCTATTCTGGGGGGGGGGTGGGGCAGGACAGCAAGGGGGAGGATTGG |
| | GAAGACAATAGCAGGCATGCTGGGGATGCGGTGGGCTCTATGGCATGGCACTAGGACTAAA |
| | GGTTGGCCAAAGTACAAGATATTTGTCTTATCTGATGACAACTCTGTGTCCTGGACTCTCT |
| | TCCAGAATAAGACCTTTCCTGCAGCACTGCTTGAACTCCTCTTAGCAAGAGGGAAACATGT |
| | GAAATGCTACCAAAATAGAATAGAAGTAAATTCTTATTATATTCCTTTGTTCACTCATATC |
| | CTGAAGTGCATCAAATCAGGTTTTCTCACCTGTATAATGCTGTATTTTACTTGAGTTGGAA |
| | TAATTTTGCTTAGAAATAAATAAGTAAAACAGCACCTG (SEQ ID NO: 1) |
| GSH2 CMV-mRuby | CATTACATCCAAGTTTAGACTCATTGAGCTCTAAATATTTGGGAAAACATATTTAAAGAA |
| | ATTATATAGGTTTGATCCAAAATCTCTTTGGCACAACTTGAAATATGGGTAATCGTCATG |
| | TGAAATTTGTGAATAGGAGAACCCACTGTAGGATACTTAACATAAATCAGCCACATAATT |
| | TCTATCACTGATATCCAGGGAATTTCAATGACAAATCTAGTGATAAAAATTGATAAAACA |
| | TTTTTGATAGTTTTGATACAAGTGAAAGTCATGGGATATCAGACTTAAAAGAAACCTCAG |
| | GACATTGATTATTGACTAGTTATTAATAGTAATCAATTACGGGGTCATTAGTTCATAGCC |
| | CATATATGGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCAATAGGGA |
| | CTTTCCATTGACGTCAATGGGTGGACTATTTACGGTAAACTGCCCACTTGGCAGTACATC |
| | AAGTGTATCATATGCCAAGTACGCCCCCTATTGACGTCAATGACGGTAAATGGCCCGCCT |
| | GGCATTATGCCCAGTACATGACCTTATGGGACTTTCCTACTTGGCAGTACATCTACGTAT |
| | TAGTCATCGCTATTACCATGGTGATGCGGTTTTGGCAGTACATCAATGGGCGTGGATAGC |
| | GGTTTGACTCACGGGGATTTCCAAGTCTCCACCCCATTGACGTCAATGGGAGTTTGTTTT |
| | GGCACCAAAATCAACGGGACTTTCCAAAATGTCGTAACAACTCCGCCCCATTGACGCAAA |
| | TGGGCGGTAGGCGTGTACGGTGGGAGGTCTATATAAGCAGAGCTCTCTGGCTAACTAGAG |
| | AACCCACTGCTTACTGGCTTATCGAAATTAATACGACTCACTATAGGGAGACCCAAGCTT |
| | GCGGCCGCCACCATGGTGCGGGGTTCTCATCATCATCATCATCATGGTATGGCTAGCATG |
| | ACTGGTGGACAGCAAATGGGTCGGGATCTGTACGACGATGACGATAAGGATCCGATGGTG |
| | TCTAAGGGCGAAGAGCTGATCAAGGAAAATATGCGTATGAAGGTGGTCATGGAAGGTTCG |
| | GTCAACGGCCACCAATTCAAATGCACAGGTGAAGGAGAAGGCAATCCGTACATGGGAACT |
| | CAAACCATGAGGATCAAAGTCATCGAGGGAGGACCCCTGCCATTTGCCTTTGACATTCTT |
| | GCCACGTCGTTCATGTATGGCAGCCGTACTTTTATCAAGTACCCGAAAGGCATTCCTGAT |
| | TTCTTTAAACAGTCCTTTCCTGAGGGTTTTACTTGGGAAAGAGTTACGAGATACGAAGAT |
| | GGTGGAGTCGTCACCGTCATGCAGGACACCAGCCTTGAGGATGGCTGTCTCGTTTACCAC |
| | GTCCAAGTCAGAGGGGTAAACTTTCCCTCCAATGGTCCCGTGATGCAGAAGAAGACCAAG |
| | GGTTGGGAGCCTAATACAGAGATGATGTATCCAGCAGATGGTGGTCTGAGGGGATACACT |
| | CATATGGCACTGAAAGTTGATGGTGGTGGCCATCTGTCTTGCTCTTTCGTAACAACTTAC |
| | AGGTCAAAAAAGACCGTCGGGAACATCAAGATGCCCGGTATCCATGCCGTTGATCACCGC |
| | CTGGAAAGGTTAGAGGAAAGTGACAATGAAATGTTCGTAGTACAACGCGAACACGCAGTT |
| | GCCAAGTTCGCCGGGCTTGGTGGTGGGATGGACGAGCTGTACAAGTAAGAATTCTGCAGA |
| | TATCCATCACACTGGCGGCCGCTCGAGCATGCATCTAGAGGGCCCTATTCTATAGTGTCA |
| | CCTAAATGCTAGAGCTCGCTGATCAGCCTCGACTGTGCCTTCTAGTTGCCAGCCATCTGT |
| | TGTTTGCCCCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTCTGGGGGG |
| | TGGGGTGGGGCAGGACAGCAAGGGGGAGGATTGGGAAGACAATAGCAGGCATGCTGGGGA |
| | TGCGGTGGGCTCTATGGTGCTATCAAGTCTGATGTCAGTAATTTTTGGAGGAGACTGAAG |
| | TGCAGTGAGACTATCCAAAGTCAGACATGGGGAAAAGCAGAGTCATCCCTCCTAGGCTGC |
| | CAAAATCCTCCCCATCCAAGCTCATCCTTGAAGCCCTCACTTAAGACAAAGTTCCTCCCA |
| | TCCCTTCTGCCTGCTCTGGCATGGTCTGAACCATTTGCCTATTAATTGCCCTGCCTGGTT |
| | TCATTTGTTCTTTTTGCTGTATTTAAACTGTGGGAATTCTATTGTTAACCTTTTTCTTGC |
| | TCAACTGAACTGTGACA (SEQ ID NO: 2) |
| GSH1 | CTGCATTTAAGTAGGATTCAATAATTTTAAAGTGCAGGGACAAAATTTCCTCATATGGCT |
| CMV-LAMB3- | CACTAGCTACATTGCAAATTTCTTGAAATCAGAACACAGAAGTGCAGTCCTGTGCTCGCA |
| T2A-GFP | ATGCAGACTTGCAGGGTGTAGAGGCATAAATGGCTCCAGAGCCAGGGACATGGGTCCAGA |
| | GGGGGGTAGTCTCCAGAAGACTCCTTTCGGGCCTATTACCATGCCTCAGAGGTCCAAGTG |
| | GGGCATGGTGAATATATTATCCTTTATATTATATTTCTTATATGTCTACAACTGCCACTT |
| | GACATTGATTATTGACTAGTTATTAATAGTAATCAATTACGGGGTCATTAGTTCATAGCC |
| | CATATATGGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCAATAGGGA |
| | CTTTCCATTGACGTCAATGGGTGGACTATTTACGGTAAACTGCCCACTTGGCAGTACATC |
| | AAGTGTATCATATGCCAAGTACGCCCCCTATTGACGTCAATGACGGTAAATGGCCCGCCT |
| | GGCATTATGCCCAGTACATGACCTTATGGGACTTTCCTACTTGGCAGTACATCTACGTAT |
| | TAGTCATCGCTATTACCATGGTGATGCGGTTTTGGCAGTACATCAATGGGCGTGGATAGC |
| | GGTTTGACTCACGGGGATTTCCAAGTCTCCACCCCATTGACGTCAATGGGAGTTTGTTTT |
| | GGCACCAAAATCAACGGGACTTTCCAAAATGTCGTAACAACTCCGCCCCATTGACGCAAA |
| | TGGGCGGTAGGCGTGTACGGTGGGAGGTCTATATAAGCAGAGCTCTCTGGCTAACTAGAG |
| | AACCCACTGCTTACTGGCTTATCGAAATTAATACGACTCACTATAGGGAGACCCAAGCTT |
| | GCGGCCGCCACCatgagaccattcttcctcttgtgttttgccctgcctggcctcctgcat |
| | gcccaacaagcctgctcccgtggggcctgctatccacctgttggggacctgcttgttggg |
| | aggacccggtttctccgagcttcatctacctgtggactgaccaagcctgagacctactgc |
| | acccagtatggcgagtggcagatgaaatgctgcaagtgtgactccaggcagcctcacaac |
| | tactacagtcaccgagtagagaatgtggcttcatcctccggccccatgcgctggtggcag |
| | tcccagaatgatgtgaaccctgtctctctgcagctggacctggacaggagattccagctt |
| | caagaagtcatgatggagttccaggggcccatgcctgccggcatgctgattgagcgctcc |
| | tcagacttcggtaagacctggcgagtgtaccagtacctggctgccgactgcacctccacc |
| | ttccctcgggtccgccagggtcggcctcagagctggcaggatgttcggtgccagtccctg |
| | cctcagaggcctaatgcacgcctaaatggggggaaggtccaacttaaccttatggattta |
| | gtgtctgggattccagcaactcaaagtcaaaaaattcaagaggtgggggagatcacaaac |
| | ttgagagtcaatttcaccaggctggcccctgtgccccaaaggggctaccaccctcccagc |
| | gcctactatgctgtgtcccagctccgtctgcaggggagctgcttctgtcacggccatgct |
| | gatcgctgcgcacccaagcctggggcctctgcaggcccctccaccgctgtgcaggtccac |
| | gatgtctgtgtctgccagcacaacactgccggcccaaattgtgagcgctgtgcacccttc |
| | tacaacaaccggccctggagaccggcggagggccaggacgcccatgaatgccaaaggtgc |
| | gactgcaatgggcactcagagacatgtcactttgaccccgctgtgtttgccgccagccag |
| | ggggcatatggaggtgtgtgtgacaattgccgggaccacaccgaaggcaagaactgtgag |
| | cggtgtcagctgcactatttccggaaccggcgcccgggagcttccattcaggagacctgc |
| | atctcctgcgagtgtgatccggatggggcagtgccaggggctccctgtgacccagtgacc |
| | gggcagtgtgtgtgcaaggagcatgtgcagggagagcgctgtgacctatgcaagccgggc |
| | ttcactggactcacctacgccaacccgcagggctgccaccgctgtgactgcaacatcctg |
| | gggtcccggagggacatgccgtgtgacgaggagagtgggcgctgcctttgtctgcccaac |
| | gtggtgggtcccaaatgtgaccagtgtgctccctaccactggaagctggccagtggccag |
| | ggctgtgaaccgtgtgcctgcgacccgcacaactccctcagcccacagtgcaaccagttc |
| | acagggcagtgcccctgtcgggaaggctttggtggcctgatgtgcagcgctgcagccatc |
| | cgccagtgtccagaccggacctatggagacgtggccacaggatgccgagcctgtgactgt |
| | gatttccggggaacagagggcccgggctgcgacaaggcatcaggccgctgcctctgccgc |
| | cctggcttgaccgggccccgctgtgaccagtgccagcgaggctactgcaatcgctacccg |
| | gtgtgcgtggcctgccacccttgcttccagacctatgatgcggacctccgggagcaggcc |
| | ctgcgctttggtagactccgcaatgccaccgccagcctgtggtcagggcctgggctggag |
| | gaccgtggcctggcctcccggatcctagatgcaaagagtaagattgagcagatccgagca |
| | gttctcagcagccccgcagtcacagagcaggaggtggctcaggtggccagtgccatcctc |
| | tccctcaggcgaactctccagggcctgcagctggatctgcccctggaggaggagacgttg |
| | tcccttccgagagacctggagagtcttgacagaagcttcaatggtctccttactatgtat |
| | cagaggaagagggagcagtttgaaaaaataagcagtgctgatccttcaggagccttccgg |
| | atgctgagcacagcctacgagcagtcagcccaggctgctcagcaggtctccgacagctcg |
| | cgccttttggaccagctcagggacagccggagagaggcagagaggctggtgcggcaggcg |
| | ggaggaggaggaggcaccggcagccccaagcttgtggccctgaggctggagatgtcttcg |
| | ttgcctgacctgacacccaccttcaacaagctctgtggcaactccaggcagatggcttgc |
| | accccaatatcatgccctggtgagctatgtccccaagacaatggcacagcctgtggctcc |
| | cgctgcaggggtgtccttcccagggccggtggggccttcttgatggcggggcaggtggct |
| | gagcagctgcggggcttcaatgcccagctccagcggaccaggcagatgattagggcagcc |
| | gaggaatctgcctcacagattcaatccagtgcccagcgcttggagacccaggtgagcgcc |
| | agccgctcccagatggaggaagatgtcagacgcacacggctcctaatccagcaggtccgg |
| | gacttcctaacagaccccgacactgatgcagccactatccaggaggtcagcgaggccgtg |
| | ctggccctgtggctgcccacagactcagctactgttctgcagaagatgaatgagatccag |
| | gccattgcagccaggctccccaacgtggacttggtgctgtcccagaccaagcaggacatt |
| | gcgcgtgcccgccggttgcaggctgaggctgaggaagccaggagccgagcccatgcagtg |
| | gagggccaggtggaagatgtggttgggaacctgcggcaggggacagtggcactgcaggaa |
| | gctcaggacaccatgcaaggcaccagccgctcccttcggcttatccaggacagggttgct |
| | gaggttcagcaggtactgcggccagcagaaaagctggtgacaagcatgaccaagcagctg |
| | ggtgacttctggacacggatggaggagctccgccaccaagcccggcagcagggggcagag |
| | gcagtccaggcccagcagcttgcggaaggtgccagcgagcaggcattgagtgcccaagag |
| | ggatttgagagaataaaacaaaagtatgctgagttgaaggaccggttgggtcagagttcc |
| | atgctgggtgagcagggtgcccggatccagagtgtgaagacagaggcagaggagctgttt |
| | ggggagaccatggagatgatggacaggatgaaagacatggagttggagctgctgcggggc |
| | agccaggccatcatgctgcgctcggcggacctgacaggactggagaagcgtgtggagcag |
| | atccgtgaccacatcaatgggcgcgtgctctactatgccacctgcaagGAGGGCAGAGGA |
| | AGTCTTCTAACATGCGGTGACGTGGAGGAGAATCCCGGCCCTAGCatggtgagcaagggc |
| | gaggagctgttcaccggggtggtgcccatcctggtcgagctggacggcgacgtaaacggc |
| | cacaagttcagcgtgtccggcgagggcgagggcgatgccacctacggcaagctgaccctg |
| | aagttcatctgcaccaccggcaagctgcccgtgccctggcccaccctcgtgaccaccctg |
| | acctacggcgtgcagtgcttcagccgctaccccgaccacatgaagcagcacgacttcttc |
| | aagtccgccatgcccgaaggctacgtccaggagcgcaccatcttcttcaaggacgacggc |
| | aactacaagacccgcgccgaggtgaagttcgagggcgacaccctggtgaaccgcatcgag |
| | ctgaagggcatcgacttcaaggaggacggcaacatcctggggcacaagctggagtacaac |
| | tacaacagccacaacgtctatatcatggccgacaagcagaagaacggcatcaaggtgaac |
| | ttcaagatccgccacaacatcgaggacggcagcgtgcagctcgccgaccactaccagcag |
| | aacacccccatcggcgacggccccgtgctgctgcccgacaaccactacctgagcacccag |
| | tccgccctgagcaaagaccccaacgagaagcgcgatcacatggtcctgctggagttcgtg |
| | accgccgccgggatcactctcggcatggacgagctgtacaagtaaagcggactagtctag |
| | caatcaacctctggattacaaaatttgtgaaagattgactggtattcttaactatgttgc |
| | tccttttacgctatgtggatacgctgctttaatgcctttgtatcatgctattgcttcccg |
| | tatggctttcattttctcctccttgtataaatcctggttagttcttgccacggcggaact |
| | catcgccgcctgccttgcccgctgctggacaggggctcggctgttgggcactgacaattc |
| | cgtggtgtttatttgtgaaatttgtgatgctattgctttatttgtaaccattctagcttt |
| | atttgtgaaatttgtgatgctattgctttatttgtaaccattataagctgcaataaacaa |
| | gttaacaacaacaattgcattcattttatgtttcaggttcagggggagatgtgggaggtt |
| | ttttaaagcCATGGCACTAGGACTAAAGGTTGGCCAAAGTACAAGATATTTGTCTTATCT |
| | GATGACAACTCTGTGTCCTGGACTCTCTTCCAGAATAAGACCTTTCCTGCAGCACTGCTT |
| | GAACTCCTCTTAGCAAGAGGGAAACATGTGAAATGCTACCAAAATAGAATAGAAGTAAAT |
| | TCTTATTATATTCCTTTGTTCACTCATATCCTGAAGTGCATCAAATCAGGTTTTCTCACC |
| | TGTATAATGCTGTATTTTACTTGAGTTGGAATAATTTTGCTTAGAAATAAATAAGTAAAA |
| | CAGCACCTG (SEQ ID NO: 3) |
| GSH2 | CATTACATCCAAGTTTAGACTCATTGAGCTCTAAATATTTGGGAAAACATATTTAAAGAA |
| CMV-LAMB3- | ATTATATAGGTTTGATCCAAAATCTCTTTGGCACAACTTGAAATATGGGTAATCGTCATG |
| T2A-GFP | TGAAATTTGTGAATAGGAGAACCCACTGTAGGATACTTAACATAAATCAGCCACATAATT |
| | TCTATCACTGATATCCAGGGAATTTCAATGACAAATCTAGTGATAAAAATTGATAAAACA |
| | TTTTTGATAGTTTTGATACAAGTGAAAGTCATGGGATATCAGACTTAAAAGAAACCTCAG |
| | GACATTGATTATTGACTAGTTATTAATAGTAATCAATTACGGGGTCATTAGTTCATAGCC |
| | CATATATGGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCAATAGGGA |
| | CTTTCCATTGACGTCAATGGGTGGACTATTTACGGTAAACTGCCCACTTGGCAGTACATC |
| | AAGTGTATCATATGCCAAGTACGCCCCCTATTGACGTCAATGACGGTAAATGGCCCGCCT |
| | GGCATTATGCCCAGTACATGACCTTATGGGACTTTCCTACTTGGCAGTACATCTACGTAT |
| | TAGTCATCGCTATTACCATGGTGATGCGGTTTTGGCAGTACATCAATGGGCGTGGATAGC |
| | GGTTTGACTCACGGGGATTTCCAAGTCTCCACCCCATTGACGTCAATGGGAGTTTGTTTT |
| | GGCACCAAAATCAACGGGACTTTCCAAAATGTCGTAACAACTCCGCCCCATTGACGCAAA |
| | TGGGCGGTAGGCGTGTACGGTGGGAGGTCTATATAAGCAGAGCTCTCTGGCTAACTAGAG |
| | AACCCACTGCTTACTGGCTTATCGAAATTAATACGACTCACTATAGGGAGACCCAAGCTT |
| | GCGGCCGCCACCatgagaccattcttcctcttgtgttttgccctgcctggcctcctgcat |
| | gcccaacaagcctgctcccgtggggcctgctatccacctgttggggacctgcttgttggg |
| | aggacccggtttctccgagcttcatctacctgtggactgaccaagcctgagacctactgc |
| | acccagtatggcgagtggcagatgaaatgctgcaagtgtgactccaggcagcctcacaac |
| | tactacagtcaccgagtagagaatgtggcttcatcctccggccccatgcgctggtggcag |
| | tcccagaatgatgtgaaccctgtctctctgcagctggacctggacaggagattccagctt |
| | caagaagtcatgatggagttccaggggcccatgcctgccggcatgctgattgagcgctcc |
| | tcagacttcggtaagacctggcgagtgtaccagtacctggctgccgactgcacctccacc |
| | ttccctcgggtccgccagggtcggcctcagagctggcaggatgttcggtgccagtccctg |
| | cctcagaggcctaatgcacgcctaaatggggggaaggtccaacttaaccttatggattta |
| | gtgtctgggattccagcaactcaaagtcaaaaaattcaagaggtgggggagatcacaaac |
| | ttgagagtcaatttcaccaggctggcccctgtgccccaaaggggctaccaccctcccagc |
| | gcctactatgctgtgtcccagctccgtctgcaggggagctgcttctgtcacggccatgct |
| | gatcgctgcgcacccaagcctggggcctctgcaggcccctccaccgctgtgcaggtccac |
| | gatgtctgtgtctgccagcacaacactgccggcccaaattgtgagcgctgtgcacccttc |
| | tacaacaaccggccctggagaccggcggagggccaggacgcccatgaatgccaaaggtgc |
| | gactgcaatgggcactcagagacatgtcactttgaccccgctgtgtttgccgccagccag |
| | ggggcatatggaggtgtgtgtgacaattgccgggaccacaccgaaggcaagaactgtgag |
| | cggtgtcagctgcactatttccggaaccggcgcccgggagcttccattcaggagacctgc |
| | atctcctgcgagtgtgatccggatggggcagtgccaggggctccctgtgacccagtgacc |
| | gggcagtgtgtgtgcaaggagcatgtgcagggagagcgctgtgacctatgcaagccgggc |
| | ttcactggactcacctacgccaacccgcagggctgccaccgctgtgactgcaacatcctg |
| | gggtcccggagggacatgccgtgtgacgaggagagtgggcgctgcctttgtctgcccaac |
| | gtggtgggtcccaaatgtgaccagtgtgctccctaccactggaagctggccagtggccag |
| | ggctgtgaaccgtgtgcctgcgacccgcacaactccctcagcccacagtgcaaccagttc |
| | acagggcagtgcccctgtcgggaaggctttggtggcctgatgtgcagcgctgcagccatc |
| | cgccagtgtccagaccggacctatggagacgtggccacaggatgccgagcctgtgactgt |
| | gatttccggggaacagagggcccgggctgcgacaaggcatcaggccgctgcctctgccgc |
| | cctggcttgaccgggccccgctgtgaccagtgccagcgaggctactgcaatcgctacccg |
| | gtgtgcgtggcctgccacccttgcttccagacctatgatgcggacctccgggagcaggcc |
| | ctgcgctttggtagactccgcaatgccaccgccagcctgtggtcagggcctgggctggag |
| | gaccgtggcctggcctcccggatcctagatgcaaagagtaagattgagcagatccgagca |
| | gttctcagcagccccgcagtcacagagcaggaggtggctcaggtggccagtgccatcctc |
| | tccctcaggcgaactctccagggcctgcagctggatctgcccctggaggaggagacgttg |
| | tcccttccgagagacctggagagtcttgacagaagcttcaatggtctccttactatgtat |
| | cagaggaagagggagcagtttgaaaaaataagcagtgctgatccttcaggagccttccgg |
| | atgctgagcacagcctacgagcagtcagcccaggctgctcagcaggtctccgacagctcg |
| | cgccttttggaccagctcagggacagccggagagaggcagagaggctggtgcggcaggcg |
| | ggaggaggaggaggcaccggcagccccaagcttgtggccctgaggctggagatgtcttcg |
| | ttgcctgacctgacacccaccttcaacaagctctgtggcaactccaggcagatggcttgc |
| | accccaatatcatgccctggtgagctatgtccccaagacaatggcacagcctgtggctcc |
| | cgctgcaggggtgtccttcccagggccggtggggccttcttgatggcggggcaggtggct |
| | gagcagctgcggggcttcaatgcccagctccagcggaccaggcagatgattagggcagcc |
| | gaggaatctgcctcacagattcaatccagtgcccagcgcttggagacccaggtgagcgcc |
| | agccgctcccagatggaggaagatgtcagacgcacacggctcctaatccagcaggtccgg |
| | gacttcctaacagaccccgacactgatgcagccactatccaggaggtcagcgaggccgtg |
| | ctggccctgtggctgcccacagactcagctactgttctgcagaagatgaatgagatccag |
| | gccattgcagccaggctccccaacgtggacttggtgctgtcccagaccaagcaggacatt |
| | gcgcgtgcccgccggttgcaggctgaggctgaggaagccaggagccgagcccatgcagtg |
| | gagggccaggtggaagatgtggttgggaacctgcggcaggggacagtggcactgcaggaa |
| | gctcaggacaccatgcaaggcaccagccgctcccttcggcttatccaggacagggttgct |
| | gaggttcagcaggtactgcggccagcagaaaagctggtgacaagcatgaccaagcagctg |
| | ggtgacttctggacacggatggaggagctccgccaccaagcccggcagcagggggcagag |
| | gcagtccaggcccagcagcttgcggaaggtgccagcgagcaggcattgagtgcccaagag |
| | ggatttgagagaataaaacaaaagtatgctgagttgaaggaccggttgggtcagagttcc |
| | atgctgggtgagcagggtgcccggatccagagtgtgaagacagaggcagaggagctgttt |
| | ggggagaccatggagatgatggacaggatgaaagacatggagttggagctgctgcggggc |
| | agccaggccatcatgctgcgctcggcggacctgacaggactggagaagcgtgtggagcag |
| | atccgtgaccacatcaatgggcgcgtgctctactatgccacctgcaagGAGGGCAGAGGA |
| | AGTCTTCTAACATGCGGTGACGTGGAGGAGAATCCCGGCCCTAGCatggtgagcaagggc |
| | gaggagctgttcaccggggtggtgcccatcctggtcgagctggacggcgacgtaaacggc |
| | cacaagttcagcgtgtccggcgagggcgagggcgatgccacctacggcaagctgaccctg |
| | aagttcatctgcaccaccggcaagctgcccgtgccctggcccaccctcgtgaccaccctg |
| | acctacggcgtgcagtgcttcagccgctaccccgaccacatgaagcagcacgacttcttc |
| | aagtccgccatgcccgaaggctacgtccaggagcgcaccatcttcttcaaggacgacggc |
| | aactacaagacccgcgccgaggtgaagttcgagggcgacaccctggtgaaccgcatcgag |
| | ctgaagggcatcgacttcaaggaggacggcaacatcctggggcacaagctggagtacaac |
| | tacaacagccacaacgtctatatcatggccgacaagcagaagaacggcatcaaggtgaac |
| | ttcaagatccgccacaacatcgaggacggcagcgtgcagctcgccgaccactaccagcag |
| | aacacccccatcggcgacggccccgtgctgctgcccgacaaccactacctgagcacccag |
| | tccgccctgagcaaagaccccaacgagaagcgcgatcacatggtcctgctggagttcgtg |
| | accgccgccgggatcactctcggcatggacgagctgtacaagtaaagcggactagtctag |
| | caatcaacctctggattacaaaatttgtgaaagattgactggtattcttaactatgttgc |
| | tccttttacgctatgtggatacgctgctttaatgcctttgtatcatgctattgcttcccg |
| | tatggctttcattttctcctccttgtataaatcctggttagttcttgccacggcggaact |
| | catcgccgcctgccttgcccgctgctggacaggggctcggctgttgggcactgacaattc |
| | cgtggtgtttatttgtgaaatttgtgatgctattgctttatttgtaaccattctagcttt |
| | atttgtgaaatttgtgatgctattgctttatttgtaaccattataagctgcaataaacaa |
| | gttaacaacaacaattgcattcattttatgtttcaggttcagggggagatgtgggaggtt |
| | ttttaaagcTGCTATCAAGTCTGATGTCAGTAATTTTTGGAGGAGACTGAAGTGCAGTGA |
| | GACTATCCAAAGTCAGACATGGGGAAAAGCAGAGTCATCCCTCCTAGGCTGCCAAAATCC |
| | TCCCCATCCAAGCTCATCCTTGAAGCCCTCACTTAAGACAAAGTTCCTCCCATCCCTTCT |
| | GCCTGCTCTGGCATGGTCTGAACCATTTGCCTATTAATTGCCCTGCCTGGTTTCATTTGT |
| | TCTTTTTGCTGTATTTAAACTGTGGGAATTCTATTGTTAACCTTTTTCTTGCTCAACTGA |
| | ACTGTGACA (SEQ ID NO: 4) |

### HEK293T and Jurkat Cell Culture, Transfection and Sorting

HEK293T cells were obtained from the American Type Culture Collection (ATCC) (#CRL-3216); the Jurkat leukemia E6-1 T cell line was obtained from ATCC (#TIB152). HEK cells were cultured in Dulbecco's Modified Eagle's Medium (DMEM) (ATCC 30-2002) supplemented with 2 mM L-glutamine (ATCC 30-2214). Jurkat cells were cultured in ATCC-modified RPMI-1640 (Thermo Fisher, #A1049101). All media were supplemented with 10% FBS, 50 U ml-1penicillin and 50 μg ml-1streptomycin. Detachment of HEK cells for passaging was performed using the TrypLE reagent (Thermo Fisher, #12605010). All cell lines were cultured at 37° C., 5% CO2 in a humidified atmosphere.

Prior to transfection of HEK293T and Jurkat gRNA molecules were assembled by mixing 4 μl of custom Alt-R crRNA (200 μM, IDT) with 4 μL of Alt-R tracrRNA (200 μM, IDT, #1072534), incubating the mix at 95° C. for 5 min and cooling it to room temperature. 2 μL of assembled gRNA molecules were mixed with 2 μL of recombinant SpCas9 (61 μM, IDT, #1081059) and incubated for >10 min at room temperature to generate Cas9 RNP complexes.

For transfection of HEK cells 100 μL format SF Cell line kit (Lonza, V4XC-2012) and electroporation program CM-130 was used on the 4D-Nucleofector. 1×10⁶ HEK cells were transfected with 2 μg of PITCh donor, 2 μl of Cas9 RNP complex against specific GSH and 2 μl of Cas9 RNP complex against PITCh plasmid to liberate MMEJ insert.

For transfection of Jurkat cells 100 μL format SE Cell line kit (Lonza, V4XC-1012) and electroporation program CL-120 was used on the 4D-Nucleofector. 1×10⁶ Jurkat cells were transfected with 2 μg of PITCh donor, 2 μl of Cas9 RNP complex against specific GSH and 2 μl of Cas9 RNP complex against PITCh plasmid to liberate MMEJ insert.

Transfected HEK and Jurkat cells were bulk sorted on day 3 and single-cell sorted on day 10 following transfection using Sony SH800S sorter. Best expressing clone was selected on day 30 and cultured for another 2 months. mRuby expression of the best expressing clone was analyzed on BD LSRFortessa Flow Cytometer on day 45, 60 and 90 following transfection.

### Human T-Cells Culture, Transfection and Sorting

Human peripheral blood mononuclear cells were purchased from Stemcell Technologies (#70025) and T cells isolated using the EasySep Human T Cell Isolation kit (Stemcell Technologies, #17951). Primary human T cells were cultured for up to 25 days in ATCC-modified RPMI (Thermo Fisher, #A1049101) supplemented with 10% FBS, 10 mM non-essential amino acids, 5011M 2-mercaptoethanol, 50 U ml-1penicillin, 50 μg ml⁻⁶ streptomycin and freshly added 20 ng ml⁻¹ recombinant human IL-2, (Peprotech, #200-02). T cells were cultured at 37° C., 5% CO2 in a humidified atmosphere. On day 1 of culture, transfection of primary T cells with Cas9 RNP complexes and GSH1/GSH2-mRuby HDR templates was performed using the 4D-Nucleofector and a 20 uL format P3 Primary Cell kit (Lonza, V4XP-3032). Briefly, gRNA molecules were assembled by mixing 4 μl of custom Alt-R crRNA (200 μM, IDT) with 4 μL of Alt-R tracrRNA (200 μM, IDT, #1072534), incubating the mix at 95° C. for 5 min and cooling it to room temperature. 2 μL of assembled gRNA molecules were mixed with 2 μL of recombinant SpCas9 (61 μM, IDT, #1081059) and incubated for >10 min at room temperature to generate Cas9 RNP complexes. 1×10⁶ primary T cells were transfected with 1 μg of HDR template, 1 μl of GHS1/GSH2 Cas9 RNP complex using the E0115 electroporation program. T cells were activated with Dynabeads™ Human T-Activator CD3/CD28 (Thermo Fischer, #11161D) 3-4 hours following transfection. mRuby-positive T-cells were bulk sorted on day 4 using Sony SH800S sorter, re-activated with the new beads on day 8, sorted again on day 11 and analyzed on BD LSRFortessa Flow Cytometer on day 20.

### Human Dermal Fibroblasts Culture, Transfection and Sorting

Neonatal human dermal fibroblasts were purchased from Coriell Institute (Catalog ID GM03377). Primary fibroblasts were cultured for up to 25 days in Prime Fibroblast media (CELLNTEC, CnT-PR-F). Cells were passaged at 70% confluency using Accutase (CELLNTEC, CnT-Accutase-100). Detached cells were centrifuged for 5 min, 200×g at room temperature and seeded at seeded at 2,000 cells per cm 2. Fibroblasts were cultured at 37° C., 5% CO2 in a humidified atmosphere. Fibroblasts were transfected using Lipofectamine™ CRISPRMAX™ Cas9 Transfection Reagent (ThermoFisher Scientific, CMAX00001). Briefly, cells were transfected at 50% confluency with 1:1 ratio of custom sgRNA (40 pmoles, Synthego) and SpCas9 (40pmoles, Synthego) and 2.5 μg of GSH1/GSH2 LAMB3-T2A-GFP HDR template. GFP-positive fibroblasts were bulk sorted on day 3 and 10 using Sony SH800S sorter and analyzed on BD LSRFortessa Flow Cytometer on day 25.

Genotypic Analysis of GSH Integration Genomic DNA was extracted from 1×10⁶ cells using PureLink Genomic DNA extraction kit (ThermoFischer Scientific, #K1820-01). 5 μL of genomic DNA extract were then used as templates for 25 μL PCR reactions using a primer with one primer residing outside of the homology arm of the integrated sequence and the other primer inside the integrated sequence. Obtained bands were gel extracted using Zymoclean Gel DNA Recovery Kit (Zymo Research, #D4001), 4 μl of eluted DNA was cloned into a TOPO-vector using Zero-blunt TOPO PCR Cloning Kit (ThermoFischer Scientific, #450245), incubated for 1 hour, transformed into NEB 5-alpha Competent E. coli cells (New England Biolabs, C2987H) and plated on agar plates containing kanamycin at 50 μg/ml. Produced clones were picked and inoculated for overnight culture in 5 ml of liquid broth supplemented with kanamycin at 50 μg/ml. Liquid cultures were mini-prepped the following morning using ZR Plasmid Miniprep—Classic kit (Zymo Research, #D4015) and Sanger sequenced by Microsynth using M13-forward and M13-reverse standard primers.

### Bulk RNA-Sequencing of HEK293T and Jurkat Cells GSH2 and WT

Following single-cell sort, the best expressing clone (GSH2) and wild-type (WT) of HEK293T and Jurkat cells were cultured for 80 days. Each of the four clones were split into 2 wells (1 and 2), cultured for an additional week, after which total RNA was extracted using PureLink RNA Mini Kit (ThermoFischer Scientific, #12183018A). Extracted total RNA was depleted of rRNA using RiboCop rRNA Depletion Kit (Lexogen, #144), first and second strands of cDNA were generated with SuperScript Double-Stranded cDNA Synthesis Kit (ThermoFischer Scientific, #11917010) using random hexamers and flow cell adapters were ligated to the produced double-stranded cDNA. DNA fragments were enriched by PCR using Q5 High-Fidelity 2× Master Mix (New England Biolabs, #M0492S) and sequenced by the Illumina NextSeq 500 system in the Genomics Facility Basel. Sequencing reads were aligned to the human reference genome (GRCh38) using Subread (v1.6.2) using unique mapping (Liao et al., 2013). Expression levels were quantified using the featureCounts function in the Rpackage Rsubread at gene-level (Liao et al.). Normalization across the samples was performed using default parameters in the Rpackage edgeR (Robinson et al., 2010). Differential expression analysis was performed using the exactTest function in the edgeR package. Gene ontology was performed by supplying those differentially expressed genes (adjusted p value<0.05) to the goana function (Young et al., 2010).

### Single-Cell RNA Sequencing of Human T-Cells

Single-cell RNA sequencing was conducted on day 25 of culture for Donor 1 WT (D1 WT) and Donor 1 GSH1 (D1 GSH1) and on day 5 for Donor 2 WT (D2 WT). Single cell 10× libraries were constructed from the isolated single cells following the Chromium Single Cell 3′ GEM, Library & Gel Bead Kit v3 (10× Genomics, PN-1000075). Briefly, single cells were co-encapsulated with gel beads (10× Genomics, 2000059) in droplets using Chromium Single Cell B Chip (10× Genomics, 1000074). Final D1 WT, D1 GSH1 and D2 WT libraries were pooled and sequenced on the Illumina NovaSeq platform (26/8/0/93 cycles). Raw sequencing files supplied to cellranger (v3.1.0) using the count argument under default parameters and the human reference genome (GRCh38-3.0.0). Filtering, normalization and transcriptome analysis was performed using a previously described pipeline in the R package Platypus (Yermanos et al.). Briefly, filtered gene expression matrices from cellranger were supplied as input into the Read10× function in the R package Seurat (Stuart et al., 2019). Cells containing more than 5% mitochondrial genes, or less than 150 unique genes detected were filtered out before using the RunPCA function and subsequent normalization using the function RunHarmony from the Harmony package under default parameters (Korsunsky et al., 2019). Uniform manifold approximation projection was performed with Seurat's RunUMAP function using the first 20 dimensions and the previously computed Harmony reduction. Clustering was performed by the Seurat functions FindNeighbors and FindClusters using the Harmony reduction and first 20 principal components and the default cluster resolution of 0.5, respectively (Satija et al., 2015). Cluster-specific genes were determined by Seurat's FindMarkers function for those genes expressed in at least 25% of cells in one of the two groups. Differential genes between samples were calculated using the FindMarkers function from Seurat using the default Wilcoxon Rank Sum Test with Bonferroni multiple hypothesis correction. The source code for the analysis of scRNA-seq data is available at https://github.com/alexyermanos/Platypus.

TABLE 5
Tested sites (see section below for tested and predicted sequences)

| | | | | | Expression in human cells | | | |
|---|---|---|---|---|---|---|---|---|
| Chromosome | Start | End | Size | ID | HEK293T | Jurkat | T cells | Fibroblasts |
| chr1 | 195338589 | 195818588 | 479999 | GSH1 | + | + | + | + |
| chr3 | 22720711 | 22761389 | 40678 | GSH2 | + | + | + | + |
| chrX | 89174426 | 89179074 | 4648 | GSH31 | + | − | N/A | N/A |
| chr7 | 145090941 | 145219513 | 128572 | GSH7 | + | − | N/A | N/A |
| chr7 | 145320384 | 145525881 | 205497 | GSH8 | − | − | N/A | N/A |

N/A—not attempted due to absence of expression in Jurkat cell line.

Guide RNAs and Homology Arms (1 kb)
GSH1-TESTED GRNA/HA
GRNA
TTAGTCCTAGTGCCATGAAG TGG (SEQ ID NO: 5)
HA LEFT
GCAAATTTTGGAATTTTGTTAAAATAGTTAAAGATAAACTATGTTACCTTTCAGAAAAGTAAAGGGAGTGGTCAG
TGACTATTAATAAAACAAATAGTGCCATCTACTGTCAAAAAATTCTATTATGAAAGTTGCCATAAACATCATTAT
TTTTCAGTGTGATAGTGCTATAGTCATTTATTTGATATTCTAAAATTTCCAAGAATTTTTATTTATTTCAAATAA
TCACAGTTAAATTTCTAGTTCTGCTAACTGGAGTGGAATTTACATGTACTTAAATCAAATGACTGCCACTTTACA
AAATCACTGTCATCAGGAAGCAATTTTTTAAAAGTGTTTCTTTGTGCTAAGAAGTACACGATGTAAAAACAACAA
CAACAACAAAATGCTTGTATCCTTTCCAAACACCATACACACACATGACTCAGTCAGAACTGCAGAAATGTAAGG
ATAACGATACTAAACAAGAGGAAAAATGAAAAAGACAGGAAAAAGCCTGGTCAAATTATTAAAGAAGTGCAAGCA
TTGATGCAACTTACTGATAAAGGTGAAACTGTAAAGTATACTTTAAAATAGATGCAGTAAGTAGAATTAGAGTTA
GCTTCCATCACCTTTTAATCTACAAATGATTTTACAGAGAAAGCAGCATTAAAGATCTTTGTGGGCAATCAAAAC
AGTAATTTGAGAATAGCATTATACACTGCATTTAAGTAGGATTCAATAATTTTAAAGTGCAGGGACAAAATTTCC
TCATATGGCTCACTAGCTACATTGCAAATTTCTTGAAATCAGAACACAGAAGTGCAGTCCTGTGCTCGCAATGCA
GACTTGCAGGGTGTAGAGGCATAAATGGCTCCAGAGCCAGGGACATGGGTCCAGAGGGGGGTAGTCTCCAGAAGA
CTCCTTTCGGGCCTATTACCATGCCTCAGAGGTCCAAGTGGGGCATGGTGAATATATTATCCTTTATATTATATT
TCTTATATGTCTACAACTGCCACTT (SEQ ID NO: 25)
HA RIGHT
CATGGCACTAGGACTAAAGGTTGGCCAAAGTACAAGATATTTGTCTTATCTGATGACAACTCTGTGTCCTGGACT
CTCTTCCAGAATAAGACCTTTCCTGCAGCACTGCTTGAACTCCTCTTAGCAAGAGGGAAACATGTGAAATGCTAC
CAAAATAGAATAGAAGTAAATTCTTATTATATTCCTTTGTTCACTCATATCCTGAAGTGCATCAAATCAGGTTTT
CTCACCTGTATAATGCTGTATTTTACTTGAGTTGGAATAATTTTGCTTAGAAATAAATAAGTAAAACAGCACCTG
CCTCCAGACCTAGGGTCCATCAGGAAAAATATAAGGTATATGAGGTGTATGCTCTAAACCCAAGGCCAACATGTA
TAGGAAAACCTTAAGTCCTTCAGTGCATGTGCTTGGATGAAGAAGGTAATACAATTGTAGGCAACTGCAAGAGCA
ATGTAGGTAAAATTCACACCTACAGGCAGTCGTGAAAATTTTCCCAATATAAACTTGCACTTCACATGCACTTTT
GTGGTGTGAAGGAGAGGAACTGGTGAGAAACTGATGAGAAATGATGATAAGCAGACTTTTACTGGAACATTGCTC
AATCCCCTCTATAAGGCAATGATGCTATGTAGACAATCAAACATGAAATTGCTAGAAAGTTTATAGATTGATATA
ATCTATTTTAATGCATCTAGGATTCAGGTAAGCTGCGAAAAAGTAGTGCCAATATGTTTATTTTATAGGGGATAT
TTAAAATTAATTTTATTCTTTTTAAAATTGCAATGGTACCCAAATTCCCTAACTTCCTATGGTAGGCTGAATAAC
AGCTCCTAAAAATATCAGGTTGTAATTCCTGGAATGTGTAAATGCTATCTTATATGGAAAATATACCAATATGTG
ATTAAATTATGGAGTTTGAAATGAAGAGATTAACCTGATTTATCTGGGTGCATCCTACATGAGATCACAATTGTT
CTTAAAAGAGGGAGGCAGAGGGAGG (SEQ ID NO: 45)
PREDICTED GSH1 GRNAS/HAS
GRNA
ATGCCTCAGAGGTCCAAGTG GGG (SEQ ID NO: 6)
HA LEFT
ATAAAAGCAAAAATGTCCCTATCACACATAATCAAAGTGATTCATCTGGTAAGCTAGATATAAGCAAATTTTGGA
ATTTTGTTAAAATAGTTAAAGATAAACTATGTTACCTTTCAGAAAAGTAAAGGGAGTGGTCAGTGACTATTAATA
AAACAAATAGTGCCATCTACTGTCAAAAAATTCTATTATGAAAGTTGCCATAAACATCATTATTTTTCAGTGTGA
TAGTGCTATAGTCATTTATTTGATATTCTAAAATTTCCAAGAATTTTTATTTATTTCAAATAATCACAGTTAAAT
TTCTAGTTCTGCTAACTGGAGTGGAATTTACATGTACTTAAATCAAATGACTGCCACTTTACAAAATCACTGTCA
TCAGGAAGCAATTTTTTAAAAGTGTTTCTTTGTGCTAAGAAGTACACGATGTAAAAACAACAACAACAACAAAAT
GCTTGTATCCTTTCCAAACACCATACACACACATGACTCAGTCAGAACTGCAGAAATGTAAGGATAACGATACTA
AACAAGAGGAAAAATGAAAAAGACAGGAAAAAGCCTGGTCAAATTATTAAAGAAGTGCAAGCATTGATGCAACTT
ACTGATAAAGGTGAAACTGTAAAGTATACTTTAAAATAGATGCAGTAAGTAGAATTAGAGTTAGCTTCCATCACC
TTTTAATCTACAAATGATTTTACAGAGAAAGCAGCATTAAAGATCTTTGTGGGCAATCAAAACAGTAATTTGAGA
ATAGCATTATACACTGCATTTAAGTAGGATTCAATAATTTTAAAGTGCAGGGACAAAATTTCCTCATATGGCTCA
CTAGCTACATTGCAAATTTCTTGAAATCAGAACACAGAAGTGCAGTCCTGTGCTCGCAATGCAGACTTGCAGGGT
GTAGAGGCATAAATGGCTCCAGAGCCAGGGACATGGGTCCAGAGGGGGGTAGTCTCCAGAAGACTCCTTTCGGGC
CTATTACCATGCCTCAGAGGTCCAA (SEQ ID NO: 26)
HA RIGHT
GTGGGGCATGGTGAATATATTATCCTTTATATTATATTTCTTATATGTCTACAACTGCCACTTCATGGCACTAGG
ACTAAAGGTTGGCCAAAGTACAAGATATTTGTCTTATCTGATGACAACTCTGTGTCCTGGACTCTCTTCCAGAAT
AAGACCTTTCCTGCAGCACTGCTTGAACTCCTCTTAGCAAGAGGGAAACATGTGAAATGCTACCAAAATAGAATA
GAAGTAAATTCTTATTATATTCCTTTGTTCACTCATATCCTGAAGTGCATCAAATCAGGTTTTCTCACCTGTATA
ATGCTGTATTTTACTTGAGTTGGAATAATTTTGCTTAGAAATAAATAAGTAAAACAGCACCTGCCTCCAGACCTA
GGGTCCATCAGGAAAAATATAAGGTATATGAGGTGTATGCTCTAAACCCAAGGCCAACATGTATAGGAAAACCTT
AAGTCCTTCAGTGCATGTGCTTGGATGAAGAAGGTAATACAATTGTAGGCAACTGCAAGAGCAATGTAGGTAAAA
TTCACACCTACAGGCAGTCGTGAAAATTTTCCCAATATAAACTTGCACTTCACATGCACTTTTGTGGTGTGAAGG
AGAGGAACTGGTGAGAAACTGATGAGAAATGATGATAAGCAGACTTTTACTGGAACATTGCTCAATCCCCTCTAT
AAGGCAATGATGCTATGTAGACAATCAAACATGAAATTGCTAGAAAGTTTATAGATTGATATAATCTATTTTAAT
GCATCTAGGATTCAGGTAAGCTGCGAAAAAGTAGTGCCAATATGTTTATTTTATAGGGGATATTTAAAATTAATT
TTATTCTTTTTAAAATTGCAATGGTACCCAAATTCCCTAACTTCCTATGGTAGGCTGAATAACAGCTCCTAAAAA
TATCAGGTTGTAATTCCTGGAATGTGTAAATGCTATCTTATATGGAAAATATACCAATATGTGATTAAATTATGG
AGTTTGAAATGAAGAGATTAACCTG (SEQ ID NO: 46)
GRNA
TTGAAATACAGTGAGCAGGG AGG (SEQ ID NO: 7)
HA LEFT
ACAATTGTAGGCAACTGCAAGAGCAATGTAGGTAAAATTCACACCTACAGGCAGTCGTGAAAATTTTCCCAATAT
AAACTTGCACTTCACATGCACTTTTGTGGTGTGAAGGAGAGGAACTGGTGAGAAACTGATGAGAAATGATGATAA
GCAGACTTTTACTGGAACATTGCTCAATCCCCTCTATAAGGCAATGATGCTATGTAGACAATCAAACATGAAATT
GCTAGAAAGTTTATAGATTGATATAATCTATTTTAATGCATCTAGGATTCAGGTAAGCTGCGAAAAAGTAGTGCC
AATATGTTTATTTTATAGGGGATATTTAAAATTAATTTTATTCTTTTTAAAATTGCAATGGTACCCAAATTCCCT
AACTTCCTATGGTAGGCTGAATAACAGCTCCTAAAAATATCAGGTTGTAATTCCTGGAATGTGTAAATGCTATCT
TATATGGAAAATATACCAATATGTGATTAAATTATGGAGTTTGAAATGAAGAGATTAACCTGATTTATCTGGGTG
CATCCTACATGAGATCACAATTGTTCTTAAAAGAGGGAGGCAGAGGGAGGTGTGACACAGACAAAAGAGAAGGCC
ATGGGAAGACAGAGCAAGAGAGGGTAAAAGATGCTGGCCTTAAATATTGGAGTAATGTAGCAACAGGCCAAGGGA
TGCCAGCAGGAGTTGTAGGAGGTCAATACCTTGATTTTGACCCAGTGATACTGACTTCAGACTTGTGGCCTCCAG
AACTGTGAAAGAATAAATTCCTGTCGTTTTAAGTCACTGACACTGATTTTGTGGTAGGTAATTTGTTACAGCAGC
CACAGGAAACTAATACAATCTGGGTGAATTTCTCTTTCTATAATGAAGGATTCTTTCATAATTAAAAATATAACT
TTAATATAGTTGGTATTATCAGCACCATGCTATAACTTCTGCAAAAAAGCTCTCTAATCCTTATATCTGTTTTCT
TGATAAATCATACTGTTCTCCTCCC (SEQ ID NO: 27)
HA RIGHT
TGCTCACTGTATTTCAACCATACCAGATCCCTTAATGGGAAAGGGTATTTCAGACCTGGGCACTGGCTGTTTCTT
CTGATTGGAGTACTTCTACCTCAGACATCATAATGATGAACTCTTTTGCCTTCTTCAAGTCTTAGATCAAATTAT
TCCTTTTTACTGTTTATATTCCAACTAGTGAGGGATAATGTCCCTCACTATCCCCTAGGAGATTGATTCCAGGAA
GCTTGAGGGTACCAAACTCTGTGGATGCTCAAGTCTCTGATAGAAAATGCCATAGTATTTACATATGATCTACAC
AAACCCTCCTGCATATTTAAAATAGTCTCTAGATTACTTATAATTACTAGATTACTTATAGTCTCTAGATTTATA
TAATCTTATAAAAGGGTAAGATTACTTATTACTTACCCTTAATACAATGTAAATATTGCGTAGCTTTTATACTGT
GAATTTTAAAATTTGTATTATTTTCATTACTATATTTTTGTTTTTTCTGCATATTTTTAATCCATGGTTGGTGAA
ATCCATGGATATGAAATCCGCTCATATGGAGGGACTGAGAACCAATCATATTCTATTCAACACTGCAACCTCCTT
TCCCACCCAGCATAACAGACTAATTGTACCCTGCTTCTTCTGCTTTATTTTCTAAATCTCATTTCAATCTCTAAA
ATGTTATGTAATTTACTTAATTGCTATGTTCATTTTATATCAACTTTCTGCCTCTTCTACTATGTTATCTTCTTG
AGAGAGAAGATTTTAATCTCTTTTGCTCACTAGTGTATTCCCAGTGCATAGAACAATATCTAGCCCATAACAGGT
ATTCAGTAATTTTATTCTTGAATGAATAATTGAAGGAAAACTTTTAAAAATCCATTACCATAAGGTAGGGATGCA
GAGAGCCTAAATCATACTAAAGTGAATTTCAGCTTTCAGTTCAAGCTGACATATTATCAAATCTTCTTATGTTTT
TATCATTTCAACTTCTGTTCTGTGC (SEQ ID NO: 47)
GRNA
GCTAGCTAAAGTCTCGAACT TGG (SEQ ID NO: 8)
HA LEFT
AACCATACCAGATCCCTTAATGGGAAAGGGTATTTCAGACCTGGGCACTGGCTGTTTCTTCTGATTGGAGTACTT
CTACCTCAGACATCATAATGATGAACTCTTTTGCCTTCTTCAAGTCTTAGATCAAATTATTCCTTTTTACTGTTT
ATATTCCAACTAGTGAGGGATAATGTCCCTCACTATCCCCTAGGAGATTGATTCCAGGAAGCTTGAGGGTACCAA
ACTCTGTGGATGCTCAAGTCTCTGATAGAAAATGCCATAGTATTTACATATGATCTACACAAACCCTCCTGCATA
TTTAAAATAGTCTCTAGATTACTTATAATTACTAGATTACTTATAGTCTCTAGATTTATATAATCTTATAAAAGG
GTAAGATTACTTATTACTTACCCTTAATACAATGTAAATATTGCGTAGCTTTTATACTGTGAATTTTAAAATTTG
TATTATTTTCATTACTATATTTTTGTTTTTTCTGCATATTTTTAATCCATGGTTGGTGAAATCCATGGATATGAA
ATCCGCTCATATGGAGGGACTGAGAACCAATCATATTCTATTCAACACTGCAACCTCCTTTCCCACCCAGCATAA
CAGACTAATTGTACCCTGCTTCTTCTGCTTTATTTTCTAAATCTCATTTCAATCTCTAAAATGTTATGTAATTTA
CTTAATTGCTATGTTCATTTTATATCAACTTTCTGCCTCTTCTACTATGTTATCTTCTTGAGAGAGAAGATTTTA
ATCTCTTTTGCTCACTAGTGTATTCCCAGTGCATAGAACAATATCTAGCCCATAACAGGTATTCAGTAATTTTAT
TCTTGAATGAATAATTGAAGGAAAACTTTTAAAAATCCATTACCATAAGGTAGGGATGCAGAGAGCCTAAATCAT
ACTAAAGTGAATTTCAGCTTTCAGTTCAAGCTGACATATTATCAAATCTTCTTATGTTTTTATCATTTCAACTTC
TGTTCTGTGCTAGCTAAAGTCTCGA (SEQ ID NO: 28)
HA RIGHT
ACTTGGCTAGGTGTAGTGGTTTATGCCTGTAATCCCTGTGCCCGGGGAAGCCAAGGCAGGAAGATCATTTGAGGC
CGGGTGTTCCAGACCAGCCTAGGCAACATAGCAAGGCCCACCATCTACAAATGATATAATAAAATAACAAAATTA
GCCAGGCATAGTGGTATGTGACCTCAGTCCCAGCTGCTCGAGAGGCTGATGAGGAAGGATCACTTGGCCCAGTAG
TTGGAGTTTGCAGTGATCTGTGATCACACCACTGTATTTCAGCCTTGGTGAGAGAGCAGACCCATCTTTGAAAAA
AAAAATTAAGTCTCAAACTTTATTAATAGTGTAACAGAATGAGCAATACTTTGGAGACATGCTGCTGCTATATAT
ATATATATATATATATATATATATATATATTTTTTTTTTTTTTTTTTTTTTTTTTTTTTTTTTGAGGCAGATTCT
CACTGTGTCACCCAGGCTGGAGTGCAGTGGCGCCACCTCGGCTCACTGCAACCTCTACCTCCAGGGTTCAAGCAA
TTCTCCTGCCTCAGCCTCCCAAGTAGCTGGGATCACAGGTGCCCACTACCACACCCAGCTAATTTTTGGTAATTT
TAGTAGAGATGAGGTTTCACTATGTTGGCTAGGCTGGTCTCAAACTCCTAACCTCAAGTGATCCACCTACCTCTG
CCTCCCAAAGTGCTAGGAGTACAAGTGTGAGCCACTGACCCCGGCCCTTATATTTTTCTAATTATCAAATAGGCT
GTGAGAGTTTCTATTTACCTAGGTCCTCAAAGTGTCTTTAGGAAAAGCTATACCCTGGACAAAGGCAGGTTGGCA
GAAATACCAGGGGTTGAGCTTCTGCAACAAGTTTGGGTCATAAAAACTGCCACTAGGCCTGTCAGGCAACTTCCT
GAGTCAATAAAATCTCACATTAATAGATATAAAGAAAAAGCAATTGAAAATTTTCCAGATAAACTGGATTCCATC
TGTAAAGAGGAAAATCTGTTTTGTG (SEQ ID NO: 48)
GRNA
GGCATGGTAATAGGCCCGAA AGG (SEQ ID NO: 9)
HA LEFT
TATACCTTCATCCAAAATTATTCTATGAAATAAAAGCAAAAATGTCCCTATCACACATAATCAAAGTGATTCATC
TGGTAAGCTAGATATAAGCAAATTTTGGAATTTTGTTAAAATAGTTAAAGATAAACTATGTTACCTTTCAGAAAA
GTAAAGGGAGTGGTCAGTGACTATTAATAAAACAAATAGTGCCATCTACTGTCAAAAAATTCTATTATGAAAGTT
GCCATAAACATCATTATTTTTCAGTGTGATAGTGCTATAGTCATTTATTTGATATTCTAAAATTTCCAAGAATTT
TTATTTATTTCAAATAATCACAGTTAAATTTCTAGTTCTGCTAACTGGAGTGGAATTTACATGTACTTAAATCAA
ATGACTGCCACTTTACAAAATCACTGTCATCAGGAAGCAATTTTTTAAAAGTGTTTCTTTGTGCTAAGAAGTACA
CGATGTAAAAACAACAACAACAACAAAATGCTTGTATCCTTTCCAAACACCATACACACACATGACTCAGTCAGA
ACTGCAGAAATGTAAGGATAACGATACTAAACAAGAGGAAAAATGAAAAAGACAGGAAAAAGCCTGGTCAAATTA
TTAAAGAAGTGCAAGCATTGATGCAACTTACTGATAAAGGTGAAACTGTAAAGTATACTTTAAAATAGATGCAGT
AAGTAGAATTAGAGTTAGCTTCCATCACCTTTTAATCTACAAATGATTTTACAGAGAAAGCAGCATTAAAGATCT
TTGTGGGCAATCAAAACAGTAATTTGAGAATAGCATTATACACTGCATTTAAGTAGGATTCAATAATTTTAAAGT
GCAGGGACAAAATTTCCTCATATGGCTCACTAGCTACATTGCAAATTTCTTGAAATCAGAACACAGAAGTGCAGT
CCTGTGCTCGCAATGCAGACTTGCAGGGTGTAGAGGCATAAATGGCTCCAGAGCCAGGGACATGGGTCCAGAGGG
GGGTAGTCTCCAGAAGACTCCTTTC (SEQ ID NO: 29)
HA RIGHT
GGGCCTATTACCATGCCTCAGAGGTCCAAGTGGGGCATGGTGAATATATTATCCTTTATATTATATTTCTTATAT
GTCTACAACTGCCACTTCATGGCACTAGGACTAAAGGTTGGCCAAAGTACAAGATATTTGTCTTATCTGATGACA
ACTCTGTGTCCTGGACTCTCTTCCAGAATAAGACCTTTCCTGCAGCACTGCTTGAACTCCTCTTAGCAAGAGGGA
AACATGTGAAATGCTACCAAAATAGAATAGAAGTAAATTCTTATTATATTCCTTTGTTCACTCATATCCTGAAGT
GCATCAAATCAGGTTTTCTCACCTGTATAATGCTGTATTTTACTTGAGTTGGAATAATTTTGCTTAGAAATAAAT
AAGTAAAACAGCACCTGCCTCCAGACCTAGGGTCCATCAGGAAAAATATAAGGTATATGAGGTGTATGCTCTAAA
CCCAAGGCCAACATGTATAGGAAAACCTTAAGTCCTTCAGTGCATGTGCTTGGATGAAGAAGGTAATACAATTGT
AGGCAACTGCAAGAGCAATGTAGGTAAAATTCACACCTACAGGCAGTCGTGAAAATTTTCCCAATATAAACTTGC
ACTTCACATGCACTTTTGTGGTGTGAAGGAGAGGAACTGGTGAGAAACTGATGAGAAATGATGATAAGCAGACTT
TTACTGGAACATTGCTCAATCCCCTCTATAAGGCAATGATGCTATGTAGACAATCAAACATGAAATTGCTAGAAA
GTTTATAGATTGATATAATCTATTTTAATGCATCTAGGATTCAGGTAAGCTGCGAAAAAGTAGTGCCAATATGTT
TATTTTATAGGGGATATTTAAAATTAATTTTATTCTTTTTAAAATTGCAATGGTACCCAAATTCCCTAACTTCCT
ATGGTAGGCTGAATAACAGCTCCTAAAAATATCAGGTTGTAATTCCTGGAATGTGTAAATGCTATCTTATATGGA
AAATATACCAATATGTGATTAAATT (SEQ ID NO: 49)
GSH2-TESTED GRNA/HA
GRNA
CATCAGACTTGATAGCACTG AGG (SEQ ID NO: 10)
HA LEFT
ACAATGCATAATATATCAATATTAGTCATTTTTCCCTTTATAAATCATATCTCAAAATGTATGCAATATTCTTTA
AATATAACACTTAAAATGCATATGCAATGATGAAATATAGCAGGATTGCCAAAACTGATGAATATCTCAGAGATA
TTGCCATTGTTTTTCCTGGAAAATACTTCTTTGGAAAAAGTGAGACATCTTTGAGATCAAAATAAACACCTGTCA
AAGGAATGTCTTGGGAACCCCAAATAGCATTTCTAAAGAGATAAAAGAATGTTGTAGTCTTCCAGAAAGGAAATC
AAGTGGAAGTAGATATTAATTCAGCACTACAGAGACAATCTGGAGAAAACAGAGGCATTGTTTTCTAAAGAAATT
GGCTTTTGTAACATAAAGGAGATACAACTCTGGGAGTGAAAGTTGTCACAGTGATACATATCACACAACGGAAAT
GCCAAACGAAACCATCACCACTTGAAATCAAACTTGATTCAAGTTACTCAGGTAAAATACCACCATGGGTGGCAT
CCTTTTTTTTTTTTTTTTTTTTTTTTTTTTTAACCAAGCAGCCTAACGGTGTTTAAGGAGGAAAACTTAATTGAT
AATGCACTTTGCTCAATTATAAATAAGCTGACTGGGAAAGAAGTGGGCATGATGGAAAGCAAAATTTGAATGAAG
CTGTTATTCTTTTAATCTATAAAAACATTACATCCAAGTTTAGACTCATTGAGCTCTAAATATTTGGGAAAACAT
ATTTAAAGAAATTATATAGGTTTGATCCAAAATCTCTTTGGCACAACTTGAAATATGGGTAATCGTCATGTGAAA
TTTGTGAATAGGAGAACCCACTGTAGGATACTTAACATAAATCAGCCACATAATTTCTATCACTGATATCCAGGG
AATTTCAATGACAAATCTAGTGATAAAAATTGATAAAACATTTTTGATAGTTTTGATACAAGTGAAAGTCATGGG
ATATCAGACTTAAAAGAAACCTCAG (SEQ ID NO: 30)
HA RIGHT
TGCTATCAAGTCTGATGTCAGTAATTTTTGGAGGAGACTGAAGTGCAGTGAGACTATCCAAAGTCAGACATGGGG
AAAAGCAGAGTCATCCCTCCTAGGCTGCCAAAATCCTCCCCATCCAAGCTCATCCTTGAAGCCCTCACTTAAGAC
AAAGTTCCTCCCATCCCTTCTGCCTGCTCTGGCATGGTCTGAACCATTTGCCTATTAATTGCCCTGCCTGGTTTC
ATTTGTTCTTTTTGCTGTATTTAAACTGTGGGAATTCTATTGTTAACCTTTTTCTTGCTCAACTGAACTGTGACA
CTGCTAGGAATGCCGAAGCAGGGTTTTAGGTTCTCAGGATGTTTAAGAGTTGGAGAAAGCACTCAATGAGTCTTG
TGAATAATTTTGTGGAAACTGCACTCCCAATGACAGGCTCTGGCATCTCACTCTAAGGTAAGTAACAGGTGAGGC
ACTGCCCTTTGATAACCAGGACGTGGATTCTAGAATTGGTTATGTCCATTCACACAATGTGTTCATCTCCTCTCT
GGCTATCCTTCTGATGACTCAGAAGTCGCAACCCCAAGTTGGTTCTTTCAGGGCCCTGGCTTGCTCATCCCCTTT
ATGATTCTCCTTTATTCTTCTGTACCTGGGTATTCATTTCATGTCCACCCTCATCATCATTCTGGAGACAAAGAT
GCAGACATGTACTGGCAGAATCTGAGCATGCAAAAACCTTCCGTAACACTCAGAGTTCTTATACCTTTCTCTCAT
CTGGCATTATTTACTATGATAGGGTGAAGGAAACAACTATGTCTGTTCTTTGCATTTGACTGCATTGTCCAGTTT
CTAGAGGCTTAAGTACACTTTTTTTTTTTTTTTTTTTGAGACAGAGTTTCACTCTTGTTGCCCAGGCTGGAGTGC
AATGACGCGATCTCAGCTCACTGCAACCTCCAACTCTCAGGTTCAAGTGATTCTCCTGCCTCAGCCTCCCAAATA
GCTGGGATTACAGGCATCTGCCACC (SEQ ID NO: 50)
PREDICTED GSH2 GRNAS/HAS
GRNA
ATATCGTGCTAATGTCAGTG GGG (SEQ ID NO: 11)
HA LEFT
GTGCACGGGTGTCCCGGATTTTCTGAGAGAGCTTTGCATAACTTCTAATTCACAACTTTGATAATTGCCAGAGTG
GCCAAGAGCTCAGAAAAGCATTTTCCCACAAGGTTTTCAAATATAGCTGCCACAAATGTCAAAGATTCTTTTAAT
TTTAGTATCATCCCAGCTAATTCTGAGCATCAGAAAAATATTTTGTTTTATTTTGGATCTACTAAAAAGGAAATG
CTAGCAACAACAACAACAAATCCTCCAGGGCATTCATTACCTACATCATGCATGGGAGAACTGAATTTAGCTTTT
AAAAGTTAAAGTGGAACTTGAAACAGAGCAAAATGAAGTCAAGTGTACTGTGAAAAATTCATGATTTCAAAATGG
AAGCAAGGTCATATTTTATTGGTAATCTTTAAATAGGTTTAGAAAGACTGCAATGTAGCATGGAGAATTTGGTAT
TTGGGCTCATCTGATTCCGCTCTTTATTCAGACAAAATCTGAGCTATAGAAATCAAATATAAAAGAGTGCTAATT
TCTTTTTGTTGTTGTTGTTGGGGGGGACAGAGTCTCGCTCTGTCGCCCAGCTGGAGTGCAGTGGCACAATCTCGG
CTCACTTCAAGCTCCGCCTCCCGGGTTCAAGCCATTCTCCTGCCTCAGCCTCCCAAATAGCTGGGACTACAGGCG
CCCGCCACCTGGCCCGGCTAATTTTTTTGTATTTTTAGTAGAGACACGGCTTCACTGTGTTAGCCAGGATAGTCT
CGATCTCCTGACCTCGTGATCCGCCTGCCTCGGCCTCCCAAAGTGCTGGGAAGGAGTGCTAATTTCAGTTGGCTT
CCAACAAGACCTTATCCAAGCTACATATTGCTATTTTCAAAAATAATTGATATGTAAATTTAAAATCAAATAATT
ACATATTTACGTGAAGTATATCTGTATGTTAAAAGCAAACACACAGCATAAACAGATACAGTGTATTAAAATGGA
TTTTATTTTCACCATCTCACCCCAC (SEQ ID NO: 31)
HA RIGHT
TGACATTAGCACGATATGACAGTAAATGTCTTTTTTGCTGGCTCAATATAACATTGTGTCAAAGGAACTAACAGC
ATACAATGCATAATATATCAATATTAGTCATTTTTCCCTTTATAAATCATATCTCAAAATGTATGCAATATTCTT
TAAATATAACACTTAAAATGCATATGCAATGATGAAATATAGCAGGATTGCCAAAACTGATGAATATCTCAGAGA
TATTGCCATTGTTTTTCCTGGAAAATACTTCTTTGGAAAAAGTGAGACATCTTTGAGATCAAAATAAACACCTGT
CAAAGGAATGTCTTGGGAACCCCAAATAGCATTTCTAAAGAGATAAAAGAATGTTGTAGTCTTCCAGAAAGGAAA
TCAAGTGGAAGTAGATATTAATTCAGCACTACAGAGACAATCTGGAGAAAACAGAGGCATTGTTTTCTAAAGAAA
TTGGCTTTTGTAACATAAAGGAGATACAACTCTGGGAGTGAAAGTTGTCACAGTGATACATATCACACAACGGAA
ATGCCAAACGAAACCATCACCACTTGAAATCAAACTTGATTCAAGTTACTCAGGTAAAATACCACCATGGGTGGC
ATCCTTTTTTTTTTTTTTTTTTTTTTTTTTTTTAACCAAGCAGCCTAACGGTGTTTAAGGAGGAAAACTTAATTG
ATAATGCACTTTGCTCAATTATAAATAAGCTGACTGGGAAAGAAGTGGGCATGATGGAAAGCAAAATTTGAATGA
AGCTGTTATTCTTTTAATCTATAAAAACATTACATCCAAGTTTAGACTCATTGAGCTCTAAATATTTGGGAAAAC
ATATTTAAAGAAATTATATAGGTTTGATCCAAAATCTCTTTGGCACAACTTGAAATATGGGTAATCGTCATGTGA
AATTTGTGAATAGGAGAACCCACTGTAGGATACTTAACATAAATCAGCCACATAATTTCTATCACTGATATCCAG
GGAATTTCAATGACAAATCTAGTGA (SEQ ID NO: 51)
GRNA
TTTGTTGCAGAAGAACTACG GGG (SEQ ID NO: 12)
HA LEFT
TTCATTTGTTCTTTTTGCTGTATTTAAACTGTGGGAATTCTATTGTTAACCTTTTTCTTGCTCAACTGAACTGTG
ACACTGCTAGGAATGCCGAAGCAGGGTTTTAGGTTCTCAGGATGTTTAAGAGTTGGAGAAAGCACTCAATGAGTC
TTGTGAATAATTTTGTGGAAACTGCACTCCCAATGACAGGCTCTGGCATCTCACTCTAAGGTAAGTAACAGGTGA
GGCACTGCCCTTTGATAACCAGGACGTGGATTCTAGAATTGGTTATGTCCATTCACACAATGTGTTCATCTCCTC
TCTGGCTATCCTTCTGATGACTCAGAAGTCGCAACCCCAAGTTGGTTCTTTCAGGGCCCTGGCTTGCTCATCCCC
TTTATGATTCTCCTTTATTCTTCTGTACCTGGGTATTCATTTCATGTCCACCCTCATCATCATTCTGGAGACAAA
GATGCAGACATGTACTGGCAGAATCTGAGCATGCAAAAACCTTCCGTAACACTCAGAGTTCTTATACCTTTCTCT
CATCTGGCATTATTTACTATGATAGGGTGAAGGAAACAACTATGTCTGTTCTTTGCATTTGACTGCATTGTCCAG
TTTCTAGAGGCTTAAGTACACTTTTTTTTTTTTTTTTTTTGAGACAGAGTTTCACTCTTGTTGCCCAGGCTGGAG
TGCAATGACGCGATCTCAGCTCACTGCAACCTCCAACTCTCAGGTTCAAGTGATTCTCCTGCCTCAGCCTCCCAA
ATAGCTGGGATTACAGGCATCTGCCACCACACCTAGCTAATTTTATATTTGTAGTAGAAGCGGGGTTTCTCCATG
TTGGTCAGGCTGGTCTCGAACTCCTGACCTCAGGTGATCCAACCGCCTCAGCCTCCCAAAGTACTGGGATTACAG
GCGTAAGCCACTGCACCCAGCACCTAGTGGCTTAAATACATTTAAAGCATCATAGCTCAACCTCTAAATTGCACT
GCAGCATACACAGCTAAATCCCCGT (SEQ ID NO: 32)
HA RIGHT
AGTTCTTCTGCAACAAAACACAGCAACCCAGCATGTTTTCTCATCTACATTCTACATTCATACCTCATTCTTAGG
GACCAATTGGGGATACTAGGCTGTACCTCCCAGAACCCTTTTTAGAACTAAAGAATTATTTTCACAGCTGCTGAA
AGTGCTGCAGGCTAGAAGTCTTCACCCTGAAGCCCTCTCTGGGACCTGCCCTTGACAAAAGATACCACCTTCTCC
AAGGTCAAGCCCCCTTCCCAGAATCAAGAATTGCTAGAATCAAGAATCAAGAGAATCAAGAATCAAGGCTTCCTA
CCCTCACTTAAACTCAGGGCAATTCTGAAGAGCCAACCCAGCTTCAGAGCTCCCCTACAGCAATGTGTTACAATT
TTGTTATAACTGCATCTTAGCATTCCAATATCTCTTTGTCTAGTCCTGGCTTCTTCCCTTCCCCACAGGGGAACA
CTACCTATAACAAGCCTTAGCATCCCTCCATTTTTAATTTTTCATATTAGTGACCTGCCTTTCATCCTCTGTTCA
TTCACTCAATGAATATTGTCAGAGTACCTACTATACACCAGGCATTATTTTGGGTGCTGGTTATTCAGCAATGAA
CAAGGCTAGAAAGGCCCTGCCATCACAGAGTTGCCATTTCAGTAAGGGAAGAAGGCAATAAACATATAATTTACT
ATCACATAGGGGTAATGCTTGAAGGAAAATTAATTGAACTGTAACAGTACAGAGTTATTGGGAGGAAGCATTTAT
TTAAACAGAAAAGACCAGAGAAGACCCCTATGGGGGGAGTGAGATGGAACTGATAACTGAATGAAGTGACAGAAT
GAACCATGCAAAGACATAAAAAATTGCATTCAAAGCAGAAGAAAGAACAAGTGCAAAGTTCCCAGGGCTCAAATA
ATATGTAATTTTGAAGAATGGTTAGGACAGTTTGGTTAGAGTAGAGTGAAAAGGGGAGAAATTGATAGGAGATAA
ACTCAAAGACAGAGGCAGCAGTTCC (SEQ ID NO: 52)
GRNA
AACAGGCATTATCCTCCTAG GGG (SEQ ID NO: 13)
HA LEFT
AGTCTTCACCCTGAAGCCCTCTCTGGGACCTGCCCTTGACAAAAGATACCACCTTCTCCAAGGTCAAGCCCCCTT
CCCAGAATCAAGAATTGCTAGAATCAAGAATCAAGAGAATCAAGAATCAAGGCTTCCTACCCTCACTTAAACTCA
GGGCAATTCTGAAGAGCCAACCCAGCTTCAGAGCTCCCCTACAGCAATGTGTTACAATTTTGTTATAACTGCATC
TTAGCATTCCAATATCTCTTTGTCTAGTCCTGGCTTCTTCCCTTCCCCACAGGGGAACACTACCTATAACAAGCC
TTAGCATCCCTCCATTTTTAATTTTTCATATTAGTGACCTGCCTTTCATCCTCTGTTCATTCACTCAATGAATAT
TGTCAGAGTACCTACTATACACCAGGCATTATTTTGGGTGCTGGTTATTCAGCAATGAACAAGGCTAGAAAGGCC
CTGCCATCACAGAGTTGCCATTTCAGTAAGGGAAGAAGGCAATAAACATATAATTTACTATCACATAGGGGTAAT
GCTTGAAGGAAAATTAATTGAACTGTAACAGTACAGAGTTATTGGGAGGAAGCATTTATTTAAACAGAAAAGACC
AGAGAAGACCCCTATGGGGGGAGTGAGATGGAACTGATAACTGAATGAAGTGACAGAATGAACCATGCAAAGACA
TAAAAAATTGCATTCAAAGCAGAAGAAAGAACAAGTGCAAAGTTCCCAGGGCTCAAATAATATGTAATTTTGAAG
AATGGTTAGGACAGTTTGGTTAGAGTAGAGTGAAAAGGGGAGAAATTGATAGGAGATAAACTCAAAGACAGAGGC
AGCAGTTCCTGCAGAGCCTTGTAGAGGTAAAGAGGTCATATTTTATTCTGAATGTGATAAGAATCTACTGCAGGA
ACAGGGGAGGACACGGTCCAATTGATGTTTGAAAAGATTATTCTGGCTATTGTATGGAAAGAAACTGAGGGGGCA
AGGGTAGGAGCAGGAAGATCCCCTA (SEQ ID NO: 33)
HA RIGHT
GGAGGATAATGCCTGTTGCCGAGGAAACACAGCCTCATTTGGGTTTCAGATAATGCCAAAAATGAAACAAAAAAA
ATTCACAGAAAATACAGAATGAGTTCAGCATGTGGACTACATTGATGTTGCCTTATACTCTGTGTCTTCATCTTC
CTTCTCCTTTTCTGTTTCTTCACTTTTGATACTAATAGCCCAGAGCTGACCTCACTGTATAAAGGACACAGTGAT
CACCACTAGGGCAGGAAAATAAAATTAACACCCAACTCTTAGAAGACCCCCCACAGGAAACAAAAGTAGCTGATG
CTGTGGTTTACAACACACAATCGCAAACAAATGGATAAGAAACAAAAAAAAAGAAATAGAAAAAAATCAGTTATA
GATAAAGAAACATCTGCTCTCAGACCCAAATATGAGCCAAAGAAGAAAACTTAAGGAAAATATTAAAATATTTTG
AACTAGTATAGCTAACCAAGAAAAAAAGAGAAGATACAAATTTGCATTGTTATAAATAAAAGAAGAACCATCACT
GCTGATTCAAGGACACGTAAAAAATAAAAAGGGGATACTACAAACAACGCTATGCCTGTTCAATAACTTAGATCA
AATGGGCTAATTCTTGAAAGACACAAACTATTGAAACTGACTCAGGGAGAAATAGATAATCTTTAAAAAAATTTT
TTTTTATTCTAAGTTCTGGGATACATGTGCAGAATGTGCAGGTTTGTTACATAGGTATACAAGTGCCATGGTGGT
TTGTTGCACCCATCAACCCATCATCTACATTAGGTATTTCTCCTAATGCTATCCCTCCTGTAGTCCCCCACCCAC
CAACAGGCCCCGGTATGTGATGTTCTCTTCCCTGTGTCCATGTGTACTCATGGTTCAACTACCACTTAAAAGTGA
GAACATGAGGTGTTTGATTTTCTGTTCTTGTGTTAGTATGCTGAGAATGACGGTTTCCAGCTTCATCCATGTCCC
TGCAAAGGACATGAACTCTTCCTTT (SEQ ID NO: 53)
GRNA
GCCCTGAAAGAACCAACTTG GGG (SEQ ID NO: 14)
HA LEFT
GCAGCCTAACGGTGTTTAAGGAGGAAAACTTAATTGATAATGCACTTTGCTCAATTATAAATAAGCTGACTGGGA
AAGAAGTGGGCATGATGGAAAGCAAAATTTGAATGAAGCTGTTATTCTTTTAATCTATAAAAACATTACATCCAA
GTTTAGACTCATTGAGCTCTAAATATTTGGGAAAACATATTTAAAGAAATTATATAGGTTTGATCCAAAATCTCT
TTGGCACAACTTGAAATATGGGTAATCGTCATGTGAAATTTGTGAATAGGAGAACCCACTGTAGGATACTTAACA
TAAATCAGCCACATAATTTCTATCACTGATATCCAGGGAATTTCAATGACAAATCTAGTGATAAAAATTGATAAA
ACATTTTTGATAGTTTTGATACAAGTGAAAGTCATGGGATATCAGACTTAAAAGAAACCTCAGTGCTATCAAGTC
TGATGTCAGTAATTTTTGGAGGAGACTGAAGTGCAGTGAGACTATCCAAAGTCAGACATGGGGAAAAGCAGAGTC
ATCCCTCCTAGGCTGCCAAAATCCTCCCCATCCAAGCTCATCCTTGAAGCCCTCACTTAAGACAAAGTTCCTCCC
ATCCCTTCTGCCTGCTCTGGCATGGTCTGAACCATTTGCCTATTAATTGCCCTGCCTGGTTTCATTTGTTCTTTT
TGCTGTATTTAAACTGTGGGAATTCTATTGTTAACCTTTTTCTTGCTCAACTGAACTGTGACACTGCTAGGAATG
CCGAAGCAGGGTTTTAGGTTCTCAGGATGTTTAAGAGTTGGAGAAAGCACTCAATGAGTCTTGTGAATAATTTTG
TGGAAACTGCACTCCCAATGACAGGCTCTGGCATCTCACTCTAAGGTAAGTAACAGGTGAGGCACTGCCCTTTGA
TAACCAGGACGTGGATTCTAGAATTGGTTATGTCCATTCACACAATGTGTTCATCTCCTCTCTGGCTATCCTTCT
GATGACTCAGAAGTCGCAACCCCAA (SEQ ID NO: 34)
HA RIGHT
GTTGGTTCTTTCAGGGCCCTGGCTTGCTCATCCCCTTTATGATTCTCCTTTATTCTTCTGTACCTGGGTATTCAT
TTCATGTCCACCCTCATCATCATTCTGGAGACAAAGATGCAGACATGTACTGGCAGAATCTGAGCATGCAAAAAC
CTTCCGTAACACTCAGAGTTCTTATACCTTTCTCTCATCTGGCATTATTTACTATGATAGGGTGAAGGAAACAAC
TATGTCTGTTCTTTGCATTTGACTGCATTGTCCAGTTTCTAGAGGCTTAAGTACACTTTTTTTTTTTTTTTTTTT
GAGACAGAGTTTCACTCTTGTTGCCCAGGCTGGAGTGCAATGACGCGATCTCAGCTCACTGCAACCTCCAACTCT
CAGGTTCAAGTGATTCTCCTGCCTCAGCCTCCCAAATAGCTGGGATTACAGGCATCTGCCACCACACCTAGCTAA
TTTTATATTTGTAGTAGAAGCGGGGTTTCTCCATGTTGGTCAGGCTGGTCTCGAACTCCTGACCTCAGGTGATCC
AACCGCCTCAGCCTCCCAAAGTACTGGGATTACAGGCGTAAGCCACTGCACCCAGCACCTAGTGGCTTAAATACA
TTTAAAGCATCATAGCTCAACCTCTAAATTGCACTGCAGCATACACAGCTAAATCCCCGTAGTTCTTCTGCAACA
AAACACAGCAACCCAGCATGTTTTCTCATCTACATTCTACATTCATACCTCATTCTTAGGGACCAATTGGGGATA
CTAGGCTGTACCTCCCAGAACCCTTTTTAGAACTAAAGAATTATTTTCACAGCTGCTGAAAGTGCTGCAGGCTAG
AAGTCTTCACCCTGAAGCCCTCTCTGGGACCTGCCCTTGACAAAAGATACCACCTTCTCCAAGGTCAAGCCCCCT
TCCCAGAATCAAGAATTGCTAGAATCAAGAATCAAGAGAATCAAGAATCAAGGCTTCCTACCCTCACTTAAACTC
AGGGCAATTCTGAAGAGCCAACCCA (SEQ ID NO: 54)
GSH7 TESTED GRNA/HA
GRNA
AGGTGCCTCCAATAAAGCAA GGG (SEQ ID NO: 15)
HA LEFT
TGCATAGGAGAGGTTCTATTACATAGTGGCTGCTCATCAAATGTTTAAAAAATATTAATGAGTTTGATATCTTAG
AATTTTCCTTTAATGTGTATTTTATCAGCATGGTTTTGATGGAGCAGTTAGAGCGGATTGTATAATTATGATTGC
AGGCTCTATTCAATTCACTGACATAAATATCATGTAAGACAAGAACAGGGTTTGATGGCAATCTATCAGGGACTC
CCTAAGGCTTCAATTAAATTCCACAAGCATTTATCAGAGCTATATATGCGGCAGGCCTGTGTAGTCATTAACAAT
ATGCTGCCCTCGAAAAATACACAGGCTACTTTTACATGGCCCTATTAATCAACACTTATTGGGTACAGCGATACA
ACTAGCTGTTAATTCATCCATCCATACCATTATTTATATCTCATTTCACCTTCTACTTATAGAGCTATTATTAGA
GATATTTGATGTAACAAGATAAACTATGCCTTCAGCATTTATTTGATCTGCCAACTTGAAAATACTATCAAGAAG
ACATTAGGATGGATTTTAATGATTCATTCTTAATAAAACCACCTCCATGAGACTGTATAGTTTTTTTATTATAAA
GGCCCGTTTTTTGTTTGTTTGTTTGTTTTAATCAGTTCCTCCAACTATTGTCCTAGTGATCTTGGATAAGTTACA
TAAATTTTCTAACTTCAACTCTTTCTTCACTTGTAAAACATAGATACAAATAGCACATGTCTGAGAGAGTTGTTC
GGTAGATAAAATAAGATAATCAACTATCTCAAGTCTGATTCCATGGGAAATGGATTCTGAGCCTCTGGGATTTGG
ATACAAGGGGTTAGTTGGAGAAATTTCAGGGAATCAACAGCTAGGAAGATTTGAAGGACTCTAGGAAATTTGAGA
TTTGTGTAGAGGGAGAGGCTGAACTGGTCTGCAACAGAAGTGGCAGATGATTCCACAGAGAACCCTGGAGCTGGG
ATATCTTCAGGTGCCTCCAATAAAG (SEQ ID NO: 35)
HA RIGHT
CAAGGGTGTGGTTCTTTGCAGTATTACCTGGACCAGTCATTGGATATAGGTAACTCCCCAGGAAGAAACCATAGC
CTTGGGTAGACAGCTTCCTTAAACAGACAGGAATAGTAGAGAGGGACTCAACTGTGAGCTGTCAACAGTCAACTA
TGCTACCACTAGGTGTACAGGCTATTACAATGCCTGTTGCATGTTAACTGAATAATAAATACTAGCTATTATTGT
TATACCTACTTATCTATTTCTTAATTTATTTATCTATTTACTTTTTAAGATCTTCTCATTTTGAATTATTCTTTG
ACCTCCATCAGAAATTATGCTAGTGAGTCCATTAAATTGATCAGTTCTCACTTTTCTTCTCTGAGAAAATTATCT
GTTTGTTTTTTAAAGCTTTATTGATTCACAGTGGACCTACAATATATTGCACCTATTTAAGATGTAGAAACTAAC
AAATTTTCTCTCTCAAACACACATACACATACACCTGTGAAACCATCACTACAATCAAGGTATGGAACATTTCCA
TCCCTTCCAAAAGAAACCTCCTGCCGATTTGTAAATAACTACCCCTCTATCCCAGTCCCCAAGTAGTAACTGATC
TGCTTTCTGTCACTATACACTAATTTGCCATTTTAAGAATTTTATATTAATGGGATTATACTTTTTGGGGAAAGG
GGTCTGGCTTCTTTGAATCAGCATGACTATTTTGAGATTCATCCAAATTACAGTGTATAGTGTATCAATAGTTTA
TAGTTTTTATTGCTGAATGGTGTTCCTTTGCATGGGTTTACTGCAATTTGTTTATCCATTCACCTGTTCATAGAT
GTTAAGATTGTTTCAACTTTTTAGCTATTATAAATAAAGCTGCTATGAACATTCAGATATAAGCTTAGTACAGAT
ATATCCTTTAGTTAGGAAAATATCTAGGGACAGAATGGTTTGTTCAGATATGTGATTTGCAAATATTTTCTCCAT
CTGTGTTTTGTTTTTTACAATATTT (SEQ ID NO: 55)
PREDICTED GSH7 GRNAS/HAS
GRNA
GGATGAGAATCGCTACTGGG AGG (SEQ ID NO: 16)
HA LEFT
GAAACCTCCTGCCGATTTGTAAATAACTACCCCTCTATCCCAGTCCCCAAGTAGTAACTGATCTGCTTTCTGTCA
CTATACACTAATTTGCCATTTTAAGAATTTTATATTAATGGGATTATACTTTTTGGGGAAAGGGGTCTGGCTTCT
TTGAATCAGCATGACTATTTTGAGATTCATCCAAATTACAGTGTATAGTGTATCAATAGTTTATAGTTTTTATTG
CTGAATGGTGTTCCTTTGCATGGGTTTACTGCAATTTGTTTATCCATTCACCTGTTCATAGATGTTAAGATTGTT
TCAACTTTTTAGCTATTATAAATAAAGCTGCTATGAACATTCAGATATAAGCTTAGTACAGATATATCCTTTAGT
TAGGAAAATATCTAGGGACAGAATGGTTTGTTCAGATATGTGATTTGCAAATATTTTCTCCATCTGTGTTTTGTT
TTTTACAATATTTAACGATGCCTCTTGACGAACAGAAATTATGAAATTAAGTCTTGTTTATCAACTTTTTCTTTT
ATGGTTTATGCTTTTGGTGTTGTATCTAAGAAATTTTTGCCTAACTCAAGGTCAAAAAGGTTTTCTGTGTTTTTT
GGCTTATTTAGGTGTATGAACCATCACTGATTTTTCTTCATAAGGTATAAATATCAAAGTTCATTTGTGGCATAT
TAATATCCAATTTTTCCAGCAGCATTTATTAAAAAGACCACCTTTTCTCCACAAAATTTGCCACTTTTCTACAAA
TAATATTTTATAAAACAGCCAAATAATGTTTTTTTTTAATAGCCAAGGCATCATTTAGTTTATATGTACCTTTTT
GAGTGTGCTTTGTTAGTGTTTTTCTTTTCTTTTCTTTTCTTTTCTTTTCTTTTCTTTTCTTTTCTTTTCTTTTCT
TTTCTTTTCTTTTCTTTTCGAGGCGAGTCTTGTTCTGTCGCTCAGGCTGGAGTGCAGAGTGCAGTGGCCCGATCT
CCTCTCACTGCAACCCCCGCCTCCC (SEQ ID NO: 36)
HA RIGHT
AGTAGCGATTCTCATCCCTCAGCCTCCCGAGTAGCTGGGCCTACAGTCGTGTGCCACCATGCCCGGCTAATTTTT
GTAGTTTTAGTAGAGATGTGGCTTTATGGTGTTGCCCAGGCTGATCTCGAACTCCTGACCTCAAGTGATATGTCC
ACTTTGGCCTCCTATAGTGCTAGGATTACAGGCGTGAGCCGCCGCACCTGGCCGGTAGTGTACATCTTTCAGGGA
ATCATTTCATCTAAATTGTCAATTATTGGCATACAATTATTAAAAATTTTCATTTTAATGCTTTGCCTATATATA
GAATCTGTGATAACATCATCTTTCTCATTTTTTTAATTGGTAATTAGTGTCTTCTCTCTTTTTTTGTTGATCATA
GTTTGTCCTATTGATCTCAAAATAATGGCTTTTGGTTTCATTAATTTTTAAAATCATTTTTCTGTTTTCTTTTTT
GTTGTTGTTGTCTGCTCTGATCTTTGCAATTTCTTATAATTTCTTTGGAATTAATTTGCTTTTTTAGTTTCTTAA
GGTAGAAACTGAGATCACTGATTTGAAGTATTTCTTCTTTTCTAATATAGGCATTTGCTGCTTTATATTACTATT
TAAATACTGCTTTAACAGTGTCCCAAGGGCCTGCATATGTTGAGTTTCAATTTTTATTTATTTACTTTTAAAAAA
TCGGTAAGTTTAAAACATTTTCCAAATATCTGGGATCGTTTAGATATCTCTGTTACTGATTTCTAATTAAATTCC
ACTGGGGTCAGAGAACATACTTTGTACAATTTAATTTTTTAAAATACCAGTGAGTCTTATTTGTGGTCCAGAAAA
TGGTTCATTTTATTAAACATTCTGTGTGCAATTGGAAAGATTGCATATTCCGCTTCTGTTGAGTGTGCTATACAC
CTCAATTAGGTCAAGCTGGTTAATAGTATTGTTCTTTATGTCCTTTCTGATTTGTATTCTATTTTTTTGAGAAGG
TGGTGTTGAAATCTCCAACTATAAT (SEQ ID NO: 56)
GRNA
GTAATAGCCTGTACACCTAG TGG (SEQ ID NO: 17)
HA LEFT
AATTCACTGACATAAATATCATGTAAGACAAGAACAGGGTTTGATGGCAATCTATCAGGGACTCCCTAAGGCTTC
AATTAAATTCCACAAGCATTTATCAGAGCTATATATGCGGCAGGCCTGTGTAGTCATTAACAATATGCTGCCCTC
GAAAAATACACAGGCTACTTTTACATGGCCCTATTAATCAACACTTATTGGGTACAGCGATACAACTAGCTGTTA
ATTCATCCATCCATACCATTATTTATATCTCATTTCACCTTCTACTTATAGAGCTATTATTAGAGATATTTGATG
TAACAAGATAAACTATGCCTTCAGCATTTATTTGATCTGCCAACTTGAAAATACTATCAAGAAGACATTAGGATG
GATTTTAATGATTCATTCTTAATAAAACCACCTCCATGAGACTGTATAGTTTTTTTATTATAAAGGCCCGTTTTT
TGTTTGTTTGTTTGTTTTAATCAGTTCCTCCAACTATTGTCCTAGTGATCTTGGATAAGTTACATAAATTTTCTA
ACTTCAACTCTTTCTTCACTTGTAAAACATAGATACAAATAGCACATGTCTGAGAGAGTTGTTCGGTAGATAAAA
TAAGATAATCAACTATCTCAAGTCTGATTCCATGGGAAATGGATTCTGAGCCTCTGGGATTTGGATACAAGGGGT
TAGTTGGAGAAATTTCAGGGAATCAACAGCTAGGAAGATTTGAAGGACTCTAGGAAATTTGAGATTTGTGTAGAG
GGAGAGGCTGAACTGGTCTGCAACAGAAGTGGCAGATGATTCCACAGAGAACCCTGGAGCTGGGATATCTTCAGG
TGCCTCCAATAAAGCAAGGGTGTGGTTCTTTGCAGTATTACCTGGACCAGTCATTGGATATAGGTAACTCCCCAG
GAAGAAACCATAGCCTTGGGTAGACAGCTTCCTTAAACAGACAGGAATAGTAGAGAGGGACTCAACTGTGAGCTG
TCAACAGTCAACTATGCTACCACTA (SEQ ID NO: 37)
HA RIGHT
GGTGTACAGGCTATTACAATGCCTGTTGCATGTTAACTGAATAATAAATACTAGCTATTATTGTTATACCTACTT
ATCTATTTCTTAATTTATTTATCTATTTACTTTTTAAGATCTTCTCATTTTGAATTATTCTTTGACCTCCATCAG
AAATTATGCTAGTGAGTCCATTAAATTGATCAGTTCTCACTTTTCTTCTCTGAGAAAATTATCTGTTTGTTTTTT
AAAGCTTTATTGATTCACAGTGGACCTACAATATATTGCACCTATTTAAGATGTAGAAACTAACAAATTTTCTCT
CTCAAACACACATACACATACACCTGTGAAACCATCACTACAATCAAGGTATGGAACATTTCCATCCCTTCCAAA
AGAAACCTCCTGCCGATTTGTAAATAACTACCCCTCTATCCCAGTCCCCAAGTAGTAACTGATCTGCTTTCTGTC
ACTATACACTAATTTGCCATTTTAAGAATTTTATATTAATGGGATTATACTTTTTGGGGAAAGGGGTCTGGCTTC
TTTGAATCAGCATGACTATTTTGAGATTCATCCAAATTACAGTGTATAGTGTATCAATAGTTTATAGTTTTTATT
GCTGAATGGTGTTCCTTTGCATGGGTTTACTGCAATTTGTTTATCCATTCACCTGTTCATAGATGTTAAGATTGT
TTCAACTTTTTAGCTATTATAAATAAAGCTGCTATGAACATTCAGATATAAGCTTAGTACAGATATATCCTTTAG
TTAGGAAAATATCTAGGGACAGAATGGTTTGTTCAGATATGTGATTTGCAAATATTTTCTCCATCTGTGTTTTGT
TTTTTACAATATTTAACGATGCCTCTTGACGAACAGAAATTATGAAATTAAGTCTTGTTTATCAACTTTTTCTTT
TATGGTTTATGCTTTTGGTGTTGTATCTAAGAAATTTTTGCCTAACTCAAGGTCAAAAAGGTTTTCTGTGTTTTT
TGGCTTATTTAGGTGTATGAACCAT (SEQ ID NO: 57)
GRNA
TGTAATCCTAGCACTATAGG AGG (SEQ ID NO: 18)
HA LEFT
ACTATTTTGAGATTCATCCAAATTACAGTGTATAGTGTATCAATAGTTTATAGTTTTTATTGCTGAATGGTGTTC
CTTTGCATGGGTTTACTGCAATTTGTTTATCCATTCACCTGTTCATAGATGTTAAGATTGTTTCAACTTTTTAGC
TATTATAAATAAAGCTGCTATGAACATTCAGATATAAGCTTAGTACAGATATATCCTTTAGTTAGGAAAATATCT
AGGGACAGAATGGTTTGTTCAGATATGTGATTTGCAAATATTTTCTCCATCTGTGTTTTGTTTTTTACAATATTT
AACGATGCCTCTTGACGAACAGAAATTATGAAATTAAGTCTTGTTTATCAACTTTTTCTTTTATGGTTTATGCTT
TTGGTGTTGTATCTAAGAAATTTTTGCCTAACTCAAGGTCAAAAAGGTTTTCTGTGTTTTTTGGCTTATTTAGGT
GTATGAACCATCACTGATTTTTCTTCATAAGGTATAAATATCAAAGTTCATTTGTGGCATATTAATATCCAATTT
TTCCAGCAGCATTTATTAAAAAGACCACCTTTTCTCCACAAAATTTGCCACTTTTCTACAAATAATATTTTATAA
AACAGCCAAATAATGTTTTTTTTTAATAGCCAAGGCATCATTTAGTTTATATGTACCTTTTTGAGTGTGCTTTGT
TAGTGTTTTTCTTTTCTTTTCTTTTCTTTTCTTTTCTTTTCTTTTCTTTTCTTTTCTTTTCTTTTCTTTTCTTTT
CTTTTCGAGGCGAGTCTTGTTCTGTCGCTCAGGCTGGAGTGCAGAGTGCAGTGGCCCGATCTCCTCTCACTGCAA
CCCCCGCCTCCCAGTAGCGATTCTCATCCCTCAGCCTCCCGAGTAGCTGGGCCTACAGTCGTGTGCCACCATGCC
CGGCTAATTTTTGTAGTTTTAGTAGAGATGTGGCTTTATGGTGTTGCCCAGGCTGATCTCGAACTCCTGACCTCA
AGTGATATGTCCACTTTGGCCTCCT (SEQ ID NO: 38)
HA RIGHT
ATAGTGCTAGGATTACAGGCGTGAGCCGCCGCACCTGGCCGGTAGTGTACATCTTTCAGGGAATCATTTCATCTA
AATTGTCAATTATTGGCATACAATTATTAAAAATTTTCATTTTAATGCTTTGCCTATATATAGAATCTGTGATAA
CATCATCTTTCTCATTTTTTTAATTGGTAATTAGTGTCTTCTCTCTTTTTTTGTTGATCATAGTTTGTCCTATTG
ATCTCAAAATAATGGCTTTTGGTTTCATTAATTTTTAAAATCATTTTTCTGTTTTCTTTTTTGTTGTTGTTGTCT
GCTCTGATCTTTGCAATTTCTTATAATTTCTTTGGAATTAATTTGCTTTTTTAGTTTCTTAAGGTAGAAACTGAG
ATCACTGATTTGAAGTATTTCTTCTTTTCTAATATAGGCATTTGCTGCTTTATATTACTATTTAAATACTGCTTT
AACAGTGTCCCAAGGGCCTGCATATGTTGAGTTTCAATTTTTATTTATTTACTTTTAAAAAATCGGTAAGTTTAA
AACATTTTCCAAATATCTGGGATCGTTTAGATATCTCTGTTACTGATTTCTAATTAAATTCCACTGGGGTCAGAG
AACATACTTTGTACAATTTAATTTTTTAAAATACCAGTGAGTCTTATTTGTGGTCCAGAAAATGGTTCATTTTAT
TAAACATTCTGTGTGCAATTGGAAAGATTGCATATTCCGCTTCTGTTGAGTGTGCTATACACCTCAATTAGGTCA
AGCTGGTTAATAGTATTGTTCTTTATGTCCTTTCTGATTTGTATTCTATTTTTTTGAGAAGGTGGTGTTGAAATC
TCCAACTATAATTGTAGATTTCTCTATTTCTCCTTGCAATTGTATCAGATTTTACCTATTTTGAAAATCTGTTAT
TAGGTGCATAAGTATTTGCAAGTATTATCTTCTCTCAATGTATTGATTCTTTTTTTGAAATTATGAAATGACACT
TTATCCCTTTTAATAATTACAGACA (SEQ ID NO: 58)
GRNA
TTGGATATAGGTAACTCCCC AGG (SEQ ID NO: 19)
HA LEFT
AATGAGTTTGATATCTTAGAATTTTCCTTTAATGTGTATTTTATCAGCATGGTTTTGATGGAGCAGTTAGAGCGG
ATTGTATAATTATGATTGCAGGCTCTATTCAATTCACTGACATAAATATCATGTAAGACAAGAACAGGGTTTGAT
GGCAATCTATCAGGGACTCCCTAAGGCTTCAATTAAATTCCACAAGCATTTATCAGAGCTATATATGCGGCAGGC
CTGTGTAGTCATTAACAATATGCTGCCCTCGAAAAATACACAGGCTACTTTTACATGGCCCTATTAATCAACACT
TATTGGGTACAGCGATACAACTAGCTGTTAATTCATCCATCCATACCATTATTTATATCTCATTTCACCTTCTAC
TTATAGAGCTATTATTAGAGATATTTGATGTAACAAGATAAACTATGCCTTCAGCATTTATTTGATCTGCCAACT
TGAAAATACTATCAAGAAGACATTAGGATGGATTTTAATGATTCATTCTTAATAAAACCACCTCCATGAGACTGT
ATAGTTTTTTTATTATAAAGGCCCGTTTTTTGTTTGTTTGTTTGTTTTAATCAGTTCCTCCAACTATTGTCCTAG
TGATCTTGGATAAGTTACATAAATTTTCTAACTTCAACTCTTTCTTCACTTGTAAAACATAGATACAAATAGCAC
ATGTCTGAGAGAGTTGTTCGGTAGATAAAATAAGATAATCAACTATCTCAAGTCTGATTCCATGGGAAATGGATT
CTGAGCCTCTGGGATTTGGATACAAGGGGTTAGTTGGAGAAATTTCAGGGAATCAACAGCTAGGAAGATTTGAAG
GACTCTAGGAAATTTGAGATTTGTGTAGAGGGAGAGGCTGAACTGGTCTGCAACAGAAGTGGCAGATGATTCCAC
AGAGAACCCTGGAGCTGGGATATCTTCAGGTGCCTCCAATAAAGCAAGGGTGTGGTTCTTTGCAGTATTACCTGG
ACCAGTCATTGGATATAGGTAACTC (SEQ ID NO: 39)
HA RIGHT
CCCAGGAAGAAACCATAGCCTTGGGTAGACAGCTTCCTTAAACAGACAGGAATAGTAGAGAGGGACTCAACTGTG
AGCTGTCAACAGTCAACTATGCTACCACTAGGTGTACAGGCTATTACAATGCCTGTTGCATGTTAACTGAATAAT
AAATACTAGCTATTATTGTTATACCTACTTATCTATTTCTTAATTTATTTATCTATTTACTTTTTAAGATCTTCT
CATTTTGAATTATTCTTTGACCTCCATCAGAAATTATGCTAGTGAGTCCATTAAATTGATCAGTTCTCACTTTTC
TTCTCTGAGAAAATTATCTGTTTGTTTTTTAAAGCTTTATTGATTCACAGTGGACCTACAATATATTGCACCTAT
TTAAGATGTAGAAACTAACAAATTTTCTCTCTCAAACACACATACACATACACCTGTGAAACCATCACTACAATC
AAGGTATGGAACATTTCCATCCCTTCCAAAAGAAACCTCCTGCCGATTTGTAAATAACTACCCCTCTATCCCAGT
CCCCAAGTAGTAACTGATCTGCTTTCTGTCACTATACACTAATTTGCCATTTTAAGAATTTTATATTAATGGGAT
TATACTTTTTGGGGAAAGGGGTCTGGCTTCTTTGAATCAGCATGACTATTTTGAGATTCATCCAAATTACAGTGT
ATAGTGTATCAATAGTTTATAGTTTTTATTGCTGAATGGTGTTCCTTTGCATGGGTTTACTGCAATTTGTTTATC
CATTCACCTGTTCATAGATGTTAAGATTGTTTCAACTTTTTAGCTATTATAAATAAAGCTGCTATGAACATTCAG
ATATAAGCTTAGTACAGATATATCCTTTAGTTAGGAAAATATCTAGGGACAGAATGGTTTGTTCAGATATGTGAT
TTGCAAATATTTTCTCCATCTGTGTTTTGTTTTTTACAATATTTAACGATGCCTCTTGACGAACAGAAATTATGA
AATTAAGTCTTGTTTATCAACTTTT (SEQ ID NO: 59)
GSH31-TESTED GRNA/HA
GRNA
ATAGGCTGTCCATAACCCGG AGG (SEQ ID NO: 20)
HA LEFT
ATAGACCAGCAGCATCAGCATCACCTAGGAAAATGTTAGAAATGCAAATTCTTGGGCCCCATCACTCAATCGCTG
AGGATGGGACACAGGTGGTTCTGATGCATGCTGATGACTGAGAATCACGGTACAGAATATACTGATGCAGGATTT
TCTGCTCCTTAGCTCACCTAAATCCGGGATCTTGTCTCATCACCAGGAAGTAGTAGGCACACGGACACAATGAAG
GGGGGTGAGGGCAGAATTTATTAAGTGAAAGGAAAGCACTCAGTAAAGAGAGGTGTCCTGCACACAGGCTTCCAC
CTCACAAATTTGAATACCAGGCCACCACGCATGAGTTGCAAAGGTAAGGCTCCTCCCCCACAAAAGGTGCAAATT
CCTGGGGGTTTGACCCTATTCTCTCAGGGTGCATGCCGACCCTTAGTCTGAGCCACTCCACATTGATTTATTTCC
ATTACTGTGCATGTGTTAAGGGATGGAATTTTTGAGTGTGGGCATATTTAGGCAACCCCCATGTGTGGAATGATT
TGGGCAGGTTGGAGGTTCTTTGAGGGCCCTTCCCTATCTGCTAGGCATTTGTCAGCTTCCTGCCTCTATCATTCC
CGCTTCTAAAGAGGTACATCTGACTGTGTTAGAATACGGATGAAGACTGATCTTAACTGCTTCCTGCTGACAGGG
GGCGCTGTTTTGGGAAGATGGCAGTCATGTCTCCCTCAGAGGCCTATCTAAGGGTCCCCAGTAAAAAGAGCCATC
ATCAGAGGCATTGTTTGCATGACCATTTAGAGTTTGGTGGCCTGAAGGTGAGAAGAGACAAACTGGGTTATTAGA
ATACATGTATCAAAACGAAACAAAAAGGGGTGGGTAAGGACAGCTCAAAAATCCCAAGACTGATGGCACACCCAG
ATAGCTGGTGGCTACAGTTATGCCTGCCAAGATTTGGGTGCATGGGACTTGGCTTTGATTAGCTCCCTTGGTCTT
ATTTTCCCGTATAAAGAAACCTCCG (SEQ ID NO: 40)
HA RIGHT
GGTTATGGACAGCCTATTTACTCGTATCACCTTGCAGGGTTTGTAGGATAATTGCCCAGAACTAGAATATTAATC
CAGACTTTTACATTACGCATCCCTTCTGTTTCTTCTGAACTGCAGCTAGACATCACTGGTTGATCCATGAAATAA
GCAGGGTTAGTTCAAAATGTGGGTAAAAAGCTTAAAAACAACTGAGTCTAGAATTTAATGACAAATGTATGGTAA
GTTTTGAAACTTATCATACAGTGGCATGCTCTCAGCTCACTGCAACCTCCACCTCTCCAGTTCAAGTGATTCTCC
TTCCTCAGCCTCCCTAGTAGCTGAATTACAGGTGCACGCCAACATGCCCAACTAATGTTTGTATTTTTAGTAGAG
ACAGGGTTTCTCCACGTTGGCCAGGCTGGTCTCAAAATCCTGGTCTCAAGTGATCCTCCCGCCTCAGCCTCCCAA
AATCCTGGGATTACAGGAGTGAGCCACCATGCCTGGTCCATTCTGTTAACACTTGTTCTGTTTGATATTTCTGAA
CATTTCAGCTATTCATTAATCCTGTATGTTTTTCCTTATTCCAATGTCATAATCTCCAAAGTTATCAGAAACCTG
AATTTGAGAGCACCTGTCGAAGTCTTATAGCTGATTATAAATCATCTTTTGAAGAGGATCAAGATGAGACAATTG
TCTGTGAATAACAAAATGTCCAGGGTAGTTACAATTAAATACACAATTGACAAGAAATTTGGTTATCACTTTGGT
TTACAATAATTTAACCTTAATTATGATTGATAGCAGCATATACTCAGACATTCGAATTTTAGAAATCCCATATAA
TTTTGGAACGTATATTAATATTATTCACTAAAATGTAACCTGAAGAAGACTGAACATCATTTTAGTAATCTCACG
TAACTAAACTTGTCAAATAATTCTGTTTACCTCTCTTTTGGATGCTCCAAGAGCCCTCTGTAGCATCCAAAAGCC
AGGGGTCAGGAAAGACAACCCTG (SEQ ID NO: 60)
PREDICTED GSH31 GRNAS/HAS
GRNA
GAGTGGCTCAGACTAAGGGT CGG (SEQ ID NO: 21)
HA LEFT
TCTCTATGCGACATCTTTAAATCTACCATCACAGTATCTGTCTTTGTCTTCCTTTTATTTGAAAAGCAAATAAAC
TACCAATTGTTTTTTAATTAATATGATCAATGATATAAAGCTCTTCATTTAGTTTCTATCTGTGTGGAAACTCAC
CCACAGCATCTTTTGCTTTCATGTGATTCCAGAAGCAAATTTTATAATAAAGTTTCCCTACAGGTTTTAAAATGA
TCAAGTTTATATTCTACCTGATTTTCATTGTACTTTATTTCTCCCTATATAATTGGAAAAAGATATTAGAAGATA
CATTGATTTTATCTGCCTCTATATAGGAAGTCTATAAACCAGATGACTGAAAAGAACAAAACGGAAAACACTTAA
ACTTCACTGATTATCAGAATCAGGAAAACAATGGTTTAGGAAATAATAGCATTCTGCAATCCACTGAAGACATGC
TGAATCAGTTTCTTCTAGGTCTGGAAACAAGAAATTTATTTTTTCTCAGCTGTTGAATTATTCCGATGTAAACAG
GTTTAGTGCTTAAATTTGGAAATCAGTGTCATAGATGAGTGGTTGTCAAAATGTTGTTCATAGACCAGCAGCATC
AGCATCACCTAGGAAAATGTTAGAAATGCAAATTCTTGGGCCCCATCACTCAATCGCTGAGGATGGGACACAGGT
GGTTCTGATGCATGCTGATGACTGAGAATCACGGTACAGAATATACTGATGCAGGATTTTCTGCTCCTTAGCTCA
CCTAAATCCGGGATCTTGTCTCATCACCAGGAAGTAGTAGGCACACGGACACAATGAAGGGGGGTGAGGGCAGAA
TTTATTAAGTGAAAGGAAAGCACTCAGTAAAGAGAGGTGTCCTGCACACAGGCTTCCACCTCACAAATTTGAATA
CCAGGCCACCACGCATGAGTTGCAAAGGTAAGGCTCCTCCCCCACAAAAGGTGCAAATTCCTGGGGGTTTGACCC
TATTCTCTCAGGGTGCATGCCGACC (SEQ ID NO: 41)
HA RIGHT
CTTAGTCTGAGCCACTCCACATTGATTTATTTCCATTACTGTGCATGTGTTAAGGGATGGAATTTTTGAGTGTGG
GCATATTTAGGCAACCCCCATGTGTGGAATGATTTGGGCAGGTTGGAGGTTCTTTGAGGGCCCTTCCCTATCTGC
TAGGCATTTGTCAGCTTCCTGCCTCTATCATTCCCGCTTCTAAAGAGGTACATCTGACTGTGTTAGAATACGGAT
GAAGACTGATCTTAACTGCTTCCTGCTGACAGGGGGCGCTGTTTTGGGAAGATGGCAGTCATGTCTCCCTCAGAG
GCCTATCTAAGGGTCCCCAGTAAAAAGAGCCATCATCAGAGGCATTGTTTGCATGACCATTTAGAGTTTGGTGGC
CTGAAGGTGAGAAGAGACAAACTGGGTTATTAGAATACATGTATCAAAACGAAACAAAAAGGGGTGGGTAAGGAC
AGCTCAAAAATCCCAAGACTGATGGCACACCCAGATAGCTGGTGGCTACAGTTATGCCTGCCAAGATTTGGGTGC
ATGGGACTTGGCTTTGATTAGCTCCCTTGGTCTTATTTTCCCGTATAAAGAAACCTCCGGGTTATGGACAGCCTA
TTTACTCGTATCACCTTGCAGGGTTTGTAGGATAATTGCCCAGAACTAGAATATTAATCCAGACTTTTACATTAC
GCATCCCTTCTGTTTCTTCTGAACTGCAGCTAGACATCACTGGTTGATCCATGAAATAAGCAGGGTTAGTTCAAA
ATGTGGGTAAAAAGCTTAAAAACAACTGAGTCTAGAATTTAATGACAAATGTATGGTAAGTTTTGAAACTTATCA
TACAGTGGCATGCTCTCAGCTCACTGCAACCTCCACCTCTCCAGTTCAAGTGATTCTCCTTCCTCAGCCTCCCTA
GTAGCTGAATTACAGGTGCACGCCAACATGCCCAACTAATGTTTGTATTTTTAGTAGAGACAGGGTTTCTCCACG
TTGGCCAGGCTGGTCTCAAAATCCT (SEQ ID NO: 61)
GRNA
GCCCCATCACTCAATCGCTG AGG (SEQ ID NO: 22)
HA LEFT
AAACTAGCTACTCTTTGAAAACTGCCTTCTTAGAAGTCATCAAAAGTCTTTCATTTTAAAAGAATCCTGCTTTAA
AATAAGCTTAAATAGAAAACCTAACAAGGTCTTTAAAAATGATTTAAAGAAATTTCATCTATCACAAAGGCTTAT
GATTGCTTTACTTTATTCTTTGTAGAAGATTCTACATATGAGTGTGAGAAAGATGAATTATACCTCTGTATGCAT
GGGAAATACCTTGATCCACTAAATCCATAGTATAGGGAAGCAATTTATAAGAGGGTTTCACTATTCTGGTTTTCT
ATTATCATCAGAATATTGTAATCTTTCTGAAGTGTCTCTGGCATTCTCTATGCGACATCTTTAAATCTACCATCA
CAGTATCTGTCTTTGTCTTCCTTTTATTTGAAAAGCAAATAAACTACCAATTGTTTTTTAATTAATATGATCAAT
GATATAAAGCTCTTCATTTAGTTTCTATCTGTGTGGAAACTCACCCACAGCATCTTTTGCTTTCATGTGATTCCA
GAAGCAAATTTTATAATAAAGTTTCCCTACAGGTTTTAAAATGATCAAGTTTATATTCTACCTGATTTTCATTGT
ACTTTATTTCTCCCTATATAATTGGAAAAAGATATTAGAAGATACATTGATTTTATCTGCCTCTATATAGGAAGT
CTATAAACCAGATGACTGAAAAGAACAAAACGGAAAACACTTAAACTTCACTGATTATCAGAATCAGGAAAACAA
TGGTTTAGGAAATAATAGCATTCTGCAATCCACTGAAGACATGCTGAATCAGTTTCTTCTAGGTCTGGAAACAAG
AAATTTATTTTTTCTCAGCTGTTGAATTATTCCGATGTAAACAGGTTTAGTGCTTAAATTTGGAAATCAGTGTCA
TAGATGAGTGGTTGTCAAAATGTTGTTCATAGACCAGCAGCATCAGCATCACCTAGGAAAATGTTAGAAATGCAA
ATTCTTGGGCCCCATCACTCAATCG (SEQ ID NO: 42)
HA RIGHT
CTGAGGATGGGACACAGGTGGTTCTGATGCATGCTGATGACTGAGAATCACGGTACAGAATATACTGATGCAGGA
TTTTCTGCTCCTTAGCTCACCTAAATCCGGGATCTTGTCTCATCACCAGGAAGTAGTAGGCACACGGACACAATG
AAGGGGGGTGAGGGCAGAATTTATTAAGTGAAAGGAAAGCACTCAGTAAAGAGAGGTGTCCTGCACACAGGCTTC
CACCTCACAAATTTGAATACCAGGCCACCACGCATGAGTTGCAAAGGTAAGGCTCCTCCCCCACAAAAGGTGCAA
ATTCCTGGGGGTTTGACCCTATTCTCTCAGGGTGCATGCCGACCCTTAGTCTGAGCCACTCCACATTGATTTATT
TCCATTACTGTGCATGTGTTAAGGGATGGAATTTTTGAGTGTGGGCATATTTAGGCAACCCCCATGTGTGGAATG
ATTTGGGCAGGTTGGAGGTTCTTTGAGGGCCCTTCCCTATCTGCTAGGCATTTGTCAGCTTCCTGCCTCTATCAT
TCCCGCTTCTAAAGAGGTACATCTGACTGTGTTAGAATACGGATGAAGACTGATCTTAACTGCTTCCTGCTGACA
GGGGGCGCTGTTTTGGGAAGATGGCAGTCATGTCTCCCTCAGAGGCCTATCTAAGGGTCCCCAGTAAAAAGAGCC
ATCATCAGAGGCATTGTTTGCATGACCATTTAGAGTTTGGTGGCCTGAAGGTGAGAAGAGACAAACTGGGTTATT
AGAATACATGTATCAAAACGAAACAAAAAGGGGTGGGTAAGGACAGCTCAAAAATCCCAAGACTGATGGCACACC
CAGATAGCTGGTGGCTACAGTTATGCCTGCCAAGATTTGGGTGCATGGGACTTGGCTTTGATTAGCTCCCTTGGT
CTTATTTTCCCGTATAAAGAAACCTCCGGGTTATGGACAGCCTATTTACTCGTATCACCTTGCAGGGTTTGTAGG
ATAATTGCCCAGAACTAGAATATTA (SEQ ID NO: 62)
GRNA
CCTTTGCAACTCATGCGTGG TGG (SEQ ID NO: 23)
HA LEFT
AGGGAAGCAATTTATAAGAGGGTTTCACTATTCTGGTTTTCTATTATCATCAGAATATTGTAATCTTTCTGAAGT
GTCTCTGGCATTCTCTATGCGACATCTTTAAATCTACCATCACAGTATCTGTCTTTGTCTTCCTTTTATTTGAAA
AGCAAATAAACTACCAATTGTTTTTTAATTAATATGATCAATGATATAAAGCTCTTCATTTAGTTTCTATCTGTG
TGGAAACTCACCCACAGCATCTTTTGCTTTCATGTGATTCCAGAAGCAAATTTTATAATAAAGTTTCCCTACAGG
TTTTAAAATGATCAAGTTTATATTCTACCTGATTTTCATTGTACTTTATTTCTCCCTATATAATTGGAAAAAGAT
ATTAGAAGATACATTGATTTTATCTGCCTCTATATAGGAAGTCTATAAACCAGATGACTGAAAAGAACAAAACGG
AAAACACTTAAACTTCACTGATTATCAGAATCAGGAAAACAATGGTTTAGGAAATAATAGCATTCTGCAATCCAC
TGAAGACATGCTGAATCAGTTTCTTCTAGGTCTGGAAACAAGAAATTTATTTTTTCTCAGCTGTTGAATTATTCC
GATGTAAACAGGTTTAGTGCTTAAATTTGGAAATCAGTGTCATAGATGAGTGGTTGTCAAAATGTTGTTCATAGA
CCAGCAGCATCAGCATCACCTAGGAAAATGTTAGAAATGCAAATTCTTGGGCCCCATCACTCAATCGCTGAGGAT
GGGACACAGGTGGTTCTGATGCATGCTGATGACTGAGAATCACGGTACAGAATATACTGATGCAGGATTTTCTGC
TCCTTAGCTCACCTAAATCCGGGATCTTGTCTCATCACCAGGAAGTAGTAGGCACACGGACACAATGAAGGGGGG
TGAGGGCAGAATTTATTAAGTGAAAGGAAAGCACTCAGTAAAGAGAGGTGTCCTGCACACAGGCTTCCACCTCAC
AAATTTGAATACCAGGCCACCACGC (SEQ ID NO: 43)
HA RIGHT
ATGAGTTGCAAAGGTAAGGCTCCTCCCCCACAAAAGGTGCAAATTCCTGGGGGTTTGACCCTATTCTCTCAGGGT
GCATGCCGACCCTTAGTCTGAGCCACTCCACATTGATTTATTTCCATTACTGTGCATGTGTTAAGGGATGGAATT
TTTGAGTGTGGGCATATTTAGGCAACCCCCATGTGTGGAATGATTTGGGCAGGTTGGAGGTTCTTTGAGGGCCCT
TCCCTATCTGCTAGGCATTTGTCAGCTTCCTGCCTCTATCATTCCCGCTTCTAAAGAGGTACATCTGACTGTGTT
AGAATACGGATGAAGACTGATCTTAACTGCTTCCTGCTGACAGGGGGCGCTGTTTTGGGAAGATGGCAGTCATGT
CTCCCTCAGAGGCCTATCTAAGGGTCCCCAGTAAAAAGAGCCATCATCAGAGGCATTGTTTGCATGACCATTTAG
AGTTTGGTGGCCTGAAGGTGAGAAGAGACAAACTGGGTTATTAGAATACATGTATCAAAACGAAACAAAAAGGGG
TGGGTAAGGACAGCTCAAAAATCCCAAGACTGATGGCACACCCAGATAGCTGGTGGCTACAGTTATGCCTGCCAA
GATTTGGGTGCATGGGACTTGGCTTTGATTAGCTCCCTTGGTCTTATTTTCCCGTATAAAGAAACCTCCGGGTTA
TGGACAGCCTATTTACTCGTATCACCTTGCAGGGTTTGTAGGATAATTGCCCAGAACTAGAATATTAATCCAGAC
TTTTACATTACGCATCCCTTCTGTTTCTTCTGAACTGCAGCTAGACATCACTGGTTGATCCATGAAATAAGCAGG
GTTAGTTCAAAATGTGGGTAAAAAGCTTAAAAACAACTGAGTCTAGAATTTAATGACAAATGTATGGTAAGTTTT
GAAACTTATCATACAGTGGCATGCTCTCAGCTCACTGCAACCTCCACCTCTCCAGTTCAAGTGATTCTCCTTCCT
CAGCCTCCCTAGTAGCTGAATTACA (SEQ ID NO: 63)
GRNA
CACACGGACACAATGAAGGG GGG (SEQ ID NO: 24)
HA LEFT
TTGCTTTACTTTATTCTTTGTAGAAGATTCTACATATGAGTGTGAGAAAGATGAATTATACCTCTGTATGCATGG
GAAATACCTTGATCCACTAAATCCATAGTATAGGGAAGCAATTTATAAGAGGGTTTCACTATTCTGGTTTTCTAT
TATCATCAGAATATTGTAATCTTTCTGAAGTGTCTCTGGCATTCTCTATGCGACATCTTTAAATCTACCATCACA
GTATCTGTCTTTGTCTTCCTTTTATTTGAAAAGCAAATAAACTACCAATTGTTTTTTAATTAATATGATCAATGA
TATAAAGCTCTTCATTTAGTTTCTATCTGTGTGGAAACTCACCCACAGCATCTTTTGCTTTCATGTGATTCCAGA
AGCAAATTTTATAATAAAGTTTCCCTACAGGTTTTAAAATGATCAAGTTTATATTCTACCTGATTTTCATTGTAC
TTTATTTCTCCCTATATAATTGGAAAAAGATATTAGAAGATACATTGATTTTATCTGCCTCTATATAGGAAGTCT
ATAAACCAGATGACTGAAAAGAACAAAACGGAAAACACTTAAACTTCACTGATTATCAGAATCAGGAAAACAATG
GTTTAGGAAATAATAGCATTCTGCAATCCACTGAAGACATGCTGAATCAGTTTCTTCTAGGTCTGGAAACAAGAA
ATTTATTTTTTCTCAGCTGTTGAATTATTCCGATGTAAACAGGTTTAGTGCTTAAATTTGGAAATCAGTGTCATA
GATGAGTGGTTGTCAAAATGTTGTTCATAGACCAGCAGCATCAGCATCACCTAGGAAAATGTTAGAAATGCAAAT
TCTTGGGCCCCATCACTCAATCGCTGAGGATGGGACACAGGTGGTTCTGATGCATGCTGATGACTGAGAATCACG
GTACAGAATATACTGATGCAGGATTTTCTGCTCCTTAGCTCACCTAAATCCGGGATCTTGTCTCATCACCAGGAA
GTAGTAGGCACACGGACACAATGAA (SEQ ID NO: 44)
HA RIGHT
GGGGGGTGAGGGCAGAATTTATTAAGTGAAAGGAAAGCACTCAGTAAAGAGAGGTGTCCTGCACACAGGCTTCCA
CCTCACAAATTTGAATACCAGGCCACCACGCATGAGTTGCAAAGGTAAGGCTCCTCCCCCACAAAAGGTGCAAAT
TCCTGGGGGTTTGACCCTATTCTCTCAGGGTGCATGCCGACCCTTAGTCTGAGCCACTCCACATTGATTTATTTC
CATTACTGTGCATGTGTTAAGGGATGGAATTTTTGAGTGTGGGCATATTTAGGCAACCCCCATGTGTGGAATGAT
TTGGGCAGGTTGGAGGTTCTTTGAGGGCCCTTCCCTATCTGCTAGGCATTTGTCAGCTTCCTGCCTCTATCATTC
CCGCTTCTAAAGAGGTACATCTGACTGTGTTAGAATACGGATGAAGACTGATCTTAACTGCTTCCTGCTGACAGG
GGGCGCTGTTTTGGGAAGATGGCAGTCATGTCTCCCTCAGAGGCCTATCTAAGGGTCCCCAGTAAAAAGAGCCAT
CATCAGAGGCATTGTTTGCATGACCATTTAGAGTTTGGTGGCCTGAAGGTGAGAAGAGACAAACTGGGTTATTAG
AATACATGTATCAAAACGAAACAAAAAGGGGTGGGTAAGGACAGCTCAAAAATCCCAAGACTGATGGCACACCCA
GATAGCTGGTGGCTACAGTTATGCCTGCCAAGATTTGGGTGCATGGGACTTGGCTTTGATTAGCTCCCTTGGTCT
TATTTTCCCGTATAAAGAAACCTCCGGGTTATGGACAGCCTATTTACTCGTATCACCTTGCAGGGTTTGTAGGAT
AATTGCCCAGAACTAGAATATTAATCCAGACTTTTACATTACGCATCCCTTCTGTTTCTTCTGAACTGCAGCTAG
ACATCACTGGTTGATCCATGAAATAAGCAGGGTTAGTTCAAAATGTGGGTAAAAAGCTTAAAAACAACTGAGTCT
AGAATTTAATGACAAATGTATGGTA (SEQ ID NO: 64)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. An engineered nucleic acid targeting vector comprising a sequence of interest flanked by homology arms, each homology arm comprising a sequence homologous to a sequence in a safe harbor site in the human genome in any one of the following loci: 1q31, 3p24, 7q35, and Xq21.

2. The vector of claim 1, wherein the safe harbor site is at position 31 on the long arm of chromosome 1 (1q31).

3. The vector of claim 2, wherein the safe harbor site is at position 31.3 on the long arm of chromosome 1 (1q31.3).

4. The vector of claim 3, wherein the safe harbor site is within coordinates 195,338,589-195,818,588[GRCh38/hg38] of 1q31.3.

5. The vector of claim 1, wherein the safe harbor site is at position 24 on the short arm of chromosome 3 (3p24).

6. The vector of claim 5, wherein the safe harbor site at position 24.3 on the short arm of chromosome 3 (3p24.3).

7. The vector of claim 6, wherein the safe harbor site is within coordinates 22,720,711-22,761,389[GRCh38/hg38] of 3p24.3.

8. The vector of claim 1, wherein the safe harbor site is at position 35 of the long arm of chromosome 7 (7q35).

9. The vector of claim 8, wherein the safe harbor site is within coordinates 145,090,941-145,219,513[GRCh38/hg38] of 7q35.

10. The vector of claim 1, wherein the safe harbor site is at position 21 in the long arm of chromosome X (Xq21).

11. The vector of claim 10, wherein the safe harbor site is at position 21.31 in the long arm of chromosome X (Xq21.31).

12. The vector of claim 11, wherein the safe harbor site is within coordinates 89,174,426-89,179,074[GRCh38/hg38] of Xq21.31.

13. The vector of any one of the preceding claims, wherein the sequence of interest comprises an open reading frame.

14. The vector of any one of the preceding claims, wherein the vector comprises a promoter operably linked to the sequence of interest.

15. The vector of any one of the preceding claims, wherein the sequence of interest is a gene of interest or a region of a gene of interest.

16. The vector of any one of the preceding claims, wherein the sequence of interest encodes a full-length or truncated protein.

17. The vector of claim 15 or 16, wherein the gene of interest is selected from Table 2.

18. The vector of any one of the preceding claims, wherein the vector is a double-stranded DNA vector, optionally wherein the sequence of interest is flanked by regions that enable circularization, preferably via trans-splicing, upon expression.

19. The vector of any one of the preceding claims, wherein each homology arm has a length of about 200 to about 500 base pairs (bp), optionally 300 bp.

20. The vector of any one of the preceding claims, wherein each homology arm is a microhomology arm having a length of about 5 to 50 bp, optionally 40 bp.

21. The vector of any one of the preceding claims, further comprising a sequence encoding at least one guide RNA that specifically targets the sequence in the safe harbor site and/or specifically targets a sequence in or near the homology arms.

22. The vector of any one of the preceding claims, further comprising a sequence encoding a programmable nuclease.

23. A delivery system, optionally a lipid nanoparticle, comprising the vector of any one of the preceding claims.

24. The delivery system of claim 23 further comprising a programmable nuclease or a nucleic acid encoding the programmable nuclease.

25. The delivery system of claim 24, wherein the programmable nuclease is selected from ZFNs, TALENs, DNA-guided nucleases, and RNA-guided nucleases.

26. The delivery system of claim 25, wherein the programmable nuclease is an RNA-guided nuclease.

27. The delivery system of claim 26, wherein the RNA-guided nuclease is a CRISPR Cas nuclease and the delivery system further comprises a guide RNA or a nucleic acid encoding the gRNA.

28. The delivery system of claim 27, wherein the CRISPR Cas nuclease is a Cas9 nuclease or a Cas12 nuclease.

29. The delivery system of claim 27 or 28, wherein the gRNA specifically targets the sequence in the safe harbor site and/or specifically targets a sequence in or near the homology arms.

30. A method comprising delivering to a human cell the delivery system of any one of the preceding claims.

31. A method comprising delivering to a human cell the engineered targeting vector any one of the preceding claims.

32. The method of claim 31 further comprising delivering to the human cell a programmable nuclease or a nucleic acid encoding the programmable nuclease.

33. The method of any one of the preceding claims further comprising incubating the human cell to modify the safe harbor site to include the sequence of interest.

34. A method comprising delivering to a subject the delivery system of any one of the preceding claims.

35. A method comprising delivering to a subject the engineered targeting vector any one of the preceding claims.

36. The method of claim 35 further comprising delivering to the subject a programmable nuclease or a nucleic acid encoding the programmable nuclease.

37. The method of any one of the preceding claims, wherein the programmable nuclease is selected from ZFNs, TALENs, DNA-guided nucleases, and RNA-guided nucleases.

38. The method of claim 37, wherein the programmable nuclease is an RNA-guided nuclease.

39. The method of claim 38, wherein the RNA-guided nuclease is a CRISPR Cas nuclease and the delivery system further comprises a guide RNA or a nucleic acid encoding the gRNA.

40. The method of claim 39, wherein the CRISPR Cas nuclease is a Cas9 nuclease or a Cas12 nuclease.

41. The method of claim 39 or 40, wherein the gRNA specifically targets the sequence in the safe harbor site and/or specifically targets a sequence in or near the homology arms.

42. The method of any one of claims 34-41, wherein the subject has a medical condition selected from Table 2.

43. The method of claim 42, wherein the gene of interest is selected from Table 2.

44. The method of claim 43, wherein the gene of interest is a variant of a gene selected from Table 2.

45. A guide RNA comprising a sequence homologous to a sequence in a safe harbor site in the human genome in any one of the following loci: 1q31, 3p24, 7q35, and Xq21.

46. A delivery system comprising the guide RNA of claim 45.

47. A method comprising genetically modifying a safe harbor site in the human genome in any one of the following loci: 1q31, 3p24, 7q35, and Xq21.

48. An engineered nucleic acid targeting vector comprising a sequence of interest flanked by homology arms, wherein each homology arm comprises a sequence homologous to a safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

49. A method comprising identifying a safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

50. A method comprising amplifying sequence from safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

51. A method comprising modifying sequence in safe harbor site in the human genome that is at least 50 kb from any known gene, at least 20 kb from an enhanced region, at least 150 kb from a lncRNA and a tRNA, at least 300 kb from any known oncogene, at least 300 kb from a miRNA, and at least 300 kb from a telomere and a centromere.

52. A method comprising introducing one or more polynucleotide into a safe harbor site in a human cell ex vivo and producing a protein encoded by the one or more polynucleotide, wherein the safe harbor site is selected from any one of Table 1, optionally 1q31, 3p24, 7q35, or Xq21.

53. The method of claim 52, wherein the one or more polynucleotide encodes a therapeutic protein.

54. The method of claim 53, wherein the therapeutic protein is an antibody, optionally selected from a human antibody, a humanized antibody, and a chimeric antibody.

55. A method comprising introducing one or more polynucleotide into a safe harbor site in a human cell ex vivo and producing a recombinant gene therapy vector or one or more components of a gene therapy vector encoded by the one or more polynucleotide, wherein the safe harbor site is selected from any one of Table 1, optionally 1q31, 3p24, 7q35, or Xq21.

56. The method of claim 55, wherein the gene therapy vector is an adenovirus vector, an adeno-associated virus (AAV) vector, a retrovirus vector, or a Herpes virus vector.

57. The method of any one of the preceding claims, wherein the human cell is a stem cell, an immune cell, or a mesenchymal cell.

58. The method of claim 55, wherein the stem cell is an induced pluripotent stem cell (iPSC).

59. The method of claim 55, wherein the immune cell is a T cell, a B cell, or an NK cell.

60. The method of claim 59, wherein the immune cell is a primary T cell.

61. The method of claim 55, wherein the mesenchymal cell is a fibroblast, optionally a primary human dermal fibroblast.

62. The method of claim 55, wherein the mesenchymal cell is a mesenchymal stem cell.61. The method of claim 55, wherein the mesenchymal cell is a hematopoietic stem cell.


SEQUENCE LISTING (88 sequences)

SEQ ID NO: 1 - DNA, 2417 bp, Artificial Sequence
  Synthetic
ctgcatttaagtaggattcaataattttaaagtgcagggacaaaatttcctcatatggctcactagctacattgcaaatttcttgaaatcagaacacagaagtgcagtcctgtgctcgcaatgcagacttgcagggtgtagaggcataaatggctccagagccagggacatgggtccagaggggggtagtctccagaagactcctttcgggcctattaccatgcctcagaggtccaagtggggcatggtgaatatattatcctttatattatatttcttatatgtctacaactgccacttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcggccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagtaagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcatggcactaggactaaaggttggccaaagtacaagatatttgtcttatctgatgacaactctgtgtcctggactctcttccagaataagacctttcctgcagcactgcttgaactcctcttagcaagagggaaacatgtgaaatgctaccaaaatagaatagaagtaaattcttattatattcctttgttcactcatatcctgaagtgcatcaaatcaggttttctcacctgtataatgctgtattttacttgagttggaataattttgcttagaaataaataagtaaaacagcacctg

SEQ ID NO: 2 - DNA, 2417 bp, Artificial Sequence
  Synthetic
cattacatccaagtttagactcattgagctctaaatatttgggaaaacatatttaaagaaattatataggtttgatccaaaatctctttggcacaacttgaaatatgggtaatcgtcatgtgaaatttgtgaataggagaacccactgtaggatacttaacataaatcagccacataatttctatcactgatatccagggaatttcaatgacaaatctagtgataaaaattgataaaacatttttgatagttttgatacaagtgaaagtcatgggatatcagacttaaaagaaacctcaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcggccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagtaagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctatcaagtctgatgtcagtaatttttggaggagactgaagtgcagtgagactatccaaagtcagacatggggaaaagcagagtcatccctcctaggctgccaaaatcctccccatccaagctcatccttgaagccctcacttaagacaaagttcctcccatcccttctgcctgctctggcatggtctgaaccatttgcctattaattgccctgcctggtttcatttgttctttttgctgtatttaaactgtgggaattctattgttaacctttttcttgctcaactgaactgtgaca

SEQ ID NO: 3 - DNA, 6009 bp, Artificial Sequence
  Synthetic
ctgcatttaagtaggattcaataattttaaagtgcagggacaaaatttcctcatatggctcactagctacattgcaaatttcttgaaatcagaacacagaagtgcagtcctgtgctcgcaatgcagacttgcagggtgtagaggcataaatggctccagagccagggacatgggtccagaggggggtagtctccagaagactcctttcgggcctattaccatgcctcagaggtccaagtggggcatggtgaatatattatcctttatattatatttcttatatgtctacaactgccacttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcggccgccaccatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcccagaatgatgtgaaccctgtctctctgcagctggacctggacaggagattccagcttcaagaagtcatgatggagttccaggggcccatgcctgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgcaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcggcggacctgacaggactggagaagcgtgtggagcagatccgtgaccacatcaatgggcgcgtgctctactatgccacctgcaaggagggcagaggaagtcttctaacatgcggtgacgtggaggagaatcccggccctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagccatggcactaggactaaaggttggccaaagtacaagatatttgtcttatctgatgacaactctgtgtcctggactctcttccagaataagacctttcctgcagcactgcttgaactcctcttagcaagagggaaacatgtgaaatgctaccaaaatagaatagaagtaaattcttattatattcctttgttcactcatatcctgaagtgcatcaaatcaggttttctcacctgtataatgctgtattttacttgagttggaataattttgcttagaaataaataagtaaaacagcacctg

SEQ ID NO: 4 - DNA, 6009 bp, Artificial Sequence
  Synthetic
cattacatccaagtttagactcattgagctctaaatatttgggaaaacatatttaaagaaattatataggtttgatccaaaatctctttggcacaacttgaaatatgggtaatcgtcatgtgaaatttgtgaataggagaacccactgtaggatacttaacataaatcagccacataatttctatcactgatatccagggaatttcaatgacaaatctagtgataaaaattgataaaacatttttgatagttttgatacaagtgaaagtcatgggatatcagacttaaaagaaacctcaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttgcggccgccaccatgagaccattcttcctcttgtgttttgccctgcctggcctcctgcatgcccaacaagcctgctcccgtggggcctgctatccacctgttggggacctgcttgttgggaggacccggtttctccgagcttcatctacctgtggactgaccaagcctgagacctactgcacccagtatggcgagtggcagatgaaatgctgcaagtgtgactccaggcagcctcacaactactacagtcaccgagtagagaatgtggcttcatcctccggccccatgcgctggtggcagtcccagaatgatgtgaaccctgtctctctgcagctggacctggacaggagattccagcttcaagaagtcatgatggagttccaggggcccatgcctgccggcatgctgattgagcgctcctcagacttcggtaagacctggcgagtgtaccagtacctggctgccgactgcacctccaccttccctcgggtccgccagggtcggcctcagagctggcaggatgttcggtgccagtccctgcctcagaggcctaatgcacgcctaaatggggggaaggtccaacttaaccttatggatttagtgtctgggattccagcaactcaaagtcaaaaaattcaagaggtgggggagatcacaaacttgagagtcaatttcaccaggctggcccctgtgccccaaaggggctaccaccctcccagcgcctactatgctgtgtcccagctccgtctgcaggggagctgcttctgtcacggccatgctgatcgctgcgcacccaagcctggggcctctgcaggcccctccaccgctgtgcaggtccacgatgtctgtgtctgccagcacaacactgccggcccaaattgtgagcgctgtgcacccttctacaacaaccggccctggagaccggcggagggccaggacgcccatgaatgccaaaggtgcgactgcaatgggcactcagagacatgtcactttgaccccgctgtgtttgccgccagccagggggcatatggaggtgtgtgtgacaattgccgggaccacaccgaaggcaagaactgtgagcggtgtcagctgcactatttccggaaccggcgcccgggagcttccattcaggagacctgcatctcctgcgagtgtgatccggatggggcagtgccaggggctccctgtgacccagtgaccgggcagtgtgtgtgcaaggagcatgtgcagggagagcgctgtgacctatgcaagccgggcttcactggactcacctacgccaacccgcagggctgccaccgctgtgactgcaacatcctggggtcccggagggacatgccgtgtgacgaggagagtgggcgctgcctttgtctgcccaacgtggtgggtcccaaatgtgaccagtgtgctccctaccactggaagctggccagtggccagggctgtgaaccgtgtgcctgcgacccgcacaactccctcagcccacagtgcaaccagttcacagggcagtgcccctgtcgggaaggctttggtggcctgatgtgcagcgctgcagccatccgccagtgtccagaccggacctatggagacgtggccacaggatgccgagcctgtgactgtgatttccggggaacagagggcccgggctgcgacaaggcatcaggccgctgcctctgccgccctggcttgaccgggccccgctgtgaccagtgccagcgaggctactgcaatcgctacccggtgtgcgtggcctgccacccttgcttccagacctatgatgcggacctccgggagcaggccctgcgctttggtagactccgcaatgccaccgccagcctgtggtcagggcctgggctggaggaccgtggcctggcctcccggatcctagatgcaaagagtaagattgagcagatccgagcagttctcagcagccccgcagtcacagagcaggaggtggctcaggtggccagtgccatcctctccctcaggcgaactctccagggcctgcagctggatctgcccctggaggaggagacgttgtcccttccgagagacctggagagtcttgacagaagcttcaatggtctccttactatgtatcagaggaagagggagcagtttgaaaaaataagcagtgctgatccttcaggagccttccggatgctgagcacagcctacgagcagtcagcccaggctgctcagcaggtctccgacagctcgcgccttttggaccagctcagggacagccggagagaggcagagaggctggtgcggcaggcgggaggaggaggaggcaccggcagccccaagcttgtggccctgaggctggagatgtcttcgttgcctgacctgacacccaccttcaacaagctctgtggcaactccaggcagatggcttgcaccccaatatcatgccctggtgagctatgtccccaagacaatggcacagcctgtggctcccgctgcaggggtgtccttcccagggccggtggggccttcttgatggcggggcaggtggctgagcagctgcggggcttcaatgcccagctccagcggaccaggcagatgattagggcagccgaggaatctgcctcacagattcaatccagtgcccagcgcttggagacccaggtgagcgccagccgctcccagatggaggaagatgtcagacgcacacggctcctaatccagcaggtccgggacttcctaacagaccccgacactgatgcagccactatccaggaggtcagcgaggccgtgctggccctgtggctgcccacagactcagctactgttctgcagaagatgaatgagatccaggccattgcagccaggctccccaacgtggacttggtgctgtcccagaccaagcaggacattgcgcgtgcccgccggttgcaggctgaggctgaggaagccaggagccgagcccatgcagtggagggccaggtggaagatgtggttgggaacctgcggcaggggacagtggcactgcaggaagctcaggacaccatgcaaggcaccagccgctcccttcggcttatccaggacagggttgctgaggttcagcaggtactgcggccagcagaaaagctggtgacaagcatgaccaagcagctgggtgacttctggacacggatggaggagctccgccaccaagcccggcagcagggggcagaggcagtccaggcccagcagcttgcggaaggtgccagcgagcaggcattgagtgcccaagagggatttgagagaataaaacaaaagtatgctgagttgaaggaccggttgggtcagagttccatgctgggtgagcagggtgcccggatccagagtgtgaagacagaggcagaggagctgtttggggagaccatggagatgatggacaggatgaaagacatggagttggagctgctgcggggcagccaggccatcatgctgcgctcggcggacctgacaggactggagaagcgtgtggagcagatccgtgaccacatcaatgggcgcgtgctctactatgccacctgcaaggagggcagaggaagtcttctaacatgcggtgacgtggaggagaatcccggccctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggactagtctagcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagctgctatcaagtctgatgtcagtaatttttggaggagactgaagtgcagtgagactatccaaagtcagacatggggaaaagcagagtcatccctcctaggctgccaaaatcctccccatccaagctcatccttgaagccctcacttaagacaaagttcctcccatcccttctgcctgctctggcatggtctgaaccatttgcctattaattgccctgcctggtttcatttgttctttttgctgtatttaaactgtgggaattctattgttaacctttttcttgctcaactgaactgtgaca

SEQ ID NO: 5 - DNA, 23 bp, Artificial Sequence
  Synthetic
ttagtcctagtgccatgaagtgg

SEQ ID NO: 6 - DNA, 23 bp, Artificial Sequence
  Synthetic
atgcctcagaggtccaagtgggg

SEQ ID NO: 7 - DNA, 23 bp, Artificial Sequence
  Synthetic
ttgaaatacagtgagcagggagg

SEQ ID NO: 8 - DNA, 23 bp, Artificial Sequence
  Synthetic
gctagctaaagtctcgaacttgg

SEQ ID NO: 9 - DNA, 23 bp, Artificial Sequence
  Synthetic
ggcatggtaataggcccgaaagg

SEQ ID NO: 10 - DNA, 23 bp, Artificial Sequence
  Synthetic
catcagacttgatagcactgagg

SEQ ID NO: 11 - DNA, 23 bp, Artificial Sequence
  Synthetic
atatcgtgctaatgtcagtgggg

SEQ ID NO: 12 - DNA, 23 bp, Artificial Sequence
  Synthetic
tttgttgcagaagaactacgggg

SEQ ID NO: 13 - DNA, 23 bp, Artificial Sequence
  Synthetic
aacaggcattatcctcctagggg

SEQ ID NO: 14 - DNA, 23 bp, Artificial Sequence
  Synthetic
gccctgaaagaaccaacttgggg

SEQ ID NO: 15 - DNA, 23 bp, Artificial Sequence
  Synthetic
aggtgcctccaataaagcaaggg

SEQ ID NO: 16 - DNA, 23 bp, Artificial Sequence
  Synthetic
ggatgagaatcgctactgggagg

SEQ ID NO: 17 - DNA, 23 bp, Artificial Sequence
  Synthetic
gtaatagcctgtacacctagtgg

SEQ ID NO: 18 - DNA, 23 bp, Artificial Sequence
  Synthetic
tgtaatcctagcactataggagg

SEQ ID NO: 19 - DNA, 23 bp, Artificial Sequence
  Synthetic
ttggatataggtaactccccagg

SEQ ID NO: 20 - DNA, 23 bp, Artificial Sequence
  Synthetic
ataggctgtccataacccggagg

SEQ ID NO: 21 - DNA, 23 bp, Artificial Sequence
  Synthetic
gagtggctcagactaagggtcgg

SEQ ID NO: 22 - DNA, 23 bp, Artificial Sequence
  Synthetic
gccccatcactcaatcgctgagg

SEQ ID NO: 23 - DNA, 23 bp, Artificial Sequence
  Synthetic
cctttgcaactcatgcgtggtgg

SEQ ID NO: 24 - DNA, 23 bp, Artificial Sequence
  Synthetic
cacacggacacaatgaagggggg

SEQ ID NO: 25 - DNA, 1000 bp, Artificial Sequence
  Synthetic
gcaaattttggaattttgttaaaatagttaaagataaactatgttacctttcagaaaagtaaagggagtggtcagtgactattaataaaacaaatagtgccatctactgtcaaaaaattctattatgaaagttgccataaacatcattatttttcagtgtgatagtgctatagtcatttatttgatattctaaaatttccaagaatttttatttatttcaaataatcacagttaaatttctagttctgctaactggagtggaatttacatgtacttaaatcaaatgactgccactttacaaaatcactgtcatcaggaagcaattttttaaaagtgtttctttgtgctaagaagtacacgatgtaaaaacaacaacaacaacaaaatgcttgtatcctttccaaacaccatacacacacatgactcagtcagaactgcagaaatgtaaggataacgatactaaacaagaggaaaaatgaaaaagacaggaaaaagcctggtcaaattattaaagaagtgcaagcattgatgcaacttactgataaaggtgaaactgtaaagtatactttaaaatagatgcagtaagtagaattagagttagcttccatcaccttttaatctacaaatgattttacagagaaagcagcattaaagatctttgtgggcaatcaaaacagtaatttgagaatagcattatacactgcatttaagtaggattcaataattttaaagtgcagggacaaaatttcctcatatggctcactagctacattgcaaatttcttgaaatcagaacacagaagtgcagtcctgtgctcgcaatgcagacttgcagggtgtagaggcataaatggctccagagccagggacatgggtccagaggggggtagtctccagaagactcctttcgggcctattaccatgcctcagaggtccaagtggggcatggtgaatatattatcctttatattatatttcttatatgtctacaactgccactt

SEQ ID NO: 26 - DNA, 1000 bp, Artificial Sequence
  Synthetic
ataaaagcaaaaatgtccctatcacacataatcaaagtgattcatctggtaagctagatataagcaaattttggaattttgttaaaatagttaaagataaactatgttacctttcagaaaagtaaagggagtggtcagtgactattaataaaacaaatagtgccatctactgtcaaaaaattctattatgaaagttgccataaacatcattatttttcagtgtgatagtgctatagtcatttatttgatattctaaaatttccaagaatttttatttatttcaaataatcacagttaaatttctagttctgctaactggagtggaatttacatgtacttaaatcaaatgactgccactttacaaaatcactgtcatcaggaagcaattttttaaaagtgtttctttgtgctaagaagtacacgatgtaaaaacaacaacaacaacaaaatgcttgtatcctttccaaacaccatacacacacatgactcagtcagaactgcagaaatgtaaggataacgatactaaacaagaggaaaaatgaaaaagacaggaaaaagcctggtcaaattattaaagaagtgcaagcattgatgcaacttactgataaaggtgaaactgtaaagtatactttaaaatagatgcagtaagtagaattagagttagcttccatcaccttttaatctacaaatgattttacagagaaagcagcattaaagatctttgtgggcaatcaaaacagtaatttgagaatagcattatacactgcatttaagtaggattcaataattttaaagtgcagggacaaaatttcctcatatggctcactagctacattgcaaatttcttgaaatcagaacacagaagtgcagtcctgtgctcgcaatgcagacttgcagggtgtagaggcataaatggctccagagccagggacatgggtccagaggggggtagtctccagaagactcctttcgggcctattaccatgcctcagaggtccaa

SEQ ID NO: 27 - DNA, 1000 bp, Artificial Sequence
  Synthetic
acaattgtaggcaactgcaagagcaatgtaggtaaaattcacacctacaggcagtcgtgaaaattttcccaatataaacttgcacttcacatgcacttttgtggtgtgaaggagaggaactggtgagaaactgatgagaaatgatgataagcagacttttactggaacattgctcaatcccctctataaggcaatgatgctatgtagacaatcaaacatgaaattgctagaaagtttatagattgatataatctattttaatgcatctaggattcaggtaagctgcgaaaaagtagtgccaatatgtttattttataggggatatttaaaattaattttattctttttaaaattgcaatggtacccaaattccctaacttcctatggtaggctgaataacagctcctaaaaatatcaggttgtaattcctggaatgtgtaaatgctatcttatatggaaaatataccaatatgtgattaaattatggagtttgaaatgaagagattaacctgatttatctgggtgcatcctacatgagatcacaattgttcttaaaagagggaggcagagggaggtgtgacacagacaaaagagaaggccatgggaagacagagcaagagagggtaaaagatgctggccttaaatattggagtaatgtagcaacaggccaagggatgccagcaggagttgtaggaggtcaataccttgattttgacccagtgatactgacttcagacttgtggcctccagaactgtgaaagaataaattcctgtcgttttaagtcactgacactgattttgtggtaggtaatttgttacagcagccacaggaaactaatacaatctgggtgaatttctctttctataatgaaggattctttcataattaaaaatataactttaatatagttggtattatcagcaccatgctataacttctgcaaaaaagctctctaatccttatatctgttttcttgataaatcatactgttctcctccc

SEQ ID NO: 28 - DNA, 1000 bp, Artificial Sequence
  Synthetic
aaccataccagatcccttaatgggaaagggtatttcagacctgggcactggctgtttcttctgattggagtacttctacctcagacatcataatgatgaactcttttgccttcttcaagtcttagatcaaattattcctttttactgtttatattccaactagtgagggataatgtccctcactatcccctaggagattgattccaggaagcttgagggtaccaaactctgtggatgctcaagtctctgatagaaaatgccatagtatttacatatgatctacacaaaccctcctgcatatttaaaatagtctctagattacttataattactagattacttatagtctctagatttatataatcttataaaagggtaagattacttattacttacccttaatacaatgtaaatattgcgtagcttttatactgtgaattttaaaatttgtattattttcattactatatttttgttttttctgcatatttttaatccatggttggtgaaatccatggatatgaaatccgctcatatggagggactgagaaccaatcatattctattcaacactgcaacctcctttcccacccagcataacagactaattgtaccctgcttcttctgctttattttctaaatctcatttcaatctctaaaatgttatgtaatttacttaattgctatgttcattttatatcaactttctgcctcttctactatgttatcttcttgagagagaagattttaatctcttttgctcactagtgtattcccagtgcatagaacaatatctagcccataacaggtattcagtaattttattcttgaatgaataattgaaggaaaacttttaaaaatccattaccataaggtagggatgcagagagcctaaatcatactaaagtgaatttcagctttcagttcaagctgacatattatcaaatcttcttatgtttttatcatttcaacttctgttctgtgctagctaaagtctcga

SEQ ID NO: 29 - DNA, 1000 bp, Artificial Sequence
  Synthetic
tataccttcatccaaaattattctatgaaataaaagcaaaaatgtccctatcacacataatcaaagtgattcatctggtaagctagatataagcaaattttggaattttgttaaaatagttaaagataaactatgttacctttcagaaaagtaaagggagtggtcagtgactattaataaaacaaatagtgccatctactgtcaaaaaattctattatgaaagttgccataaacatcattatttttcagtgtgatagtgctatagtcatttatttgatattctaaaatttccaagaatttttatttatttcaaataatcacagttaaatttctagttctgctaactggagtggaatttacatgtacttaaatcaaatgactgccactttacaaaatcactgtcatcaggaagcaattttttaaaagtgtttctttgtgctaagaagtacacgatgtaaaaacaacaacaacaacaaaatgcttgtatcctttccaaacaccatacacacacatgactcagtcagaactgcagaaatgtaaggataacgatactaaacaagaggaaaaatgaaaaagacaggaaaaagcctggtcaaattattaaagaagtgcaagcattgatgcaacttactgataaaggtgaaactgtaaagtatactttaaaatagatgcagtaagtagaattagagttagcttccatcaccttttaatctacaaatgattttacagagaaagcagcattaaagatctttgtgggcaatcaaaacagtaatttgagaatagcattatacactgcatttaagtaggattcaataattttaaagtgcagggacaaaatttcctcatatggctcactagctacattgcaaatttcttgaaatcagaacacagaagtgcagtcctgtgctcgcaatgcagacttgcagggtgtagaggcataaatggctccagagccagggacatgggtccagaggggggtagtctccagaagactcctttc

SEQ ID NO: 30 - DNA, 1000 bp, Artificial Sequence
  Synthetic
acaatgcataatatatcaatattagtcatttttccctttataaatcatatctcaaaatgtatgcaatattctttaaatataacacttaaaatgcatatgcaatgatgaaatatagcaggattgccaaaactgatgaatatctcagagatattgccattgtttttcctggaaaatacttctttggaaaaagtgagacatctttgagatcaaaataaacacctgtcaaaggaatgtcttgggaaccccaaatagcatttctaaagagataaaagaatgttgtagtcttccagaaaggaaatcaagtggaagtagatattaattcagcactacagagacaatctggagaaaacagaggcattgttttctaaagaaattggcttttgtaacataaaggagatacaactctgggagtgaaagttgtcacagtgatacatatcacacaacggaaatgccaaacgaaaccatcaccacttgaaatcaaacttgattcaagttactcaggtaaaataccaccatgggtggcatcctttttttttttttttttttttttttttttaaccaagcagcctaacggtgtttaaggaggaaaacttaattgataatgcactttgctcaattataaataagctgactgggaaagaagtgggcatgatggaaagcaaaatttgaatgaagctgttattcttttaatctataaaaacattacatccaagtttagactcattgagctctaaatatttgggaaaacatatttaaagaaattatataggtttgatccaaaatctctttggcacaacttgaaatatgggtaatcgtcatgtgaaatttgtgaataggagaacccactgtaggatacttaacataaatcagccacataatttctatcactgatatccagggaatttcaatgacaaatctagtgataaaaattgataaaacatttttgatagttttgatacaagtgaaagtcatgggatatcagacttaaaagaaacctcag

SEQ ID NO: 31 - DNA, 1000 bp, Artificial Sequence
  Synthetic
gtgcacgggtgtcccggattttctgagagagctttgcataacttctaattcacaactttgataattgccagagtggccaagagctcagaaaagcattttcccacaaggttttcaaatatagctgccacaaatgtcaaagattcttttaattttagtatcatcccagctaattctgagcatcagaaaaatattttgttttattttggatctactaaaaaggaaatgctagcaacaacaacaacaaatcctccagggcattcattacctacatcatgcatgggagaactgaatttagcttttaaaagttaaagtggaacttgaaacagagcaaaatgaagtcaagtgtactgtgaaaaattcatgatttcaaaatggaagcaaggtcatattttattggtaatctttaaataggtttagaaagactgcaatgtagcatggagaatttggtatttgggctcatctgattccgctctttattcagacaaaatctgagctatagaaatcaaatataaaagagtgctaatttctttttgttgttgttgttgggggggacagagtctcgctctgtcgcccagctggagtgcagtggcacaatctcggctcacttcaagctccgcctcccgggttcaagccattctcctgcctcagcctcccaaatagctgggactacaggcgcccgccacctggcccggctaatttttttgtatttttagtagagacacggcttcactgtgttagccaggatagtctcgatctcctgacctcgtgatccgcctgcctcggcctcccaaagtgctgggaaggagtgctaatttcagttggcttccaacaagaccttatccaagctacatattgctattttcaaaaataattgatatgtaaatttaaaatcaaataattacatatttacgtgaagtatatctgtatgttaaaagcaaacacacagcataaacagatacagtgtattaaaatggattttattttcaccatctcaccccac

SEQ ID NO: 32 - DNA, 1000 bp, Artificial Sequence
  Synthetic
ttcatttgttctttttgctgtatttaaactgtgggaattctattgttaacctttttcttgctcaactgaactgtgacactgctaggaatgccgaagcagggttttaggttctcaggatgtttaagagttggagaaagcactcaatgagtcttgtgaataattttgtggaaactgcactcccaatgacaggctctggcatctcactctaaggtaagtaacaggtgaggcactgccctttgataaccaggacgtggattctagaattggttatgtccattcacacaatgtgttcatctcctctctggctatccttctgatgactcagaagtcgcaaccccaagttggttctttcagggccctggcttgctcatcccctttatgattctcctttattcttctgtacctgggtattcatttcatgtccaccctcatcatcattctggagacaaagatgcagacatgtactggcagaatctgagcatgcaaaaaccttccgtaacactcagagttcttatacctttctctcatctggcattatttactatgatagggtgaaggaaacaactatgtctgttctttgcatttgactgcattgtccagtttctagaggcttaagtacactttttttttttttttttttgagacagagtttcactcttgttgcccaggctggagtgcaatgacgcgatctcagctcactgcaacctccaactctcaggttcaagtgattctcctgcctcagcctcccaaatagctgggattacaggcatctgccaccacacctagctaattttatatttgtagtagaagcggggtttctccatgttggtcaggctggtctcgaactcctgacctcaggtgatccaaccgcctcagcctcccaaagtactgggattacaggcgtaagccactgcacccagcacctagtggcttaaatacatttaaagcatcatagctcaacctctaaattgcactgcagcatacacagctaaatccccgt

SEQ ID NO: 33 - DNA, 1000 bp, Artificial Sequence
  Synthetic
agtcttcaccctgaagccctctctgggacctgcccttgacaaaagataccaccttctccaaggtcaagcccccttcccagaatcaagaattgctagaatcaagaatcaagagaatcaagaatcaaggcttcctaccctcacttaaactcagggcaattctgaagagccaacccagcttcagagctcccctacagcaatgtgttacaattttgttataactgcatcttagcattccaatatctctttgtctagtcctggcttcttcccttccccacaggggaacactacctataacaagccttagcatccctccatttttaatttttcatattagtgacctgcctttcatcctctgttcattcactcaatgaatattgtcagagtacctactatacaccaggcattattttgggtgctggttattcagcaatgaacaaggctagaaaggccctgccatcacagagttgccatttcagtaagggaagaaggcaataaacatataatttactatcacataggggtaatgcttgaaggaaaattaattgaactgtaacagtacagagttattgggaggaagcatttatttaaacagaaaagaccagagaagacccctatggggggagtgagatggaactgataactgaatgaagtgacagaatgaaccatgcaaagacataaaaaattgcattcaaagcagaagaaagaacaagtgcaaagttcccagggctcaaataatatgtaattttgaagaatggttaggacagtttggttagagtagagtgaaaaggggagaaattgataggagataaactcaaagacagaggcagcagttcctgcagagccttgtagaggtaaagaggtcatattttattctgaatgtgataagaatctactgcaggaacaggggaggacacggtccaattgatgtttgaaaagattattctggctattgtatggaaagaaactgagggggcaagggtaggagcaggaagatccccta

SEQ ID NO: 34 - DNA, 1000 bp, Artificial Sequence
  Synthetic
gcagcctaacggtgtttaaggaggaaaacttaattgataatgcactttgctcaattataaataagctgactgggaaagaagtgggcatgatggaaagcaaaatttgaatgaagctgttattcttttaatctataaaaacattacatccaagtttagactcattgagctctaaatatttgggaaaacatatttaaagaaattatataggtttgatccaaaatctctttggcacaacttgaaatatgggtaatcgtcatgtgaaatttgtgaataggagaacccactgtaggatacttaacataaatcagccacataatttctatcactgatatccagggaatttcaatgacaaatctagtgataaaaattgataaaacatttttgatagttttgatacaagtgaaagtcatgggatatcagacttaaaagaaacctcagtgctatcaagtctgatgtcagtaatttttggaggagactgaagtgcagtgagactatccaaagtcagacatggggaaaagcagagtcatccctcctaggctgccaaaatcctccccatccaagctcatccttgaagccctcacttaagacaaagttcctcccatcccttctgcctgctctggcatggtctgaaccatttgcctattaattgccctgcctggtttcatttgttctttttgctgtatttaaactgtgggaattctattgttaacctttttcttgctcaactgaactgtgacactgctaggaatgccgaagcagggttttaggttctcaggatgtttaagagttggagaaagcactcaatgagtcttgtgaataattttgtggaaactgcactcccaatgacaggctctggcatctcactctaaggtaagtaacaggtgaggcactgccctttgataaccaggacgtggattctagaattggttatgtccattcacacaatgtgttcatctcctctctggctatccttctgatgactcagaagtcgcaaccccaa

SEQ ID NO: 35 - DNA, 1000 bp, Artificial Sequence
  Synthetic
tgcataggagaggttctattacatagtggctgctcatcaaatgtttaaaaaatattaatgagtttgatatcttagaattttcctttaatgtgtattttatcagcatggttttgatggagcagttagagcggattgtataattatgattgcaggctctattcaattcactgacataaatatcatgtaagacaagaacagggtttgatggcaatctatcagggactccctaaggcttcaattaaattccacaagcatttatcagagctatatatgcggcaggcctgtgtagtcattaacaatatgctgccctcgaaaaatacacaggctacttttacatggccctattaatcaacacttattgggtacagcgatacaactagctgttaattcatccatccataccattatttatatctcatttcaccttctacttatagagctattattagagatatttgatgtaacaagataaactatgccttcagcatttatttgatctgccaacttgaaaatactatcaagaagacattaggatggattttaatgattcattcttaataaaaccacctccatgagactgtatagtttttttattataaaggcccgttttttgtttgtttgtttgttttaatcagttcctccaactattgtcctagtgatcttggataagttacataaattttctaacttcaactctttcttcacttgtaaaacatagatacaaatagcacatgtctgagagagttgttcggtagataaaataagataatcaactatctcaagtctgattccatgggaaatggattctgagcctctgggatttggatacaaggggttagttggagaaatttcagggaatcaacagctaggaagatttgaaggactctaggaaatttgagatttgtgtagagggagaggctgaactggtctgcaacagaagtggcagatgattccacagagaaccctggagctgggatatcttcaggtgcctccaataaag

SEQ ID NO: 36 - DNA, 1000 bp, Artificial Sequence
  Synthetic
gaaacctcctgccgatttgtaaataactacccctctatcccagtccccaagtagtaactgatctgctttctgtcactatacactaatttgccattttaagaattttatattaatgggattatactttttggggaaaggggtctggcttctttgaatcagcatgactattttgagattcatccaaattacagtgtatagtgtatcaatagtttatagtttttattgctgaatggtgttcctttgcatgggtttactgcaatttgtttatccattcacctgttcatagatgttaagattgtttcaactttttagctattataaataaagctgctatgaacattcagatataagcttagtacagatatatcctttagttaggaaaatatctagggacagaatggtttgttcagatatgtgatttgcaaatattttctccatctgtgttttgttttttacaatatttaacgatgcctcttgacgaacagaaattatgaaattaagtcttgtttatcaactttttcttttatggtttatgcttttggtgttgtatctaagaaatttttgcctaactcaaggtcaaaaaggttttctgtgttttttggcttatttaggtgtatgaaccatcactgatttttcttcataaggtataaatatcaaagttcatttgtggcatattaatatccaatttttccagcagcatttattaaaaagaccaccttttctccacaaaatttgccacttttctacaaataatattttataaaacagccaaataatgtttttttttaatagccaaggcatcatttagtttatatgtacctttttgagtgtgctttgttagtgtttttcttttcttttcttttcttttcttttcttttcttttcttttcttttcttttcttttcttttcttttcttttcgaggcgagtcttgttctgtcgctcaggctggagtgcagagtgcagtggcccgatctcctctcactgcaacccccgcctccc

SEQ ID NO: 37 - DNA, 1000 bp, Artificial Sequence
  Synthetic
aattcactgacataaatatcatgtaagacaagaacagggtttgatggcaatctatcagggactccctaaggcttcaattaaattccacaagcatttatcagagctatatatgcggcaggcctgtgtagtcattaacaatatgctgccctcgaaaaatacacaggctacttttacatggccctattaatcaacacttattgggtacagcgatacaactagctgttaattcatccatccataccattatttatatctcatttcaccttctacttatagagctattattagagatatttgatgtaacaagataaactatgccttcagcatttatttgatctgccaacttgaaaatactatcaagaagacattaggatggattttaatgattcattcttaataaaaccacctccatgagactgtatagtttttttattataaaggcccgttttttgtttgtttgtttgttttaatcagttcctccaactattgtcctagtgatcttggataagttacataaattttctaacttcaactctttcttcacttgtaaaacatagatacaaatagcacatgtctgagagagttgttcggtagataaaataagataatcaactatctcaagtctgattccatgggaaatggattctgagcctctgggatttggatacaaggggttagttggagaaatttcagggaatcaacagctaggaagatttgaaggactctaggaaatttgagatttgtgtagagggagaggctgaactggtctgcaacagaagtggcagatgattccacagagaaccctggagctgggatatcttcaggtgcctccaataaagcaagggtgtggttctttgcagtattacctggaccagtcattggatataggtaactccccaggaagaaaccatagccttgggtagacagcttccttaaacagacaggaatagtagagagggactcaactgtgagctgtcaacagtcaactatgctaccacta

SEQ ID NO: 38 - DNA, 1000 bp, Artificial Sequence
  Synthetic
actattttgagattcatccaaattacagtgtatagtgtatcaatagtttatagtttttattgctgaatggtgttcctttgcatgggtttactgcaatttgtttatccattcacctgttcatagatgttaagattgtttcaactttttagctattataaataaagctgctatgaacattcagatataagcttagtacagatatatcctttagttaggaaaatatctagggacagaatggtttgttcagatatgtgatttgcaaatattttctccatctgtgttttgttttttacaatatttaacgatgcctcttgacgaacagaaattatgaaattaagtcttgtttatcaactttttcttttatggtttatgcttttggtgttgtatctaagaaatttttgcctaactcaaggtcaaaaaggttttctgtgttttttggcttatttaggtgtatgaaccatcactgatttttcttcataaggtataaatatcaaagttcatttgtggcatattaatatccaatttttccagcagcatttattaaaaagaccaccttttctccacaaaatttgccacttttctacaaataatattttataaaacagccaaataatgtttttttttaatagccaaggcatcatttagtttatatgtacctttttgagtgtgctttgttagtgtttttcttttcttttcttttcttttcttttcttttcttttcttttcttttcttttcttttcttttcttttcttttcgaggcgagtcttgttctgtcgctcaggctggagtgcagagtgcagtggcccgatctcctctcactgcaacccccgcctcccagtagcgattctcatccctcagcctcccgagtagctgggcctacagtcgtgtgccaccatgcccggctaatttttgtagttttagtagagatgtggctttatggtgttgcccaggctgatctcgaactcctgacctcaagtgatatgtccactttggcctcct

SEQ ID NO: 39 - DNA, 1000 bp, Artificial Sequence
  Synthetic
aatgagtttgatatcttagaattttcctttaatgtgtattttatcagcatggttttgatggagcagttagagcggattgtataattatgattgcaggctctattcaattcactgacataaatatcatgtaagacaagaacagggtttgatggcaatctatcagggactccctaaggcttcaattaaattccacaagcatttatcagagctatatatgcggcaggcctgtgtagtcattaacaatatgctgccctcgaaaaatacacaggctacttttacatggccctattaatcaacacttattgggtacagcgatacaactagctgttaattcatccatccataccattatttatatctcatttcaccttctacttatagagctattattagagatatttgatgtaacaagataaactatgccttcagcatttatttgatctgccaacttgaaaatactatcaagaagacattaggatggattttaatgattcattcttaataaaaccacctccatgagactgtatagtttttttattataaaggcccgttttttgtttgtttgtttgttttaatcagttcctccaactattgtcctagtgatcttggataagttacataaattttctaacttcaactctttcttcacttgtaaaacatagatacaaatagcacatgtctgagagagttgttcggtagataaaataagataatcaactatctcaagtctgattccatgggaaatggattctgagcctctgggatttggatacaaggggttagttggagaaatttcagggaatcaacagctaggaagatttgaaggactctaggaaatttgagatttgtgtagagggagaggctgaactggtctgcaacagaagtggcagatgattccacagagaaccctggagctgggatatcttcaggtgcctccaataaagcaagggtgtggttctttgcagtattacctggaccagtcattggatataggtaactc

SEQ ID NO: 40 - DNA, 1000 bp, Artificial Sequence
  Synthetic
atagaccagcagcatcagcatcacctaggaaaatgttagaaatgcaaattcttgggccccatcactcaatcgctgaggatgggacacaggtggttctgatgcatgctgatgactgagaatcacggtacagaatatactgatgcaggattttctgctccttagctcacctaaatccgggatcttgtctcatcaccaggaagtagtaggcacacggacacaatgaaggggggtgagggcagaatttattaagtgaaaggaaagcactcagtaaagagaggtgtcctgcacacaggcttccacctcacaaatttgaataccaggccaccacgcatgagttgcaaaggtaaggctcctcccccacaaaaggtgcaaattcctgggggtttgaccctattctctcagggtgcatgccgacccttagtctgagccactccacattgatttatttccattactgtgcatgtgttaagggatggaatttttgagtgtgggcatatttaggcaacccccatgtgtggaatgatttgggcaggttggaggttctttgagggcccttccctatctgctaggcatttgtcagcttcctgcctctatcattcccgcttctaaagaggtacatctgactgtgttagaatacggatgaagactgatcttaactgcttcctgctgacagggggcgctgttttgggaagatggcagtcatgtctccctcagaggcctatctaagggtccccagtaaaaagagccatcatcagaggcattgtttgcatgaccatttagagtttggtggcctgaaggtgagaagagacaaactgggttattagaatacatgtatcaaaacgaaacaaaaaggggtgggtaaggacagctcaaaaatcccaagactgatggcacacccagatagctggtggctacagttatgcctgccaagatttgggtgcatgggacttggctttgattagctcccttggtcttattttcccgtataaagaaacctccg

SEQ ID NO: 41 - DNA, 1000 bp, Artificial Sequence
  Synthetic
tctctatgcgacatctttaaatctaccatcacagtatctgtctttgtcttccttttatttgaaaagcaaataaactaccaattgttttttaattaatatgatcaatgatataaagctcttcatttagtttctatctgtgtggaaactcacccacagcatcttttgctttcatgtgattccagaagcaaattttataataaagtttccctacaggttttaaaatgatcaagtttatattctacctgattttcattgtactttatttctccctatataattggaaaaagatattagaagatacattgattttatctgcctctatataggaagtctataaaccagatgactgaaaagaacaaaacggaaaacacttaaacttcactgattatcagaatcaggaaaacaatggtttaggaaataatagcattctgcaatccactgaagacatgctgaatcagtttcttctaggtctggaaacaagaaatttattttttctcagctgttgaattattccgatgtaaacaggtttagtgcttaaatttggaaatcagtgtcatagatgagtggttgtcaaaatgttgttcatagaccagcagcatcagcatcacctaggaaaatgttagaaatgcaaattcttgggccccatcactcaatcgctgaggatgggacacaggtggttctgatgcatgctgatgactgagaatcacggtacagaatatactgatgcaggattttctgctccttagctcacctaaatccgggatcttgtctcatcaccaggaagtagtaggcacacggacacaatgaaggggggtgagggcagaatttattaagtgaaaggaaagcactcagtaaagagaggtgtcctgcacacaggcttccacctcacaaatttgaataccaggccaccacgcatgagttgcaaaggtaaggctcctcccccacaaaaggtgcaaattcctgggggtttgaccctattctctcagggtgcatgccgacc

SEQ ID NO: 42 - DNA, 1000 bp, Artificial Sequence
  Synthetic
aaactagctactctttgaaaactgccttcttagaagtcatcaaaagtctttcattttaaaagaatcctgctttaaaataagcttaaatagaaaacctaacaaggtctttaaaaatgatttaaagaaatttcatctatcacaaaggcttatgattgctttactttattctttgtagaagattctacatatgagtgtgagaaagatgaattatacctctgtatgcatgggaaataccttgatccactaaatccatagtatagggaagcaatttataagagggtttcactattctggttttctattatcatcagaatattgtaatctttctgaagtgtctctggcattctctatgcgacatctttaaatctaccatcacagtatctgtctttgtcttccttttatttgaaaagcaaataaactaccaattgttttttaattaatatgatcaatgatataaagctcttcatttagtttctatctgtgtggaaactcacccacagcatcttttgctttcatgtgattccagaagcaaattttataataaagtttccctacaggttttaaaatgatcaagtttatattctacctgattttcattgtactttatttctccctatataattggaaaaagatattagaagatacattgattttatctgcctctatataggaagtctataaaccagatgactgaaaagaacaaaacggaaaacacttaaacttcactgattatcagaatcaggaaaacaatggtttaggaaataatagcattctgcaatccactgaagacatgctgaatcagtttcttctaggtctggaaacaagaaatttattttttctcagctgttgaattattccgatgtaaacaggtttagtgcttaaatttggaaatcagtgtcatagatgagtggttgtcaaaatgttgttcatagaccagcagcatcagcatcacctaggaaaatgttagaaatgcaaattcttgggccccatcactcaatcg

SEQ ID NO: 43 - DNA, 1000 bp, Artificial Sequence
  Synthetic
agggaagcaatttataagagggtttcactattctggttttctattatcatcagaatattgtaatctttctgaagtgtctctggcattctctatgcgacatctttaaatctaccatcacagtatctgtctttgtcttccttttatttgaaaagcaaataaactaccaattgttttttaattaatatgatcaatgatataaagctcttcatttagtttctatctgtgtggaaactcacccacagcatcttttgctttcatgtgattccagaagcaaattttataataaagtttccctacaggttttaaaatgatcaagtttatattctacctgattttcattgtactttatttctccctatataattggaaaaagatattagaagatacattgattttatctgcctctatataggaagtctataaaccagatgactgaaaagaacaaaacggaaaacacttaaacttcactgattatcagaatcaggaaaacaatggtttaggaaataatagcattctgcaatccactgaagacatgctgaatcagtttcttctaggtctggaaacaagaaatttattttttctcagctgttgaattattccgatgtaaacaggtttagtgcttaaatttggaaatcagtgtcatagatgagtggttgtcaaaatgttgttcatagaccagcagcatcagcatcacctaggaaaatgttagaaatgcaaattcttgggccccatcactcaatcgctgaggatgggacacaggtggttctgatgcatgctgatgactgagaatcacggtacagaatatactgatgcaggattttctgctccttagctcacctaaatccgggatcttgtctcatcaccaggaagtagtaggcacacggacacaatgaaggggggtgagggcagaatttattaagtgaaaggaaagcactcagtaaagagaggtgtcctgcacacaggcttccacctcacaaatttgaataccaggccaccacgc

SEQ ID NO: 44 - DNA, 1000 bp, Artificial Sequence
  Synthetic
ttgctttactttattctttgtagaagattctacatatgagtgtgagaaagatgaattatacctctgtatgcatgggaaataccttgatccactaaatccatagtatagggaagcaatttataagagggtttcactattctggttttctattatcatcagaatattgtaatctttctgaagtgtctctggcattctctatgcgacatctttaaatctaccatcacagtatctgtctttgtcttccttttatttgaaaagcaaataaactaccaattgttttttaattaatatgatcaatgatataaagctcttcatttagtttctatctgtgtggaaactcacccacagcatcttttgctttcatgtgattccagaagcaaattttataataaagtttccctacaggttttaaaatgatcaagtttatattctacctgattttcattgtactttatttctccctatataattggaaaaagatattagaagatacattgattttatctgcctctatataggaagtctataaaccagatgactgaaaagaacaaaacggaaaacacttaaacttcactgattatcagaatcaggaaaacaatggtttaggaaataatagcattctgcaatccactgaagacatgctgaatcagtttcttctaggtctggaaacaagaaatttattttttctcagctgttgaattattccgatgtaaacaggtttagtgcttaaatttggaaatcagtgtcatagatgagtggttgtcaaaatgttgttcatagaccagcagcatcagcatcacctaggaaaatgttagaaatgcaaattcttgggccccatcactcaatcgctgaggatgggacacaggtggttctgatgcatgctgatgactgagaatcacggtacagaatatactgatgcaggattttctgctccttagctcacctaaatccgggatcttgtctcatcaccaggaagtagtaggcacacggacacaatgaa

SEQ ID NO: 45 - DNA, 1000 bp, Artificial Sequence
  Synthetic
catggcactaggactaaaggttggccaaagtacaagatatttgtcttatctgatgacaactctgtgtcctggactctcttccagaataagacctttcctgcagcactgcttgaactcctcttagcaagagggaaacatgtgaaatgctaccaaaatagaatagaagtaaattcttattatattcctttgttcactcatatcctgaagtgcatcaaatcaggttttctcacctgtataatgctgtattttacttgagttggaataattttgcttagaaataaataagtaaaacagcacctgcctccagacctagggtccatcaggaaaaatataaggtatatgaggtgtatgctctaaacccaaggccaacatgtataggaaaaccttaagtccttcagtgcatgtgcttggatgaagaaggtaatacaattgtaggcaactgcaagagcaatgtaggtaaaattcacacctacaggcagtcgtgaaaattttcccaatataaacttgcacttcacatgcacttttgtggtgtgaaggagaggaactggtgagaaactgatgagaaatgatgataagcagacttttactggaacattgctcaatcccctctataaggcaatgatgctatgtagacaatcaaacatgaaattgctagaaagtttatagattgatataatctattttaatgcatctaggattcaggtaagctgcgaaaaagtagtgccaatatgtttattttataggggatatttaaaattaattttattctttttaaaattgcaatggtacccaaattccctaacttcctatggtaggctgaataacagctcctaaaaatatcaggttgtaattcctggaatgtgtaaatgctatcttatatggaaaatataccaatatgtgattaaattatggagtttgaaatgaagagattaacctgatttatctgggtgcatcctacatgagatcacaattgttcttaaaagagggaggcagagggagg

SEQ ID NO: 46 - DNA, 1000 bp, Artificial Sequence
  Synthetic
gtggggcatggtgaatatattatcctttatattatatttcttatatgtctacaactgccacttcatggcactaggactaaaggttggccaaagtacaagatatttgtcttatctgatgacaactctgtgtcctggactctcttccagaataagacctttcctgcagcactgcttgaactcctcttagcaagagggaaacatgtgaaatgctaccaaaatagaatagaagtaaattcttattatattcctttgttcactcatatcctgaagtgcatcaaatcaggttttctcacctgtataatgctgtattttacttgagttggaataattttgcttagaaataaataagtaaaacagcacctgcctccagacctagggtccatcaggaaaaatataaggtatatgaggtgtatgctctaaacccaaggccaacatgtataggaaaaccttaagtccttcagtgcatgtgcttggatgaagaaggtaatacaattgtaggcaactgcaagagcaatgtaggtaaaattcacacctacaggcagtcgtgaaaattttcccaatataaacttgcacttcacatgcacttttgtggtgtgaaggagaggaactggtgagaaactgatgagaaatgatgataagcagacttttactggaacattgctcaatcccctctataaggcaatgatgctatgtagacaatcaaacatgaaattgctagaaagtttatagattgatataatctattttaatgcatctaggattcaggtaagctgcgaaaaagtagtgccaatatgtttattttataggggatatttaaaattaattttattctttttaaaattgcaatggtacccaaattccctaacttcctatggtaggctgaataacagctcctaaaaatatcaggttgtaattcctggaatgtgtaaatgctatcttatatggaaaatataccaatatgtgattaaattatggagtttgaaatgaagagattaacctg

SEQ ID NO: 47 - DNA, 1000 bp, Artificial Sequence
  Synthetic
tgctcactgtatttcaaccataccagatcccttaatgggaaagggtatttcagacctgggcactggctgtttcttctgattggagtacttctacctcagacatcataatgatgaactcttttgccttcttcaagtcttagatcaaattattcctttttactgtttatattccaactagtgagggataatgtccctcactatcccctaggagattgattccaggaagcttgagggtaccaaactctgtggatgctcaagtctctgatagaaaatgccatagtatttacatatgatctacacaaaccctcctgcatatttaaaatagtctctagattacttataattactagattacttatagtctctagatttatataatcttataaaagggtaagattacttattacttacccttaatacaatgtaaatattgcgtagcttttatactgtgaattttaaaatttgtattattttcattactatatttttgttttttctgcatatttttaatccatggttggtgaaatccatggatatgaaatccgctcatatggagggactgagaaccaatcatattctattcaacactgcaacctcctttcccacccagcataacagactaattgtaccctgcttcttctgctttattttctaaatctcatttcaatctctaaaatgttatgtaatttacttaattgctatgttcattttatatcaactttctgcctcttctactatgttatcttcttgagagagaagattttaatctcttttgctcactagtgtattcccagtgcatagaacaatatctagcccataacaggtattcagtaattttattcttgaatgaataattgaaggaaaacttttaaaaatccattaccataaggtagggatgcagagagcctaaatcatactaaagtgaatttcagctttcagttcaagctgacatattatcaaatcttcttatgtttttatcatttcaacttctgttctgtgc

SEQ ID NO: 48 - DNA, 1000 bp, Artificial Sequence
  Synthetic
acttggctaggtgtagtggtttatgcctgtaatccctgtgcccggggaagccaaggcaggaagatcatttgaggccgggtgttccagaccagcctaggcaacatagcaaggcccaccatctacaaatgatataataaaataacaaaattagccaggcatagtggtatgtgacctcagtcccagctgctcgagaggctgatgaggaaggatcacttggcccagtagttggagtttgcagtgatctgtgatcacaccactgtatttcagccttggtgagagagcagacccatctttgaaaaaaaaaattaagtctcaaactttattaatagtgtaacagaatgagcaatactttggagacatgctgctgctatatatatatatatatatatatatatatatatatattttttttttttttttttttttttttttttttttgaggcagattctcactgtgtcacccaggctggagtgcagtggcgccacctcggctcactgcaacctctacctccagggttcaagcaattctcctgcctcagcctcccaagtagctgggatcacaggtgcccactaccacacccagctaatttttggtaattttagtagagatgaggtttcactatgttggctaggctggtctcaaactcctaacctcaagtgatccacctacctctgcctcccaaagtgctaggagtacaagtgtgagccactgaccccggcccttatatttttctaattatcaaataggctgtgagagtttctatttacctaggtcctcaaagtgtctttaggaaaagctataccctggacaaaggcaggttggcagaaataccaggggttgagcttctgcaacaagtttgggtcataaaaactgccactaggcctgtcaggcaacttcctgagtcaataaaatctcacattaatagatataaagaaaaagcaattgaaaattttccagataaactggattccatctgtaaagaggaaaatctgttttgtg

SEQ ID NO: 49 - DNA, 1000 bp, Artificial Sequence
  Synthetic
gggcctattaccatgcctcagaggtccaagtggggcatggtgaatatattatcctttatattatatttcttatatgtctacaactgccacttcatggcactaggactaaaggttggccaaagtacaagatatttgtcttatctgatgacaactctgtgtcctggactctcttccagaataagacctttcctgcagcactgcttgaactcctcttagcaagagggaaacatgtgaaatgctaccaaaatagaatagaagtaaattcttattatattcctttgttcactcatatcctgaagtgcatcaaatcaggttttctcacctgtataatgctgtattttacttgagttggaataattttgcttagaaataaataagtaaaacagcacctgcctccagacctagggtccatcaggaaaaatataaggtatatgaggtgtatgctctaaacccaaggccaacatgtataggaaaaccttaagtccttcagtgcatgtgcttggatgaagaaggtaatacaattgtaggcaactgcaagagcaatgtaggtaaaattcacacctacaggcagtcgtgaaaattttcccaatataaacttgcacttcacatgcacttttgtggtgtgaaggagaggaactggtgagaaactgatgagaaatgatgataagcagacttttactggaacattgctcaatcccctctataaggcaatgatgctatgtagacaatcaaacatgaaattgctagaaagtttatagattgatataatctattttaatgcatctaggattcaggtaagctgcgaaaaagtagtgccaatatgtttattttataggggatatttaaaattaattttattctttttaaaattgcaatggtacccaaattccctaacttcctatggtaggctgaataacagctcctaaaaatatcaggttgtaattcctggaatgtgtaaatgctatcttatatggaaaatataccaatatgtgattaaatt

SEQ ID NO: 50 - DNA, 1000 bp, Artificial Sequence
  Synthetic
tgctatcaagtctgatgtcagtaatttttggaggagactgaagtgcagtgagactatccaaagtcagacatggggaaaagcagagtcatccctcctaggctgccaaaatcctccccatccaagctcatccttgaagccctcacttaagacaaagttcctcccatcccttctgcctgctctggcatggtctgaaccatttgcctattaattgccctgcctggtttcatttgttctttttgctgtatttaaactgtgggaattctattgttaacctttttcttgctcaactgaactgtgacactgctaggaatgccgaagcagggttttaggttctcaggatgtttaagagttggagaaagcactcaatgagtcttgtgaataattttgtggaaactgcactcccaatgacaggctctggcatctcactctaaggtaagtaacaggtgaggcactgccctttgataaccaggacgtggattctagaattggttatgtccattcacacaatgtgttcatctcctctctggctatccttctgatgactcagaagtcgcaaccccaagttggttctttcagggccctggcttgctcatcccctttatgattctcctttattcttctgtacctgggtattcatttcatgtccaccctcatcatcattctggagacaaagatgcagacatgtactggcagaatctgagcatgcaaaaaccttccgtaacactcagagttcttatacctttctctcatctggcattatttactatgatagggtgaaggaaacaactatgtctgttctttgcatttgactgcattgtccagtttctagaggcttaagtacactttttttttttttttttttgagacagagtttcactcttgttgcccaggctggagtgcaatgacgcgatctcagctcactgcaacctccaactctcaggttcaagtgattctcctgcctcagcctcccaaatagctgggattacaggcatctgccacc

SEQ ID NO: 51 - DNA, 1000 bp, Artificial Sequence
  Synthetic
tgacattagcacgatatgacagtaaatgtcttttttgctggctcaatataacattgtgtcaaaggaactaacagcatacaatgcataatatatcaatattagtcatttttccctttataaatcatatctcaaaatgtatgcaatattctttaaatataacacttaaaatgcatatgcaatgatgaaatatagcaggattgccaaaactgatgaatatctcagagatattgccattgtttttcctggaaaatacttctttggaaaaagtgagacatctttgagatcaaaataaacacctgtcaaaggaatgtcttgggaaccccaaatagcatttctaaagagataaaagaatgttgtagtcttccagaaaggaaatcaagtggaagtagatattaattcagcactacagagacaatctggagaaaacagaggcattgttttctaaagaaattggcttttgtaacataaaggagatacaactctgggagtgaaagttgtcacagtgatacatatcacacaacggaaatgccaaacgaaaccatcaccacttgaaatcaaacttgattcaagttactcaggtaaaataccaccatgggtggcatcctttttttttttttttttttttttttttttaaccaagcagcctaacggtgtttaaggaggaaaacttaattgataatgcactttgctcaattataaataagctgactgggaaagaagtgggcatgatggaaagcaaaatttgaatgaagctgttattcttttaatctataaaaacattacatccaagtttagactcattgagctctaaatatttgggaaaacatatttaaagaaattatataggtttgatccaaaatctctttggcacaacttgaaatatgggtaatcgtcatgtgaaatttgtgaataggagaacccactgtaggatacttaacataaatcagccacataatttctatcactgatatccagggaatttcaatgacaaatctagtga

SEQ ID NO: 52 - DNA, 1000 bp, Artificial Sequence
  Synthetic
agttcttctgcaacaaaacacagcaacccagcatgttttctcatctacattctacattcatacctcattcttagggaccaattggggatactaggctgtacctcccagaaccctttttagaactaaagaattattttcacagctgctgaaagtgctgcaggctagaagtcttcaccctgaagccctctctgggacctgcccttgacaaaagataccaccttctccaaggtcaagcccccttcccagaatcaagaattgctagaatcaagaatcaagagaatcaagaatcaaggcttcctaccctcacttaaactcagggcaattctgaagagccaacccagcttcagagctcccctacagcaatgtgttacaattttgttataactgcatcttagcattccaatatctctttgtctagtcctggcttcttcccttccccacaggggaacactacctataacaagccttagcatccctccatttttaatttttcatattagtgacctgcctttcatcctctgttcattcactcaatgaatattgtcagagtacctactatacaccaggcattattttgggtgctggttattcagcaatgaacaaggctagaaaggccctgccatcacagagttgccatttcagtaagggaagaaggcaataaacatataatttactatcacataggggtaatgcttgaaggaaaattaattgaactgtaacagtacagagttattgggaggaagcatttatttaaacagaaaagaccagagaagacccctatggggggagtgagatggaactgataactgaatgaagtgacagaatgaaccatgcaaagacataaaaaattgcattcaaagcagaagaaagaacaagtgcaaagttcccagggctcaaataatatgtaattttgaagaatggttaggacagtttggttagagtagagtgaaaaggggagaaattgataggagataaactcaaagacagaggcagcagttcc

SEQ ID NO: 53 - DNA, 1000 bp, Artificial Sequence
  Synthetic
ggaggataatgcctgttgccgaggaaacacagcctcatttgggtttcagataatgccaaaaatgaaacaaaaaaaattcacagaaaatacagaatgagttcagcatgtggactacattgatgttgccttatactctgtgtcttcatcttccttctccttttctgtttcttcacttttgatactaatagcccagagctgacctcactgtataaaggacacagtgatcaccactagggcaggaaaataaaattaacacccaactcttagaagaccccccacaggaaacaaaagtagctgatgctgtggtttacaacacacaatcgcaaacaaatggataagaaacaaaaaaaaagaaatagaaaaaaatcagttatagataaagaaacatctgctctcagacccaaatatgagccaaagaagaaaacttaaggaaaatattaaaatattttgaactagtatagctaaccaagaaaaaaagagaagatacaaatttgcattgttataaataaaagaagaaccatcactgctgattcaaggacacgtaaaaaataaaaaggggatactacaaacaacgctatgcctgttcaataacttagatcaaatgggctaattcttgaaagacacaaactattgaaactgactcagggagaaatagataatctttaaaaaaatttttttttattctaagttctgggatacatgtgcagaatgtgcaggtttgttacataggtatacaagtgccatggtggtttgttgcacccatcaacccatcatctacattaggtatttctcctaatgctatccctcctgtagtcccccacccaccaacaggccccggtatgtgatgttctcttccctgtgtccatgtgtactcatggttcaactaccacttaaaagtgagaacatgaggtgtttgattttctgttcttgtgttagtatgctgagaatgacggtttccagcttcatccatgtccctgcaaaggacatgaactcttccttt

SEQ ID NO: 54 - DNA, 1000 bp, Artificial Sequence
  Synthetic
gttggttctttcagggccctggcttgctcatcccctttatgattctcctttattcttctgtacctgggtattcatttcatgtccaccctcatcatcattctggagacaaagatgcagacatgtactggcagaatctgagcatgcaaaaaccttccgtaacactcagagttcttatacctttctctcatctggcattatttactatgatagggtgaaggaaacaactatgtctgttctttgcatttgactgcattgtccagtttctagaggcttaagtacactttttttttttttttttttgagacagagtttcactcttgttgcccaggctggagtgcaatgacgcgatctcagctcactgcaacctccaactctcaggttcaagtgattctcctgcctcagcctcccaaatagctgggattacaggcatctgccaccacacctagctaattttatatttgtagtagaagcggggtttctccatgttggtcaggctggtctcgaactcctgacctcaggtgatccaaccgcctcagcctcccaaagtactgggattacaggcgtaagccactgcacccagcacctagtggcttaaatacatttaaagcatcatagctcaacctctaaattgcactgcagcatacacagctaaatccccgtagttcttctgcaacaaaacacagcaacccagcatgttttctcatctacattctacattcatacctcattcttagggaccaattggggatactaggctgtacctcccagaaccctttttagaactaaagaattattttcacagctgctgaaagtgctgcaggctagaagtcttcaccctgaagccctctctgggacctgcccttgacaaaagataccaccttctccaaggtcaagcccccttcccagaatcaagaattgctagaatcaagaatcaagagaatcaagaatcaaggcttcctaccctcacttaaactcagggcaattctgaagagccaaccca

SEQ ID NO: 55 - DNA, 1000 bp, Artificial Sequence
  Synthetic
caagggtgtggttctttgcagtattacctggaccagtcattggatataggtaactccccaggaagaaaccatagccttgggtagacagcttccttaaacagacaggaatagtagagagggactcaactgtgagctgtcaacagtcaactatgctaccactaggtgtacaggctattacaatgcctgttgcatgttaactgaataataaatactagctattattgttatacctacttatctatttcttaatttatttatctatttactttttaagatcttctcattttgaattattctttgacctccatcagaaattatgctagtgagtccattaaattgatcagttctcacttttcttctctgagaaaattatctgtttgttttttaaagctttattgattcacagtggacctacaatatattgcacctatttaagatgtagaaactaacaaattttctctctcaaacacacatacacatacacctgtgaaaccatcactacaatcaaggtatggaacatttccatcccttccaaaagaaacctcctgccgatttgtaaataactacccctctatcccagtccccaagtagtaactgatctgctttctgtcactatacactaatttgccattttaagaattttatattaatgggattatactttttggggaaaggggtctggcttctttgaatcagcatgactattttgagattcatccaaattacagtgtatagtgtatcaatagtttatagtttttattgctgaatggtgttcctttgcatgggtttactgcaatttgtttatccattcacctgttcatagatgttaagattgtttcaactttttagctattataaataaagctgctatgaacattcagatataagcttagtacagatatatcctttagttaggaaaatatctagggacagaatggtttgttcagatatgtgatttgcaaatattttctccatctgtgttttgttttttacaatattt

SEQ ID NO: 56 - DNA, 1000 bp, Artificial Sequence
  Synthetic
agtagcgattctcatccctcagcctcccgagtagctgggcctacagtcgtgtgccaccatgcccggctaatttttgtagttttagtagagatgtggctttatggtgttgcccaggctgatctcgaactcctgacctcaagtgatatgtccactttggcctcctatagtgctaggattacaggcgtgagccgccgcacctggccggtagtgtacatctttcagggaatcatttcatctaaattgtcaattattggcatacaattattaaaaattttcattttaatgctttgcctatatatagaatctgtgataacatcatctttctcatttttttaattggtaattagtgtcttctctctttttttgttgatcatagtttgtcctattgatctcaaaataatggcttttggtttcattaatttttaaaatcatttttctgttttcttttttgttgttgttgtctgctctgatctttgcaatttcttataatttctttggaattaatttgcttttttagtttcttaaggtagaaactgagatcactgatttgaagtatttcttcttttctaatataggcatttgctgctttatattactatttaaatactgctttaacagtgtcccaagggcctgcatatgttgagtttcaatttttatttatttacttttaaaaaatcggtaagtttaaaacattttccaaatatctgggatcgtttagatatctctgttactgatttctaattaaattccactggggtcagagaacatactttgtacaatttaattttttaaaataccagtgagtcttatttgtggtccagaaaatggttcattttattaaacattctgtgtgcaattggaaagattgcatattccgcttctgttgagtgtgctatacacctcaattaggtcaagctggttaatagtattgttctttatgtcctttctgatttgtattctatttttttgagaaggtggtgttgaaatctccaactataat

SEQ ID NO: 57 - DNA, 1000 bp, Artificial Sequence
  Synthetic
ggtgtacaggctattacaatgcctgttgcatgttaactgaataataaatactagctattattgttatacctacttatctatttcttaatttatttatctatttactttttaagatcttctcattttgaattattctttgacctccatcagaaattatgctagtgagtccattaaattgatcagttctcacttttcttctctgagaaaattatctgtttgttttttaaagctttattgattcacagtggacctacaatatattgcacctatttaagatgtagaaactaacaaattttctctctcaaacacacatacacatacacctgtgaaaccatcactacaatcaaggtatggaacatttccatcccttccaaaagaaacctcctgccgatttgtaaataactacccctctatcccagtccccaagtagtaactgatctgctttctgtcactatacactaatttgccattttaagaattttatattaatgggattatactttttggggaaaggggtctggcttctttgaatcagcatgactattttgagattcatccaaattacagtgtatagtgtatcaatagtttatagtttttattgctgaatggtgttcctttgcatgggtttactgcaatttgtttatccattcacctgttcatagatgttaagattgtttcaactttttagctattataaataaagctgctatgaacattcagatataagcttagtacagatatatcctttagttaggaaaatatctagggacagaatggtttgttcagatatgtgatttgcaaatattttctccatctgtgttttgttttttacaatatttaacgatgcctcttgacgaacagaaattatgaaattaagtcttgtttatcaactttttcttttatggtttatgcttttggtgttgtatctaagaaatttttgcctaactcaaggtcaaaaaggttttctgtgttttttggcttatttaggtgtatgaaccat

SEQ ID NO: 58 - DNA, 1000 bp, Artificial Sequence
  Synthetic
atagtgctaggattacaggcgtgagccgccgcacctggccggtagtgtacatctttcagggaatcatttcatctaaattgtcaattattggcatacaattattaaaaattttcattttaatgctttgcctatatatagaatctgtgataacatcatctttctcatttttttaattggtaattagtgtcttctctctttttttgttgatcatagtttgtcctattgatctcaaaataatggcttttggtttcattaatttttaaaatcatttttctgttttcttttttgttgttgttgtctgctctgatctttgcaatttcttataatttctttggaattaatttgcttttttagtttcttaaggtagaaactgagatcactgatttgaagtatttcttcttttctaatataggcatttgctgctttatattactatttaaatactgctttaacagtgtcccaagggcctgcatatgttgagtttcaatttttatttatttacttttaaaaaatcggtaagtttaaaacattttccaaatatctgggatcgtttagatatctctgttactgatttctaattaaattccactggggtcagagaacatactttgtacaatttaattttttaaaataccagtgagtcttatttgtggtccagaaaatggttcattttattaaacattctgtgtgcaattggaaagattgcatattccgcttctgttgagtgtgctatacacctcaattaggtcaagctggttaatagtattgttctttatgtcctttctgatttgtattctatttttttgagaaggtggtgttgaaatctccaactataattgtagatttctctatttctccttgcaattgtatcagattttacctattttgaaaatctgttattaggtgcataagtatttgcaagtattatcttctctcaatgtattgattctttttttgaaattatgaaatgacactttatcccttttaataattacagaca

SEQ ID NO: 59 - DNA, 1000 bp, Artificial Sequence
  Synthetic
cccaggaagaaaccatagccttgggtagacagcttccttaaacagacaggaatagtagagagggactcaactgtgagctgtcaacagtcaactatgctaccactaggtgtacaggctattacaatgcctgttgcatgttaactgaataataaatactagctattattgttatacctacttatctatttcttaatttatttatctatttactttttaagatcttctcattttgaattattctttgacctccatcagaaattatgctagtgagtccattaaattgatcagttctcacttttcttctctgagaaaattatctgtttgttttttaaagctttattgattcacagtggacctacaatatattgcacctatttaagatgtagaaactaacaaattttctctctcaaacacacatacacatacacctgtgaaaccatcactacaatcaaggtatggaacatttccatcccttccaaaagaaacctcctgccgatttgtaaataactacccctctatcccagtccccaagtagtaactgatctgctttctgtcactatacactaatttgccattttaagaattttatattaatgggattatactttttggggaaaggggtctggcttctttgaatcagcatgactattttgagattcatccaaattacagtgtatagtgtatcaatagtttatagtttttattgctgaatggtgttcctttgcatgggtttactgcaatttgtttatccattcacctgttcatagatgttaagattgtttcaactttttagctattataaataaagctgctatgaacattcagatataagcttagtacagatatatcctttagttaggaaaatatctagggacagaatggtttgttcagatatgtgatttgcaaatattttctccatctgtgttttgttttttacaatatttaacgatgcctcttgacgaacagaaattatgaaattaagtcttgtttatcaactttt

SEQ ID NO: 60 - DNA, 998 bp, Artificial Sequence
  Synthetic
ggttatggacagcctatttactcgtatcaccttgcagggtttgtaggataattgcccagaactagaatattaatccagacttttacattacgcatcccttctgtttcttctgaactgcagctagacatcactggttgatccatgaaataagcagggttagttcaaaatgtgggtaaaaagcttaaaaacaactgagtctagaatttaatgacaaatgtatggtaagttttgaaacttatcatacagtggcatgctctcagctcactgcaacctccacctctccagttcaagtgattctccttcctcagcctccctagtagctgaattacaggtgcacgccaacatgcccaactaatgtttgtatttttagtagagacagggtttctccacgttggccaggctggtctcaaaatcctggtctcaagtgatcctcccgcctcagcctcccaaaatcctgggattacaggagtgagccaccatgcctggtccattctgttaacacttgttctgtttgatatttctgaacatttcagctattcattaatcctgtatgtttttccttattccaatgtcataatctccaaagttatcagaaacctgaatttgagagcacctgtcgaagtcttatagctgattataaatcatcttttgaagaggatcaagatgagacaattgtctgtgaataacaaaatgtccagggtagttacaattaaatacacaattgacaagaaatttggttatcactttggtttacaataatttaaccttaattatgattgatagcagcatatactcagacattcgaattttagaaatcccatataattttggaacgtatattaatattattcactaaaatgtaacctgaagaagactgaacatcattttagtaatctcacgtaactaaacttgtcaaataattctgtttacctctcttttggatgctccaagagccctctgtagcatccaaaagccaggggtcaggaaagacaaccctg

SEQ ID NO: 61 - DNA, 1000 bp, Artificial Sequence
  Synthetic
cttagtctgagccactccacattgatttatttccattactgtgcatgtgttaagggatggaatttttgagtgtgggcatatttaggcaacccccatgtgtggaatgatttgggcaggttggaggttctttgagggcccttccctatctgctaggcatttgtcagcttcctgcctctatcattcccgcttctaaagaggtacatctgactgtgttagaatacggatgaagactgatcttaactgcttcctgctgacagggggcgctgttttgggaagatggcagtcatgtctccctcagaggcctatctaagggtccccagtaaaaagagccatcatcagaggcattgtttgcatgaccatttagagtttggtggcctgaaggtgagaagagacaaactgggttattagaatacatgtatcaaaacgaaacaaaaaggggtgggtaaggacagctcaaaaatcccaagactgatggcacacccagatagctggtggctacagttatgcctgccaagatttgggtgcatgggacttggctttgattagctcccttggtcttattttcccgtataaagaaacctccgggttatggacagcctatttactcgtatcaccttgcagggtttgtaggataattgcccagaactagaatattaatccagacttttacattacgcatcccttctgtttcttctgaactgcagctagacatcactggttgatccatgaaataagcagggttagttcaaaatgtgggtaaaaagcttaaaaacaactgagtctagaatttaatgacaaatgtatggtaagttttgaaacttatcatacagtggcatgctctcagctcactgcaacctccacctctccagttcaagtgattctccttcctcagcctccctagtagctgaattacaggtgcacgccaacatgcccaactaatgtttgtatttttagtagagacagggtttctccacgttggccaggctggtctcaaaatcct

SEQ ID NO: 62 - DNA, 1000 bp, Artificial Sequence
  Synthetic
ctgaggatgggacacaggtggttctgatgcatgctgatgactgagaatcacggtacagaatatactgatgcaggattttctgctccttagctcacctaaatccgggatcttgtctcatcaccaggaagtagtaggcacacggacacaatgaaggggggtgagggcagaatttattaagtgaaaggaaagcactcagtaaagagaggtgtcctgcacacaggcttccacctcacaaatttgaataccaggccaccacgcatgagttgcaaaggtaaggctcctcccccacaaaaggtgcaaattcctgggggtttgaccctattctctcagggtgcatgccgacccttagtctgagccactccacattgatttatttccattactgtgcatgtgttaagggatggaatttttgagtgtgggcatatttaggcaacccccatgtgtggaatgatttgggcaggttggaggttctttgagggcccttccctatctgctaggcatttgtcagcttcctgcctctatcattcccgcttctaaagaggtacatctgactgtgttagaatacggatgaagactgatcttaactgcttcctgctgacagggggcgctgttttgggaagatggcagtcatgtctccctcagaggcctatctaagggtccccagtaaaaagagccatcatcagaggcattgtttgcatgaccatttagagtttggtggcctgaaggtgagaagagacaaactgggttattagaatacatgtatcaaaacgaaacaaaaaggggtgggtaaggacagctcaaaaatcccaagactgatggcacacccagatagctggtggctacagttatgcctgccaagatttgggtgcatgggacttggctttgattagctcccttggtcttattttcccgtataaagaaacctccgggttatggacagcctatttactcgtatcaccttgcagggtttgtaggataattgcccagaactagaatatta

SEQ ID NO: 63 - DNA, 1000 bp, Artificial Sequence
  Synthetic
atgagttgcaaaggtaaggctcctcccccacaaaaggtgcaaattcctgggggtttgaccctattctctcagggtgcatgccgacccttagtctgagccactccacattgatttatttccattactgtgcatgtgttaagggatggaatttttgagtgtgggcatatttaggcaacccccatgtgtggaatgatttgggcaggttggaggttctttgagggcccttccctatctgctaggcatttgtcagcttcctgcctctatcattcccgcttctaaagaggtacatctgactgtgttagaatacggatgaagactgatcttaactgcttcctgctgacagggggcgctgttttgggaagatggcagtcatgtctccctcagaggcctatctaagggtccccagtaaaaagagccatcatcagaggcattgtttgcatgaccatttagagtttggtggcctgaaggtgagaagagacaaactgggttattagaatacatgtatcaaaacgaaacaaaaaggggtgggtaaggacagctcaaaaatcccaagactgatggcacacccagatagctggtggctacagttatgcctgccaagatttgggtgcatgggacttggctttgattagctcccttggtcttattttcccgtataaagaaacctccgggttatggacagcctatttactcgtatcaccttgcagggtttgtaggataattgcccagaactagaatattaatccagacttttacattacgcatcccttctgtttcttctgaactgcagctagacatcactggttgatccatgaaataagcagggttagttcaaaatgtgggtaaaaagcttaaaaacaactgagtctagaatttaatgacaaatgtatggtaagttttgaaacttatcatacagtggcatgctctcagctcactgcaacctccacctctccagttcaagtgattctccttcctcagcctccctagtagctgaattaca

SEQ ID NO: 64 - DNA, 1000 bp, Artificial Sequence
  Synthetic
ggggggtgagggcagaatttattaagtgaaaggaaagcactcagtaaagagaggtgtcctgcacacaggcttccacctcacaaatttgaataccaggccaccacgcatgagttgcaaaggtaaggctcctcccccacaaaaggtgcaaattcctgggggtttgaccctattctctcagggtgcatgccgacccttagtctgagccactccacattgatttatttccattactgtgcatgtgttaagggatggaatttttgagtgtgggcatatttaggcaacccccatgtgtggaatgatttgggcaggttggaggttctttgagggcccttccctatctgctaggcatttgtcagcttcctgcctctatcattcccgcttctaaagaggtacatctgactgtgttagaatacggatgaagactgatcttaactgcttcctgctgacagggggcgctgttttgggaagatggcagtcatgtctccctcagaggcctatctaagggtccccagtaaaaagagccatcatcagaggcattgtttgcatgaccatttagagtttggtggcctgaaggtgagaagagacaaactgggttattagaatacatgtatcaaaacgaaacaaaaaggggtgggtaaggacagctcaaaaatcccaagactgatggcacacccagatagctggtggctacagttatgcctgccaagatttgggtgcatgggacttggctttgattagctcccttggtcttattttcccgtataaagaaacctccgggttatggacagcctatttactcgtatcaccttgcagggtttgtaggataattgcccagaactagaatattaatccagacttttacattacgcatcccttctgtttcttctgaactgcagctagacatcactggttgatccatgaaataagcagggttagttcaaaatgtgggtaaaaagcttaaaaacaactgagtctagaatttaatgacaaatgtatggta

SEQ ID NO: 65 - DNA, 23 bp, Artificial Sequence
  Synthetic
gcatcgtacgcgtacgtgtttgg

SEQ ID NO: 66 - DNA, 23 bp, Artificial Sequence
  Synthetic
tgtggaaccatgaatccgaaggg

SEQ ID NO: 67 - DNA, 23 bp, Artificial Sequence
  Synthetic
gtaggccgttggtaacgtgatgg

SEQ ID NO: 68 - DNA, 10 bp, Artificial Sequence
  Synthetic
actgccactt

SEQ ID NO: 69 - DNA, 10 bp, Artificial Sequence
  Synthetic
tagtgccatg

SEQ ID NO: 70 - DNA, 23 bp, Artificial Sequence
  Synthetic
ccaaacacgtacgcgtacgatgc

SEQ ID NO: 71 - DNA, 33 bp, Artificial Sequence
  Synthetic
ttcaccgtcaggtttgtgcatgcgcatgctacg

SEQ ID NO: 72 - DNA, 33 bp, Artificial Sequence
  Synthetic
cgtagcatgcgcatgcacaaacctgacggtgaa

SEQ ID NO: 73 - RNA, 20 nt, Artificial Sequence
  Synthetic
uugugcaugcgcaugcuacg

SEQ ID NO: 74 - DNA, 33 bp, Artificial Sequence
  Synthetic
cgtagcatgcgcatgcacaaaccatcacggtac

SEQ ID NO: 75 - DNA, 33 bp, Artificial Sequence
  Synthetic
gtaccgtgatggtttgtgcatgcgcatgctacg

SEQ ID NO: 76 - DNA, 16 bp, Artificial Sequence
  Synthetic
gtttggactgccactt

SEQ ID NO: 77 - DNA, 16 bp, Artificial Sequence
  Synthetic
aagtggcagtccaaac

SEQ ID NO: 78 - DNA, 16 bp, Artificial Sequence
  Synthetic
catggcactaccaaac

SEQ ID NO: 79 - DNA, 16 bp, Artificial Sequence
  Synthetic
gtttggtagtgccatg

SEQ ID NO: 80 - RNA, 20 nt, Artificial Sequence
  Synthetic
gaaguaccgugauccugauu

SEQ ID NO: 81 - DNA, 60 bp, Artificial Sequence
  Synthetic
atatttcttatatgtctacaactgccacttcatggcactaggactaaaggttggccaaag

SEQ ID NO: 82 - DNA, 60 bp, Artificial Sequence
  Synthetic
ctttggccaacctttagtcctagtgccatgaagtggcagttgtagacatataagaaatat

SEQ ID NO: 83 - DNA, 23 bp, Artificial Sequence
  Synthetic
  misc_feature (21)..(23): n is a or g or c or t/u, unknown, or other
atatttcttatatgtctacannn

SEQ ID NO: 84 - DNA, 23 bp, Artificial Sequence
  Synthetic
  misc_feature (1)..(3): n is a or g or c or t/u, unknown, or other
nnntgtagacatataagaaatat

SEQ ID NO: 85 - DNA, 10 bp, Artificial Sequence
  Synthetic
aagtggcagt

SEQ ID NO: 86 - DNA, 10 bp, Artificial Sequence
  Synthetic
catggcacta

SEQ ID NO: 87 - DNA, 23 bp, Artificial Sequence
  Synthetic
  misc_feature (1)..(3): n is a or g or c or t/u, unknown, or other
nnnggactaaaggttggccaaag

SEQ ID NO: 88 - DNA, 23 bp, Artificial Sequence
  Synthetic
  misc_feature (21)..(23): n is a or g or c or t/u, unknown, or other
ctttggccaacctttagtccnnn